SEQUENCE LISTING (145 sequences)

SEQ ID NO: 1 - DNA, 1005 bp, Pasteurella multocida
atggagaacaagcccttggtgtcggtcctcatttgcgcatacaacgtcgagaagtacattgaagaatgcattaatgcagtgatcaatcagacgtacaagaacttagaaattattattgtgaacgatggtagttcggataacacgtattttttgctgaagaagttggccgaaaaagacaatcgcataaaaatattaaatttcaacaaccacattgggataatctcggccttaaacgagggcttgaaagagatagcaggggagtatattgcacgaacggacagtgacgatataactaagccggattggattgagaaaatattaacttgcatgcaaaatgatcccaagatcatcgcaatggggtcgtatctcactgtcttgtcggaagaaaataatggtagcgtgttggcaaatcaccacaagaataaagttgaatggaagaatccactggaacacaaggacattgtcgagaagatgttgtttggtaaccccatccataataacagtatggttatgcggagtgagatctatacgaagtaccacctgatctatgacccagactatcattatgccgaggattataagttctggctcgaggttagccgaatcgggaagttggcgaactatcccgagagtcttgtttattaccgattgcaccgaaatcaaactagttcgattcataactcgcaacaagaaataaatggcaaaaagctgcgtttacaggccttgaactattacttgaaagacttgggcattgactaccagttaccagagaagtttttattcaaggacatagccctgctccaggagatattctacgaacgaggtatgtttagagaaaatataatcaggcgcatcatctacgaatgctacctctccctcggagagtacaattataaagacatttactactttctgataaataaaaataactttctctcgataaaagacaagttcaagatcataaaaaaatacctccgtccagacaagtactcgtcgacttactag

SEQ ID NO: 2 - DNA, 1050 bp, Neisseria meningitidis
atgccgtcagaagcgtttagacggcaccgggcgtaccgggagaataaattacaacctcttgtgagcgtattaatctgcgcgtataacgtagaaaaatactttgcgcagtcacttgcggcggtggttaatcagacatggcgcaacttagacatcttaatcgtggacgatggctcgacggatgggacgcttgcgatcgcgcaacggtttcaggaacaagatggccgcattcgcattttagcccaaccgcgcaatagcgggctgatcccatcacttaacattggactggacgaattagcaaagtcgggaggaggaggagaatacatcgcccgcaccgacgcagatgacatcgcggcgccagattggatcgagaaaattgttggggagatggaaaaagatcgcagcattattgccatgggggcctggctggaagttttatcggaagaaaaggatggcaaccggctggcccggcatcatgaacatggcaaaatctggaaaaaaccgaccagacatgaagatatcgcggatttctttccttttggcaacccaatacataacaacacgatgatcatgcgacgcagcgtgatcgatggcgggttacggtataacaccgagcgggactgggccgaagactatcagttctggtatgacgtgagcaaattaggccgactggcttactacccagaagcgttagtgaaatatcgccttcatgcgaatcaagtttcaagcaaatatagcattcgccagcatgaaattgcccagggcattcagaaaaccgcgagaaacgacttcttacaatcaatggggttcaaaacccggtttgatagccttgaatatcgccagataaaagcagtagcctacgaattactggagaaacacttaccggaagaagacttcgaacgcgcgcgccggttcttatatcagtgctttaaacggacggatacgctgccagcgggcgcctggctggacttcgccgcagatggccgaatgcggcggctgttcaccttacgacagtattttggcatcttacatagattactgaaaaaccggtga

SEQ ID NO: 3 - DNA, 1050 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcaaacttggcgcaacttggatattttgattgtcgatgacggctcgacggacggtacgcttgccattgcccagcgtttccaagaacaggacggccgcatccgcatcctcgcgcagccgcgcaattccggtctgattccctctttaaacatcgggctggatgaattggcaaagtcgggaggcggtggggaatatattgcgcgcaccgatgcagacgatattgccgcccccgactggattgagaaaatcgtgggtgagatggaaaaagaccgcagcatcatcgcgatgggtgcgtggctggaagttttgtcggaagaaaaggacggcaaccggctggcgcggcaccacgaacacggcaaaatttggaaaaaaccgacccgacacgaagacattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgcgtggctggattttgcggcagacggcaggatgcggcggctgtttaccttgaggcaatacttcggcattttgcaccgattgctgaaaaaccgttga

SEQ ID NO: 4 - DNA, 1050 bp, Neisseria meningitidis
atgcccagcgaggctttccgtagacatagagcctatagagaaaacaagctgcagcccctcgtttcggtcttgatttgtgcttacaatgtcgagaagtatttcgcccaaagtctcgctgccgttgtcaaccaaacttggcgaaatctcgatattctcattgtcgatgacggttctactgacggaaccttagctatagcccagagattccaagagcaggacggtagaatacgtatattggctcagcctcgtaactcgggtctaattcctagtctcaatatcgggctagatgagttggctaaaagtggtgggggcggggagtatattgcgaggacggatgctgacgatatagccgcccctgactggattgaaaagatagtgggagaaatggagaaggacaggtctataatcgcgatgggagcgtggttggaggtgctcagtgaggagaaagacggaaatagattggcaaggcaccacgagcacggtaagatatggaagaagccaacgcgacacgaggacatagccgactttttcccattcggtaatcctattcacaataatactatgattatgaggcgatctgttattgacggtggattgagatacaatacggaacgagattgggcagaggattaccaattttggtacgatgtttccaagctcggacggctcgcatattatcctgaggctctcgtcaagtacaggctccacgccaaccaggtcagttcaaagtactcaatccgtcaacacgagatcgcacaaggtatccaaaagacggcaaggaatgattttcttcagagcatgggatttaagacgagattcgactcgctcgagtaccgtcaaattaaggctgtggcatatgagcttcttgaaaagcatttgcccgaggaggattttgagagggcacgtagatttttgtaccaatgtttcaagcgaacagacactttacctgcaggggcgtggttagattttgctgccgacggaaggatgcgtcgcttatttacgttgcggcaatacttcggaattcttcacaggctcctcaagaatagataa

SEQ ID NO: 5 - DNA, 1050 bp, Neisseria meningitidis
atgccaagtgaggcatttcgaaggcaccgcgcataccgagaaaacaagctccaacccttggtttcagtcttaatctgtgcgtataatgtcgagaagtactttgcgcagagtttggcagcggttgtaaaccagacatggcgtaatctcgacatccttatagtagacgatggtagtacagatggcactctcgcaatcgcacaacgctttcaggagcaagatggtcgtattcgtattcttgctcaacctaggaactctgggttaatccctagcttgaatataggtttggacgagcttgcgaaaagtggggggggcggagagtacatagctcgtactgacgctgatgacatcgctgcacctgattggatcgaaaagattgtcggggaaatggagaaggatcgttcaataattgcaatgggggcatggcttgaggtcttatccgaggagaaagatggtaatcgattagcccgccatcatgagcatggtaagatatggaagaagcccacgaggcatgaggatatcgcggatttctttccatttgggaatcctattcataataataccatgatcatgcgaagatctgttatcgatgggggattacgctataatacagaaagagactgggcagaggactatcagttctggtatgacgtatccaagttaggtagactagcctactaccctgaggctttagttaagtatcgactccatgctaaccaagtctcgagtaagtattccattagacagcatgagattgcccagggtattcagaagacggcgcgaaatgacttcttacaatcgatgggcttcaagacgcgatttgattcgctcgagtataggcagatcaaggcggtcgcctacgagcttctagaaaagcaccttcctgaggaggacttcgagcgagcgcgtaggttcctatatcagtgttttaagcgaactgatactctccctgcaggagcatggctcgacttcgctgctgatggaagaatgagaagacttttcactctaagacagtattttgggatccttcataggttattaaagaatcgctaa

SEQ ID NO: 6 - DNA, 1050 bp, Neisseria meningitidis
atgccttcggaggcttttcgacgtcaccgagcataccgtgaaaacaagttacaacccctcgttagtgtgctcatctgtgcatataatgttgagaagtactttgctcagagcttggcagcagtggttaaccagacatggcgtaatttagacatcctaatcgttgacgatggtagtacagatggcaccctcgcaatcgctcaacgctttcaggagcaagatggtcgtattcgtattcttgcacaacctaggaactctggattaatcccgtccttgaatattggtttggacgagcttgctaaaagtgggggtggcggagagtacatcgcccgtacggacgccgatgacatcgcggctccagattggatcgaaaagattgtcggagaaatggagaaggatcgtagtattattgccatgggggcatggttggaggtattaagtgaggagaaagatggaaatagattggcacgacatcatgagcatggaaagatctggaagaagcccacgcggcatgaggatatcgcggatttctttccatttggtaatccgatccataataataccatgatcatgagaagatcggttatagatggggggctacgctataatacggaacgagactgggctgaggactatcagttctggtatgacgtttcgaagttaggtcgactcgcatactacccagaggcactcgtgaagtatcgtttacatgctaaccaagtcagctcgaagtattctatacgtcagcatgagatagcacagggaattcagaagactgctaggaatgacttcttacaatccatgggcttcaagacacgctttgattcactcgagtatcgtcagattaaggcggtggcatacgagttattagaaaagcaccttcccgaggaggacttcgagcgtgcacgtagattcctctatcagtgttttaagcgaactgataccttgcctgctggggcttggctagacttcgcagccgatggtagaatgcgaaggctcttcacattaagacagtattttggtatcttacatagattactaaagaatcgctga

SEQ ID NO: 7 - DNA, 1047 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggagattttgattgtcgatgacggctcgacagacggtacgcttgccattgccaaggattttcaaaagcgggacagccgtatcaaaatccttgcacaagctcaaaattccggcctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcatcgcgatgggcgcgtggctggaagttttgtcggaagaaaaggacggcaaccggctggcgcggcatcacaggcacggcaaaatttggaaaaagccgacccgacccgaagatattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgcgtggctggattttgcggcagacggcaggatgcggcggctgtttaccttgaggcaatacttcggcattttgcaccgattgctgaaaaaccgttga

SEQ ID NO: 8 - DNA, 1041 bp, Neisseria gonorrhoeae
atgcaaccacttgtgagcgtactgatctgcgcgtataacgtagaaaaatattttgcgcagtcacttgcggcggtggttaatcaaacatggcgcaacctggacatcctgatcgtggacgatggctcgacagatggcacattagcgatcgcgaaggactttcagaagcgggatagccggattaaaattttagctcaggctcagaatagcggcctgatcccgtcacttaacattggactggatgaactggctaagtcgggaggaggaggatatatcgcccgcaccgacgcggatgacatcgcgagcccgggctggatcgagaaaattgttggcgagatggaaaaagatcgcagcattatcgccatgggcgcctggctggaaatcctgtcggaagaaaaggatggcaaccggctggcccggcatcataaacatggcaaaatctggaaaaagccgacccggcatgaagatattgcggcgttttttccatttggcaacccgatacataacaacacgatgatcatgcggcgcagcgtgatcgatggcgggctgcggtataacaccggacgggactgggccgaagactatcagttttggtatgacgtgagcaaactgggccgactggcttattatccggaagcgctggtgaaatatcgcttacatgcgaatcaagtttcaagcaaacatagcgtgcgccagcatgaaattgcccagggcattcagaaaaccgcgagaaacgactttctgcaatcaatggggtttaaaacccggtttgatagcctagaatatcgccagacaaaagctgccgcctatgaactgccggagaaggacctgccggaagaagactttgaacgcgcgcgccggtttctgtatcagtgctttaaacggacggatacgccgccgagcggcgcctggctggactttgccgctgatggccgaatgcgacggctgtttaccctgcgacagtattttggcatcctgtatcggctgatcaaaaaccgccggcaagcccggtcggactcggctggaaaagaacaagagatctaa

SEQ ID NO: 9 - DNA, 1047 bp, Neisseria gonorrhoeae
atgcaaccacttgtgagcgtactgatctgcgcgtataacgtagaaaaatattttgcgcagtcacttgcggcggtggttaatcaaacatggcgcaacctggacatcctgatcgtggacgatggctcgacagatggcacattagcgatcgcgaaggactttcagaagcgggatagccggattaaaattttagctcaggctcagaatagcggcctgatcccgtcacttaacattggactggatgaactggctaagtcgggaggaggaggaggagaatatatcgcccgcaccgacgcggatgacatcgcgagcccgggctggatcgagaaaattgttggcgagatggaaaaagatcgcagcattatcgccatgggcgcctggctggaagttctgtcggaagaaaaggatggcaaccggctggcccggcatcataaacatggcaaaatctggaaaaagccgacccggcatgaagatattgcggcgttttttccatttggcaacccgatacataacaacacgatgatcatgcggcgcagcgtgatcgatggcgggctgcggtatgataccgagcgggactgggccgaagactatcagttttggtatgacgtgagcaaactgggccgactggcttattatccggaagcgctggtgaaatatcgcttacatgcgaatcaagtttcaagcaaacatagcgtgcgccagcatgaaattgcccagggcattcagaaaaccgcgagaaacgactttctgcaatcaatggggtttaaaacccggtttgatagcctagaatatcgccagacaaaagctgccgcctatgaactgccggagaaggacctgccggaagaagactttgaacgcgcgcgccggtttctgtatcagtgctttaaacggacggatacgccgccgagcggcgcctggctggactttgccgctgatggccgaatgcgacggctgtttaccctgcgacagtattttggcatcctgtatcggctgatcaaaaaccgccggcaagcccggtcggactcggctggaaaagaacaagagatctaa

SEQ ID NO: 10 - DNA, 1047 bp, Neisseria lactamica
atgccgtcagaagcgtttagacggcaccgggcgtatcgggagaataaactgcaaccactcgtgagcgtactgatctgcgcgtataacgtagaaaaatattttgcgcagtcactcgcggcggtggttaatcaaacatggcgcaacctggagatcctgatcgtggacgatggctcgacagatggcacgttagcgatcgcgaaagactttcagaagcgggatagccggattaaaattttagctcaggctcagaatagcggcctgatcccgtcactcaacattggactggatgaactggctaagtcgggaggaggagaatatatcgcccgcaccgacgcggatgacatcgcggcgccggattggatcgagaaaattgttggggagatggaaaaagatcgcagcattatcgccatgggcgcctggctggaagttctgtcggaagaaaaggatggcaaccggctggcccggcatcatgaacatggcaaaatctggaaaaagccgacccggcatgaagatatcgcggcggtttttccatttggcaacccgatacataacaacacgatgatcatgcggcgcagcgtgatcgatggcgggctgcggtataacaccgagcgggactgggccgaagactatcagttttggtatgacgtgagcaaactgggcagactggcttattatccggaagcgctggtgaaatatcgcttacatgcgaatcaagtttcaagcaaacatagcgtgcgccagcatgaaattgcccaggatattcagaaaaccgcgagaaacgactttctgcaatcaatggggtttaaaacccggtttgatagcttagaatatcgccagataaaagctgtagcctatgaactgctggagaaggacctgccggaagaagactttgaacgcgcgcgccggtttctgtatcagtgttttaaacggacggatacgccgccggcgggcgcctggctggactttgccgctgatggcagaatgcggcggctgtttaccctgagacagtattttggcatcctgcgcagactgctgaaaaaccggtaa

SEQ ID NO: 11 - DNA, 1047 bp, Neisseria lactamica
atgccgtcagaagcgtttagacggcaccgggcgtatcgggagaataaactgcaaccactcgtgagcgtactgatctgcgcgtataacgtagaaaaatattttgcgcagtcactcgcggcggtggttaatcaaacatggcgcaacctggacatcctgatcgtggacgatggctcgacagatgggacgttagcgatcgctaaggactttcagaagcgggatagccggattaaaattttagctcaggctcagaatagcggcctgatcccgtcactcaacattggactggacgaactggctaagtcgggaggaggagaatatatcgcccgcaccgacgctgatgacatcgcggcgccggattggatcgagaaaattgttggggagatggaaaaagatcgcagcattatcgccatgggcgcctggctggaagttctgtcggaagaaaaggatggcaaccggctggcccggcatcatgaacatggcaaaatctggaaaaaaccgacccggcatgaagatatcgcggcggtttttccatttggcaacccgatacataacaacacgatgatcatgcggcgcagcgtgatcgatggcggactgcggtataacaccgagcgggactgggccgaagactatcagttttggtatgacgtgagcaaactgggcagactggcttattatccggaagcgctggtgaaatatcgcttacatgcgaatcaagtttcaagcaaatatagcattcgccagcatgaaattgcccagggcattcagaaaaccgcgagaaacgactttctgcaatcaatggggtttaaaacccggtttgatagcttagaatatcgccagataaaagctgtagcctatgaactgctggagaaacacctgccggaagaagactttgaacgcgctcgccggtttctgtatcagtgttttaaacggacggatacgccgccggcgggcgcctggctggactttgccgctgatggcagaatgcggcggctgtttaccctgagacagtattttggcatcctgcgcagactgctgaaaaaccggtaa

SEQ ID NO: 12 - DNA, 1005 bp, Pasteurella dagmatis
atgctcgaactgccactggtgagcgtgttaatttgcgcatataacgtggaaaaatatattgaagaatgcataaacgcggtgattaaccagacctataagaacctggaaattatagtggtgaacgatggcagcagcgataacacctattttctcctgaaacagctggcaaaaaaagataaccgcataaaaatactgaatctggagaacaacgtgggaataataagcgctctgaacgaaggactgaaagaagtgagcggcgagtatattgcacgaaccgattcagacgatataaccaaaccagattggattgagaaaatactgacttgcatggaaaacgatccagaaattattgctatgggaagctatttaaccgtactgtcagaagaaaacaacggcagcgtgttagcaaaccaccataaagatgctgtggaatggaaaaacccactggaacataaagatatagtggaaaaaatgctgtttgggaaccccattcataacaatagcatgataatgcgaagcaagatatataccaagcataaactgatttatgatctggattatccatatgcagaagattataagttttggttagaagtgtcgcgaattggcaaactggctaactacccagaaagcctcgtatattatcgattacaccataaccagaccagcagcattcatcatgatcagcaggaacgaattagcaaaaaactccgattagaagcattaaactattatctgaaagcattaggcattgattactgtctgccagagaaatttctgttcaaagatatagctaccctgcatgaaatattttataaaaacggagcactgcaggaaaacataataaaacgcataatatacgaatgctatttaagcttaaccgaatatcattataaagatatttattgctttctgataagcgagaacaactgctttagcataaaggagaggtttaaaattataaagaagttcttacgcccgagcaaatatagcagcacctattag

SEQ ID NO: 13 - DNA, 1089 bp, Neisseria gonorrhoeae
atgccgtcagaagcgtttcgacgggcttcaaattatcgggagtcaaaactgcaaccacttgtgagcgtactgatctgcgcgtataacgtagaaaaatattttgcgcagtcacttgcggcggtggttaatcaaacatggcgcaacctggacatcctgatcgtggacgatggctcgacagatggcacattagcgatcgcgaaggactttcagaagcgggatagccggattaaaattttagctcaggctcagaatagcggcctgatcccgtcacttaacattggactggatgaactggctaagtcgggaggaggaggatatatcgcccgcaccgacgcggatgacatcgcgagcccgggctggatcgagaaaattgttggcgagatggaaaaagatcgcagcattatcgccatgggcgcctggctggaaatcctgtcggaagaaaaggatggcaaccggctggcccggcatcataaacatggcaaaatctggaaaaagccgacccggcatgaagatattgcggcgttttttccatttggcaacccgatacataacaacacgatgatcatgcggcgcagcgtgatcgatggcgggctgcggtataacaccggacgggactgggccgaagactatcagttttggtatgacgtgagcaaactgggccgactggcttattatccggaagcgctggtgaaatatcgcttacatgcgaatcaagtttcaagcaaacatagcgtgcgccagcatgaaattgcccagggcattcagaaaaccgcgagaaacgactttctgcaatcaatggggtttaaaacccggtttgatagcctagaatatcgccagacaaaagctgccgcctatgaactgccggagaaggacctgccggaagaagactttgaacgcgcgcgccggtttctgtatcagtgctttaaacggacggatacgccgccgagcggcgcctggctggactttgccgctgatggccgaatgcgacggctgtttaccctgcgacagtattttggcatcctgtatcggctgatcaaaaaccgccggcaagcccggtcggactcggctggaaaagaacaagagatctaa

SEQ ID NO: 14 - DNA, 1044 bp, Neisseria gonorrhoeae
atgcaaccacttgtgagcgtactgatctgcgcgtataacgtagaaaaatattttgcgcagtcacttgcggcggtggttaatcaaacatggcgcaacctggacatcctgatcgtggacgatggctcgacagatggcacattagcgatcgcgaaggactttcagaagcgggatagccggattaaaattttagctcaggctcagaatagcggcctgatcccgtcacttaacattggactggatgaactggctaagtcgggaggaggaggagaatatatcgcccgcaccgacgcggatgacatcgcgagcccgggctggatcgagaaaattgttggcgagatggaaaaagatcgcagcattatcgccatgggcgcctggctggaaatcctgtcggaagaaaaggatggcaaccggctggcccggcaccataaacatggcaaaatctggaaaaagccgacccggcatgaagatattgcggcgttttttccatttggcaacccgatacataacaacacgatgatcatgcggcgcagcgtgatcgatggcgggctgcggtataacaccggacgggactgggccgaagactatcagttttggtatgacgtgagcaaactgggccgactggcttattatccggaagcgctggtgaaatatcgcttacatgcgaatcaagtttcaagcaaacatagcgtgcgccagcatgaaattgcccagggcattcagaaaaccgcgagaaacgactttctgcaatcaatggggtttaaaacccggtttgatagcctagaatatcgccagacaaaagctgccgcctatgaactgccggagaaggacctgccggaagaagactttgaacgcgcgcgccggtttctgtatagatgctttaaacggacggatacgccgccgagcggcgcctggctggactttgccgctgatggccgaatgcgacggctgtttaccctgcgacagtattttggcatcctgtatcggctgatcaaaaaccgccggcaagcccggtcggactcggctggaaaagaacaagagatctaa

SEQ ID NO: 15 - DNA, 1047 bp, Neisseria meningitidis
atgccgtcagaagcgtttagacggcaccgggcgtatcgggagaataaactgcaaccattggtgagcgtactgatctgcgcgtataacgtagaaaaatattttgcgcagtcattggcggcggtggttaatcaaacatggcgcaacctggagatcctgatcgtggatgatggctcgacagatgggacgttagcgatcgcgaaggattttcagaagcgggatagccggattaaaattttagctcaggctcagaatagcggcctgatcccgtcattgaacattggactggatgaactggctaagtcaggaatgggagaatatatcgctcgcaccgatgcggatgatatcgcggcgccggattggatcgagaaaattgttggcgagatggaaaaagatcgcagcattattgccatgggcgcctggctggaagttctgtcggaagaaaaggatggcaaccggctggcccggcaccatcgacatggcaaaatctggaaaaagccgaccagaccggaagatatcgcggcgttttttccatttggcaacccaatacataacaacacgatgatcatgcgacgcagcgtgatcgatggcgggctgcggtataacaccgagcgggattgggccgaagattatcagttttggtatgatgtgagcaaactgggccgactggcttattatccggaagcgctggtgaaatatcgcttacatgcgaatcaagtttcaagcaaatatagcattcgccagcatgaaattgcccagggcattcagaaaaccgcgagaaacgattttctgcaatcaatggggtttaaaacccggtttgatagcttagaatatcgccagataaaagctgtagcctatgaactgctggagaaacacctgccggaagaagattttgaacgcgcgcgccggtttctgtatcagtgctttaaacggacggatacgctgccggcggatgcctggctggattttgccgctgatggccgaatgcggcggctgtttaccctgcgacagtattttggcatcctgcatagactgctgaaaaaccggtaa

SEQ ID NO: 16 - DNA, 1047 bp, Neisseria meningitidis
atgccgtcagaagcgtttagacggcaccgggcgtatcgggagaataaactgcaaccactcgtgagcgtactgatctgcgcgtataacgctgaaaaatattttgcgcagtcactcgcggcggtggttaatcaaacatggcgcaacctggagatcctgatcgtggacgatggctcgacagatggcacattagcgatcgcgaaggactttcagaagtgggatagccggattaaaattttagctcaggctcagaatagcggcctgatcccgtcactcaacattggactggatgaactggctaagtcaggaatgggagaatatatcgctcgcaccgacgcggatgacatcgcggcgccggattggatcgagaaaattgttggcgagatggaaaaagatcgcagcattatcgcgatgggggcctggctggaagttctgtcggaagaaaaggatggcaaccggctggcccggcaccatagacatggcaaaatctggaaaaagccgaccagacatgaagacatcgcggatttttttccatttggcaacccgatacataacaacacgatgatcatgaggcgcagcgtgatcgatggcgggctgcggtataacaccgagcgggactgggccgaagactatcagttttggtatgacgtgagcaaactgggcagactggcttattatccggaagcgctggtgaaatatcgcttacatgcgaatcaagtttcaagcaaatatagcattcgccagcatgaaattgcccagggcattcagaaaaccgcgagaaacgactttctgcaatcaatggggtttaaaacccggtttgatagcttagaatatcgccagataaaagctgtagcctatgaactgctggagaaacacctgccggaagaagactttgaacgcgcgcgccggtttctgtatcagtgctttaaacggacggatacgctgccggcggatgcctggctggactttgccgctgatggcagaatgcggcggctgtttaccctgagacagtattttggcatcctgcatagactgctgaaaaaccggtaa

SEQ ID NO: 17 - DNA, 1089 bp, Neisseria meningitidis
atgccgtcagaagcgtttagacggcaccgggcgtatcgggagaataaactgcaaccattggtgagcgtactgatctgcgcgtataacgctgaaaaatattttgcgcagtcattggcggcggtggttaatcaaacatggcgcaacctggagatcctgatcgtggatgatggctcgacagatggcacattagcgatcgcgaaggattttcagaagtgggatagccggattaaaattttagctcaggctcagaatagcggcctgatcccgtcattgaacattggactggatgaactggctaagtcaggaatgggagaatatatcgctcgcaccgatgcggatgatatcgcggcgccggattggatcgagaaaattgttggcgagatggaaaaagatcgcagcattattgccatgggcgcctggctggaagttctgtcggaagaaaaggatggcaaccggctggcccggcaccatcgacatggcaaaatctggaaaaagccgaccagacatgaagatatcgcggatttttttccatttggcaacccgatacataacaacacgatgatcatgcgacgcagcgtgatcgatggcgggctgcggtataacaccgagcgggattgggccgaagattatcagttttggtatgatgtgagcaaactgggccgactggcttattatccggaagcgctggtgaaatatcgcttacatgcgaatcaagtttcaagcaaatatagcattcgccagcatgaaattgcccagggcattcagaaaaccgcgagaaacgattttctgcaatcaatggggtttaaaacccggtttgatagcttagaatatcgccagataaaagctgtagcctatgaactgctggagaaacacctgccggaagaagattttgaacgcgcgcgccggtttctgtatcagtgctttaaacggacggatacgctgccggcgggcgcctggctggattttgccgctgatggcaagatgcgacggctgtttaccatgcgacagtattttagcatcctgcatcggctgatcaaaaaccgccggcaagcccggtcggattcggctggaaaagaacaagagatctaa

SEQ ID NO: 18 - DNA, 1047 bp, Neisseria meningitidis
atgccgtcagaagcgtttagacggcaccgggcgtatcgggagaataaactgcaaccattggtgagcgtactgatctgcgcgtataacgtagaaaaatattttgcgcagtcattggcggcggtggttaatcaaacatggcgcaacctggagatcctgatcgtggatgatggctcgacagatgggacgttagcgatcgcgaaggattttcagaagcgggatagccggattaaaattttagctcaggctcagaatagcgggctgatcccgtcattgaacattggactggatgaactggctaagtcgggaggaggagaatatatcgcccgcaccgatgctgatgatatcgcggcgccggattggatcgagaaaattgttggggagatggaaaaagatcgcagcattattgctatgggcgcctggctggaagttctgtcggaagaaaaggatggcaaccggctggcccggcatcatgaacatggcaaaatctggaaaaagccgacccggcatgaagatattgcggcgttttttccatttggcaacccgatacataacaacacgatgatcatgcggcgcagcgtgatcgatggcgggctgcggtataacaccgagcgggattgggccgaagattatcagttttggtatgatgtgagcaaactgggccgactggcttattatccggaagcgctggtgaaatatcgcttacatgcgaatcaagtttcaagcaaatatagcattcgccagcatgaaattgcccagggcattcagaaaaccgcgagaaacgattttctgcaatcaatggggtttaaaacccggtttgatagcttagaatatcgccagataaaagctgtagcctatgaactgctggagaaacacctgccggaagaagattttgaacgcgcgcgccggtttctgtatcagtgttttaaacggacggatacgccgccggcgggcgtttggctggattttgccgctgatggccgaatgcggcggctgtttaccctgcgacagtattttggcatcctgcatagactgctgaaaaaccggtaa

SEQ ID NO: 19 - DNA, 1047 bp, Artificial sequence
  Synthetic polynucleotide
atgccgtctgaagccttcagacggcatcgtgcctatcgggagaataaattgcaaccattggtgagcgtactgatctgcgcgtataacgtagaaaaatattttgcgcagtcattggcggcggtggttaatcaaacatggcgcaacctggagatcctgatcgtggatgatggctcgacagatgggacgttagcgatcgcgaaggattttcagaagcgggatagccggattaaaattttagctcaggctcagaatagcggcctgatcccgtcattgaacattggactggatgaactggctaagtcaggaatgggagaatatatcgctcgcaccgatgcggatgatatcgcggcgccggattggatcgagaaaattgttggcgagatggaaaaagatcgcagcattattgccatgggcgcctggctggaagttctgtcggaagaaaaggatggcaaccggctggcccggcaccatcgacatggcaaaatctggaaaaagccgacccggcatgaagatatcgcggatttttttccatttggcaacccgatacataacaacacgatgatcatgcgacgcagcgtgatcgatggcgggctgcggtataacaccgagcgggattgggccgaagattatcagttttggtatgatgtgagcaaactgggccgactggcttattatccggaagcgctggtgaaatatcgcttacatgcgaatcaagtttcaagcaaatatagcattcgccagcatgaaattgcccagggcattcagaaaaccgcgagaaacgattttctgcaatcaatggggtttaaaacccggtttgatagcttagaatatcgccagataaaagctgtagcctatgaactgctggagaaacacctgccggaagaagattttgaacgcgcgcgccggtttctgtatcagtgctttaaacggacggatacgctgccggcgggcgcctggctggattttgccgctgatggccgaatgcggcggctgtttaccctgcgacagtattttggcatcctgcatagactgctgaaaaaccggtaa

SEQ ID NO: 20 - DNA, 1047 bp, Artificial sequence
  Synthetic polynucleotide
atgccgtctgaagccttcagacggcatcgtgcctatcgggagaataaattgcaaccattggtgagcgtactgatctgcgcgtataacgtagaaaaatattttgcgcagtcattggcggcggtggttaatcaaacatggcgcaacctggagatcctgatcgtggatgatggctcgacagatgggacgttagcgatcgcgaaggattttcagaagcgggatagccggattaaaattttagctcaggctcagaatagcggcctgatcccgtcattgaacattggactggatgaactggctaagtcaggaatgggagaatatatcgctcgcaccgatgcggatgatatcgcgagcccgggctggatcgagaaaattgttggcgagatggaaaaagatcgcagcattattgccatgggcgcctggctggaagttctgtcggaagaaaaggatggcaaccggctggcccggcaccatcgacatggcaaaatctggaaaaagccgacccggcatgaagatatcgcggatttttttccatttggcaacccgatacataacaacacgatgatcatgcgacgcagcgtgatcgatggcgggctgcggtataacaccgagcgggattgggccgaagattatcagttttggtatgatgtgagcaaactgggccgactggcttattatccggaagcgctggtgaaatatcgcttacatgcgaatcaagtttcaagcaaatatagcattcgccagcatgaaattgcccagggcattcagaaaaccgcgagaaacgattttctgcaatcaatggggtttaaaacccggtttgatagcttagaatatcgccagataaaagctgtagcctatgaactgctggagaaacacctgccggaagaagattttgaacgcgcgcgccggtttctgtatcagtgctttaaacggacggatacgctgccggcgggcgcctggctggattttgccgctgatggccgaatgcggcggctgtttaccctgcgacagtattttggcatcctgcatagactgctgaaaaaccggtaa

SEQ ID NO: 21 - DNA, 1005 bp, Artificial sequence
  Synthetic polynucleotide
atggagaacaagccattggtgagcgtactgatctgcgcgtataacgtagaaaaatattttgcgcagtcattggcggcggtggttaatcaaacatggcgcaacctggagatcctgatcgtggatgatggctcgacagatgggacgttagcgatcgcgaaggattttcagaagcgggatagccggattaaaattttagctcaggctcagaatagcggcctgatcccgtcattgaacattggactggatgaactggctaagtcaggaatgggagaatatatcgctcgcaccgatgcggatgatatcgcggcgccggattggatcgagaaaattgttggcgagatggaaaaagatcgcagcattattgccatgggcgcctggctggaagttctgtcggaagaaaaggatggcaaccggctggcccggcaccatcgacatggcaaaatctggaaaaagccgacccggcatgaagatatcgcggatttttttccatttggcaacccgatacataacaacacgatgatcatgcgacgcagcgtgatcgatggcgggctgcggtataacaccgagcgggattgggccgaagattatcagttttggtatgatgtgagcaaactgggccgactggcttattatccggaagcgctggtgaaatatcgcttacatgcgaatcaagtttcaagcaaatatagcattcgccagcatgaaattgcccagggcattcagaaaaccgcgagaaacgattttctgcaatcaatggggtttaaaacccggtttgatagcttagaatatcgccagataaaagctgtagcctatgaactgctggagaaacacctgccggaagaagattttgaacgcgcgcgccggtttctgtatcagtgctttaaacggacggatacgctgccggcgggcgcctggctggattttgccgctgatggccgaatgcggcggctgtttaccctgcgacagtattttggcatcctgcatagactgctgaaaaaccggtaa

SEQ ID NO: 22 - DNA, 1005 bp, Artificial sequence
  Synthetic polynucleotide
atgctcgaactgccattggtgagcgtactgatctgcgcgtataacgtagaaaaatattttgcgcagtcattggcggcggtggttaatcaaacatggcgcaacctggagatcctgatcgtggatgatggctcgacagatgggacgttagcgatcgcgaaggattttcagaagcgggatagccggattaaaattttagctcaggctcagaatagcggcctgatcccgtcattgaacattggactggatgaactggctaagtcaggaatgggagaatatatcgctcgcaccgatgcggatgatatcgcggcgccggattggatcgagaaaattgttggcgagatggaaaaagatcgcagcattattgccatgggcgcctggctggaagttctgtcggaagaaaaggatggcaaccggctggcccggcaccatcgacatggcaaaatctggaaaaagccgacccggcatgaagatatcgcggatttttttccatttggcaacccgatacataacaacacgatgatcatgcgacgcagcgtgatcgatggcgggctgcggtataacaccgagcgggattgggccgaagattatcagttttggtatgatgtgagcaaactgggccgactggcttattatccggaagcgctggtgaaatatcgcttacatgcgaatcaagtttcaagcaaatatagcattcgccagcatgaaattgcccagggcattcagaaaaccgcgagaaacgattttctgcaatcaatggggtttaaaacccggtttgatagcttagaatatcgccagataaaagctgtagcctatgaactgctggagaaacacctgccggaagaagattttgaacgcgcgcgccggtttctgtatcagtgctttaaacggacggatacgctgccggcgggcgcctggctggattttgccgctgatggccgaatgcggcggctgtttaccctgcgacagtattttggcatcctgcatagactgctgaaaaaccggtaa

SEQ ID NO: 23 - DNA, 1047 bp, Artificial sequence
  Synthetic polynucleotide
atgccgtcagaagcgtttagaagacatcgggcgtatagagagaataaactgcaaccgttggtgagcgttctgatctgcgcgtataacgttgaaaaatattttgcgcagtcattggcggcggtggtgaatcaaacttggcgcaacctggagatcctgatcgtggatgatggctcaacagatgggactttggcgatcgcgaaggattttcagaagagagatagccgcattaaaattttggctcaggctcagaattcgggcctgatcccgtcattgaacattggactggatgaactggctaagtcgggaggaggagaatatatcgctcgcaccgatgcggatgatatcgcggcgccagattggatcgagaaaattgtgggcgagatggaaaaagatcgcagcattatcgctatgggcgcttggctggaagtgctgtcagaagaaaaggatggcaacagactggctagacatcatgaacatggcaaaatctggaaaaagccgacccaacatgaagatatcgcggcggtgtttccgtttggcaacccaatacataacaacactatgatcatgaggcgcagcgtgatcgatggcgggctgcggtataacaccgagagagattgggctgaagattatcagttttggtatgatgtgagcaaactgggcaggctggcttattatccagaagcgctggtgaaatatcgcttgcatgcgaatcaagtgtcatcgaaatatagcgtgcgccagcatgaaattgctcagggcattcagaaaaccgcgagaaacgattttctgcaatcaatggggtttaaaaccagatttgatagcttggaatatcgccagataaaagctgttgcttatgaactgctggagaaacatctgccggaagaagattttgaacgcgcgcgcagatttctgtatcagtgttttaaaagaactgatactccgccagcgggcgcttggctggattttgctgctgatggcaggatgagaagactgtttaccctgaggcagtattttggcatcctgcgcagactgctgaaaaaccggtga

SEQ ID NO: 24 - DNA, 1005 bp, Artificial sequence
  Synthetic polynucleotide
atggagaacaagccactcgtgagcgtactgatctgcgcgtataacgtagaaaaatattttgcgcagtcactcgcggcggtggttaatcaaacatggcgcaacctggagatcctgatcgtggacgatggctcgacagatggcacgttagcgatcgcgaaagactttcagaagcgggatagccggattaaaattttagctcaggctcagaatagcggcctgatcccgtcactcaacattggactggatgaactggctaagtcgggaggaggagaatatatcgcccgcaccgacgcggatgacatcgcggcgccggattggatcgagaaaattgttggggagatggaaaaagatcgcagcattatcgccatgggcgcctggctggaagttctgtcggaagaaaaggatggcaaccggctggcccggcatcatgaacatggcaaaatctggaaaaagccgacccggcatgaagatatcgcggcggtttttccatttggcaacccgatacataacaacacgatgatcatgcggcgcagcgtgatcgatggcgggctgcggtataacaccgagcgggactgggccgaagactatcagttttggtatgacgtgagcaaactgggcagactggcttattatccggaagcgctggtgaaatatcgcttacatgcgaatcaagtttcaagcaaacatagcgtgcgccagcatgaaattgcccaggatattcagaaaaccgcgagaaacgactttctgcaatcaatggggtttaaaacccggtttgatagcttagaatatcgccagataaaagctgtagcctatgaactgctggagaaggacctgccggaagaagactttgaacgcgcgcgccggtttctgtatcagtgttttaaacggacggatacgccgccggcgggcgcctggctggactttgccgctgatggcagaatgcggcggctgtttaccctgagacagtattttggcatcctgcgcagactgctgaaaaaccggtaa

SEQ ID NO: 25 - DNA, 1005 bp, Artificial sequence
  Synthetic polynucleotide
atgctcgaactgccactcgtgagcgtactgatctgcgcgtataacgtagaaaaatattttgcgcagtcactcgcggcggtggttaatcaaacatggcgcaacctggagatcctgatcgtggacgatggctcgacagatggcacgttagcgatcgcgaaagactttcagaagcgggatagccggattaaaattttagctcaggctcagaatagcggcctgatcccgtcactcaacattggactggatgaactggctaagtcgggaggaggagaatatatcgcccgcaccgacgcggatgacatcgcggcgccggattggatcgagaaaattgttggggagatggaaaaagatcgcagcattatcgccatgggcgcctggctggaagttctgtcggaagaaaaggatggcaaccggctggcccggcatcatgaacatggcaaaatctggaaaaagccgacccggcatgaagatatcgcggcggtttttccatttggcaacccgatacataacaacacgatgatcatgcggcgcagcgtgatcgatggcgggctgcggtataacaccgagcgggactgggccgaagactatcagttttggtatgacgtgagcaaactgggcagactggcttattatccggaagcgctggtgaaatatcgcttacatgcgaatcaagtttcaagcaaacatagcgtgcgccagcatgaaattgcccaggatattcagaaaaccgcgagaaacgactttctgcaatcaatggggtttaaaacccggtttgatagcttagaatatcgccagataaaagctgtagcctatgaactgctggagaaggacctgccggaagaagactttgaacgcgcgcgccggtttctgtatcagtgttttaaacggacggatacgccgccggcgggcgcctggctggactttgccgctgatggcagaatgcggcggctgtttaccctgagacagtattttggcatcctgcgcagactgctgaaaaaccggtaa

SEQ ID NO: 26 - DNA, 1008 bp, Artificial sequence
  Synthetic polynucleotide
atgctcgaactgcctttagtcagcgtattgatttgcgcctacaacgtagaaaaatatttcgcccaatcattagccgccgtcgtgaatcaaacttggcgcaacttggatattttgattgtcgatgacggctcgacggacggtacgcttgccattgcccagcgtttccaagaacaggacggccgcatccgcatcctcgcgcagccgcgcaattccggtctgattccctctttaaacatcgggctggatgaattggcaaagtcgggaggcggtggggaatatattgcgcgcaccgatgcagacgatattgccgcccccgactggattgagaaaatcgtgggtgagatggaaaaagaccgcagcatcatcgcgatgggtgcgtggctggaagttttgtcggaagaaaaggacggcaaccggctggcgcggcaccacgaacacggcaaaatttggaaaaaaccgacccgacacgaagacattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgcgtggctggattttgcggcagacggcaggatgcggcggctgtttaccttgaggcaatacttcggcattttgcaccgattgctgaaaaaccgttga

SEQ ID NO: 27 - DNA, 1005 bp, Pasteurella multocida
atgttaaataaacctttagtttcagttttgatttgtgcttataatgttgaaaaatatattgaagaatgtatcaatgcagtcattaatcaaacatataagaatttagaaattatcgtagttaatgatggttcttctgacaatacttattttctattaaaacaattagctaaaaaagataatcgtatcaaaatattaaacttagagaataatgtaggaataatatctgcgttaaatgaaggattaaaagaagttagtggtgagtatattgctcgaactgattccgacgatataactaaacctgattggattgagaaaatattaacctgtatggaaaatgatcctgaaattattgcgatgggatcttatcttactgtcttatcggaagaaaataatggtagtgtgcttgctaatcaccataaagatgcggttgaatggaaaaatcctttagaacataaagatatagtggaaaaaatgttatttggcaatcccattcataataactcaatgataatgaggagtaagatatatactaagcataaattaatttatgatttagattatccttatgctgaagattataaattttggttggaagtgagtcgcattggtaaattagcgaattaccctgaatctctagtctattatagattgcatcataatcagacatcttctattcatcatcatcaacaagaaagaattggtaaaaaactacgattacaagccctcaattattatttaaaagatcttagtattgattaccagctgcctgagaaatttttattcaaagatgtagaaaagttacaagatatattttatagtcatggtattttacaagaaaagataataaaacgaattatctacgaatgttacctttctttagcagagtatcattataaagatatttattatttcataaggaataagagtgattttctttctataaagcaaaaatctaaaataataaaaaaatatcttagacctggtaaatatttatctacgtattag

SEQ ID NO: 28 - DNA, 1047 bp, Neisseria lactamica
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggatattttgattgtcgatgacggctcgacagacggtacgcttgccattgctaaggattttcaaaagcgggacagccgtatcaaaatccttgcacaagctcaaaattccggcctgattccctctttaaacatcgggctggacgaattggcaaagtcaggcggcggtgagtatattgcgcgcaccgatgcagacgatattgccgcccccgactggattgagaaaatcgtgggtgagatggaaaaagaccgcagcatcattgcgatgggcgcgtggctggaagttttgtcggaagaaaaggacggcaaccggctggcgcggcaccacgaacacggcaaaatttggaaaaagccgacccggcacgaagacatcgccgcctttttcccttttggcaaccctatacacaacaacacgatgattatgcggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaacacagcgtccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaggatttgccggaagaagatgttgaacgcgcccgccggtttttgtaccaatgtttcaaacggacggacacgccgcccgccggcgcgtggctggattttgcggcagacggcaggatgcggcggctgtttaccttgaggcaatacttcggcattttgcaccgattgctgaaaaaccgttga

SEQ ID NO: 29 - DNA, 1047 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggagattttgattgtcgatgacggctcgacagacggtacgcttgccattgccaaggattttcaaaagcgggacagccgtatcaaaatccttgcacaagctcaaaattccggcctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcattgccatgggtgcgtggctggaagttttgtcggaagaaaaggacggcaaccggctggcgcggcaccacgaacacggcaaaatttggaaaaagccgatccggcacgaagatattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgccgcccgccggcgtgtggctggattttgcggcagacggcaggatgcggcggctgtttaccttgaggcaatacttcggcattttgcaccgattgctgaaaaaccgttga

SEQ ID NO: 30 - DNA, 1050 bp, Neisseria polysaccharea
atgccgtctgaagccttcagacggcatcgtgcctatcgggagaataaattgcagcctttagtcagcgtattgatttgcgcctacaacgtagaaaaatatttcgcccaatcattagccgccgtcgtgaatcaaacttggcgcaacttggatattttgattgtcgatgacggctcgacggacggtacgcttgccattgcccagcgtttccaagaacaggacggtcgcatccgcatcctcgcgcagccgcgcaattccggtctgattccctctttaaacatcgggctggacgaattggcaaagtcaggtggcggcggagaatatattgcgcgcaccgatgcagacgatattgccgcccccgactggattgagaaaatcgtgggtgagatggaaaaagaccgcagcatcatcgcaatgggcgcgtggctggaagttttgtcggaagaaaaggacggcaaccggctggcgcggcaccacgaacacggcaaaatttggaaaaagccgacccggcacgaagacatcgccgcctttttcccttttggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcgtccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtatcaatgtttcaaacggacggacacgccgcccgccggcgcgtggctggattttgcggcagacggcaggatgcggcggctgtttaccttgaggcaatacttcggcattttgcgccgattgctgaaaaaccgttga

SEQ ID NO: 31 - DNA, 1047 bp, Neisseria subflava
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggatattttgattgttgatgacggctcgacggacggcacacttgccattgcccggcgtttccaagaacaggacggccgcatccgcatcctcgcgcagccgcgcaattccggcctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcatcgcgatgggcgcgtggctggaagttttgtcggaagaaaaggacggcaaccggctggcgcggcatcacaggcacggcaaaatttggaaaaagccgacccgacacgaagatattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcgtccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgcgtggctggattttgcggcagacggcaggatgcggcggctgtttaccttgaggcaatacttcggcattttgcaccgattgctgaaaaaccgttga

SEQ ID NO: 32 - DNA, 1047 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggatattttgattgttgatgacggctcgacggacggcacacttgccattgcccggcgtttccaagaacaggacggccgcatccgcatcctcgcgcagccgcgcaattccggcctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcatcgcgatgggcgcgtggctggaagttttgtcggaagaaaaggacggcaaccggctggcgcggcatcacaggcacggcaaaatttggaaaaagccgacccgacacgaagatattgccgactttttccctttcggcaaccccatacacaacaacacgatgattatgaggcgcagcgtcattgacggcggtttgcgttacaacaccgagcgggattgggcggaagattaccaattttggtacgatgtcagtaaattgggcaggctggcttattatcccgaagccttggtcaaataccgccttcacgccaatcaggtttcatccaaatacagcgtccgccaacacgaaatcgcgcagggcatccaaaaaaccgccagaaacgattttttgcagtctatgggttttaaaacccggttcgacagccttgaataccgccaaataaaagcagtagcgtatgaattgctggagaaggatttgtcggaagaagattttgaacgcgtccgccggtttttgtaccaatgcttcaaacggacggacacgccgcccgccggtgcgtggctggatttcgcggcagacggcaggatgaggcggctgtttaccatgaggcaatacttcggcattttgcaccgattgctgaaaaaccgttga

SEQ ID NO: 33 - DNA, 1047 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcaaacttggcgcaacttggatattttgattgtcgatgacggctcgacggacggtacgcttgccattgcccagcgtttccaagaacaggacggccgcatccgcatcctcgcgcagccgcgcaattccggtctgattccctctttaaacatcgggctggatgaattggcaaagagcggtggcggtgaatatattgcgcgcaccgatgcagacgatattgccgcccccgactggattgagaaaatcgtgggtgagatggaaaaagaccgcagcatcatcgcgatgggtgcgtggctggaagttttgtcggaagaaaaggacggcaaccggctggcgcggcaccacgaacacggcaaaatttggaaaaaaccgacccgacacgaagacattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgcgtggctggattttgcggcagacggcaggatgcggcggctgtttaccttgaggcaatacttcggcattttgcaccgattgctgaaaaaccgttga

SEQ ID NO: 34 - DNA, 1047 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcaaacttggtgcaacttggatattttgattgtcgatgacggctcgacggacggtacgcttgccattgcccagcgtttccaagaacaggacggccgcatcaaaatccttgcacaagctcaaaattccggtctgattccttctttaaacatcgggctggacgaattggcaaagtcaggaatgggggaatatattgcacgcaccgatgcagacgatattgccgcccccgattggattgagaaaatcgtgggcgagatggaaaaagaccgcagcatcattgcgatgggcgcgtggctggaagttttgtcggaagaaaaggacggcaaccggctggcgcggcaccacgaacacggcaaaatttggaaaaagccgatccggcacgaagatattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgccgcccgccggcgtgtggctggattttgcggcagacggcaggatgcggcggctgtttaccttgaggcaatacttcggcattttgcaccgattgctgaaaaaccgttga

SEQ ID NO: 35 - DNA, 999 bp, Neisseria meningitidis
atgcagcctttagtcagcgtattgatttgcgcctacaacgtagaaaaatatttcgcccaatcattagccgccgtcgtgaatcaaacttggtgcaacttggatattttgattgtcgatgacggctcgacggacggtacgcttgccattgcccagcgtttccaagaacaggacggccgcatcaaaatccttgcacaagctcaaaattccggtctgattccttctttaaacatcgggctggacgaattggcaaagtcaggaatgggggaatatattgcacgcaccgatgcagacgatattgccgcccccgactggattgagaaaatcgtgggcgagatggaaaaagaccgcagcatcattgcgatgggtgcgtggctggaagttttgtcggaagaaaaggacggcaaccggctggcgcggcaccacgaacacggcaaaatttggaaaaagccgacccgacatgaagacattgccgactttttccctttcggcaaccccatacacaacaacacgatgattatgcggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgtgtggctggattttgcggcagacggcaggatgcggcggctgtttaccttgaggcaatacttcggcattttgcaccgattgctgaaaaaccgttga

SEQ ID NO: 36 - DNA, 1215 bp, Helicobacter pylori
atgacttcagcttcaagccattcttttaaagaacaagattttcatatccctatcgcttttgcttttgacaagaattacctcattcctgcgggcgcatgcatttattccttgttagagagcatcgctaaggctaataaaaaaatccgttacaccctacacgctttagtggtaggcttgaatgaagaagataaagcaaagcttaaccaaatcacagagccttttaaagaatttgctgttttagaagtaaaagatattgaaccttttttagacgctatccccaacccttttgatgaggatttcaccaagcgtttttctaaaatggtgttagtgaagtattttctagcggatttattccccaaatattctaaaatggtgtggagcgatgtggacgttatcttttgcaatgaatttagtgctgatttcttaaacattaaagaaaatgatgagaattatttttatggggttttagaagttgaaaagcaccacatgatggaagggtttttgttttgcaatttagattaccaacgcaagaaaaatttcaccttaagaatgcatgatcttttaaaggggaatgaggctaaaggggagttggatttcacgaaatggtgttggcctaacatgaaggctttagggattgaatattgcgttttcccttattattacaccattaaagatttttctaacgcgtatttaaacgagaattacaagaaaaccattttagaggcgcgagaaaaccctaccattatccactatgacgcttggtggggggcagtgaagccttgggactatccttttggtttaaaagcggatttgtggctgaacgctttggctaaaaccccctttatgagcgattacactaagaaaatgcacaccaatgagagcttttataccacaaaaatggctgagcagcattatttttcttcagtaaaatcttcaaaagaaattcttttcaaaaccccgtatttgtttttcaaatcgtatctgtttgttgtgtttaaagaaaggaaaatccattcaagagtttttgaattaacttgtaatttagtgaaaaaatttttcaataagcttatttattttgggtttttgatgcctaaagcgttagctaaaagggtggttagtaagatccttagagttttagggcttcatgggattgttaaaaaaatcttaatccaactcaagctcttgaaaaaggagctgttcctgtggggcctttga

SEQ ID NO: 37 - DNA, 1311 bp, Helicobacter pylori
atgacttcagcttcaagccattcatttaaagaacaagattttcatattcctatcgcttttgcttttgataagaattacctcattcctgcgggcgcgtgtctttattccttgctagaaagcatcgctaaagccaataaaaaaatccgttacaccctacacgctttagtggtaggcttgaatgaagaagataaagcaaagcttaaccaaattacagagccttttaaagaatttgctgctttagaagtaaaagatattgaaccttttttagacgctatccctaacccttttgatgaggatttcactaagcgtttttctaaaatggtgttagtgaaatattttctagcggatttgttccccaaatattctaaaatggtgtggagcgatgtggatgttatcttttgcaatgaatttagcgctgatttcttaaacattaaagaaaatgatgagaattatttttatggggttttaggaggtggagagcgccatgtgttggaagggtttttgttttgtaatttagattaccagcgcaagaaccattttgtctcaaaaatccatgcacttttggaagaaaacaaggctacagaagaattggatttcacccaatggtgttggcctaacatgaaagctttagggattgagtattgcgttttccctttctattacaccgttaaagatttttctgcatccttgcctgagaattattttttgcatgagatttataagaaaaccatctcaaacgcgctcaaaaatcctataatcatccattatgatgtttggtggggggcggtgaagccttgggattatccttttggtttaaaagcggatttgtggctgaacgctttggctaaaaccccttttatgagcgactggattgattcgatcgctagggtggaaataggcagcgaaaaatggcatcgttaccacagcattgttgcctatcactactactttcccctatggaagactgaagagcagatcgcccatgacgcgctcaagacctttttagaccattatttttcgtgcatccatgccacaatcaagcaagaaaatctcggaatgttcttgaaccactacttctcgcatgcccatgcagagatcaaagaaaactcccttgaagcgtttctcaatcattatttctctcacgtttataggctccctaaaaacgcgcgaaagaaactctttagggtgtttgtcaaacattgcatcctcataccactcaagagccttattagtaaaacccttaaaatcctaggacttcatgggcttgttaaaaaaatcctaatccaactcaagctcttaaaaaagagctag

SEQ ID NO: 38 - DNA, 1197 bp, Helicobacter pylori
atgacttcagcttcaagccattcttttaaagaacaagattttcatattcctatcgcttttgcttttgataagaattacctcattcctgcgggcgcgtgtctttattccttgctagaaagcatcgctaaagccaataaaaaaatccgttacaccctacacgctttagtggtgggcttgaatgaagaagataaagcaaagctccacaaaatcgcagagccttttaaagaatttgctgttttagaagtaaaagatattgaaccttttttagacactatccctaacccttttgatgaggatttcactaagcgtttttctaaaatggtgttagtgaagtattttctagcggatttattccccaaatattctaaaatggtgtggagcgatgtggatgttatcttttgcaatgaatttagcgctgatttcttaaacattaaagaagatgatgagaattatttttatggggttttggaagttgaaaagcaccacatgatggaagggtttttgttttgtaatttagattaccaacgcaagaaaaatttcaccttaagaatgcatgatcttttaaaggggaatgaggctaaaggggagttggattttacgaaatggtgttggcctaacatgaaagctttagggattgaatattgcgttttcccttattattacaccattaaagatttttctaacgcgtatttaaacgagaattacaagaaaaccattttagaggcgcgagaaaaccctactattatccactatgacgcttggtggggggcggtgaagccttgggattacccttttggtttaaaagcggatttgtggttgaacgctttggctaaaaccccatttatgagcgattacactaagaaaatgcacaccaatgagagcttttacaccacaaaaatggctgagcagcattatttttcttcagtaaagtcttcaaaagaaattcttttcaaaaccccgtatttgtttttcaaatcgtatctgcttgttgtgtttaaagaaaggaaaatccatttaaggatttttgcattaatgggtggtttagtgaaaaaatttttcaataagcttatttattttgggtttttgatgcctaaagcgttagttaaaagggcggttagtaagatccttagagttttagggcttcatgggattgtgaaaaaaatcttaatcaaactcaagctcttaagaaagggctag

SEQ ID NO: 39 - DNA, 1197 bp, Helicobacter pylori
atgacttcagcttcaagccattcttttaaagaacaagattttcatattcctatcgctttcgcttttgataagaattaccttattcctgcgggtgcgtgtctttattccttgctagaaagcatcgctaaggctaataaaaaaatccattacaccctacacgctttagtggtaggcttgaatgaagaagataaagcaaagcttaaccaaatcacagagccttttaaagaatttgctactttagaagtgagagatattgaaccttttttagacgctatccctaacccttttgatgaggatttcaccaagcgtttttctaaaatggtgttagtgaagtattttctagcggatttattccctaaatattctaaaatggtgtggagcgatgtggacgttatcttttgcaatgaatttagcgctgatttcttaagtattaaagaagatgatgagaattatttttatggggttttagaagttgaaaagcaccacatgatggaagggtttttgttttgcaatttagattaccagcgcaagaaaaatttcaccttaagaatgcatgatcttttaaaggggaatgaggctaaaggggagttggattttacgaaatggtgttggcctaacatgaaggctttagggattgaatattgcgttttcccttattattacaccattaaagatttttctaacgcatacttaaacgagaattataagaaaaccattttagaggcgcgagaaaaccctaccattatccactatgacgcttggtggggggcggtgaaaccttgggactatccttttggtttaaaagcggatttgtggctgaacgctttggctaaaacctcttttatgagcgattacactaaaaaaatgcacaccaatgagagcttttataccacaaaaatggctgagcagcattatttttcttcagtaaaatcttcaaaagaaattcttttcaaaaccccgtatttgtttttcaaatcgtatctgtttgttgtgtttaaagaaaggaaaatccattcaagagtttttgaattaatttgtggtttagtgaaaaaatttttcaataagcttatttattttgggtttttgatgcctaaagcgttagttaaaagggcggttagtaagatccttagagttttagggcttcatgggattgttaaaaaaatcttaatcaaactcaaactcttgaaaaagggctag

SEQ ID NO: 40 - DNA, 1197 bp, Helicobacter pylori
atgacttcatcttcaaaccattcttttagagaacaagattttcatatccctatcgcttttgcttttgataagaattacctcattcctgcgggcgcgtgtctttattccttgctagaaagcatcgctaaagccaataaaaaaatccgttacaccctacacgctttagtggtaggcttgaatgaagaagataaaacaaagcttaatcaaatcacagagccttttaaagaatttgttgctttagaagtaaaagatattgaaccttttttagacactatccctaacccttttgatgaggatttcaccaagcgtttttctaaaatggtgttagtgaagtattttttggcggatttgttccccaaatattccaaaatggtgtggagcgatgtggatgttatcttttgtaatgaatttagcgctgatttcttaaacattaaagaagatgatgagaattatttttatggggttttggaagttgaaaagcaccacatgatggaagggtttttgttttgtaatttagattaccaacgcaagaaaaatttcaccttaagaatgcatgagattttaaaggggaatgagactaaaggggagttggattttacgaaatggtgttggcccaatatgaaagctttaggggttgagtattgcgttttcccttggtattacaccattaaagatttttctaacgcgtatttaaacgagaattacaagaaaaccattttagaggcgcgagaaaaccctaccattatccactatgacgcttggtggggggcggtgaagccttgggattacccttttggtttaaaagcggatttatggctgaacgctttggctaaaaccccatttatgagcgattacactaagaaaatgcacaccaatgagagcttttataccacaaaaatggctgagcagcattatttttcttcagtaaaatcttcaaaagaaattcttttcaaggctccgtatttgtttttcaaatcgtatctgtttgttgtgtttaaagaaaggaaaatccattcaagagtttttgaattaacttgtaatttagtgaaagaatttttcaataagcttatttattttgggtttttgatgcctaaagcgttagctaaaagggtggttagtaagatccttaggattttagggcttcatgggattgtgaaaaaaatcctaatccaactcaagcttttaagaaagggctag

SEQ ID NO: 41 - DNA, 1191 bp, Helicobacter pylori
atgacttcagcttcaaaccattcttttaaagaacaagattttcatattcctatcgcttttgcttttgataagaattacctcattcctgcgggtgcgtgtctttattccttgctagaaagcatcgctaaagccaataaaaaaatccgttacaccctacacgctttagtggtaggcttgaatgaagaagataaagcaaagcttaatcaaatcgcagagccttttaaagaatttgctgttttagaagtaaaagatattgaaccttttttagacactatccctaacccttttgatgaggatttcaccaagcgtttttctaaaatggtgttagtgaagtattttttggcggatttgttccccaaatattccaaaatggtgtggagcgatgtggatgtcatcttttgcaatgaatttagtgctgatttcttaaacattgaagaaaatgatgagaattatttttatggggttttagaagttgaaaagcaccacatgatggaagggtttttgttttgtaatttagattaccaacgcaagaaaaatttcaccttaagaatgcatgagattttaaaggggaatgagactaaaggggagttggattttacgaaatggtgttggcccaatatgaaagctttaggggttgagtattgcgttttcccttggtattacaccattaaagatttttctaacgcgtatttaaacgagaattacaagaaaaccattttagaggcgcttaaaaaccctaccattatccactatgacgcttggtggggggcggtgaagccttgggattacccttttggtttaaaagcggatttatggctgaacgctttggctaaaaccccttttatgagcgattacactaaaaaaatgcacaccaatgagagcttttacaccacaaaaatagctcaacaacattatttttcttcaataaagtcttcaaaagaaattctttttaaggttccgtatttgtttttcaaatcgtatttgtttgttgtgtttaaagaaaggaaaatccattcaagagtttttgaattaacttgtggtttggtgaaaaaatttttcaataagcttatttactttgggttttttatgcctaaagcgttagctaaaagggtggttagtaagatccttatagttttagggcttcatgggattgttaaaaaaatcttaatcaagctcttaaaaaagggatag

SEQ ID NO: 42 - DNA, 1185 bp, Helicobacter pylori
atgacttcagcttcaagccattcttttaaagaacaagattttcatatccctatcgcttttgcttttgataagaattacctcattcctgcaggtgcatgtatttattccttgctagaaagcatcgctaaagccaataaaaaaattcgttacaccctatacgctttagtggtgggcttggatgaagaagatgaagcaaagctccaccaaattacagagccttttaaagaatttgccactttagaagtgaaagacattgaaccttttttagacaccatccctaacccttttgatgaggatttcaccaagcgtttttctaaaatggtgttagtgaagtattttctagcggatttgttccccaaatattccaaaatggtgtggagtgatgtggatgtcatcttttgcaatgaatttagcgctgatttcttaaacattaaagaagatgatgagaattatttttatggggttttagaagttgaaaagcaccacatgatggaagggtttttgttttgtcatttagattaccagcgcaagaaaaatttcaccttaagaatgcatgatcttttaaaggggaatgaggctaaaggggagttggattttacaaaatggtgttggcctaacatgaaagctttagggattgagtattgcgttttccccttctattacaccattaaagatttttctaacgcgtatttaaacgagaattacaagaaaaccattttagaggtgctcaaaaaccctaccattatccactatgacgcttggtggggagcagtgaagccttgggattacccttttggtttaaaagcggatttatggctgaacgctttgtctaaaaccccttttatgagcgattacactaaaaaaatgcacaccaatgagagcttttacaccacaaaaatggctcagcaacattatttttcttcaacagcgccttcaaaagaaattctttttaaggctccgtatgtgtttttcaaatcgtatttgtttgttgtgtttaaagaaaggaaaatccattcaagagtttttgaattaacttgtggtttagtgaaaaaatttttcaataagcttatttactttgggttttttatgcctaaagcgttagctaaaagggtggttagtaagatccttaaaatcctagggcttcatgggattgttaaaaaaatcctactcaagctcttaaaatag

SEQ ID NO: 43 - DNA, 1197 bp, Helicobacter pylori
atgacttcagcttcaagccattctcttaaagaacaagattttcatatccctatcgcttttgcttttgataagaattatctcattgctgcgggcgcgtgcatttattccttgctagaaagcatcgctaaagccaataaaaaaattcgttacaccctatacgctttagtggtaggcttggatgaagaagataaagcaaagctccaccaaattacagagccttttaaagaatttgctgttttagaagtgaaagatattgaaccttttttagacactatccctaacccttttgatgaggatttcaccaagcgtttttctaaaatggtgttagtgaagtattttttggcggatttgttccccaaatattccaaaatggtgtggagcgatgtggatgtcatcttttgcaatgaatttagcgctgatttcttaaacattaaagaagatgatgagaattatttttatggggttttggaagttgaaaagcaccacatgatggaagggtttttgttttgtcatttagattaccagcgcaagaaaaatttcaccttaagaatgcatgatcttttaaaggggaatggggctaaagaggagttggattttacgaaatggtgttggcctaacatgaaagctttagggattgagtattgcgttttccctttgtattacaccattaaagatttttctaatgtgtatttaaacgagaattacaagaaaaccattttagaggcactcaaaaaccctataatcatccactatgacgcttggtggggggcggtgaagccttgggattacccttttggtttaaaagcggatttatggctgaacgctttgtctaaaaccccttttatgagcgattacactaaaaaaatgcacaccaatgagagcttttacaccacaaaaatggctcaacaacattatttttcttcaacagcgccttcaaaagaaattctttttaaggctccgtatttgtttttcaaatcgtatttgtttgttgtgtttaaagaaaggaaaatccattcaagagtttttgaattaacttgtggtttagtgaaaaaatttttcaataagcttatttactttgggttttttatgcctaaatcgttagctaaaagggtggttagtaagatccttagagttttagggcttcatgggattgctaaaaaaattctaatcaaactcaagctcttaaaaaagagctag

SEQ ID NO: 44 - DNA, 1311 bp, Helicobacter pylori
atggattcagcttcaaaccattcttttaaagaacaagattttcatattcctatcgcttttgcttttgataagaattacctcattcctgcgggcgcgtgtctttattccttgctagaaagcatcgctaaagccaataaaaaaatccgttacaccctacacgctttagtggtgggcttgaatgaagaagataaagcaaagcttaatcaaatcgcagagccttttaaagaatttgctgttttagaagtaaaagatattgaaccttttttagacgctatccctaacccttttgatgaggatttcactaagcgtttttctaaaatggtgttagtgaaatattttctagcggatttattccccaaatattctaaaatggtgtggagcgatgtggacattatcttttgcaatgaatttagcgctgatttcttaaacattaaagaagatgatgagaattatttttatggggttttaggaggtggagagcaccatgtgttggaagggtttttgttttgtaatttagattaccagcgcaagaaccattttgtctcaaaaacccatgcacttttggaagaaaacaaggctacagaagaattggatttcacccaatggtgttggcctaacatgaaagctttagggattgagtattgcgttttcccttattattacactgtcaaagatttttctgcatccttgcctgagaattattttttgcatgagatttataagaaaaccatctcaaacgcgctcaaaaaccctataatcatccattatgatgtttggtggggggcggtgaagccttgggattatccttttggtttaaaagcggatttgtggctgaacgctttggctaaaaccccatttatgagcgactggattgattcgattgctagggtggaaataggcagtgaaaaatggaatcgctaccacagcatcgttgcctatcactactactttcccctatggaagactgaagagcagatcgcccatgacgcgctcaagacctttttagaccattatttttcgtgcatccatgccacaatcaagcaagaaaatctcggaatgttcttgaaccactacttctcgcatgcccatgcagagatcaaagagaactcccttgaagtgtttctcaatcattattttagccacgtttataggctccctaaaaacgcgcaaaagaaactctttagggtgtttgtcaaacattgcatcctcataccactcaagagccttattagtaaaacccttaaaattttaaaactccatgcactggtgaaaaaaatcctaatccaactcaagctcttaagaaagagctag

SEQ ID NO: 45 - DNA, 1371 bp, Helicobacter pylori
atgacttcagcttcaaaccattcttttaaagaacaggattttcatatccctatcgcttttgcttttgataagaattacctcattcctgcgggcgcgtgtctttattccttgctagaaagcattgctaaagccaataaaaaaatccgttacaccctacacgctttagtggtaggcttgaatgaagaagataaagcaaagcttaatcaaatcacagagccttttaaagaatttgctgctttagaagtaaaaaatattgaaccttttttagacactatccctaacccttttgatgaggatttcactaagcgtttttctaaaatggtgttagtgaagtattttctagcggatttattccccaaatattctaaaatggtgtggagcgatgtggatgtcatcttttgcaatgaatttagcgctgatttcttaaacattaaagaaaatgatgagaattatttttatggggttttaggaggtggagagcgccatgtgttggaagggtttttgttttgtaatttagattaccagcgcaagaaccattttgtctcaaaaacccatgcacttttggaagaaaacaaggctacagaagaattggatttcacccaatggtgttggcccaatatgaaagctttagggattgaatattgcgttttcccttattattacactgtcaaagatttttctgcatccttgcctgagaattattttttgcatgagatttataagaaaaccatctcaaacgcgctcaaaaaccctataatcatccattatgatgtttggtggggggcggtgaagccttgggattatccttttggtttaaaagcggatttgtggctgaacgctttggctaaaaccccctttatgagcgattggattgattcgatcgctagggtggaaataggcagcgaaaaatggcatcgttaccacagcatcgttgcctatcactactactttcccctatggaagactgaagagcagattgcccatgacgcgcttaagacctttttagaccattatttttcgtgcattcatgccacaatcaagcaagaaaatctcggaatgtttctgaatcattatttctcacacgcgcacgctgaaatcaaggagaatcctctggaagcctttctcaatcattattttagccacgcgcacgcggaaattaaagagaattcactggaagcatttctgaatcattattttagccacgtttatagactccctaaaaacgcgcgaaagagactccttagggtgtttgtcaaacattgcatcctcataccactcaagagccttgtgggtaagactctgcgattcttaaaactccatgcactggtgaaaaaaatcctaatccaactcaagctcttaaaaaagagctag

SEQ ID NO: 46 - DNA, 1311 bp, Helicobacter pylori
atgacttcatcttcaagccattcttttaaagaacaagattttcatattcctatcgcttttgcttttgataagaactacctcattcctgcgggcgcgtgtctttattccttgctagaaagcatcgctaaagccaataaaaaaatccgttacaccctacacgctttagtggtaggcttgaatgaagaagataaagcaaaacttaatcaaatcgcagagccttttaaagaatttgctgttttagaagtgagagatattgaaccttttttagacactatccctaacccttttgatgaggatttcactaagcgtttttctaaaatggtgttagtgaagtattttctagcggatttattccccaaatattctaaaatggtgtggagcgatgtggacgttatcttttgcaatgaatttagcgctgatttcttaaacattaaagaagatgatgagaattatttttatggggttttaggaggtggagagtgccatgtgttggaagggtttttgttttgtaatttagattaccagcgcaagaaccattttgtctcaaaaacccatgcacttttggaagaaaacaaggctacagaagaattggatttcacccaatggtgttggcccaatatgaaagctttagggattgaatattgcgttttcccttattattacactgtcaaagatttttctgcatccttgcctgagaattattttttgcacgagatttataagaaaaccatctcaaacgcgctcaaaaaccctataatcatccattatgatgtttggtggggggcggtgaagccttgggactatccttttggtttaaaagcggatttgtggctgaacgctttggctaaaaccccttttatgagcgactggattgattcgatcgctagggtggaaataggcagcgaaaaatggcatcgttaccacagcatcgttgcctatcactactactttcccctatggaagactgaagagcagatcgcccacgacgcgctcaagacctttttagaccattatttttcgtgcatccatgccacaatcaagcaagaaaatctcggaatgttcttgaaccactacttctcgcatgcccatgcagagatcaaagaaaactcccttgaagcgtttctgaatcattattttagccacgtttataggctccctaaaaacgtgcgaaagaaactctttagggtgtttatcaaacactgcatcctcataccactcaagagccttgtgggtaagactctgcgattcttaaaactccatgcactggtgaaaaacatcctaatccaactcaagctcttaaaaaagagctag

SEQ ID NO: 47 - DNA, 1167 bp, Helicobacter cetorum
atgatttccacagttattaccccgccgccggaaaaaactcaacaaagtgagttaaatgagtattctcatattcctatagcttttgcttttgacaaaaattattgcattccttttgcagcatgccttcaatcctttttagaatgtattgctagggcaaacaaacaagtcttttactccttgcatgctctagtagtgggtttagatgaagatgatattcaaaagctccaccaaattgcagagccctttaaagaaatggctactttagaaataaaaaatattgagccttttttggacactatcaaaaacccatttgatgaaagttttaccaaacgattttcaaaaatggttttaattaaatactttttagcagatttgtttcctaaatattccaaaatagtttggagtgatgtagatatcattttttgtgaagaatttagcaaagattttttaaaaataacagaagatgacaaaaattatttgtatggtgtctttgatggcaaacaacatgtattagagggatttttattctgcaacttagctttacaaagaaaaaatcattttacacaaaaaatccaagaaattttgcatgctcaagaagttaccgaagaaccgcacttgactgactggtgttggccatatatagggcaacttggcatagagtattgtgtttttccctcatactatatcttaaatcaagagaatgaactttttctttatgaaagactttatgataactattcaaaacatgtccaacaagccttaaaaaaccccattattatccactacgatggttggatacatgcaataaaaccttgggataatcccttaagcatgaaagctaatctatggctaaacactttagctaagactcctttctttacagattacacagaattattgcaaaaaaatacaaatttttatggcaaaattattagcaaagactattactttccttattacgcaagctatacagataccattttaaaacctctctacctattttttcaaaactattctgcttatttgaaagaacgggttttttgtgaagaattttatatgagagtaattaaaatccctttaagcaaaactcttaaaagacttaaaattcatagggctttaaaaaagatattactagcactaaaaattatcaaatcaaatgcaaactaa

SEQ ID NO: 48 - DNA, 1047 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcaaacttggtgcaacttggatattttgattgtcgatgacggctcgacagacggtacgcttgccattgccaaggattttcaaaagcgggacagccgtatcaaaatccttgcacaagctcaaaattccggcctgattccctctttaaacatcgggctggacgaattggcaaagtcgggcggcggtgaatatattgcacgcaccgatgccgacgatattgccgcccccgactggattgagaaaatcgtgggcgagatggaaaaagaccgcagcatcatcgcgatgggcgcgtggctggaagtgttgtcggaagaaaaggacggcaaccggctggcgcggcatcacaggcacggcaaaatttggaaaaagccgacccggcacgaagatattgccgactttttccctttcggcaaccccatacacaacaacacgatgattatgcggcgcagcgtcattgacggcggtttgcgttacaacaccgagcgggattgggcggaagattaccaattttggtacgatgtcagtaaattgggcaggctggcttattatcccgaagccttggtcaaataccgccttcacgccaatcaggtttcatccaaatacagcgtccgccaacacgaaatcgcgcagggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgctgcccgccggcgcgtggctggattttgcggcagacggcaggatgcggcggctgtttaccttgaggcaatacttcggcattttgcaccgattgctgaaaaaccgttga

SEQ ID NO: 49 - DNA, 1047 bp, Neisseria meningitidis
atgccgtctgaagccttcagacggcatcgtgcctatcgggagaataaattgcagtctttagtcagcgtattgatttgcgcctacaacgtagaaaaatatttcgcccaatcattagccgccgtcgtgaatcagacttggcgcaacttggagattttgattgtcgatgacggctcgacagacggtacgcttgccattgccaaggattttcaaaagcgggacagccgtatcaaaatccttgcacaagctcaaaattccggcctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcatcgcgatgggcgcgtggctggaagttttgtcggaagaaaaggacggcaaccggctggcgcggcatcacaggcacggcaaaatttggaaaaagccgacccgacacgaagatattgccgactttttccctttcggcaaccccatacacaacaacacgatgattatgaggcgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgccgcccgccggcgcgtggctggattttgcggcagacggcaggatgcggcggctgtttaccttgaggcaatacttcggcattttgcgccgattgctgaaaaaccgttga

SEQ ID NO: 50 - DNA, 1047 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggatattttgattgttgatgacggctcgacggacggcacacttgccattgcccggcgtttccaagaacaggacggccgcatccgtatcctcgcgcagccgcgcaattccggcctgattccctctttaaacatcgggctggacgaattggcaaagtcaggaatgggggaatatattgcacgcaccgatgcagacgatattgccgcccccgattggattgagaaaatcgtgggcgagatggaaaaagaccgcagcatcattgcgatgggtgcgtggctggaagttttgtcggaagaaaaggacggcaaccggctggcgcggcatcacgaacacggcaaaatttggaaaaagccgacccggcatgaagacattgccgccgtttttcctttcggcaaccctatacacaacaacaccatgattatgagacgcagcgtcattgacggcggtttgcgttacaacaccgagcgggattgggcggaagattaccaattttggtacgatgtcagcaaattgggcaggctggcttattatcccgaagccttggtcaaataccgccttcacgccaatcaggtttcatccaaatacagcgtccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgtttcaaacggacggacacgccgcccgccggcgcgtggctggattttgcggcagacggcaggatgcggcggctgtttaccttgaggcaatacttcggcattttgcgccgattgctgaaaaaccgttga

SEQ ID NO: 51 - DNA, 1089 bp, Neisseria meningitidis
atgccgtctgaagccttcagacggcatcgtgcctatcgggagaataaattgcagcctttagtcagcgtattgatttgcgcctacaacgtagaaaaatatttcgcccaatcattagccgccgtcgtgaatcagacttggcgcaacttggatattttgattgtcgatgacggctcgacagacggtacgcttgccattgcccagcgtttccaagaacaggacggtcgtatccgcatcctcgcgcagccacgcaattccggtctgattccctctttaaacatcgggctggacgaattggcaaagtcaggaatgggggaatatattgcacgcaccgatgccgacgatattgccgcccccgactggattgagaaaatcgtgggcgagatggaaaaagaccgcagcatcattgccatgggtgcgtggctggaagttttgtcggaagaaaaggacggcaaccggctggcgcggcatcacaggcacggcaaaatttggaaaaagccgacccgacacgaagatattgccgactttttccctttcggcaaccccatacacaacaacacgatgattatgaggcgcagcgtcattgacggcggattgcgttacaacaccgagcgggattgggcggaagattaccaattttggtacgatgtcagcaaattgggcaggctggcttattatcccgaagccttggtcaaataccgccttcacgccaatcaggtttcatccaaatacagcatccgccaacacgaaatcgcgcaaggcatccaaaaaaccgccagaaacgattttttgcagtctatgggttttaaaacccggttcgacagccttgaataccgccaaataaaagcagtagcgtatgaattgctggagaaacatttgccggaagaagattttgaacgcgcccgccggtttttgtaccaatgcttcaaacggacggacacgccgcccgccggcgcgtggctggatttcgcggcagacggcaagatgaggcggctgtttaccatgaggcaatacttcggcattttgcaccggctgattaaaaaccgccggcaggcgcggtcggattcggcagggaaagaacaggagatttaa

SEQ ID NO: 52 - DNA, 1491 bp, Helicobacter pylori
atgacttcagtttcaagccattcttttaaaaaacaagattttcatatccctatcgcttttgcttttgataagaattacctcattcctgcgggcacgtgtctttattccttgctagaaagcatcgctaaagccaataaaaaaatccgttacaccatacacgctttagtggtaggcttgaatgaagaagataaagcaaggcttaatcaaatcgcagagccttttaaagaatttgccgctttggaagtgagagatatcgaaccttttttagacgctatccctaacccttttgatgaggatttcaccaagcgtttttctaaaatggtgttagtgaagtattttctagcggatctatttcccaaatattccaaaatggtgtggagcgatgtggatgttatcttttgcaatgaatttagcgctgatttcttaaacattgaagaaaatgatgagaattatttttatggggttttaggaggtggagagtgccatgtgttggaagggtttttgttctgtaacttagattaccagcgcaagaaccatttcgtttcaaaaacccatgcgcttttggaagaaaacagggctacagaagaattggattttacaaaatggtgttggcctaacatgaaagctttagggattgagtattgcgtttttccttattattacactgtcaaggatttttctgcatccttgcctgagaactattttttgcatgagatttataagaaaaccatctcgaacgcgctcaaaaatcctatagtcatccattatgatgtttggtgggaagcggtgaaaccttgggattacccttttggtttaaaagcggatttgtggctgaatgctttggctaaaaccccttttatgagagactggattgattcgatcgctagggtggaaatcggcagcgaaaaatggcatcgttaccacagcatcgttgcctatcactactactttcccctatggaagactgaagagcagatcgcccatgacgcgctcaagacctttttagatcattacttttcccgcatccatgccacaatcaagcaagaaagtctcggaatgtttttgaatcattacttctcgcacgcacatgccgaaatcaaagagaactccttggaaatgtttcttaaccattatttctcgcatgcacacgccgagataaaagagaactcattggagatgtttcttaaccactacttctcccatgcccatgcagaaatcaaagaaaactcgcttgaaatgtttttgaaccactacttctcgcatgcacatgccgaaattaaagaaaactcccttgaaatgtttttagaccactatttctcgcatgtttataggctccctaaaaaagcacggaagagactctttagggtgtttgtcaaacactgcattctcataccgcttaagagtcttattagtaaaacccttaaaatcctagggcttcatgggcttgtgaaaaaaatcctaatccaactcaagctcttaaaaaagagctaa

SEQ ID NO: 53 - DNA, 1296 bp, Helicobacter pylori
atgacttcagcttcaaaccattctttcaaagaacaagattttcatattcctatcgctttcgcttttgataagaattaccttattcctgcaggcgcatgcatttattccttgctagaaagcatcgctaaggctaataaaaaaatccgttacaccctacacgctttagtggtaggattgaatgaagaagataaagcaaagcttaaccaaatcacagagccttttaaagaatttgctgttttagaaataaaagatattgaaccttttttagacgctatccctaacccttttgatgaggatttcaccaagcgtttttctaaaatggtgttagtgaagtattttctagcggatttattccccaaatattctaaaatggtgtggagcgatgtggacgttatcttttgtaatgaatttagcgctgatttcttaaacattaaagaagatgatgagaattatttttacggggttttagaaggtggagagcgccatgtgttggaagggtttttgttttgtaatttagattaccagcgcaagaaccattttgtctcaaaaacccatgcacttttggaagaaaacaaggctacagaagaattggatttcacccaatggtgttggcccaatatgaaagctttagggattgaatattgcgttttcccttattattacaccattaaagatttttctaacgcgtatttaaacgagaattacaagaaaaccattttagaggcgcgagaaaaccctaccattatccactatgacgcttggtggggggcggtgaagccttgggattacccttttggtttgaaagcggatttatggctgaacgctttggctaaaaccccatttatgagcgactggattgattcgattgctagggtggaaatcggcagtgaaaaatggcatcgctaccatagcatcgttgcctatcactactactttcccctatggaagactgaagagcagatcgcccacgacgcgctcaagacctttttagaccattatttttcgtgcatccatgccacaatcaagcaagaaaatctcggaatgttcttgaaccattacttctcgcacgcccacgcagagatcaaagaaaactcccttgaagcgttcttgaaccactatttctcgcatgtttataggctccctaaaaacgcgcgaaagaaactctttggggtgtttgtcaaacattgcatcctcataccactcaagagccttattagtaaaacccttaaaatactaggacttcatgcgctagccaaaaaaatcctaatccaactcaagctcttgaaaaagagctag

SEQ ID NO: 54 - DNA, 1371 bp, Helicobacter pylori
atgacttcagcttcaaaccatagcttaaaagaacaagattttcatatccctatcgctttcgcttttgataagaattaccttattcctgcgggtgcatgcatttactccttgctagaaagcatcgctaaggctaataaaaaaattcgttacaccttacacgctttagtggtaggcttgaatgaagaagataaagcaaagcttaaccaaatcgcagagccttttaaagaatttgctgttttagaagtaaaagatattgaaccttttttagacactatccctaacccttttgatgaggatttcaccaagcgtttttctaaaatggtgttagtgaagtattttttggcggatttgttccccaaatattccaaaatggtgtggagcgatgtggatgtgatcttttgtaatgaatttagcgctgatttcttaaacattaaggaaaatgatgagaattatttttatggggttttaggaggtggagagtgccatgtgttggaagggtttttgttttgtaatttggattaccagcgcaagaaccattttgtctcaaaaacccatgcacttttggaagaaaacaaggctacagaagaattggatttcacccaatggtgttggcctaacatgaaagctttagggattgagtattgcgttttcccttattattacactgtcaaagatttttctgcatccttgcctgagaattattttttacatgagatttataaaaaaaccatctcaaacgcgctcaaaaacccgataatcatccattatgatgtttggtggggggcggtgaagccttgggattatccttttggtttaaaagcggatttatggctgaacgctttggctaaaaccccttttatgagcgactggattgattcgatcgctagggtggaaataggcagtgaaaaatggcatcgctaccacagcatcgttgcctatcactactactttcccctatggaagactgaagagcagatcgcccatgacgcgctcaagacctttttagaccattatttttcgtgcatccatgccacaatcaagcaagaaaatctcggaatgttcttgaatcattacttctcgcatgcacacgccgaaatcaaagaaaactcgttggaggcgttccttaaccactacttctcccatgcccatgcagagatcaaagaaaactcccttgaagcgttcttgaaccactacttctcgcatgtttataggctccctaaaaacgcacaaaagaaactctttagggtgtttatcaaacactgcatcctcataccactcaagagccttgtgggtaagactctgcgattcttaaaactccatgcactggtgaaaaaaatcctaatccaactcaagctcttaaaaaagtcttag

SEQ ID NO: 55 - DNA, 1371 bp, Helicobacter pylori
atgacttcagcttcaagccattctctcaaagaacaagattttcatatccctatcgcttttgcttttgataagaattacctcattcctgcgggcgcgtgtctttattccttgctagaaagcatcgctaaagccaataaaaaaatccgttacaccctacacgctttagtggtgggcttgaatgaagaagataaagcaaagcttaatcaaatcgcagaaccttttaaagaatttgccgctttggaagtgagagatattgagtcttttttagacactatccctaacccttttgatgaggatttcaccaagcgtttttctaaaatggtgttagtgaaatattttctagcggatttattccccaaatattctaaaatggtgtggagcgatgtggatgttatcttttgcaatgaatttagtgctgatttcttaaacattaaagaaaatgatgagaattatttttatggcgttttaggaggtggagagcgccatgtgttggaagggtttttgttttgtaatttagattaccagcgcaaaaaccattttgtctcaaaaacccatgcacttttggaggaaaacaaggctacagaagaattggatttcacccaatggtgttggcccaatatgaaagctttagggattgagtattgcgttttcccttattattatactgtcaaagatttttctgcatccttgcctgagaattattttttgcatgaaatttataagaaaaccatctcaaacgcgctcaagaaccctataatcatccattatgatgtttggtggggggcggtgaagccttgggattatccttttggtttaaaagcggatttgtggctgaacgctttggctaaaaccccatttatgagcgattggattgattcgatcgctagggtggaaatagggagcgaaaaatggcatcgttaccacagcatcgttgcctatcactactactttcccctatggaagactgaagagcagatcgcccatgacgcgctcaagacctttttagaccattatttttcgcgcatccatgccgcaatcaagcaagaaaatttgggaatgttcttaaaccattatttctcgcatgcacacgccgaaatcaaagagaactccttgaaagcgttccttaaccactacttctcgcatgcccatgcagagatcaaagaaaactcccttaaagcgttcttgaaccactatttctcgcatgtttataggctccctaaaaacgcgcaaaagagactctttagggtgtttgtcaaacactgcatcctcataccactcaagagtcttattagtaaaacccttaaaatcctaggacttcatgggcttgtgaaaaaaatcctaatccaactcaagctcttaaaaaagagctaa

SEQ ID NO: 56 - DNA, 1311 bp, Helicobacter pylori
atgacttcagcttcaaaccattcttttaaagaacaagattttcatattcctatcgcttttgcttttgataagaattacctcattcctgcgggcgcgtgtctttattccttgctagaaagcatcgctaaagctaataaaaaaatccgttacaccctacacgctctagtggtaggcttgaatgaagaagataaagcaaagcttaatcaaatcgcagagccttttaaagaatttgctgctttagaagtaaaagatattgaaccttttttagacactatccctaacccttttgatgaggatttcaccaagcgtttttctaaaatggtgttagtgaagtattttttggcggatttgttccccaaatattctaaaatggtgtggagcgatgtggacgttatcttttgcaatgaatttagtgctgatttcttaaacattaaagaaaatgatgagaattatttttatggggttttaggaggtggagagcgccatgtgttggaagggtttttgttttgtaatttagattaccagcgcaagaaccattttgtctcaaaaacccatgcacttttggaagaaaacaaggctacagaagaattggatttcacccaatggtgttggcccaatatgaaagctttagggattgaatattgcgttttcccttattattacactgtcaaagatttttctgcatccttgcctgagaattattttttgcatgagatttataagaaaaccatctcaaacgcgctcaaaaatcctataatcatccattatgatgtttggtggggggcggtgaagccttgggattatccttttggtttaaaagcggatttgtggctgaacgctttggctaaaaccccttttatgagcgactggattgattcgatcgctagggtggaaataggcagcgaaaaatggcatcgttaccacagcattgttgcctatcactactactttcccctatggaagactgaagagcagatcgcccacgacgcactcaagacctttttagaccattatttttcgtgcatccatgccacaatcaagcaagaaaatctcggaatgttcttgaaccattacttctcgcatgcccatgcagagatcaaagaaaactcccttgaagcgttcttgaaccactacttctcgcatgtttataggctccctaaaaatgcgcgaaagaaactctttagggtgtttatcaaacactgcatcctcataccactcaagagccttgtgggtaagactctgcgattcttaaaactccatgcactggtgaaaaaaatcctaatccaactcaaactcttaaaaaagagctag

SEQ ID NO: 57 - DNA, 894 bp, Escherichia coli
atgttttcaattataatacctagctttaatcgagagaaacaacttcgtaatgcattggccgaactaaataagcaactctacaaagagtttgaggttattgttgttgatgatgcttcggacagtgaatatgatttgagcttggatctttatcattttgaactgaaatatttcagaagcaataaaaatttaggaccatcaggtgctagaaatattggagttagtttagctcgttatgattggatcatttttttggacgatgatgattatttcatgccggaaaaactgatgatcttgagggagactatattaaataatgatattgactttatctatcatagagctcgtatatttatgatcaatgaaaaaattagttatatttctagtcaaaaggatatagataaaattccaggtgaggtatatcaacatatgctttctggtaattttattgggggccctccaaattttgcaataaaaaaaagcctgtttaacaaacttcatggcttttctgataatgtacgagcaattgaagattacgagtttctactaaggttagtccgtgacgttagtaaaaaaagaattatttttgtagataatgtgcttactgggtgtaattatataacaaaatcaaccagcgtgtctaaaaatataaacaatttaaaacaagcatgtacatatatatcagaggaatatattcgagacaaccgcgaatttaatctttttaaaataaatacctcgttgatgactgcacatgctttgttaatgagtttaaatcgtaaatctgctttcttttattttaaagcagggattgttatgtttaatataaaatatattgtatctgggattatttcttttatatctccagttttgatgattaagcttaggggcaaggtttaa

SEQ ID NO: 58 - DNA, 798 bp, Escherichia coli
atgattattgatgaagcggaaagcgcggaaagcacccatccagtggtgagcgtgattctgccggtgaataaaaaaaacccgtttctggatgaagcgattaatagcattctgagccagacctttagcagctttgaaattattattgtggcgaactgctgcaccgatgatttttataacgaactgaaacataaagtgaacgataaaattaaactgattcgcaccaatattgcgtatctgccgtatagcctgaataaagcgattgatctgagcaacggcgaatttattgcgcgcatggatagcgatgatattagccatccggatcgctttaccaaacaagtggattttctgaaaaataacccgtatgttgatgttgtgggcaccaacgcgatttttattgatgataaaggccgcgaaattaataaaaccaaactgccggaagaaaacctggatattgtgaaaaacctgccgtataaatgctgcattgtgcatccgagcgtgatgtttcgcaaaaaagtgattgcgagcattggcggctatatgtttagcaactatagcgaagattatgaactgtggaaccgcctgagcctggcgaaaattaaatttcagaacctgccggaatatctgttttattatcgcctgcatgaaggccagagcaccgcgaaaaaaaacctgtatatggtgatggtgaacgatctggtgattaaaatgaaatgcttttttctgaccggcaatattaactatctgtttggcggcattcgcaccattgcgagctttatttattgcaaatatattaaataa

SEQ ID NO: 59 - DNA, 798 bp, Escherichia coli
atgataatcgatgaagctgaatctgccgaatcaactcatcctgttgtttctgttattctgccagttaataaaaaaaacccttttcttgatgaggcaataaatagtattttatcgcaaacattttcgtcattcgagataataatagttgcaaattgttgtacggatgatttttataatgagttgaaacacaaagttaatgacaaaattaagttgattcgtacaaatattgcttatttaccgtactcattaaataaagccatcgatttgtccaatggtgagtttattgcaaggatggattccgatgatatttctcatcctgatagattcacgaaacaagttgattttttaaaaaataatccttatgtggatgtcgtcggtactaatgcaatatttattgatgataaaggtcgagaaataaacaaaacaaagctacctgaagaaaatttggatattgtaaaaaacttaccgtataaatgttgcattgttcatccatctgtaatgtttaggaagaaagtaatcgcttcaattggcggttatatgttttcaaactattctgaggattatgagttatggaatagattaagtttagcaaaaataaaatttcaaaatttaccggaatatttattctattacaggttgcatgaaggtcagtcaactgctaaaaaaaacttgtatatggttatggtaaatgatttggtaataaagatgaaatgcttttttttgacaggtaatatcaactatctcttcggagggattagaactattgcttcttttatatactgtaaatacataaagtga

SEQ ID NO: 60 - DNA, 828 bp, Pseudogulbenkiania ferrooxidans
atggacaaaataaagcaaggatcagcgtcactagtggtaggcgaccaacaagagaagcatccagtagtatcagtactcctacccgtaaatagggtagaccggttcttcattcccgctgtagaaagcatactaacccagacactccaagacttcgagctaattataatagctaatggctgtagcaccgaacacctaaacaagataaggctcacatacggcgatcacaatagggtaaggattctgaacacagagattaagggactacccttcgcgctaaatctaggagtacacaacgctcgcggactatacatagcgaggatggacgcggatgatataagtataccagagaggttagaaaagcagctcaataccctagagcagaataagaagataggagtggtaagcagcggggtagacttcatagacgagaacgatcaagctataagggagggaaagttccccgagctaacggataaggatcacaggaggctgctacccctcatttgctgcatagctcaccccacagtgatggtaaggaaagagataattaataaactcggaggatactcattcggaagcttttcagaggactacgacctatggctaaggataatgcgcgaactgccagaagtagagttttatcgcataccggagagcctactcaagtacaggaggcacggaaatcaagctacctcatcaaagaatataaaaaagataagggcgtacaacagtgctctaaaaataagggaactattcctaagcaggaagctaaaattcataataggaattatattacccgcgaggatggtgacgctatggaggaaatga

SEQ ID NO: 61 - DNA, 828 bp, Pseudogulbenkiania ferrooxidans
atggataagatcaaacaggggtctgccagtttggtcgtgggagatcagcaggaaaaacaccctgttgtttccgttcttcttcctgttaacagagtcgataggttttttatacctgccgttgagtcaattctcacgcaaactttgcaggattttgaacttatcattatcgccaacggttgctctacagagcatttgaataaaattcgccttacttatggtgaccataaccgagttcggatccttaatacggaaatcaaagggctcccttttgccctcaacttgggggttcataatgcacgtggcttgtatatcgctagaatggatgccgacgacatttccattcctgaacgcttggagaaacaacttaacactctggaacaaaacaaaaaaatcggggtcgtttcgtctggagtggattttatcgatgaaaatgaccaggcaatccgagaagggaaatttccagaattaaccgacaaagaccatcgtagattgttacctcttatctgttgtattgcccatcctacggtaatggtccgaaaggaaatcatcaacaagcttgggggctattcgtttgggagtttcagcgaagattatgatttgtggcttagaatcatgcgtgagttaccggaggtcgaattctaccgtattccagaatcgttgcttaaatatcgtcgacatgggaaccaggcaacatccagcaaaaacattaagaaaatccgagcttataattccgccctcaagatccgcgagttatttttatctcgaaaattgaagtttatcatcgggataattcttcctgcacggatggtcaccttgtggcgtaagtag

SEQ ID NO: 62 - DNA, 828 bp, Pseudogulbenkiania ferrooxidans
atggataagattaaacaggggagcgcttctctcgttgtcggtgatcagcaggaaaaacacccggttgtttctgtccttcttcctgttaacagagtggatagattttttatcccggccgtggagagtattcttacgcaaactcttcaggattttgaattgatcatcattgcaaacgggtgctccacggagcatttgaataaaatccgtcttacttatggtgaccataaccgagttagaatcttgaatacggaaatcaaagggctcccatttgccttgaacctcggggtccataatgcacgggggctctatatcgccagaatggatgccgacgacatttccatccccgaacgattggagaaacaacttaacacacttgaacaaaacaaaaaaatcggggtagtgtcttctggagtcgattttattgatgaaaatgaccaggccattcgagaagggaaatttccagaactcaccgacaaagaccatcgacgacttttgccattgatctgttgtatcgcacatccgacggtaatggttagaaaggaaattatcaacaagcttggtgggtatagttttgggtcattctcggaagattatgatctctggcttcgaattatgcgtgagctacccgaggtcgaattctaccgtatccctgaatcacttttgaaatatagacggcatgggaaccaggcaactagtagtaaaaacattaagaaaatccgcgcatataattccgcgttaaagattagagagttgtttctttcccgaaaactcaagtttattattggtataattttgcctgctagaatggtaacattatggcgaaagtag

SEQ ID NO: 63 - DNA, 795 bp, Salmonella enterica
atgttaacagaagtcagacctgtttctactactaaaccgctagtatcagttattttacctgtaaataaatttaatccttatttagatagggcaattcatagtattttatcacaatcttatccttcaattgaattgattataatagcaaataattgcacaaatgatttttttgatgctttaaagaaaagagaatgtgaaactataaaggtgcttagaacaaatatcgcatatttaccatattgtttaaataaggggcttgatctttgtaatggagattttgttgcccgcatggattctgatgatatatcacatcctgaaaggattgatcgacaagttgactttttaataaataatccggatatagatgtcgttggcaccaatgcagtatatattgatgaggacgatattgaacttgagaaaagtaatttgcctgtaaataacaatgccataagaaaaatgctaccctacaaatgctgtttagtacatccttcagtaatgtttagaaaaaatgtggtaataacctctggtgggtatatgtttgcaaattattctgaagattatgagttatggaatagattggcagtggagggaaggaatttttataatttatctgaatatttgctttattatcgacttcataataatcaaagtacatcaaagaataacttatttatggttatggccaatgatgtggcaattaaagttaaatattttttgttaactaaaaaaataagttatcttttggggattattaggactgttttttctgttttttattgtaaatatataaagtga

SEQ ID NO: 64 - DNA, 795 bp, Salmonella enterica
atgttgacggaggttcggcccgtgagtacgacgaagcctctcgtttctgtaatcctccccgttaacaagttcaacccatacctcgacagagcgatccactccatcttgtctcagtcctaccccagtatcgagctcataattatcgccaacaactgtaccaacgacttcttcgacgccttgaaaaagagggagtgcgagacaatcaaagtcctcaggactaacattgcctacctcccttactgcttgaacaaaggactcgacctctgcaacggggacttcgtagctcggatggactccgacgacatttctcaccccgagcggatcgaccggcaggtggatttcctaatcaacaacccagacatcgacgttgtaggtacaaacgctgtctacatcgacgaagatgacatagagctcgaaaagtccaacttaccagtgaacaataacgcaatcaggaagatgttgccttataagtgttgccttgttcacccaagcgtcatgttccggaagaacgtagtcattacaagtggcggatacatgttcgcgaactacagcgaggactacgaactctggaaccgactcgccgtcgaaggtcggaacttctacaacctttccgagtacctcctctactacagattgcacaacaaccagtcgactagtaaaaacaatcttttcatggtcatggcaaacgacgtagctataaaggtgaagtacttcttattgacgaagaagatctcttacctcttaggtataatacgaacagtcttcagtgtattctactgcaagtacatcaaatag

SEQ ID NO: 65 - DNA, 795 bp, Salmonella enterica
atgttgacggaggtgaggccagtgtccacgacgaagcctttagtcagcgtaatcttgcccgtcaacaagttcaacccatacctcgacagagctatccactcgatactcagtcagagttacccaagtatcgagttaatcatcattgccaacaactgtactaacgacttcttcgacgcattgaaaaagcgagagtgcgagacaattaaagttctccgaactaacattgcttacttgccttactgcctgaacaaaggactcgacctctgcaacggggacttcgtcgctcggatggacagtgacgacatttcgcacccagagcgaatcgacaggcaggtcgatttcctcattaacaacccagacatcgacgtagtcggtacaaacgccgtctacatcgacgaagatgacatcgagctcgaaaagtccaacctccccgtcaacaataacgctatcaggaagatgttgccatataagtgttgcctcgtccacccctctgtcatgttccgaaagaacgtcgtcattacatccggcggctacatgttcgctaactacagtgaggactacgaattgtggaaccggcttgctgttgaagggcgaaacttctacaaccttagtgagtacttactctactacagactccacaacaaccagtcgactagtaaaaacaatcttttcatggtgatggctaacgacgttgctatcaaggtaaagtacttcctcttgacaaagaagatctcgtacctccttggtatcatcagaacagtattctcagtcttctactgcaagtacatcaaatag

SEQ ID NO: 66 - DNA, 825 bp, Corynebacterium glutamicum
atgacttcaagcgccccgctcatatcagtagtaattccgacaattgcctacgatgaatactgctcacaatctatcaaatcagtttgcgaacaaaactacgaaaactggcaaatagttctcgttctggatggagccccaatcaaagatgttccacaatgggtaaaagaacacgaacgcatcaaaatcgttgaacaaaaaatcagacaaggtaccccaacatcactcaacaatgggattaaagcctctgatgggcagctcatcgcgcggctcgattcagatgatctggccgccccatcacgcctcagcaaacaagaagaattcctgagaaaccacccgtatatcatctgcgttgcgacaaaaacaaaacatatcaatgaacacggaaaaattttcgggcaatcagccgatttaccgactagccaggatattagacaaattctgctcgtgaaaaacccgattatccatagctcagttatgtatcgcaaacaagttgttgaacagattggagggtattctctggaaatgactcggagccaggattacgaactctttttaagactgagcaaaatcggggcgattgggtatttagatgaatcattatcatcatatcgcatccacggtggccagcattcaagaaaaaccagcccgttcaaaaaatacacctggattatactcaaacggagaatggaattagcctcatttctcaaacgatcaccggttcggcaaatatttctcaatttcatatggtacggcgcccaagttactcggtatctgggactgcggaaagcggggtttatgacagggtataccccagatcgcatttaa

SEQ ID NO: 67 - DNA, 828 bp, Neisseria meningitidis
atgcagaaccatgttattagcctggcaagcgcagcagaacgtcgtgcacatattaccgatacctttggtgttcgtggtattccgtttcagttttttgatgcactgatgccgagcgaacgtctggaacaggttatggcagaactggttccgggtctgagcgcacatccgtatctgagcggtgttgaaaaagcatgttttatgagccatgcagttctgtggaaacaggcactggatgaaggtctgccgtatattaccgtttttgaagatgatgttctgctgggtgaaggtgcagaaaaatttctggcagaagatgcctggctgcaagaacgttttgatccggatagcgcatttattgttcgtctggaaaccatgtttatgcatgttctgaccagcccgagcggtgtggcagattatggtggtcgtgcatttccgctgctggaaagcgaacattggggcaccgcaggttatatcattagcaaaaaagccatccgcttttttctggatcgttttgcagcactgcctccggaaggcctgcatccggttgatctgatgatgtttagcgatttttttgatcgtgaaggtgttccggtttgtcagctgaatccggcactgtgtgcacaagaactgcactatgcaaaatttcatgatcagaatagcgcactgggtagcctgattgaacatgatcgtctgctgaatcgtaaacagcagtggcgtgatagtccggcaaatacctttaaacgtcgtctgattcgtgcactgaccaaaattagccgtgaacgtgaaaaacgtcgccagcgtcgtgaacagtttattgtgccttttcagtaa

SEQ ID NO: 68 - DNA, 828 bp, Neisseria meningitidis
atgcagaaccatgttattagccttgctagcgcggcagaacgccgagcccatatcgcggacacctttggccgacatggcattccgttccaattctttgatgcactgatgccgtcagaacgactggaacaagcaatggccgaattagtgccaggcttatcggcccatccatacttaagcggagttgaaaaagcgtgcttcatgagccatgcggtattatggaagcaagcattagatgaagggctgccgtacattaccgtattcgaggatgatgttcttttaggcgaaggggccgaaaaatttcttgcggaagatgcttggctgcaggaacgcttcgatccggacaccgcgttcattgtgcgcttagaaacgatgttcatgcatgtgctgacctcgccaagcggcgttgccgactattgcggacgcgcgttcccgctgttagaaagcgaacattggggaacggccggctacattatcagcagaaaagccatgcggttctttctggatcgattcgcggcgctgccgccagaaggactgcatccagtggacctgatgatgtttagcgacttctttgatcgagaaggaatgccggtttgccaattaaatccagcgttatgcgcgcaggagctgcactacgcgaagttccatgatcagaacagcgcattaggcagcctgattgaacatgatcgcttactgaaccgcaaacaacagcgacgcgacagcccagcgaacacatttaaacatcgcctgattcgcgcgttaaccaaaattagccgagaacgagaaaaacgccggcagcgacgcgaacaatttatcgttccttttcagtaa

SEQ ID NO: 69 - DNA, 828 bp, Neisseria meningitidis
atgcaaaatcacgtcatctcgttagcatcggccgctgagagaagagcgcacattgccgatacgttcggtaggcacggaatcccatttcagtttttcgacgctttaatgccatccgagcgtcttgagcaggctatggcggagctcgttcctggtctgtctgcgcacccctatttgtcaggggtcgagaaggcatgttttatgtcacacgccgtcttgtggaaacaggctctagacgagggattgccctatataacggtctttgaagacgacgtcttgctcggtgagggcgcggagaagttcctcgccgaggacgcgtggttgcaagagcgttttgaccctgatactgcctttatcgttcgtttggagactatgtttatgcacgtacttactagtcctagtggtgtcgcagattactgtgggagagcatttcctttactcgagtcggagcactggggtacagctggttatataatatctcgaaaggcgatgcgatttttcttagacagatttgccgccctaccccctgaggggttacaccctgttgatttgatgatgttctcagattttttcgacagagaggggatgccagtctgtcagcttaaccccgcactctgtgcccaagaattacattatgctaaatttcacgaccaaaattccgccttgggttcccttatcgagcacgaccgtttgttgaataggaagcagcaaaggcgtgattcccctgcaaatactttcaagcaccgtttaatacgggcactgacaaagatatctagagagagagagaagcgtaggcaaagacgtgagcagttcattgtcccattccaatag

SEQ ID NO: 70 - DNA, 828 bp, Neisseria meningitidis
atgcaaaatcacgtcatctcgttagcgtccgccgccgagaggcgggcacacatagcagatactttcggtaggcacgggatcccttttcagtttttcgacgcccttatgcccagtgagaggttagagcaggctatggctgagcttgttcccgggttgagtgcacacccttatctctctggggtagagaaggcctgttttatgtcgcacgcagtcctttggaaacaggccttggacgagggacttccatatatcacagtctttgaagacgacgtcctcctcggtgagggtgcagagaagttcctcgccgaggacgcctggcttcaagagcgttttgacccagatacggcctttatcgtccgtcttgagacaatgtttatgcacgttttaacgagtccttcgggtgtagcggattactgtgggagggcctttcctctcttggagtccgagcactggggcacagcaggttatataatatctaggaaggcaatgagatttttcttagacagatttgccgcactcccccctgagggtttgcaccccgtcgatttaatgatgttctcggattttttcgaccgggagggtatgcccgtatgtcagttgaaccccgccctttgtgcacaagaattacattatgcaaaatttcacgaccaaaattcggctcttgggagtttgatcgagcacgaccgtcttctcaataggaagcagcaaaggcgtgattctcccgcaaatactttcaagcaccgtttaatccgtgctcttacgaagatcagtcgggagagggagaagaggagacaacggcgtgagcagttcattgtaccattccaatga

SEQ ID NO: 71 - DNA, 948 bp, Streptococcus agalactiae
atgataaagaaaatagaaaaggatctgataagcgttattgttccgatttataacgtggaagattatctggtggaatgcatagaatcattaattgtgcagacctatcggaacattgaaatactgttaattaacgatggctcaacagataactgcgctaccattgctaaagaatttagcgaacgggattgccgggtgatatatatagaaaagtcaaacggcggattaagcgaagcgcggaattatggtatttatcatagcaagggtaaatacctgacctttgtggatagcgatgataaagtgtcaagcgattacatagctaacttatataacgcgattcagaaacatgattcaagcatagcgataggcggatatctggaattttatgaacggcataactcaatacggaactatgaatatctggacaaagtaataccggtggaagaagcgttgttgaatatgtatgacattaaaacctatggcagcatttttattaccgcgtggggtaaactgttccataaaagcatattcaacgatctggaatttgcgctgaacaagtatcatgaggatgaattctttaattataaagcgtacctgaaagcgaacagcataacctacatagacaagccgctatatcattatcgcatacgggttggcagcataatgaacaacagcgataacgtgataattgcgcggaagaaattagatgtgctgagcgcgctggacgagcggataaaattaataaccagcctgcgcaagtattcagtgtttctgcagaaaaccgaaatattttatgtaaaccagtattttcgaaccaaaaaatttctgaaacagcagagcgttatgtttaaagaagataattatatagatgcgtatcggatgtatggtagactgttacggaaagtaaagctggtggataaactgaaattaataaaaaaccgatttttctga

SEQ ID NO: 72 - DNA, 822 bp, Helicobacter pylori
atgcgtgtttttatcatttctttaaatcaaaaagtgtgcgatacatttggtttggtttttagagacaccacgactttactcaataatattaatgccacccaccaccaagcgcaaatttttgatgcgatttattctaaaacttttgaaggcgggttgcaccccttagtgaaaaagcatttacacccttatttcatcacgcaaaacatcaaagacatggggattacaaccaatctcatcagtgaggtttctaagttttattacgctttaaaataccatgcgaagtttatgagcttgggggagcttgggtgctatgcgagccattattccttgtgggaaaaatgcatagaactcaatgaagcgatctgtattttagaagacgatataaccttaaaagaggattttaaagagggattggattttttagaaaaacacatccaagagttaggctatgttcgcttgatgcatttattatatgaccccaatgttaaaagtgagccattgaaccataaaaaccacgagatacaagagcgtgtggggatcattaaagcttatagtcatggggtggggacgcaaggctatgtgatcacgcccaagattgccaaagtttttaaaaaacacagccgaaaatgggttgttcctgtggatacgataatggacgctacttttatccatggcgtgaaaaatctggtgttacaaccttttgtgatcgctgatgatgagcagatctctacgatagcacgaaaagaagaaccttatagccctaaaatcgccttaatgagagaactccattttaaatatttgaaatattggcagtttgtatag

SEQ ID NO: 73 - DNA, 822 bp, Helicobacter pylori
atgcgtgtttttgccatttctttaaatcaaaaagtgtgcgatacatttggtttagtttttagagacaccacaactttactcaatagcatcaatgccacccaccaccaagcgcaaatttttgatgcgatttattctaaaacttttgaaggcgggttgcaccccttagtgaaaaagcatttacacccttatttcatcacgcaaaacatcaaagacatggggattacaaccaatctcatcagtgaggtttctaagttttattacgctttaaaataccatgcgaagtttatgagcttgggggagcttgggtgctatgcgagtcattattccttgtgggaaaaatgcatagaactcaatgaagcgatctgtattttagaagacgatataaccttgaaagaggattttaaagagggcttggattttttagaaaaacacatccaagagttaggctatatccgcttgatgcatttattgtatgatgccagtgtaaaaagtgagccattgagccataaaaaccacgagatacaagagcgtgtggggatcattaaagcttatagcgaaggggtggggactcaaggctatgtgatcacgcctaagattgccaaagtttttttgaaatgcagccgaaaatgggttgttcctgtggatacgataatggacgctacttttatccatggcgtgaaaaatctggtgttacaaccttttgtgatcgctgatgatgagcaaatctctacgatagcacgaaaagaagaaccttatagccctaaaatcgccttaatgagagaactccattttaaatatttgaaatattggcagtttgtataa

SEQ ID NO: 74 - DNA, 849 bp, Aggregatibacter aphrophilus
atgcattttattgaaaataaaaactttgttatcagcattcccactgcggataaacgtcgaaatcacatcatccaacaatttggtcagaagaaaattccctttgaattttttgacgcgtttaccccttctgagcgactcaatgaccatttacagcgctatctcccaaatgttgctgccacacctaggcttaccatgggagaaaaaggctgtctaatgagccatttcatgttgtggaaaaagtgtgttgatgatggtttagattacattacgctttttgaggacgatattttgcttggtgagaatgccgaacagtttcttgctgaagatgaatggcttaaagtgcggttcaattttcaagagatttttgtcttgcgtttagaaacctttttgatgcccgttaagattgaaaaacagcaaggaattttaccttttcaacagagagaaattgatattttgaaatcaaaacattttggtacggcgggatatgtgatttctcatggtgcagctaagtatctaattgaagtgtttgaaaaattttcctcagaagaagttaaacccattgacgaaattatgtttaatcaactgattgatatttctgggtatcaggtatatcaacttaatccggcgatttgtgtgcaagaattgcagttaaatcaagaaaatagtgtattagagagcgggcttcagaaagagcgcaaaaagaatacagtcagccatacaaagaaaacgttaaaataccgcttaactcggatgaaagaaaatattctaagagcgttgaataagaagaaatgggaagagcggcagtatattaaaggactgcagggtaaaaacattattttgtttatatag

SEQ ID NO: 75 - DNA, 849 bp, Pasteurella multocida
atgtcgggcgagcattatgttatttcgttgtcatcggccgtcgagcgcaggcagcacatcaggaaccaattttcgcaaaaaaacatcccgtttcagttttttgatgcgatttcgccatcaccgctgttggatcagcttgttttacagtttttcccgcgcctggcagattcgtcgctgacggggggggaaaaggcgtgctttatgagccatctgtcactgtggcataagtgcgtcgaggagaacctgccatatatagttgtttttgaagatgacatcgttttagggaaagatgcggataaatttttaattggcgatgaatggttgttttcgcgctttgatccggaagaaatttttattattcgcctggaaacgtttctgcagaaagtcgtctgcgaatcgacgcatattgcgccatatacgcatcgggattttctgtcattaaagtcggcacattttggtacggcagggtatgttatttcgcagggcgcagccaagtttctgctggatatttttaaaaacattagcaacgaacatattgcgccaattgatgaattaatttttaaccagtttctggttaaaaactcgtttaacgtctatcaattgtcgccagcaatttgcgttcaggagctgcaactaaataacgaatcatcggcgttacaaagccaactggaactggaacggaacaagtttcggaataaaaaaagcgaagagctgaaacggaaccggaaaaactttatagagaaatttatttatattttgaagaaaccaaaacggatgctagataacaacaaacggaaacgcgaggaatcgaaaatagaaaatgataagatgattattgaatttaaataa

SEQ ID NO: 76 - DNA, 825 bp, Helicobacter pylori
atgttgcgtgtttttatcatttctttaaatcaaaaagtgtgcgatacatttggtttggtttttagagacaccacgactttactcaataatattaatgccacccaccaccaagcgcaaatttttgatgcgatttattctaaaacttttgaaggcgggttgcaccccttagtgaaaaagcatttacacccttatttcatcacgcaaaacatcaaagacatggggattacaaccaatctcatcagtgaggtttctaagttttattacgctttaaaataccatgcgaagtttatgagcttgggggagcttgggtgctatgcgagccattattccttgtgggaaaaatgcatagaactcaatgaagcgatctgtattttagaagacgatataaccttaaaagaggattttaaagagggattggattttttagaaaaacacatccaagagttaggctatgttcgcttgatgcatttattatatgaccccaatgttaaaagtgagccattgaaccataaaaaccacgagatacaagagcgtgtggggatcattaaagcttatagtcatggggtggggacgcaaggctatgtgatcacgcccaagattgccaaagtttttaaaaaacacagccgaaaatgggttgttcctgtggatacgataatggacgctacttttatccatggcgtgaaaaatctggtgttacaaccttttgtgatcgctgatgatgagcagatctctacgatagcacgaaaagaagaaccttatagccctaaaatcgccttaatgagagaactccattttaaatatttgaaatattggcagtttgtatag

SEQ ID NO: 77 - DNA, 843 bp, Pasteurella multocida
atgtcaagcgcgttccattatgttattagcttggcgtcggcagtcgagcggaggcaacatatcagcgaacaattttcgcaatacgatattccgtttcagttttttgatgcgatttcgccatcgccgctgttgaaccaattggtttcacagtttttcccatcgttagcagattcgagcttaacggatggcgaaaaagggtgctttattagccatctgtcactgtggcataaatgcgtcgagaaaaacctgccatatatagttgtttttgaagatgacatccttttagggaagaacgcggataaatttttaatagaggatgaatggtttttttcgcgctttaacacgaacgatgtttttattgttcgcctggaaacgtttctacagaaagtttattgccaaccgtcgtatatcaaatcgtattataacagggaactgctgacgctaaaatcgactcattttggcacggcagggtatataatttcgctgggcgcggcgaagtttctgctttcgttgtttaacaaaatgcatattgaggaagttgcaccaattgatgagctgctatttaacaaatttctagaacggaaagatttcacggtctaccagttttcgccggcattgtgcattcaggaattacaactgaacaaaagcgatgcagtcctgctgagccagctggaactggagaggagcaaatgccggattatgacggagtcaaggattgggcgggaaaagaagaaattaaaagataagattatccatgttctgacgaaaccaaagcggatgctagagaagaaacggcagcggaacgaagacaaaaaaattacgatgataattgagtttgaatag

SEQ ID NO: 78 - DNA, 834 bp, Kingella denitrificans
atggaaaattatgtagtatcaattcgcacagccgcccagagaaggcaacacgtggccgccgaattcaacaaacaccagatcgctttccacttcttcgacgcggtaacgcccgaaacattagctgaaagcatagccgaacactgccccaatctagcggatgctttcctgacgggcggggaaaaaggctgctttatgagccacgtatgcctatgggcgaaatgcgtagcggacgacttaccctatattggaatcttcgaagacgatgtaatattcggacaaaacagcagccggttcttaaatgacacgaaatggttagacgaacggttccaaaaccagagcttcatcattcggatggaaacgttcctaaaagcgaaccccgtagccttatcaaaaagcggggtacgccccttcaacggcagaaagatcctacggttacagtcatttgggttcggcacggcggcgtatctaatcagccaacagaccgcgattacgctattaaattggatcagagaagtagccccagaaaaactagaaccgatcgacaatatgctgttcaatgctgctagcgaaatacccgaaatccagatgtatcagattagcccagccctatgtatccaagaactacaattaaaccgcgcggactcaagcttaagcagcacattagaggacggaaggttagcccggcaccaacagctggatggcggaaaaacccaacccgaacagacccaggaaaatcggaatatattcgcttgggccaagaataaaattgtaaaagaatacaagcgcgtgaagcggagatggacggacgacaaaaaaattgtaccgttcaaatag

SEQ ID NO: 79 - protein, 334 aa, Pasteurella multocida
MENKPLVSVLICAYNVEKYIEECINAVINQTYKNLEIIIVNDGSSDNTYFLLKKLAEKDNRIKILNFNNHIGIISALNEGLKEIAGEYIARTDSDDITKPDWIEKILTCMQNDPKIIAMGSYLTVLSEENNGSVLANHHKNKVEWKNPLEHKDIVEKMLFGNPIHNNSMVMRSEIYTKYHLIYDPDYHYAEDYKFWLEVSRIGKLANYPESLVYYRLHRNQTSSIHNSQQEINGKKLRLQALNYYLKDLGIDYQLPEKFLFKDIALLQEIFYERGMFRENIIRRIIYECYLSLGEYNYKDIYYFLINKNNFLSIKDKFKIIKKYLRPDKYSSTY

SEQ ID NO: 80 - protein, 349 aa, Neisseria meningitidis
MPSEAFRRHRAYRENKLQPLVSVLICAYNVEKYFAQSLAAVVNQTWRNLDILIVDDGSTDGTLAIAQRFQEQDGRIRILAQPRNSGLIPSLNIGLDELAKSGGGGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 81 - protein, 348 aa, Neisseria meningitidis
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 82 - protein, 346 aa, Neisseria gonorrhoeae
MQPLVSVLICAYNVEKYFAQSLAAVVNQTWRNLDILIVDDGSTDGTLAIAKDFQKRDSRIKILAQAQNSGLIPSLNIGLDELAKSGGGGYIARTDADDIASPGWIEKIVGEMEKDRSIIAMGAWLEILSEEKDGNRLARHHKHGKIWKKPTRHEDIAAFFPFGNPIHNNTMIMRRSVIDGGLRYNTGRDWAEDYQFWYDVSKLGRLAYYPEALVKYRLHANQVSSKHSVRQHEIAQGIQKTARNDFLQSMGFKTRFDSLEYRQTKAAAYELPEKDLPEEDFERARRFLYQCFKRTDTPPSGAWLDFAADGRMRRLFTLRQYFGILYRLIKNRRQARSDSAGKEQEI

SEQ ID NO: 83 - protein, 348 aa, Neisseria gonorrhoeae
MQPLVSVLICAYNVEKYFAQSLAAVVNQTWRNLDILIVDDGSTDGTLAIAKDFQKRDSRIKILAQAQNSGLIPSLNIGLDELAKSGGGGGEYIARTDADDIASPGWIEKIVGEMEKDRSIIAMGAWLEVLSEEKDGNRLARHHKHGKIWKKPTRHEDIAAFFPFGNPIHNNTMIMRRSVIDGGLRYDTERDWAEDYQFWYDVSKLGRLAYYPEALVKYRLHANQVSSKHSVRQHEIAQGIQKTARNDFLQSMGFKTRFDSLEYRQTKAAAYELPEKDLPEEDFERARRFLYQCFKRTDTPPSGAWLDFAADGRMRRLFTLRQYFGILYRLIKNRRQARSDSAGKEQEI

SEQ ID NO: 84 - protein, 348 aa, Neisseria lactamica
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTRHEDIAAVFPFGNPIHNNTMIMRRSVIDGGLRYNTERDWAEDYQFWYDVSKLGRLAYYPEALVKYRLHANQVSSKHSVRQHEIAQDIQKTARNDFLQSMGFKTRFDSLEYRQIKAVAYELLEKDLPEEDFERARRFLYQCFKRTDTPPAGAWLDFAADGRMRRLFTLRQYFGILRRLLKNR

SEQ ID NO: 85 - protein, 348 aa, Neisseria lactamica
MPSEAFRRHRAYRENKLQPLVSVLICAYNVEKYFAQSLAAVVNQTWRNLDILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTRHEDIAAVFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 86 - protein, 334 aa, Pasteurella dagmatis
MLELPLVSVLICAYNVEKYIEECINAVINQTYKNLEIIVVNDGSSDNTYFLLKQLAKKDNRIKILNLENNVGIISALNEGLKEVSGEYIARTDSDDITKPDWIEKILTCMENDPEIIAMGSYLTVLSEENNGSVLANHHKDAVEWKNPLEHKDIVEKMLFGNPIHNNSMIMRSKIYTKHKLIYDLDYPYAEDYKFWLEVSRIGKLANYPESLVYYRLHHNQTSSIHHDQQERISKKLRLEALNYYLKALGIDYCLPEKFLFKDIATLHEIFYKNGALQENIIKRIIYECYLSLTEYHYKDIYCFLISENNCFSIKERFKIIKKFLRPSKYSSTY

SEQ ID NO: 87 - protein, 362 aa, Neisseria gonorrhoeae
MPSEAFRRASNYRESKLQPLVSVLICAYNVEKYFAQSLAAVVNQTWRNLDILIVDDGSTDGTLAIAKDFQKRDSRIKILAQAQNSGLIPSLNIGLDELAKSGGGGYIARTDADDIASPGWIEKIVGEMEKDRSIIAMGAWLEILSEEKDGNRLARHHKHGKIWKKPTRHEDIAAFFPFGNPIHNNTMIMRRSVIDGGLRYNTGRDWAEDYQFWYDVSKLGRLAYYPEALVKYRLHANQVSSKHSVRQHEIAQGIQKTARNDFLQSMGFKTRFDSLEYRQTKAAAYELPEKDLPEEDFERARRFLYQCFKRTDTPPSGAWLDFAADGRMRRLFTLRQYFGILYRLIKNRRQARSDSAGKEQEI

SEQ ID NO: 88 - protein, 347 aa, Neisseria gonorrhoeae
MQPLVSVLICAYNVEKYFAQSLAAVVNQTWRNLDILIVDDGSTDGTLAIAKDFQKRDSRIKILAQAQNSGLIPSLNIGLDELAKSGGGGEYIARTDADDIASPGWIEKIVGEMEKDRSIIAMGAWLEILSEEKDGNRLARHHKHGKIWKKPTRHEDIAAFFPFGNPIHNNTMIMRRSVIDGGLRYNTGRDWAEDYQFWYDVSKLGRLAYYPEALVKYRLHANQVSSKHSVRQHEIAQGIQKTARNDFLQSMGFKTRFDSLEYRQTKAAAYELPEKDLPEEDFERARRFLYRCFKRTDTPPSGAWLDFAADGRMRRLFTLRQYFGILYRLIKNRRQARSDSAGKEQEI

SEQ ID NO: 89 - protein, 348 aa, Neisseria meningitidis
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRPEDIAAFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPADAWLDFAADGRMRRLFTLRQYFGILHRLLKNR

SEQ ID NO: 90 - protein, 348 aa, Neisseria meningitidis
MPSEAFRRHRAYRENKLQPLVSVLICAYNAEKYFAQSLAAVVNQTWRNLEILIVDDGSTDGTLAIAKDFQKWDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPADAWLDFAADGRMRRLFTLRQYFGILHRLLKNR

SEQ ID NO: 91 - protein, 362 aa, Neisseria meningitidis
MPSEAFRRHRAYRENKLQPLVSVLICAYNAEKYFAQSLAAVVNQTWRNLEILIVDDGSTDGTLAIAKDFQKWDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGKMRRLFTMRQYFSILHRLIKNRRQARSDSAGKEQEI

SEQ ID NO: 92 - protein, 348 aa, Neisseria meningitidis
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTRHEDIAAFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGVWLDFAADGRMRRLFTLRQYFGILHRLLKNR

SEQ ID NO: 93 - protein, 348 aa, Artificial sequence
  Synthetic polypeptide
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 94 - protein, 348 aa, Artificial sequence
  Synthetic polypeptide
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIASPGWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 95 - protein, 334 aa, Artificial sequence
  Synthetic polypeptide
MENKPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 96 - protein, 334 aa, Artificial sequence
  Synthetic polypeptide
MLELPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 97 - protein, 348 aa, Artificial sequence
  Synthetic polypeptide
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTQHEDIAAVFPFGNPIHNNTMIMRRSVIDGGLRYNTERDWAEDYQFWYDVSKLGRLAYYPEALVKYRLHANQVSSKYSVRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 98 - protein, 334 aa, Artificial sequence
  Synthetic polypeptide
MENKPLVSVLICAYNVEKYFAQSLAAVVNQTWRNLEILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTRHEDIAAVFPFGNPIHNNTMIMRRSVIDGGLRYNTERDWAEDYQFWYDVSKLGRLAYYPEALVKYRLHANQVSSKHSVRQHEIAQDIQKTARNDFLQSMGFKTRFDSLEYRQIKAVAYELLEKDLPEEDFERARRFLYQCFKRTDTPPAGAWLDFAADGRMRRLFTLRQYFGILRRLLKNR

SEQ ID NO: 99 - protein, 334 aa, Artificial sequence
  Synthetic polypeptide
MLELPLVSVLICAYNVEKYFAQSLAAVVNQTWRNLEILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTRHEDIAAVFPFGNPIHNNTMIMRRSVIDGGLRYNTERDWAEDYQFWYDVSKLGRLAYYPEALVKYRLHANQVSSKHSVRQHEIAQDIQKTARNDFLQSMGFKTRFDSLEYRQIKAVAYELLEKDLPEEDFERARRFLYQCFKRTDTPPAGAWLDFAADGRMRRLFTLRQYFGILRRLLKNR

SEQ ID NO: 100 - protein, 335 aa, Artificial sequence
  Synthetic polypeptide
MLELPLVSVLICAYNVEKYFAQSLAAVVNQTWRNLDILIVDDGSTDGTLAIAQRFQEQDGRIRILAQPRNSGLIPSLNIGLDELAKSGGGGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 101 - protein, 334 aa, Pasteurella multocida
MLNKPLVSVLICAYNVEKYIEECINAVINQTYKNLEIIVVNDGSSDNTYFLLKQLAKKDNRIKILNLENNVGIISALNEGLKEVSGEYIARTDSDDITKPDWIEKILTCMENDPEIIAMGSYLTVLSEENNGSVLANHHKDAVEWKNPLEHKDIVEKMLFGNPIHNNSMIMRSKIYTKHKLIYDLDYPYAEDYKFWLEVSRIGKLANYPESLVYYRLHHNQTSSIHHHQQERIGKKLRLQALNYYLKDLSIDYQLPEKFLFKDVEKLQDIFYSHGILQEKIIKRIIYECYLSLAEYHYKDIYYFIRNKSDFLSIKQKSKIIKKYLRPGKYLSTY

SEQ ID NO: 102 - protein, 348 aa, Neisseria lactamica
MPSEAFRRHRAYRENKLQPLVSVLICAYNVEKYFAQSLAAVVNQTWRNLDILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHEHGKIWKKPTRHEDIAAFFPFGNPIHNNTMIMRRSVIDGGLRYNTERDWAEDYQFWYDVSKLGRLAYYPEALVKYRLHANQVSSKHSVRQHEIAQGIQKTARNDFLQSMGFKTRFDSLEYRQIKAVAYELLEKDLPEEDVERARRFLYQCFKRTDTPPAGAWLDFAADGRMRRLFTLRQYFGILHRLLKNR

SEQ ID NO: 103 - protein, 348 aa, Neisseria meningitidis
MPSEAFRRHRAYRENKLQPLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHEHGKIWKKPIRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGVWLDFAADGRMRRLFTLRQYFGILHRLLKNR

SEQ ID NO: 104 - protein, 349 aa, Neisseria polysaccharea
MPSEAFRRHRAYRENKLQPLVSVLICAYNVEKYFAQSLAAVVNQTWRNLDILIVDDGSTDGTLAIAQRFQEQDGRIRILAQPRNSGLIPSLNIGLDELAKSGGGGEYIARTDADDIAAPDWIEKIVGEMEKDRSIIAMGAWLEVLSEEKDGNRLARHHEHGKIWKKPTRHEDIAAFFPFGNPIHNNTMIMRRSVIDGGLRYNTERDWAEDYQFWYDVSKLGRLAYYPEALVKYRLHANQVSSKYSVRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 105 - protein, 348 aa, Neisseria subflava
MPSEAFRRHRAYRENKLQPLVSVLICAYNVEKYFAQSLAAVVNQTWRNLDILIVDDGSTDGTLAIARRFQEQDGRIRILAQPRNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSVRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 106 - protein, 348 aa, Neisseria meningitidis
MPSEAFRRHRAYRENKLQPLVSVLICAYNVEKYFAQSLAAVVNQTWRNLDILIVDDGSTDGTLAIARRFQEQDGRIRILAQPRNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSVRQHEIAQGIQKTARNDFLQSMGFKTRFDSLEYRQIKAVAYELLEKDLSEEDFERVRRFLYQCFKRTDTPPAGAWLDFAADGRMRRLFTMRQYFGILHRLLKNR

SEQ ID NO: 107 - protein, 348 aa, Neisseria meningitidis
MPSEAFRRHRAYRENKLQPLVSVLICAYNVEKYFAQSLAAVVNQTWRNLDILIVDDGSTDGTLAIAQRFQEQDGRIRILAQPRNSGLIPSLNIGLDELAKSGGGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 108 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPIRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGVWLDFAADGRMRRLFTLRQYFGILHRLLKNR

SEQ ID NO: 109 - protein, 332 aa, Neisseria meningitidis
MQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAADGRMRRLFTLRQYFGILHRLLKNR

SEQ ID NO: 110 - protein, 404 aa, Helicobacter pylori
MTSASSHSFKEQDFHIPIAFAFDKNYLIPAGACIYSLLESIAKANKKIRYTLHALVVGLNEEDKAKLNQITEPFKEFAVLEVKDIEPFLDAIPNPFDEDFTKRFSKMVLVKYFLADLFPKYSKMVWSDVDVIFCNEFSADFLNIKENDENYFYGVLEVEKHHMMEGFLFCNLDYQRKKNFTLRMHDLLKGNEAKGELDFTKWCWPNMKALGIEYCVFPYYYTIKDFSNAYLNENYKKTILEARENPTIIHYDAWWGAVKPWDYPFGLKADLWLNALAKTPFMSDYTKKMHTNESFYTTKMAEQHYFSSVKSSKEILFKTPYLFFKSYLFVVFKERKIHSRVFELTCNLVKKFFNKLIYFGFLMPKALAKRVVSKILRVLGLHGIVKKILIQLKLLKKELFLWGL

SEQ ID NO: 111 - protein, 436 aa, Helicobacter pylori
MTSASSHSFKEQDFHIPIAFAFDKNYLIPAGACLYSLLESIAKANKKIRYTLHALVVGLNEEDKAKLNQITEPFKEFAALEVKDIEPFLDAIPNPFDEDFTKRFSKMVLVKYFLADLFPKYSKMVWSDVDVIFCNEFSADFLNIKENDENYFYGVLGGGERHVLEGFLFCNLDYQRKNHFVSKIHALLEENKATEELDFTQWCWPNMKALGIEYCVFPFYYTVKDFSASLPENYFLHEIYKKTISNALKNPIIIHYDVWWGAVKPWDYPFGLKADLWLNALAKTPFMSDWIDSIARVEIGSEKWHRYHSIVAYHYYFPLWKTEEQIAHDALKTFLDHYFSCIHATIKQENLGMFLNHYFSHAHAEIKENSLEAFLNHYFSHVYRLPKNARKKLFRVFVKHCILIPLKSLISKTLKILGLHGLVKKILIQLKLLKKS

SEQ ID NO: 112 - protein, 398 aa, Helicobacter pylori
MTSASSHSFKEQDFHIPIAFAFDKNYLIPAGACLYSLLESIAKANKKIRYTLHALVVGLNEEDKAKLHKIAEPFKEFAVLEVKDIEPFLDTIPNPFDEDFTKRFSKMVLVKYFLADLFPKYSKMVWSDVDVIFCNEFSADFLNIKEDDENYFYGVLEVEKHHMMEGFLFCNLDYQRKKNFTLRMHDLLKGNEAKGELDFTKWCWPNMKALGIEYCVFPYYYTIKDFSNAYLNENYKKTILEARENPTIIHYDAWWGAVKPWDYPFGLKADLWLNALAKTPFMSDYTKKMHTNESFYTTKMAEQHYFSSVKSSKEILFKTPYLFFKSYLLVVFKERKIHLRIFALMGGLVKKFFNKLIYFGFLMPKALVKRAVSKILRVLGLHGIVKKILIKLKLLRKG

SEQ ID NO: 113 - protein, 398 aa, Helicobacter pylori
MTSASSHSFKEQDFHIPIAFAFDKNYLIPAGACLYSLLESIAKANKKIHYTLHALVVGLNEEDKAKLNQITEPFKEFATLEVRDIEPFLDAIPNPFDEDFTKRFSKMVLVKYFLADLFPKYSKMVWSDVDVIFCNEFSADFLSIKEDDENYFYGVLEVEKHHMMEGFLFCNLDYQRKKNFTLRMHDLLKGNEAKGELDFTKWCWPNMKALGIEYCVFPYYYTIKDFSNAYLNENYKKTILEARENPTIIHYDAWWGAVKPWDYPFGLKADLWLNALAKTSFMSDYTKKMHTNESFYTTKMAEQHYFSSVKSSKEILFKTPYLFFKSYLFVVFKERKIHSRVFELICGLVKKFFNKLIYFGFLMPKALVKRAVSKILRVLGLHGIVKKILIKLKLLKKG

SEQ ID NO: 114 - protein, 398 aa, Helicobacter pylori
MTSSSNHSFREQDFHIPIAFAFDKNYLIPAGACLYSLLESIAKANKKIRYTLHALVVGLNEEDKTKLNQITEPFKEFVALEVKDIEPFLDTIPNPFDEDFTKRFSKMVLVKYFLADLFPKYSKMVWSDVDVIFCNEFSADFLNIKEDDENYFYGVLEVEKHHMMEGFLFCNLDYQRKKNFTLRMHEILKGNETKGELDFTKWCWPNMKALGVEYCVFPWYYTIKDFSNAYLNENYKKTILEARENPTIIHYDAWWGAVKPWDYPFGLKADLWLNALAKTPFMSDYTKKMHTNESFYTTKMAEQHYFSSVKSSKEILFKAPYLFFKSYLFVVFKERKIHSRVFELTCNLVKEFFNKLIYFGFLMPKALAKRVVSKILRILGLHGIVKKILIQLKLLRKG

SEQ ID NO: 115 - protein, 396 aa, Helicobacter pylori
MTSASNHSFKEQDFHIPIAFAFDKNYLIPAGACLYSLLESIAKANKKIRYTLHALVVGLNEEDKAKLNQIAEPFKEFAVLEVKDIEPFLDTIPNPFDEDFTKRFSKMVLVKYFLADLFPKYSKMVWSDVDVIFCNEFSADFLNIEENDENYFYGVLEVEKHHMMEGFLFCNLDYQRKKNFTLRMHEILKGNETKGELDFTKWCWPNMKALGVEYCVFPWYYTIKDFSNAYLNENYKKTILEALKNPTIIHYDAWWGAVKPWDYPFGLKADLWLNALAKTPFMSDYTKKMHTNESFYTTKIAQQHYFSSIKSSKEILFKVPYLFFKSYLFVVFKERKIHSRVFELTCGLVKKFFNKLIYFGFFMPKALAKRVVSKILIVLGLHGIVKKILIKLLKKG

SEQ ID NO: 116 - protein, 394 aa, Helicobacter pylori
MTSASSHSFKEQDFHIPIAFAFDKNYLIPAGACIYSLLESIAKANKKIRYTLYALVVGLDEEDEAKLHQITEPFKEFATLEVKDIEPFLDTIPNPFDEDFTKRFSKMVLVKYFLADLFPKYSKMVWSDVDVIFCNEFSADFLNIKEDDENYFYGVLEVEKHHMMEGFLFCHLDYQRKKNFTLRMHDLLKGNEAKGELDFTKWCWPNMKALGIEYCVFPFYYTIKDFSNAYLNENYKKTILEVLKNPTIIHYDAWWGAVKPWDYPFGLKADLWLNALSKTPFMSDYTKKMHTNESFYTTKMAQQHYFSSTAPSKEILFKAPYVFFKSYLFVVFKERKIHSRVFELTCGLVKKFFNKLIYFGFFMPKALAKRVVSKILKILGLHGIVKKILLKLLK

SEQ ID NO: 117 - protein, 398 aa, Helicobacter pylori
MTSASSHSLKEQDFHIPIAFAFDKNYLIAAGACIYSLLESIAKANKKIRYTLYALVVGLDEEDKAKLHQITEPFKEFAVLEVKDIEPFLDTIPNPFDEDFTKRFSKMVLVKYFLADLFPKYSKMVWSDVDVIFCNEFSADFLNIKEDDENYFYGVLEVEKHHMMEGFLFCHLDYQRKKNFTLRMHDLLKGNGAKEELDFTKWCWPNMKALGIEYCVFPLYYTIKDFSNVYLNENYKKTILEALKNPIIIHYDAWWGAVKPWDYPFGLKADLWLNALSKTPFMSDYTKKMHTNESFYTTKMAQQHYFSSTAPSKEILFKAPYLFFKSYLFVVFKERKIHSRVFELTCGLVKKFFNKLIYFGFFMPKSLAKRVVSKILRVLGLHGIAKKILIKLKLLKKS

SEQ ID NO: 118 - protein, 436 aa, Helicobacter pylori
MDSASNHSFKEQDFHIPIAFAFDKNYLIPAGACLYSLLESIAKANKKIRYTLHALVVGLNEEDKAKLNQIAEPFKEFAVLEVKDIEPFLDAIPNPFDEDFTKRFSKMVLVKYFLADLFPKYSKMVWSDVDIIFCNEFSADFLNIKEDDENYFYGVLGGGEHHVLEGFLFCNLDYQRKNHFVSKTHALLEENKATEELDFTQWCWPNMKALGIEYCVFPYYYTVKDFSASLPENYFLHEIYKKTISNALKNPIIIHYDVWWGAVKPWDYPFGLKADLWLNALAKTPFMSDWIDSIARVEIGSEKWNRYHSIVAYHYYFPLWKTEEQIAHDALKTFLDHYFSCIHATIKQENLGMFLNHYFSHAHAEIKENSLEVFLNHYFSHVYRLPKNAQKKLFRVFVKHCILIPLKSLISKTLKILKLHALVKKILIQLKLLRKS

SEQ ID NO: 119 - protein, 456 aa, Helicobacter pylori
MTSASNHSFKEQDFHIPIAFAFDKNYLIPAGACLYSLLESIAKANKKIRYTLHALVVGLNEEDKAKLNQITEPFKEFAALEVKNIEPFLDTIPNPFDEDFTKRFSKMVLVKYFLADLFPKYSKMVWSDVDVIFCNEFSADFLNIKENDENYFYGVLGGGERHVLEGFLFCNLDYQRKNHFVSKTHALLEENKATEELDFTQWCWPNMKALGIEYCVFPYYYTVKDFSASLPENYFLHEIYKKTISNALKNPIIIHYDVWWGAVKPWDYPFGLKADLWLNALAKTPFMSDWIDSIARVEIGSEKWHRYHSIVAYHYYFPLWKTEEQIAHDALKTFLDHYFSCIHATIKQENLGMFLNHYFSHAHAEIKENPLEAFLNHYFSHAHAEIKENSLEAFLNHYFSHVYRLPKNARKRLLRVFVKHCILIPLKSLVGKTLRFLKLHALVKKILIQLKLLKKS

SEQ ID NO: 120 - protein, 436 aa, Helicobacter pylori
MTSSSSHSFKEQDFHIPIAFAFDKNYLIPAGACLYSLLESIAKANKKIRYTLHALVVGLNEEDKAKLNQIAEPFKEFAVLEVRDIEPFLDTIPNPFDEDFTKRFSKMVLVKYFLADLFPKYSKMVWSDVDVIFCNEFSADFLNIKEDDENYFYGVLGGGECHVLEGFLFCNLDYQRKNHFVSKTHALLEENKATEELDFTQWCWPNMKALGIEYCVFPYYYTVKDFSASLPENYFLHEIYKKTISNALKNPIIIHYDVWWGAVKPWDYPFGLKADLWLNALAKTPFMSDWIDSIARVEIGSEKWHRYHSIVAYHYYFPLWKTEEQIAHDALKTFLDHYFSCIHATIKQENLGMFLNHYFSHAHAEIKENSLEAFLNHYFSHVYRLPKNVRKKLFRVFIKHCILIPLKSLVGKTLRFLKLHALVKNILIQLKLLKKS

SEQ ID NO: 121 - protein, 388 aa, Helicobacter cetorum
MISTVITPPPEKTQQSELNEYSHIPIAFAFDKNYCIPFAACLQSFLECIARANKQVFYSLHALVVGLDEDDIQKLHQIAEPFKEMATLEIKNIEPFLDTIKNPFDESFTKRFSKMVLIKYFLADLFPKYSKIVWSDVDIIFCEEFSKDFLKITEDDKNYLYGVFDGKQHVLEGFLFCNLALQRKNHFTQKIQEILHAQEVTEEPHLTDWCWPYIGQLGIEYCVFPSYYILNQENELFLYERLYDNYSKHVQQALKNPIIIHYDGWIHAIKPWDNPLSMKANLWLNTLAKTPFFTDYTELLQKNTNFYGKIISKDYYFPYYASYTDTILKPLYLFFQNYSAYLKERVFCEEFYMRVIKIPLSKTLKRLKIHRALKKILLALKIIKSNAN

SEQ ID NO: 122 - protein, 348 aa, Neisseria meningitidis
MPSEAFRRHRAYRENKLQPLVSVLICAYNVEKYFAQSLAAVVNQTWCNLDILIVDDGSTDGTLAIAKDFQKRDSRIKILAQAQNSGLIPSLNIGLDELAKSGGGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSVRQHEIAQGIQKTARNDFLQSMGFKTRFDSLEYRQIKAVAYELLEKHLPEEDFERARRFLYQCFKRTDTLPAGAWLDFAADGRMRRLFTLRQYFGILHRLLKNR

SEQ ID NO: 123 - protein, 348 aa, Neisseria meningitidis
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 124 - protein, 348 aa, Neisseria meningitidis
MPSEAFRRHRAYRENKLQPLVSVLICAYNVEKYFAQSLAAVVNQTWRNLDILIVDDGSTDGTLAIARRFQEQDGRIRILAQPRNSGLIPSLNIGLDELAKSGMGEYIARTDADDIAAPDWIEKIVGEMEKDRSIIAMGAWLEVLSEEKDGNRLARHHEHGKIWKKPTRHEDIAAVFPFGNPIHNNTMIMRRSVIDGGLRYNTERDWAEDYQFWYDVSKLGRLAYYPEALVKYRLHANQVSSKYSVRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 125 - protein, 362 aa, Neisseria meningitidis
MPSEAFRRHRAYRENKLQPLVSVLICAYNVEKYFAQSLAAVVNQTWRNLDILIVDDGSTDGTLAIAQRFQEQDGRIRILAQPRNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGKMRRLFTMRQYFGILHRLIKNRRQARSDSAGKEQEI

SEQ ID NO: 126 - protein, 496 aa, Helicobacter pylori
MTSVSSHSFKKQDFHIPIAFAFDKNYLIPAGTCLYSLLESIAKANKKIRYTIHALVVGLNEEDKARLNQIAEPFKEFAALEVRDIEPFLDAIPNPFDEDFTKRFSKMVLVKYFLADLFPKYSKMVWSDVDVIFCNEFSADFLNIEENDENYFYGVLGGGECHVLEGFLFCNLDYQRKNHFVSKTHALLEENRATEELDFTKWCWPNMKALGIEYCVFPYYYTVKDFSASLPENYFLHEIYKKTISNALKNPIVIHYDVWWEAVKPWDYPFGLKADLWLNALAKTPFMRDWIDSIARVEIGSEKWHRYHSIVAYHYYFPLWKTEEQIAHDALKTFLDHYFSRIHATIKQESLGMFLNHYFSHAHAEIKENSLEMFLNHYFSHAHAEIKENSLEMFLNHYFSHAHAEIKENSLEMFLNHYFSHAHAEIKENSLEMFLDHYFSHVYRLPKKARKRLFRVFVKHCILIPLKSLISKTLKILGLHGLVKKILIQLKLLKKS

SEQ ID NO: 127 - protein, 431 aa, Helicobacter pylori
MTSASNHSFKEQDFHIPIAFAFDKNYLIPAGACIYSLLESIAKANKKIRYTLHALVVGLNEEDKAKLNQITEPFKEFAVLEIKDIEPFLDAIPNPFDEDFTKRFSKMVLVKYFLADLFPKYSKMVWSDVDVIFCNEFSADFLNIKEDDENYFYGVLEGGERHVLEGFLFCNLDYQRKNHFVSKTHALLEENKATEELDFTQWCWPNMKALGIEYCVFPYYYTIKDFSNAYLNENYKKTILEARENPTIIHYDAWWGAVKPWDYPFGLKADLWLNALAKTPFMSDWIDSIARVEIGSEKWHRYHSIVAYHYYFPLWKTEEQIAHDALKTFLDHYFSCIHATIKQENLGMFLNHYFSHAHAEIKENSLEAFLNHYFSHVYRLPKNARKKLFGVFVKHCILIPLKSLISKTLKILGLHALAKKILIQLKLLKKS

SEQ ID NO: 128 - protein, 456 aa, Helicobacter pylori
MTSASNHSLKEQDFHIPIAFAFDKNYLIPAGACIYSLLESIAKANKKIRYTLHALVVGLNEEDKAKLNQIAEPFKEFAVLEVKDIEPFLDTIPNPFDEDFTKRFSKMVLVKYFLADLFPKYSKMVWSDVDVIFCNEFSADFLNIKENDENYFYGVLGGGECHVLEGFLFCNLDYQRKNHFVSKTHALLEENKATEELDFTQWCWPNMKALGIEYCVFPYYYTVKDFSASLPENYFLHEIYKKTISNALKNPIIIHYDVWWGAVKPWDYPFGLKADLWLNALAKTPFMSDWIDSIARVEIGSEKWHRYHSIVAYHYYFPLWKTEEQIAHDALKTFLDHYFSCIHATIKQENLGMFLNHYFSHAHAEIKENSLEAFLNHYFSHAHAEIKENSLEAFLNHYFSHVYRLPKNAQKKLFRVFIKHCILIPLKSLVGKTLRFLKLHALVKKILIQLKLLKKS

SEQ ID NO: 129 - protein, 456 aa, Helicobacter pylori
MTSASSHSLKEQDFHIPIAFAFDKNYLIPAGACLYSLLESIAKANKKIRYTLHALVVGLNEEDKAKLNQIAEPFKEFAALEVRDIESFLDTIPNPFDEDFTKRFSKMVLVKYFLADLFPKYSKMVWSDVDVIFCNEFSADFLNIKENDENYFYGVLGGGERHVLEGFLFCNLDYQRKNHFVSKTHALLEENKATEELDFTQWCWPNMKALGIEYCVFPYYYTVKDFSASLPENYFLHEIYKKTISNALKNPIIIHYDVWWGAVKPWDYPFGLKADLWLNALAKTPFMSDWIDSIARVEIGSEKWHRYHSIVAYHYYFPLWKTEEQIAHDALKTFLDHYFSRIHAAIKQENLGMFLNHYFSHAHAEIKENSLKAFLNHYFSHAHAEIKENSLKAFLNHYFSHVYRLPKNAQKRLFRVFVKHCILIPLKSLISKTLKILGLHGLVKKILIQLKLLKKS

SEQ ID NO: 130 - protein, 436 aa, Helicobacter pylori
MTSASNHSFKEQDFHIPIAFAFDKNYLIPAGACLYSLLESIAKANKKIRYTLHALVVGLNEEDKAKLNQIAEPFKEFAALEVKDIEPFLDTIPNPFDEDFTKRFSKMVLVKYFLADLFPKYSKMVWSDVDVIFCNEFSADFLNIKENDENYFYGVLGGGERHVLEGFLFCNLDYQRKNHFVSKTHALLEENKATEELDFTQWCWPNMKALGIEYCVFPYYYTVKDFSASLPENYFLHEIYKKTISNALKNPIIIHYDVWWGAVKPWDYPFGLKADLWLNALAKTPFMSDWIDSIARVEIGSEKWHRYHSIVAYHYYFPLWKTEEQIAHDALKTFLDHYFSCIHATIKQENLGMFLNHYFSHAHAEIKENSLEAFLNHYFSHVYRLPKNARKKLFRVFIKHCILIPLKSLVGKTLRFLKLHALVKKILIQLKLLKKS

SEQ ID NO: 131 - protein, 297 aa, Escherichia coli
MFSIIIPSFNREKQLRNALAELNKQLYKEFEVIVVDDASDSEYDLSLDLYHFELKYFRSNKNLGPSGARNIGVSLARYDWIIFLDDDDYFMPEKLMILRETILNNDIDFIYHRARIFMINEKISYISSQKDIDKIPGEVYQHMLSGNFIGGPPNFAIKKSLFNKLHGFSDNVRAIEDYEFLLRLVRDVSKKRIIFVDNVLTGCNYITKSTSVSKNINNLKQACTYISEEYIRDNREFNLFKINTSLMTAHALLMSLNRKSAFFYFKAGIVMFNIKYIVSGIISFISPVLMIKLRGKV

SEQ ID NO: 132 - protein, 265 aa, Escherichia coli
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 133 - protein, 275 aa, Pseudogulbenkiania ferrooxidans
MDKIKQGSASLVVGDQQEKHPVVSVLLPVNRVDRFFIPAVESILTQTLQDFELIIIANGCSTEHLNKIRLTYGDHNRVRILNTEIKGLPFALNLGVHNARGLYIARMDADDISIPERLEKQLNTLEQNKKIGVVSSGVDFIDENDQAIREGKFPELTDKDHRRLLPLICCIAHPTVMVRKEIINKLGGYSFGSFSEDYDLWLRIMRELPEVEFYRIPESLLKYRRHGNQATSSKNIKKIRAYNSALKIRELFLSRKLKFIIGIILPARMVTLWRK

SEQ ID NO: 134 - protein, 264 aa, Salmonella enterica
MLTEVRPVSTTKPLVSVILPVNKFNPYLDRAIHSILSQSYPSIELIIIANNCTNDFFDALKKRECETIKVLRTNIAYLPYCLNKGLDLCNGDFVARMDSDDISHPERIDRQVDFLINNPDIDVVGTNAVYIDEDDIELEKSNLPVNNNAIRKMLPYKCCLVHPSVMFRKNVVITSGGYMFANYSEDYELWNRLAVEGRNFYNLSEYLLYYRLHNNQSTSKNNLFMVMANDVAIKVKYFLLTKKISYLLGIIRTVFSVFYCKYIK

SEQ ID NO: 135 - protein, 274 aa, Corynebacterium glutamicum
MTSSAPLISVVIPTIAYDEYCSQSIKSVCEQNYENWQIVLVLDGAPIKDVPQWVKEHERIKIVEQKIRQGTPTSLNNGIKASDGQLIARLDSDDLAAPSRLSKQEEFLRNHPYIICVATKTKHINEHGKIFGQSADLPTSQDIRQILLVKNPIIHSSVMYRKQVVEQIGGYSLEMTRSQDYELFLRLSKIGAIGYLDESLSSYRIHGGQHSRKTSPFKKYTWIILKRRMELASFLKRSPVRQIFLNFIWYGAQVTRYLGLRKAGFMTGYTPDRI

SEQ ID NO: 136 - protein, 275 aa, Neisseria meningitidis
MQNHVISLASAAERRAHITDTFGVRGIPFQFFDALMPSERLEQVMAELVPGLSAHPYLSGVEKACFMSHAVLWKQALDEGLPYITVFEDDVLLGEGAEKFLAEDAWLQERFDPDSAFIVRLETMFMHVLTSPSGVADYGGRAFPLLESEHWGTAGYIISKKAIRFFLDRFAALPPEGLHPVDLMMFSDFFDREGVPVCQLNPALCAQELHYAKFHDQNSALGSLIEHDRLLNRKQQWRDSPANTFKRRLIRALTKISREREKRRQRREQFIVPFQ

SEQ ID NO: 137 - protein, 275 aa, Neisseria meningitidis
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 138 - protein, 315 aa, Streptococcus agalactiae
MIKKIEKDLISVIVPIYNVEDYLVECIESLIVQTYRNIEILLINDGSTDNCATIAKEFSERDCRVIYIEKSNGGLSEARNYGIYHSKGKYLTFVDSDDKVSSDYIANLYNAIQKHDSSIAIGGYLEFYERHNSIRNYEYLDKVIPVEEALLNMYDIKTYGSIFITAWGKLFHKSIFNDLEFALNKYHEDEFFNYKAYLKANSITYIDKPLYHYRIRVGSIMNNSDNVIIARKKLDVLSALDERIKLITSLRKYSVFLQKTEIFYVNQYFRTKKFLKQQSVMFKEDNYIDAYRMYGRLLRKVKLVDKLKLIKNRFF

SEQ ID NO: 139 - protein, 273 aa, Helicobacter pylori
MRVFIISLNQKVCDTFGLVFRDTTTLLNNINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYVRLMHLLYDPNVKSEPLNHKNHEIQERVGIIKAYSHGVGTQGYVITPKIAKVFKKHSRKWVVPVDTIMDATFIHGVKNLVLQPFVIADDEQISTIARKEEPYSPKIALMRELHFKYLKYWQFV

SEQ ID NO: 140 - protein, 273 aa, Helicobacter pylori
MRVFAISLNQKVCDTFGLVFRDTTTLLNSINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYIRLMHLLYDASVKSEPLSHKNHEIQERVGIIKAYSEGVGTQGYVITPKIAKVFLKCSRKWVVPVDTIMDATFIHGVKNLVLQPFVIADDEQISTIARKEEPYSPKIALMRELHFKYLKYWQFV

SEQ ID NO: 141 - protein, 282 aa, Aggregatibacter aphrophilus
MHFIENKNFVISIPTADKRRNHIIQQFGQKKIPFEFFDAFTPSERLNDHLQRYLPNVAATPRLTMGEKGCLMSHFMLWKKCVDDGLDYITLFEDDILLGENAEQFLAEDEWLKVRFNFQEIFVLRLETFLMPVKIEKQQGILPFQQREIDILKSKHFGTAGYVISHGAAKYLIEVFEKFSSEEVKPIDEIMFNQLIDISGYQVYQLNPAICVQELQLNQENSVLESGLQKERKKNTVSHTKKTLKYRLTRMKENILRALNKKKWEERQYIKGLQGKNIILFI

SEQ ID NO: 142 - protein, 282 aa, Pasteurella multocida
MSGEHYVISLSSAVERRQHIRNQFSQKNIPFQFFDAISPSPLLDQLVLQFFPRLADSSLTGGEKACFMSHLSLWHKCVEENLPYIVVFEDDIVLGKDADKFLIGDEWLFSRFDPEEIFIIRLETFLQKVVCESTHIAPYTHRDFLSLKSAHFGTAGYVISQGAAKFLLDIFKNISNEHIAPIDELIFNQFLVKNSFNVYQLSPAICVQELQLNNESSALQSQLELERNKFRNKKSEELKRNRKNFIEKFIYILKKPKRMLDNNKRKREESKIENDKMIIEFK

SEQ ID NO: 143 - protein, 274 aa, Helicobacter pylori
MLRVFIISLNQKVCDTFGLVFRDTTTLLNNINATHHQAQIFDAIYSKTFEGGLHPLVKKHLHPYFITQNIKDMGITTNLISEVSKFYYALKYHAKFMSLGELGCYASHYSLWEKCIELNEAICILEDDITLKEDFKEGLDFLEKHIQELGYVRLMHLLYDPNVKSEPLNHKNHEIQERVGIIKAYSHGVGTQGYVITPKIAKVFKKHSRKWVVPVDTIMDATFIHGVKNLVLQPFVIADDEQISTIARKEEPYSPKIALMRELHFKYLKYWQFV

SEQ ID NO: 144 - protein, 280 aa, Pasteurella multocida
MSSAFHYVISLASAVERRQHISEQFSQYDIPFQFFDAISPSPLLNQLVSQFFPSLADSSLTDGEKGCFISHLSLWHKCVEKNLPYIVVFEDDILLGKNADKFLIEDEWFFSRFNTNDVFIVRLETFLQKVYCQPSYIKSYYNRELLTLKSTHFGTAGYIISLGAAKFLLSLFNKMHIEEVAPIDELLFNKFLERKDFTVYQFSPALCIQELQLNKSDAVLLSQLELERSKCRIMTESRIGREKKKLKDKIIHVLTKPKRMLEKKRQRNEDKKITMIIEFE

SEQ ID NO: 145 - protein, 277 aa, Kingella denitrificans
MENYVVSIRTAAQRRQHVAAEFNKHQIAFHFFDAVTPETLAESIAEHCPNLADAFLTGGEKGCFMSHVCLWAKCVADDLPYIGIFEDDVIFGQNSSRFLNDTKWLDERFQNQSFIIRMETFLKANPVALSKSGVRPFNGRKILRLQSFGFGTAAYLISQQTAITLLNWIREVAPEKLEPIDNMLFNAASEIPEIQMYQISPALCIQELQLNRADSSLSSTLEDGRLARHQQLDGGKTQPEQTQENRNIFAWAKNKIVKEYKRVKRRWTDDKKIVPFK

(19) United States
(12) Patent Application Publication
(10) Pub. No.: US 2024/0368651 A1
(43) Pub. Date: Nov. 7, 2024

(54) CELLULAR PRODUCTION OF DI- AND/OR OLIGOSACCHARIDES

(71) Applicant: INBIOSE N.V., Zwijnaarde (BE)

(72) Inventors: Sofie Aesaert, Zwijnaarde (BE); Joeri Beauprez, Zwijnaarde (BE); Pieter Coussement, Zwijnaarde (BE); Thomas Decoene, Zwijnaarde (BE); Nausicaä Lannoo, Zwijnaarde (BE); Gert Peters, Zwijnaarde (BE); Kristof Vandewalle, Zwijnaarde (BE); Annelies Vercauteren, Zwijnaarde (BE)

(21) Appl. No.: 18/041,154

(22) PCT Filed: Aug. 10, 2021
(86) PCT No.: PCT/EP2021/072275

§ 371 Date: Oct. 20, 2023

(30) Foreign Application Priority Data
Aug. 10, 2020 (EP) 20190204.6
Apr. 16, 2021 (EP) 21168997.1
Jul. 16, 2021 (EP) 21186203.2
Aug. 10, 2021 (WO) PCT/EP2021/072275

(51) Int. Cl. C12P 19/04; C12N 1/20; C12N 9/10; C12N 15/70; C12P 19/12; C12P 19/26

(52) U.S. Cl. CPC C12P 19/04; C12N 1/20; C12N 9/1051; C12N 9/1085; C12N 15/70; C12P 19/12; C12P 19/26; C12N 2510/00; C12Y 204/01038; C12Y 204/01146; C12Y 204/99004; C12Y 205/01056

(57) ABSTRACT

The disclosure is in the technical field of synthetic biology and metabolic engineering. More particularly, the disclosure is in the technical field of metabolically engineered cells and use of the cells in a cultivation or fermentation. The disclosure describes a cell and a method for production of a di- and/or oligosaccharide. The cell comprises a pathway for production of the di- and/or oligosaccharide and is genetically modified for expression and/or overexpression of at least one set of multiple coding DNA sequences wherein the multiple coding DNA sequences within one set differ in nucleotide sequence and each encode a polypeptide, wherein the polypeptides have the same function and/or activity of interest. Furthermore, the disclosure provides for purification of the di- and/or oligosaccharide from the cultivation.

### CROSS-REFERENCE TO RELATED APPLICATIONS

This application is a national phase entry under 35 U.S.C. § 371 of International Patent Application PCT/EP2021/072275, filed Aug. 10, 2021, designating the United States of America and published as International Patent Publication WO 2022/034081 A1 on Feb. 17, 2022, which claims the benefit under Article 8 of the Patent Cooperation Treaty to European Patent Application Serial No. 20190204.6, filed Aug. 10, 2020, European Patent Application Serial No. 21168997.1, filed Apr. 16, 2021, and European Patent Application Serial No. 21186203.2, filed Jul. 16, 2021.

### STATEMENT ACCORDING TO 37 C.F.R. § 1.821(c) or (e)—SEQUENCE LISTING SUBMITTED AS A TXT AND PDF FILES

Pursuant to 37 C.F.R. § 1.821(c) or (e), a Sequence Listing ASCII text file entitled “4006-P17267US (042-PCT-US) US Sequence Listing_ST25.txt,” 320,104 bytes in size, generated Jan. 23, 2023, has been submitted, the contents of which are hereby incorporated by reference.

### TECHNICAL FIELD

The disclosure is in the technical field of synthetic biology and metabolic engineering. More particularly, the disclosure is in the technical field of metabolically engineered cells and use of the cells in a cultivation or fermentation. The disclosure describes a cell and a method for production of a di- and/or oligosaccharide. The cell comprises a pathway for production of the di- and/or oligosaccharide and is genetically modified for expression and/or overexpression of at least one set of multiple coding DNA sequences wherein the multiple coding DNA sequences within one set differ in nucleotide sequence and each encode a polypeptide, wherein the polypeptides have the same function and/or activity of interest. Furthermore, the disclosure provides for purification of the di- and/or oligosaccharide from the cultivation.

### BACKGROUND

In recent years, the interest in fermentative synthesis of di- and/or oligosaccharides using metabolically engineered cells has increased significantly due to the multiple, often vital phenomena these molecules are involved in. Di- and oligosaccharides, frequently present as glyco-conjugated forms to proteins and lipids, play a major role in differentiation, development and biological recognition processes related to the development and progress of fertilization, embryogenesis, inflammation, metastasis and host pathogen adhesion. Oligosaccharides present as unconjugated glycans in body fluids and mammalian milk also modulate important developmental and immunological processes. Fermentative approaches to produce a di- and/or oligosaccharide using cells require 1) one or more glycosyltransferases that are expressed and/or over-expressed by the cells and that catalyze the selective transfer of a sugar moiety from an activated nucleotide-sugar donor onto one or more saccharide acceptors, 2) within the cells an available pool of one or more activated nucleotide-sugar donors for the glycosyltransferases, 3) an available pool of one or more appropriate saccharide acceptors being delivered to and/or synthesized within/by the cells, 4) optimal growth of the cells and 5) an efficient way to separate and preferably to purify the produced di- and/or oligosaccharide from the cells during and/or after cultivation. However, metabolic engineering of cells toward efficient production hosts for a di- and/or oligosaccharide often result in cells that suffer from clonal instability, clonal heterogeneity or transgene silencing by the introduction of multiple coding DNA sequences that encode polypeptides that are involved in the production of a di- and/or oligosaccharide, ultimately leading to a non-efficient production system for di- and/or oligosaccharides.

### BRIEF SUMMARY

Provided are tools and methods by means of which a di- and/or oligosaccharide can be produced by a cell and preferably in an efficient, time and cost-effective way and which yields high amounts of the desired di- and/or oligosaccharide

Provided are a cell and a method for the production of a di- and/or oligosaccharide wherein the cell of disclosure comprises a pathway for the production of the di- and/or oligosaccharide and is genetically modified for expression and/or overexpression of at least one set of multiple coding DNA sequences wherein the multiple coding DNA sequences within one set differ in nucleotide sequence and each encode a polypeptide, wherein the polypeptides have the same function and/or activity of interest. Surprisingly, it has now been found that the cell of disclosure does not suffer from clonal instability, clonal heterogeneity or transgene silencing by the introduction of the at least one set of multiple coding DNA sequences. The expression and/or overexpression of at least one set of the multiple coding DNA sequences in the cell of disclosure preferably has a positive effect on (fermentative) production of the di- and/or oligosaccharide, and even more preferably, provides a better yield, productivity, specific productivity and/or growth speed of the cell when compared to a cell with the same genetic background but lacking the set(s) of multiple coding DNA sequences as defined in the disclosure. The disclosure also provides a method for the production of a di- and/or oligosaccharide. The method comprises the steps of providing a cell comprising a pathway for the production of a di- and/or oligosaccharide, wherein the cell is genetically modified with at least one set of multiple coding DNA sequences wherein each coding DNA sequence differs in nucleotide sequence and encodes a polypeptide, wherein the polypeptides have the same function and/or activity of interest and cultivating the cell under conditions permissive to produce the di- and/or oligosaccharide. The polypeptides encoded by a set of multiple coding DNA sequences can be chosen from the list comprising, amongst others, enzymes that are directly involved in the synthesis of (i) a nucleotide-activated sugar, wherein the nucleotide-activated sugar is to be used in the production of the di- and/or oligosaccharide, (ii) glycosyltransferases or (iii) membrane transporter proteins. The disclosure also provides methods to separate the di- and/or oligosaccharide.

### Definitions

The words used in this specification to describe the disclosure and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the disclosure disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the specification, there have been disclosed embodiments of the disclosure, and although specific terms are employed, the terms are used in a descriptive sense only and not for purposes of limitation, the scope of the disclosure being set forth in the following claims. It must be understood that the illustrated embodiments have been set forth only for the purposes of example and that it should not be taken as limiting the disclosure. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims that follow, reference characters used to designate claim steps are provided for convenience of description only, and are not intended to imply any particular order for performing the steps.

In this document and in its claims, the verb “to comprise” and its conjugations is used in its non-limiting sense to mean that items following the word are included, but items not specifically mentioned are not excluded. Throughout the disclosure, the verb “to comprise” may be replaced by “to consist” or “to consist essentially of” and vice versa. In addition the verb “to consist” may be replaced by “to consist essentially of” meaning that a composition as defined herein may comprise additional component(s) than the ones specifically identified, the additional component(s) not altering the unique characteristic of the disclosure. In addition, reference to an element by the indefinite article “a” or “an” does not exclude the possibility that more than one of the element is present, unless the context clearly requires that there be one and only one of the elements. The indefinite article “a” or “an” thus usually means “at least one.” Throughout the disclosure, unless explicitly stated otherwise, the articles “a” and “an” are preferably replaced by “at least two,” more preferably by “at least three,” even more preferably by “at least four,” even more preferably by “at least five,” even more preferably by “at least six,” most preferably by “at least two.”

Throughout the disclosure, unless explicitly stated otherwise, the expressions “capable of . . . <verb>” and “capable to . . . <verb>” are preferably replaced with the active voice of the verb and vice versa. For example, the expression “capable of expressing” is preferably replaced with “expresses” and vice versa, i.e., “expresses” is preferably replaced with “capable of expressing.”

Throughout the disclosure, unless explicitly stated otherwise, the features “synthesize,” “synthesized” and “synthesis” are interchangeably used with the features “produce,” “produced” and “production,” respectively.

Each embodiment as identified herein may be combined together unless otherwise indicated. All publications, patents, and patent applications mentioned in this specification are herein incorporated by reference to the same extent as if each individual publication, patent, or patent application was specifically and individually indicated to be incorporated by reference. The full content of the priority application EP21186203, EP21168997 and EP20190204 are also incorporated by reference to the same extent as if the priority applications were specifically and individually indicated to be incorporated by reference.

According to the disclosure, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the disclosure. Moreover, DNAs or RNAs comprising unusual bases, such as inosine, or modified bases, such as tritylated bases, are to be understood to be covered by the term “polynucleotides.” It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid sidechains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulphide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

The term “polynucleotide encoding a polypeptide” as used herein encompasses polynucleotides that include a sequence encoding a polypeptide of the disclosure. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated,” as the term is employed herein. Similarly, a “synthetic” sequence, as the term is used herein, means any sequence that has been generated synthetically and not directly isolated from a natural source. “Synthesized,” as the term is used herein, means any synthetically generated sequence and not directly isolated from a natural source.

The terms “recombinant” or “transgenic” or “metabolically engineered” or “genetically modified,” as used herein with reference to a cell or host cell are used interchangeably and indicates that the cell replicates a heterologous nucleic acid, or expresses a peptide or protein encoded by a heterologous nucleic acid (i.e., a sequence “foreign to the cell” or a sequence “foreign to the location or environment in the cell”). Such cells are described to be transformed with at least one heterologous or exogenous gene, or are described to be transformed by the introduction of at least one heterologous or exogenous gene. Metabolically engineered or recombinant or transgenic cells can contain genes that are not found within the native (non-recombinant) form of the cell. Recombinant cells can also contain genes found in the native form of the cell wherein the genes are modified and re-introduced into the cell by artificial means. The terms also encompass cells that contain a nucleic acid endogenous to the cell that has been modified or its expression or activity has been modified without removing the nucleic acid from the cell; such modifications include those obtained by gene replacement, replacement of a promoter; site-specific mutation; and related techniques. Accordingly, a “recombinant polypeptide” is one that has been produced by a recombinant cell. A “heterologous sequence” or a “heterologous nucleic acid,” as used herein, is one that originates from a source foreign to the particular cell (e.g., from a different species), or, if from the same source, is modified from its original form or place in the genome. Thus, a heterologous nucleic acid operably linked to a promoter is from a source different from that from which the promoter was derived, or, if from the same source, is modified from its original form or place in the genome. The heterologous sequence may be stably introduced, e.g., by transfection, transformation, conjugation or transduction, into the genome of the host microorganism cell, wherein techniques may be applied that will depend on the cell and the sequence that is to be introduced. Various techniques are known to a person skilled in the art and are, e.g., disclosed in Sambrook et al., Molecular Cloning: A Laboratory Manual, 2nd Ed., Cold Spring Harbor Laboratory Press, Cold Spring Harbor, N.Y. (1989). The term “mutant” cell or microorganism as used within the context of the disclosure refers to a cell or microorganism that is genetically modified.

The term “endogenous” within the context of the disclosure refers to any polynucleotide, polypeptide or protein sequence that is a natural part of a cell and is occurring at its natural location in the cell chromosome and of which the control of expression has not been altered compared to the natural control mechanism acting on its expression. The term “exogenous” refers to any polynucleotide, polypeptide or protein sequence that originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast a “homologous” polynucleotide, gene, nucleic acid, polypeptide, or enzyme is used herein to denote a polynucleotide, gene, nucleic acid, polypeptide, or enzyme that is derived from the host organism species. When referring to a gene regulatory sequence or to an auxiliary nucleic acid sequence used for maintaining or manipulating a gene sequence (e.g., a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e., in the genome of a non-genetically engineered organism) is referred to herein as a “heterologous promoter,” even though the promoter may be derived from the same species (or, in some cases, the same organism) as the gene to which it is linked.

The term “modified activity” of a protein or an enzyme relates to a change in activity of the protein or the enzyme compared to the wild type, i.e., natural, activity of the protein or enzyme. The modified activity can either be an abolished, impaired, reduced or delayed activity of the protein or enzyme compared to the wild type activity of the protein or the enzyme but can also be an accelerated or an enhanced activity of the protein or the enzyme compared to the wild type activity of the protein or the enzyme. A modified activity of a protein or an enzyme is obtained by modified expression of the protein or enzyme or is obtained by expression of a modified, i.e., mutant form of the protein or enzyme. A modified activity of an enzyme further relates to a modification in the apparent Michaelis constant Km and/or the apparent maximal velocity (Vmax) of the enzyme.

The term “modified expression” of a gene relates to a change in expression compared to the wild type expression of the gene in any phase of the production process of the desired di- and/or oligosaccharide. The modified expression is either a lower or higher expression compared to the wild type, wherein the term “higher expression” is also defined as “overexpression” of the gene in the case of an endogenous gene or “expression” in the case of a heterologous gene that is not present in the wild type strain. Lower expression is obtained by means of common well-known technologies for a skilled person (such as the usage of siRNA, CrispR, CrispRi, riboswitch, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e., statistically significantly ‘less-able’ compared to a functional wild-type gene) or completely unable (such as knocked-out genes) to produce functional final products. The term “riboswitch” as used herein is defined to be part of the messenger RNA that folds into intricate structures that block expression by interfering with translation. Binding of an effector molecule induces conformational change(s) permitting regulated expression post-transcriptionally. Next to changing the gene of interest in such a way that lower expression is obtained as described above, lower expression can also be obtained by changing the transcription unit, the promoter, an untranslated region, the ribosome binding site, the Shine Dalgarno sequence or the transcription terminator. Lower expression or reduced expression can, for instance, be obtained by mutating one or more base pairs in the promoter sequence or changing the promoter sequence fully to a constitutive promoter with a lower expression strength compared to the wild type or an inducible promoter, which result in regulated expression or a repressible promoter, which results in regulated expression. Overexpression or expression is obtained by means of common well-known technologies for a skilled person (such as the usage of artificial transcription factors, de novo design of a promoter sequence, ribosome engineering, introduction or re-introduction of an expression module at euchromatin, usage of high-copy-number plasmids), wherein the gene is part of an “expression cassette,” which relates to any sequence in which a promoter sequence, untranslated region sequence (containing either a ribosome binding sequence, Shine Dalgarno or Kozak sequence), a coding sequence and optionally a transcription terminator is present, and leading to the expression of a functional active protein. The expression is either constitutive or regulated.

The term “constitutive expression” is defined as expression that is not regulated by transcription factors other than the subunits of RNA polymerase (e.g., bacterial sigma factors like σ⁷⁰, σ⁵⁴, or related σ-factors and the yeast mitochondrial RNA polymerase specificity factor MTF1 that co-associate with the RNA polymerase core enzyme) under certain growth conditions. Non-limiting examples of such transcription factors are CRP, LacI, ArcA, Cra, IclR in E. coli, or, Aft2p, Crz1p, Skn7 in Saccharomyces cerevisiae, or, DeoR, GntR, Fur in B. subtilis. The RNA polymerase is the catalytic machinery for the synthesis of RNA from a DNA template. RNA polymerase binds a specific DNA sequence to initiate transcription, for instance, via a sigma factor in prokaryotic hosts or via MTF1 in yeasts. Constitutive expression offers a constant level of expression with no need for induction or repression.

The term “regulated expression” is defined as a facultative or regulatory or tuneable expression of a gene that is only expressed upon a certain natural condition of the host (e.g., mating phase of budding yeast, stationary phase of bacteria), as a response to an inducer or repressor such as but not limited to glucose, allo-lactose, lactose, galactose, glycerol, arabinose, rhamnose, fucose, IPTG, methanol, ethanol, acetate, formate, aluminum, copper, zinc, nitrogen, phosphates, xylene, carbon or nitrogen depletion, or substrates or the produced product or chemical repression, as a response to an environmental change (e.g., anaerobic or aerobic growth, oxidative stress, pH shifts, temperature changes like e.g., heat-shock or cold-shock, osmolarity, light conditions, starvation) or dependent on the position of the developmental stage or the cell cycle of the host cell including but not limited to apoptosis and autophagy. Regulated expression allows for control as to when a gene is expressed. The term “inducible expression by a natural inducer” is defined as a facultative or regulatory expression of a gene that is only expressed upon a certain natural condition of the host (e.g., organism being in labor, or during lactation), as a response to an environmental change (e.g., including but not limited to hormone, heat, cold, pH shifts, light, oxidative or osmotic stress/signaling), or dependent on the position of the developmental stage or the cell cycle of the host cell including but not limited to apoptosis and autophagy. The term “inducible expression upon chemical treatment” is defined as a facultative or regulatory expression of a gene that is only expressed upon treatment with a chemical inducer or repressor, wherein the inducer and repressor comprise but are not limited to an alcohol (e.g., ethanol, methanol), a carbohydrate (e.g., glucose, galactose, glycerol, lactose, arabinose, rhamnose, fucose, allo-lactose), metal ions (e.g., aluminum, copper, zinc), nitrogen, phosphates, IPTG, acetate, formate, xylene.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a pre-sequence or secretory leader may be operably linked to DNA for a polypeptide if it is expressed as a pre-protein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. The control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of the polynucleotide to a polypeptide.

Generally, “operably linked” means that the DNA sequences being linked are contiguous, and, in the case of a secretory leader, contiguous and in reading phase. However, enhancers do not have to be contiguous.

The terms “co-expression module” and “gene co-expression networks” are used interchangeably and refer to groups of genes with similar and/or identical expression profiles at the same time or under the same conditions. The genes can be positively as well as negatively co-expressed: positively co-expressed genes are all expressed or have up-regulated expression (i.e., are over-expressed) at the same time or in a particular condition, whereas negatively co-expressed genes are all repressed or have down-regulated expression at the same time or in a particular condition. The term “operon” refers to a segment of DNA that contains a cluster of related genes under the control of a single promoter and a shared operator. The related genes are transcribed together to give a single messenger RNA (mRNA), which encodes multiple proteins. Transcription is initiated by binding of RNA polymerase to the promoter region, but the operator either allows or prevents transcription of the related genes into mRNA. The operator can be located within the promoter or between the promoter and the related genes. Operon regulation can be either negative or positive. Negative control involves turning off the operon in the presence of a regulatory protein being a repressor; this can be either repressible or inducible. Positive control involves turning on the operon in the presence of a regulatory protein being an inducer; this can be either repressible or inducible. The term “regulon” refers to a group of operons that are controlled by the same regulatory protein. The members of a regulon have separate promoters and are widely separated on the chromosome. The term “stimulon” refers to a regulon that is regulated by specific environmental stimuli like e.g., oxygen or nitric oxide levels. The term “modulon” refers to a regulon that is regulated in response to changes in overall conditions or stresses like e.g., quorum sensing. The term “biosynthetic gene cluster” refers to a physically grouping of all the genes that encode a biosynthetic pathway for the production of a secondary metabolite, including its chemical variants, like e.g., saccharides, terpenes, polyketides, alkaloids, bacteriocins, non-ribosomal peptides.

The term “wild type” refers to the commonly known genetic or phenotypical situation as it occurs in nature.

The term “modified expression of a protein” as used herein refers to i) higher expression or overexpression of an endogenous protein, ii) expression of a heterologous protein or iii) expression and/or overexpression of a variant protein that has a higher activity compared to the wild-type (i.e., native) protein.

As used herein, the term “mammary cell(s)” generally refers to mammary epithelial cell(s), mammary-epithelial luminal cell(s), or mammalian epithelial alveolar cell(s), or any combination thereof. As used herein, the term “mammary-like cell(s)” generally refers to cell(s) having a phenotype/genotype similar (or substantially similar) to natural mammary cell(s) but is/are derived from non-mammary cell source(s). Such mammary-like cell(s) may be engineered to remove at least one undesired genetic component and/or to include at least one predetermined genetic construct that is typical of a mammary cell. Non-limiting examples of mammary-like cell(s) may include mammary epithelial-like cell(s), mammary epithelial luminal-like cell(s), non-mammary cell(s) that exhibits one or more characteristics of a cell of a mammary cell lineage, or any combination thereof. Further non-limiting examples of mammary-like cell(s) may include cell(s) having a phenotype similar (or substantially similar) to natural mammary cell(s), or more particularly a phenotype similar (or substantially similar) to natural mammary epithelial cell(s). A cell with a phenotype or that exhibits at least one characteristic similar to (or substantially similar to) a natural mammary cell or a mammary epithelial cell may comprise a cell (e.g., derived from a mammary cell lineage or a non-mammary cell lineage) that exhibits either naturally, or has been engineered to, be capable of expressing at least one milk component.

As used herein, the term “non-mammary cell(s)” may generally include any cell of non-mammary lineage. In the context of the disclosure, a non-mammary cell can be any mammalian cell capable of being engineered to express at least one milk component. Non-limiting examples of such non-mammary cell(s) include hepatocyte(s), blood cell(s), kidney cell(s), cord blood cell(s), epithelial cell(s), epidermal cell(s), myocyte(s), fibroblast(s), mesenchymal cell(s), or any combination thereof. In some instances, molecular biology and genome editing techniques can be engineered to eliminate, silence, or attenuate myriad genes simultaneously.

“Variant(s)” as the term is used herein, is a polynucleotide or polypeptide that differs from a reference polynucleotide or polypeptide respectively but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

The term “derivative” of a polypeptide, as used herein, is a polypeptide, which may contain deletions, additions or substitutions of amino acid residues within the amino acid sequence of the polypeptide, but which result in a silent change, thus producing a functionally equivalent polypeptide. Amino acid substitutions may be made based on similarity in polarity, charge, solubility, hydrophobicity, hydrophilicity, and/or the amphipathic nature of the residues involved. For example, nonpolar (hydrophobic) amino acids include alanine, leucine, isoleucine, valine, proline, phenylalanine, tryptophan, and methionine; planar neutral amino acids include glycine, serine, threonine, cysteine, tyrosine, asparagine, and glutamine; positively charged (basic) amino acids include arginine, lysine, and histidine; and negatively charged (acidic) amino acids include aspartic acid and glutamic acid. Within the context of this disclosure, a derivative polypeptide as used herein, refers to a polypeptide capable of exhibiting a substantially similar in vitro and/or in vivo activity as the original polypeptide as judged by any of a number of criteria, including but not limited to enzymatic activity, and which may be differentially modified during or after translation. Furthermore, non-classical amino acids or chemical amino acid analogues can be introduced as a substitution or addition into the original polypeptide sequence.

In some embodiments, the disclosure contemplates making functional variants by modifying the structure of a protein of interest as used in the disclosure. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide.

“Fragment,” with respect to a polynucleotide, refers to a clone or any part of a polynucleotide molecule, particularly a part of a polynucleotide that retains a usable, functional characteristic of the full-length polynucleotide molecule. Useful fragments include oligonucleotides and polynucleotides that may be used in hybridization or amplification technologies or in the regulation of replication, transcription or translation. A “polynucleotide fragment” refers to any subsequence of a polynucleotide SEQ ID NO (or Genbank NO.), typically, comprising or consisting of at least about 9, 10, 11, 12 consecutive nucleotides from the polynucleotide SEQ ID NO (or Genbank NO.), for example, at least about 30 nucleotides or at least about 50 nucleotides of any of the polynucleotide sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide. As such, a fragment of a polynucleotide SEQ ID NO (or Genbank NO.) preferably means a nucleotide sequence, which comprises or consists of the polynucleotide SEQ ID NO (or Genbank NO.) wherein no more than 200, 10, 100, 50 or 25 consecutive nucleotides are missing, preferably no more than 50 consecutive nucleotides are missing, and which retains a usable, functional characteristic (e.g., activity) of the full-length polynucleotide molecule, which can be assessed by the skilled person through routine experimentation. Alternatively, a fragment of a polynucleotide SEQ ID NO (or Genbank NO.) preferably means a nucleotide sequence, which comprises or consists of an amount of consecutive nucleotides from the polynucleotide SEQ ID NO and wherein the amount of consecutive nucleotides is at least 50.0%, 60.0%, 70.0%, 80.0%, 81.0% 82.0%, 83.0% 84.0% 85.0% 86.0%, 87.0% 88.0% 89.0% 90.0% 91.0% 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0% of the full-length of the polynucleotide SEQ ID NO (or Genbank NO.) and retains a usable, functional characteristic (e.g., activity) of the full-length polynucleotide molecule. As such, a fragment of a polynucleotide SEQ ID NO (or Genbank NO.) preferably means a nucleotide sequence, which comprises or consists of the polynucleotide SEQ ID NO (or Genbank NO.), wherein an amount of consecutive nucleotides is missing and wherein the amount is no more than 50.0%, 40.0%, 30.0% of the full-length of the polynucleotide SEQ ID NO (or Genbank NO.), preferably no more than 20.0%, 15.0%, 10.0%, 90.0%, 8.0%, 70.0%, 60.0%, 5.0%, 4.5%, 4.0%, 3.5%, 30.0%, 2.5%, 2.0%, 1.5%, 1.0%, 0.5%, more preferably no more than 15%, even more preferably no more than 10%, even more preferably no more than 5.0%, most preferably no more than 2.5%, of the full-length of the polynucleotide SEQ ID NO (or Genbank NO.) and wherein the fragment retains a usable, functional characteristic (e.g., activity) of the full-length polynucleotide molecule, which can be routinely assessed by the skilled person.

Throughout the disclosure, the sequence of a polynucleotide can be represented by a SEQ ID NO or alternatively by a GenBank NO. Therefore, the terms “polynucleotide SEQ ID NO” and “polynucleotide GenBank NO.” can be interchangeably used, unless explicitly stated otherwise.

“Fragment,” with respect to a polypeptide, refers to a subsequence of the polypeptide, which performs at least one biological function of the intact polypeptide in substantially the same manner, or to a similar extent, as does the intact polypeptide. A “subsequence of the polypeptide” as defined herein refers to a sequence of contiguous amino acid residues derived from the polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example, at least about 20 amino acid residues in length, for example, at least about 30 amino acid residues in length. As such, a fragment of a polypeptide SEQ ID NO (or UniProt ID or Genbank NO.) preferably means a polypeptide sequence, which comprises or consists of the polypeptide SEQ ID NO (or UniProt ID or Genbank NO.) wherein no more than 80, 60, 50, 40, 30, 20 or 15 consecutive amino acid residues are missing, preferably no more than 40 consecutive amino acid residues are missing, and performs at least one biological function of the intact polypeptide in substantially the same manner, preferably to a similar or greater extent, as does the intact polypeptide, which can be routinely assessed by the skilled person. Alternatively, a fragment of a polypeptide SEQ ID NO (or UniProt ID or Genbank NO.) preferably means a polypeptide sequence, which comprises or consists of an amount of consecutive amino acid residues from the polypeptide SEQ ID NO (or UniProt ID or Genbank NO.) and wherein the amount of consecutive amino acid residues is at least 50.0%, 60.0%, 70.0%, 80.0%, 81.0%, 82.0%, 83.0%, 84.0%, 85.0%, 86.0%, 87.0%, 88.0%, 89.0%, 90.0%, 91.0%, 92.0%, 93.0%, 94.0%, 95.0%, 95.5%, 96.0%, 96.5%, 97.0%, 97.5%, 98.0%, 98.5%, 99.0%, 99.5%, 100%, preferably at least 80.0%, more preferably at least 87.0%, even more preferably at least 90.0%, even more preferably at least 95.0%, most preferably at least 97.0% of the full-length of the polypeptide SEQ ID NO (or UniProt ID or Genbank NO.) and which performs at least one biological function of the intact polypeptide in substantially the same manner, preferably to a similar or greater extent, as does the intact polypeptide, which can be routinely assessed by the skilled person. As such, a fragment of a polypeptide SEQ ID NO (or UniProt ID or Genbank NO.) preferably means a polypeptide sequence, which comprises or consists of the polypeptide SEQ ID NO (or UniProt ID or Genbank NO.), wherein an amount of consecutive amino acid residues is missing and wherein the amount is no more than 50.0%, 40.0%, 30.0% of the full-length of the polypeptide SEQ ID NO (or UniProt ID or Genbank NO.), preferably no more than 20.0%, 15.0%, 10.0%, 9.0%, 8.0%, 7.0%, 6.0%, 5.0%, 4.5%, 4.0%, 3.5%, 3.0%, 2.5%, 2.0%, 1.5%, 1.0%, 0.5%, more preferably no more than 15.0%, even more preferably no more than 10.0%, even more preferably no more than 5.0%, most preferably no more than 2.5%, of the full-length of the polypeptide SEQ ID NO (or UniProt ID or Genbank NO.) and which performs at least one biological function of the intact polypeptide in substantially the same manner, preferably to a similar or greater extent, as does the intact polypeptide, which can be routinely assessed by the skilled person.

Throughout the disclosure, the sequence of a polypeptide can be represented by a SEQ ID NO or alternatively by an UniProt ID or GenBank NO. Therefore, the terms “polypeptide SEQ ID NO” and “polypeptide UniProt ID” and “polypeptide GenBank NO.” can be interchangeably used, unless explicitly stated otherwise. A “functional fragment” of a polypeptide has at least one property or activity of the polypeptide from which it is derived, preferably to a similar or greater extent. A functional fragment can, for example, include a functional domain or conserved domain of a polypeptide. It is understood that a polypeptide or a fragment thereof may have conservative amino acid substitutions, which have substantially no effect on the polypeptide's activity. By conservative substitutions is intended substitutions of one hydrophobic amino acid for another or substitution of one polar amino acid for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa.

Homologous sequences as used herein describes those nucleotide sequences that have sequence similarity and encode polypeptides that share at least one functional characteristic such as a biochemical activity. More specifically, the term “functional homolog” as used herein describes those polypeptides that have sequence similarity (in other words, homology) and at the same time have at least one functional similarity such as a biochemical activity (Altenhoff et al., PLoS Comput. Biol. 8 (2012) e1002514).

Functional homologs are sometimes referred to as orthologs, where “ortholog” refers to a homologous gene or protein that is the functional equivalent of the referenced gene or protein in another species. Orthologous sequences are homologous sequences in different species that originate by vertical descent from a single sequence of the last common ancestor, wherein the sequence and its main function are conserved. A homologous sequence is a sequence inherited in two species by a common ancestor. The term “ortholog” when used in reference to an amino acid or nucleotide/nucleic acid sequence from a given species refers to the same amino acid or nucleotide/nucleic acid sequence from a different species. It should be understood that two sequences are orthologs of each other when they are derived from a common ancestor sequence via linear descent and/or are otherwise closely related in terms of both their sequence and their biological function. Orthologs will usually have a high degree of sequence identity but may not (and often will not) share 100% sequence identity. Paralogous sequences are homologous sequences that originate by a sequence duplication event. Paralogous sequences often belong to the same species, but this is not necessary. Paralogs can be split into in-paralogs (paralogous pairs that arose after a speciation event) and out-paralogs (paralogous pairs that arose before a speciation event). Between species out-paralogs are pairs of paralogs that exist between two organisms due to duplication before speciation. Within species out-paralogs are pairs of paralogs that exist in the same organism, but whose duplication event happened after speciation. Paralogs typically have the same or similar function.

Functional homologs will typically give rise to the same characteristics to a similar, but not necessarily the same, degree. Functionally homologous polypeptides give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events.

Functional homologs can be identified by analysis of nucleotide and polypeptide sequence alignments. For example, performing a query on a database of nucleotide or polypeptide sequences can identify homologs of the polypeptide of interest like e.g., a biomass-modulating polypeptide, a glycosyltransferase, a protein involved in nucleotide-activated sugar synthesis or a membrane transporter protein. Sequence analysis can involve BLAST, Reciprocal BLAST, or PSI-BLAST analysis of non-redundant databases using amino acid sequence of a biomass-modulating polypeptide, a glycosyltransferase, a protein involved in nucleotide-activated sugar synthesis or a membrane transporter protein, respectively, as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide, a glycosyltransferase, a protein involved in nucleotide-activated sugar synthesis or a membrane transporter protein, respectively. Amino acid sequence similarity allows for conservative amino acid substitutions, such as substitution of one hydrophobic residue for another or substitution of one polar residue for another or substitution of one acidic amino acid for another or substitution of one basic amino acid for another etc. Preferably, by conservative substitutions is intended combinations such as glycine by alanine and vice versa; valine, isoleucine and leucine by methionine and vice versa; aspartate by glutamate and vice versa; asparagine by glutamine and vice versa; serine by threonine and vice versa; lysine by arginine and vice versa; cysteine by methionine and vice versa; and phenylalanine and tyrosine by tryptophan and vice versa. If desired, manual inspection of such candidates can be carried out to narrow the number of candidates to be further evaluated. Manual inspection can be performed by selecting those candidates that appear to have domains present in productivity-modulating polypeptides, e.g., conserved functional domains.

A domain can be characterized, for example, by a Pfam (El-Gebali et al., Nucleic Acids Res. 47 (2019) D427-D432), an IPR (InterPro domain) (Mitchell et al., Nucleic Acids Res. 47 (2019) D351-D360), a protein fingerprint domain (PRINTS) (Attwood et al., Nucleic Acids Res. 31 (2003) 400-402), a SUBFAM domain (Gough et al., J. Mol. Biol. 313 (2001) 903-919), a TIGRFAM domain (Selengut et al., Nucleic Acids Res. 35 (2007) D260-D264), a Conserved Domain Database (CDD) designation (www.ncbi.nlm.nih.gov/cdd) (Lu et al., Nucleic Acids Res. 48 (2020) D265-D268), a PTHR domain (www.pantherdb.org) (Mi et al., Nucleic Acids. Res. 41 (2013) D377-D386; Thomas et al., Genome Research 13 (2003) 2129-2141) or a PATRIC identifier or PATRIC DB global family domain (www.patricbrc.org/) (Davis et al., Nucleic Acids Res. 48(D1) (2020) D606-D612). It should be understood for those skilled in the art that for the databases used herein, comprising Pfam 32.0 (released September 2018), CDD v3.17 (released 3 Apr. 2019), eggnogdb 4.5.1 (released September 2016), InterPro 75.0 (released 4 Jul. 2019), TCDB (released 17 Jun. 2019) and PATRIC 3.6.9 (released March 2020), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

Protein or polypeptide sequence information and functional information can be provided by a comprehensive resource for protein sequence and annotation data like e.g., the Universal Protein Resource (UniProt) (www.uniprot.org) (Nucleic Acids Res. 2021, 49(D1), D480-D489). UniProt comprises the expertly and richly curated protein database called the UniProt Knowledgebase (UniProtKB), together with the UniProt Reference Clusters (UniRef) and the UniProt Archive (UniParc). The UniProt identifiers (UniProt ID) are unique for each protein present in the database. UniProt IDs as used herein are the UniProt IDs in the UniProt database version of 5 May 2021. Proteins that do not have an UniProt ID are referred herein using the respective GenBank Accession number (GenBank NO.) as present in the NIH genetic sequence database (www.ncbi.nlm.nih.gov/genbank/) (Nucleic Acids Res. 2013, 41(D1), D36-D42) version of 5 May 2021.

The terms “identical” or “percent identity” or “% identity” in the context of two or more nucleic acid or polypeptide sequences, refer to two or more sequences or subsequences that are the same or have a specified percentage of nucleotides or amino acid residues that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity may be calculated globally over the full-length sequence of the reference sequence, resulting in a global percent identity score. Alternatively, percent identity may be calculated over a partial sequence of the reference sequence, resulting in a local percent identity score. Using the full-length of the reference sequence in a local sequence alignment results in a global percent identity score between the test and the reference sequence.

Percent identity can be determined using different algorithms like, for example, BLAST and PSI-BLAST (Altschul et al., 1990, J Mol Biol 215:3, 403-410; Altschul et al., 1997, Nucleic Acids Res 25: 17, 3389-402), the Clustal Omega method (Sievers et al., 2011, Mol. Syst. Biol. 7:539), the MatGAT method (Campanella et al., 2003, BMC Bioinformatics, 4:29) or EMBOSS Needle.

The BLAST (Basic Local Alignment Search Tool)) method of alignment is an algorithm provided by the National Center for Biotechnology Information (NCBI) to compare sequences using default parameters. The program compares nucleotide or protein sequences to sequence databases and calculates the statistical significance. PSI-BLAST (Position-Specific Iterative Basic Local Alignment Search Tool) derives a position-specific scoring matrix (PSSM) or profile from the multiple sequence alignment of sequences detected above a given score threshold using protein-protein BLAST (BLASTp). The BLAST method can be used for pairwise or multiple sequence alignments. Pairwise Sequence Alignment is used to identify regions of similarity that may indicate functional, structural and/or evolutionary relationships between two biological sequences (protein or nucleic acid). The web interface for BLAST is available at: blast.ncbi.nlm.nih.gov/Blast.cgi.

Clustal Omega (Clustal W) is a multiple sequence alignment program that uses seeded guide trees and HMM profile-profile techniques to generate alignments between three or more sequences. It produces biologically meaningful multiple sequence alignments of divergent sequences. The web interface for Clustal W is available at www.ebi.ac.uk/Tools/msa/clustalo/. Default parameters for multiple sequence alignments and calculation of percent identity of protein sequences using the Clustal W method are: enabling de-alignment of input sequences: FALSE; enabling mbed-like clustering guide-tree: TRUE; enabling mbed-like clustering iteration: TRUE; Number of (combined guide-tree/HIMM) iterations: default(0); Max Guide Tree Iterations: default [−1]; Max HMM Iterations: default [−1]; order: aligned.

MatGAT (Matrix Global Alignment Tool) is a computer application that generates similarity/identity matrices for DNA or protein sequences without needing pre-alignment of the data. The program performs a series of pairwise alignments using the Myers and Miller global alignment algorithm, calculates similarity and identity, and then places the results in a distance matrix. The user may specify which type of alignment matrix (e.g., BLOSUM50, BLOSUM62, and PAM250) to employ with their protein sequence examination.

EMBOSS Needle (galaxy-iuc.github.io/emboss-5.0-docs/needle.html) uses the Needleman-Wunsch global alignment algorithm to find the optimal alignment (including gaps) of two sequences when considering their entire length. The optimal alignment is ensured by dynamic programming methods by exploring all possible alignments and choosing the best. The Needleman-Wunsch algorithm is a member of the class of algorithms that can calculate the best score and alignment in the order of mn steps, (where ‘n’ and ‘m’ are the lengths of the two sequences). The gap open penalty (default 10.0) is the score taken away when a gap is created. The default value assumes you are using the EBLOSUM62 matrix for protein sequences. The gap extension (default 0.5) penalty is added to the standard gap penalty for each base or residue in the gap. This is how long gaps are penalized.

As used herein, a polypeptide having an amino acid sequence having at least 80% sequence identity to the full-length sequence of a reference polypeptide sequence is to be understood as that the sequence has 80%, 81%, 82%, 83%, 84%, 85%, 86%, 87%, 88%, 89%, 90%, 91%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, 99.60%, 99.70%, 99.80%, 99.90%, 100% sequence identity to the full-length of the amino acid sequence of the reference polypeptide sequence and having the same function and/or activity of interest as that reference polypeptide. Throughout the disclosure, unless explicitly specified otherwise, a polypeptide comprising, consisting or having an amino acid sequence having at least 80% sequence identity to the full-length amino acid sequence of a reference polypeptide, usually indicated with a SEQ ID NO, UniProt ID or Genbank NO., preferably has at least 85.0%, 90.0%, 91.0%, 92.0%, 93.0%, 94.0%, 95.0%, 96.0%, 97.0%, 98.0% or 99.0%, more preferably has at least 85.0%, even more preferably has at least 90.0%, most preferably has at least 95.0%, sequence identity to the full length reference sequence. Additionally, unless explicitly specified otherwise, a polynucleotide sequence comprising/consisting/having a nucleotide sequence having at least 80.0% sequence identity to the full-length nucleotide sequence of a reference polynucleotide sequence, usually indicated with a SEQ ID NO, UniProt ID or Genbank NO., preferably has at least 85.0%, 90.0%, 91.0%, 92.0%, 93.0%, 94.0%, 95.0%, 96.0%, 97.0%, 98.0% or 99.0%, more preferably has at least 85.0%, even more preferably has at least 90.0%, most preferably has at least 95.0%, sequence identity to the full length reference sequence.

For the purposes of this disclosure, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM65. In a preferred embodiment, sequence identity is calculated based on the full-length sequence of a given SEQ ID NO, i.e., the reference sequence, or a part thereof. Part thereof preferably means at least 50%, 60%, 70%, 80%, 90% or 95% of the complete reference sequence.

The terms “mannose-6-phosphate isomerase,” “phosphomannose isomerase,” “mannose phosphate isomerase,” “phosphohexoisomerase,” “phosphomannoisomerase,” “phosphomannose-isomerase,” “phosphohexomutase,” “D-mannose-6-phosphate ketol-isomerase”and “manA” are used interchangeably and refer to an enzyme that catalyzes the reversible conversion of D-fructose 6-phosphate to D-mannose 6-phosphate.

The terms “phosphomannomutase,” “mannose phosphomutase,” “phosphomannose mutase,” “D-mannose 1,6-phosphomutase” and “manB” are used interchangeably and refer to an enzyme that catalyzes the reversible conversion of D-mannose 6-phosphate to D-mannose 1-phosphate.

The terms “mannose-1-phosphate guanylyltransferase,” “GTP-mannose-1-phosphate guanylyltransferase,” “PIM-GMP (phosphomannose isomerase-guanosine 5′-diphospho-D-mannose pyrophosphorylase),” “GDP-mannose pyrophosphorylase,” “guanosine 5′-diphospho-D-mannose pyrophosphorylase,” “guanosine diphosphomannose pyrophosphorylase,” “guanosine triphosphate-mannose 1-phosphate guanylyltransferase,” “mannose 1-phosphate guanylyltransferase (guanosine triphosphate)” and “manC” are used interchangeably and refer to an enzyme that converts D-mannose-1-phosphate using GTP into GDP-mannose and diphosphate.

The terms “GDP-mannose 4,6-dehydratase,” “guanosine 5′-diphosphate-D-mannose oxidoreductase,” “guanosine diphosphomannose oxidoreductase,” “guanosine diphosphomannose 4,6-dehydratase,” “GDP-D-mannose dehydratase,” “GDP-D-mannose 4,6-dehydratase,” “GDP-mannose 4,6-hydro-lyase,” “GDP-mannose 4,6-hydro-lyase (GDP-4-dehydro-6-deoxy-D-mannose-forming)” and “gmd” are used interchangeably and refer to an enzyme that forms the first step in the biosynthesis of GDP-rhamnose and GDP-fucose.

The terms “GDP-L-fucose synthase,” “GDP-4-keto-6-deoxy-D-mannose-3,5-epimerase-4-reductase,” “GDP-L-fucose:NADP+ 4-oxidoreductase (3,5-epimerizing)” and “fcl” are used interchangeably and refer to an enzyme that forms the second step in the biosynthesis of GDP-fucose.

The terms “L-fucokinase/GDP-fucose pyrophosphorylase,” “L-fucokinase/L-fucose-1-P guanylyltransferase,” “GDP-fucose pyrophosphorylase,” “GDP-L-fucose pyrophosphorylase,” and “fkp” are used interchangeably and refer to an enzyme that catalyzes the conversion of L-fucose-1-phosphate into GDP-fucose using GTP.

The terms “L-glutamine-D-fructose-6-phosphate aminotransferase,” “glutamine-fructose-6-phosphate transaminase (isomerizing),” “hexosephosphate aminotransferase,” “glucosamine-6-phosphate isomerase (glutamine-forming),” “glutamine-fructose-6-phosphate transaminase (isomerizing),” “D-fructose-6-phosphate amidotransferase,” “glucosaminephosphate isomerase,” “glucosamine 6-phosphate synthase,” “GlcN6P synthase,” “GFA” and “glmS” are used interchangeably and refer to an enzyme that catalyzes the conversion of D-fructose-6-phosphate into D-glucosamine-6-phosphate using L-glutamine.

The terms “glucosamine-6-P deaminase,” “glucosamine-6-phosphate deaminase,” “GlcN6P deaminase,” “glucosamine-6-phosphate isomerase,” “glmD” and “nagB” are used interchangeably and refer to an enzyme that catalyzes the reversible isomerization-deamination of glucosamine-6-phosphate (GlcN6P) to form fructose-6-phosphate and an ammonium ion.

The terms “phosphoglucosamine mutase” and “glmM” are used interchangeably and refer to an enzyme that catalyzes the conversion of glucosamine-6-phosphate to glucosamine-1-phosphate. Phosphoglucosamine mutase can also catalyze the formation of glucose-6-P from glucose-1-P, although at a 1400-fold lower rate.

The terms “N-acetylglucosamine-6-P deacetylase,” “N-acetylglucosamine-6-phosphate deacetylase” and “nagA” are used interchangeably and refer to an enzyme that catalyzes the hydrolysis of the N-acetyl group of N-acetylglucosamine-6-phosphate (GlcNAc-6-P) to yield glucosamine-6-phosphate (GlcN6P) and acetate.

An N-acylglucosamine 2-epimerase is an enzyme that catalyzes the reaction N-acyl-D-glucosamine=N-acyl-D-mannosamine. Alternative names for this enzyme comprise N-acetylglucosamine 2-epimerase, N-acetyl-D-glucosamine 2-epimerase, GlcNAc 2-epimerase, N-acyl-D-glucosamine 2-epimerase and N-acetylglucosamine epimerase.

An UDP-N-acetylglucosamine 2-epimerase is an enzyme that catalyzes the reaction N-acetyl-D-glucosamine=N-acetylmannosamine. Alternative names for this enzyme comprise UDP-N-acylglucosamine 2-epimerase, UDP-GlcNAc-2-epimerase and UDP-N-acetyl-D-glucosamine 2-epimerase.

An N-acetylmannosamine-6-phosphate 2-epimerase is an enzyme that catalyzes the reaction N-acetyl-D-glucosamine 6-phosphate=N-acetyl-D-mannosamine 6-phosphate.

A bifunctional UDP-GlcNAc 2-epimerase/kinase is a bifunctional enzyme that catalyzes the reaction UDP-N-acetyl-D-glucosamine=N-acetyl-D-mannosamine and the reaction N-acetyl-D-mannosamine+ATP=ADP+N-acetyl-D-mannosamine 6-phosphate.

A glucosamine 6-phosphate N-acetyltransferase is an enzyme that catalyzes the transfer of an acetyl group from acetyl-CoA to D-glucosamine-6-phosphate thereby generating a free CoA and N-acetyl-D-glucosamine 6-phosphate. Alternative names comprise aminodeoxyglucosephosphate acetyltransferase, D-glucosamine-6-P N-acetyltransferase, glucosamine 6-phosphate acetylase, glucosamine 6-phosphate N-acetyltransferase, glucosaminephosphate N-acetyltransferase, glucosamine-6-phosphate acetylase, N-acetylglucosamine-6-phosphate synthase, phosphoglucosamine acetylase, phosphoglucosamine N-acetylase phosphoglucosamine N-acetylase, phosphoglucosamine transacetylase, GNA and GNA1.

The term “N-acetylglucosamine-6-phosphate phosphatase” refers to an enzyme that dephosphorylates N-acetylglucosamine-6-phosphate (GlcNAc-6-P) hereby synthesizing N-acetylglucosamine (GlcNAc).

The term “N-acetylmannosamine-6-phosphate phosphatase” refers to an enzyme that dephosphorylates N-acetylmannosamine-6-phosphate (ManNAc-6P) to N-acetylmannosamine (ManNAc).

The terms “N-acetylmannosamine-6-phosphate 2-epimerase,” “ManNAc-6-P isomerase,” “ManNAc-6-P 2-epimerase,” N-acetylglucosamine-6P 2-epimerase and “nanE” are used interchangeably and refer to an enzyme that converts ManNAc-6-P to N-acetylglucosamine-6-phosphate (GlcNAc-6-P).

The terms “phosphoacetylglucosamine mutase,” “acetylglucosamine phosphomutase,” “acetylaminodeoxyglucose phosphomutase,” “phospho-N-acetylglucosamine mutase” and “N-acetyl-D-glucosamine 1,6-phosphomutase” are used interchangeably and refer to an enzyme that catalyzes the conversion of N-acetyl-glucosamine 1-phosphate into N-acetylglucosamine 6-phosphate.

The terms “N-acetylglucosamine 1-phosphate uridylyltransferase,” “N-acetylglucosamine-1-phosphate uridyltransferase,” “UDP-N-acetylglucosamine diphosphorylase,” “UDP-N-acetylglucosamine pyrophosphorylase,” “uridine diphosphoacetylglucosamine pyrophosphorylase,” “UTP:2-acetamido-2-deoxy-alpha-D-glucose-1-phosphate uridylyltransferase,” “UDP-GlcNAc pyrophosphorylase,” “GlmU uridylyltransferase,” “Acetylglucosamine 1-phosphate uridylyltransferase,” “UDP-acetylglucosamine pyrophosphorylase,” “uridine diphosphate-N-acetylglucosamine pyrophosphorylase,” “uridine diphosphoacetylglucosamine phosphorylase,” and “acetylglucosamine 1-phosphate uridylyltransferase” are used interchangeably and refer to an enzyme that catalyzes the conversion of N-acetylglucosamine 1-phosphate (GlcNAc-1-P) into UDP-N-acetylglucosamine (UDP-GlcNAc) by the transfer of uridine 5-monophosphate (from uridine 5-triphosphate (UTP)).

The term glucosamine-1-phosphate acetyltransferase refers to an enzyme that catalyzes the transfer of the acetyl group from acetyl coenzyme A to glucosamine-1-phosphate (GlcN-1-P) to produce N-acetylglucosamine-1-phosphate (GlcNAc-1-P).

The term “glmU” refers to a bifunctional enzyme that has both N-acetylglucosamine-1-phosphate uridyltransferase and glucosamine-1-phosphate acetyltransferase activity and that catalyzes two sequential reactions in the de novo biosynthetic pathway for UDP-GlcNAc. The C-terminal domain catalyzes the transfer of acetyl group from acetyl coenzyme A to GlcN-1-P to produce GlcNAc-1-P, which is converted into UDP-GlcNAc by the transfer of uridine 5-monophosphate, a reaction catalyzed by the N-terminal domain.

The terms “NeunAc synthase,” “N-acetylneuraminic acid synthase,” “N-acetylneuraminate synthase,” “sialic acid synthase,” “NeuAc synthase,” “NeuB,” “NeuB1,” “NeuNAc synthase,” “NANA condensing enzyme,” “N-acetylneuraminate lyase synthase,” “N-acetylneuraminic acid condensing enzyme” as used herein are used interchangeably and refer to an enzyme capable to synthesize sialic acid from N-acetylmannosamine (ManNAc) in a reaction using phosphoenolpyruvate (PEP).

The terms “N-acetylneuraminate lyase,” “Neu5Ac lyase,” “N-acetylneuraminate pyruvate-lyase,” “N-acetylneuraminic acid aldolase,” “NALase,” “sialate lyase,” “sialic acid aldolase,” “sialic acid lyase” and “nanA” are used interchangeably and refer to an enzyme that degrades N-acetylneuraminate into N-acetylmannosamine (ManNAc) and pyruvate.

The terms “N-acylneuraminate-9-phosphate synthase,” “N-acylneuraminate-9-phosphate synthetase,” “NANA synthase,” “NANAS,” “NANS,” “NmeNANAS,” “N-acetylneuraminate pyruvate-lyase (pyruvate-phosphorylating)” as used herein are used interchangeably and refer to an enzyme capable to synthesize N-acylneuraminate-9-phosphate from N-acetylmannosamine-6-phosphate (ManNAc-6-phosphate) in a reaction using phosphoenolpyruvate (PEP).

The term “N-acylneuraminate-9-phosphatase” refers to an enzyme capable to dephosphorylate N-acylneuraminate-9-phosphate to synthesize N-acylneuraminate.

The terms “CMP-sialic acid synthase,” “N-acylneuraminate cytidylyltransferase,” “CMP-sialate synthase,” “CMP-NeuAc synthase,” “NeuA” and “CMP-N-acetylneuraminic acid synthase” as used herein are used interchangeably and refer to an enzyme capable to synthesize CMP-N-acetylneuraminate from N-acetylneuraminate using CTP in the reaction.

The terms “galactose-1-epimerase,” “aldose 1-epimerase,” “mutarotase,” “aldose mutarotase,” “galactose mutarotase,” “galactose 1-epimerase” and “D-galactose 1-epimerase” are used interchangeably and refer to an enzyme that catalyzes the conversion of beta-D-galactose into alpha-D-galactose.

The terms “galactokinase,” “galactokinase (phosphorylating)” and “ATP:D-galactose-1-phosphotransferase” are used interchangeably and refer to an enzyme that catalyzes the conversion of alpha-D-galactose into alpha-D-galactose 1-phosphate using ATP.

The terms glucokinase, and “glucokinase (phosphorylating)” are used interchangeably and refer to an enzyme that catalyzes the conversion of D-glucose into D-glucose 6-phosphate using ATP.

The terms “galactose-1-phosphate uridylyltransferase,” “Gal-1-P uridylyltransferase,” “UDP-glucose-hexose-1-phosphate uridylyltransferase,” “uridyl transferase,” “hexose-1-phosphate uridylyltransferase,” “uridyltransferase”; “hexose 1-phosphate uridyltransferase,” “UDP-glucose:alpha-D-galactose-1-phosphate uridylyltransferase,” “galB” and “galT” are used interchangeably and refer to an enzyme that catalyzes the reaction D-galactose 1-phosphate+UDP-D-glucose=D-glucose 1-phosphate+UDP-D-galactose.

The terms “UDP-glucose 4-epimerase,” “UDP-galactose 4-epimerase,” “uridine diphosphoglucose epimerase,” “galactowaldenase,” “UDPG-4-epimerase,” “uridine diphosphate galactose 4-epimerase,” “uridine diphospho-galactose-4-epimerase,” “UDP-glucose epimerase,” “4-epimerase,” “uridine diphosphoglucose 4-epimerase,” “uridine diphosphate glucose 4-epimerase” and “UDP-D-galactose 4-epimerase” are used interchangeably and refer to an enzyme that catalyzes the conversion of UDP-D-glucose into UDP-galactose.

The terms “glucose-1-phosphate uridylyltransferase,” “UTP-glucose-1-phosphate uridylyltransferase,” “UDP glucose pyrophosphorylase,” “UDPG phosphorylase,” “UDPG pyrophosphorylase,” “uridine 5′-diphosphoglucose pyrophosphorylase,” “uridine diphosphoglucose pyrophosphorylase,” “uridine diphosphate-D-glucose pyrophosphorylase,” “uridine-diphosphate glucose pyrophosphorylase” and “galU” are used interchangeably and refer to an enzyme that catalyzes the conversion of D-glucose-1-phosphate into UDP-glucose using UTP.

The terms “phosphoglucomutase (alpha-D-glucose-1,6-bisphosphate-dependent),” “glucose phosphomutase (ambiguous)” and “phosphoglucose mutase (ambiguous)” are used interchangeably and refer to an enzyme that catalyzes the conversion of D-glucose 1-phosphate into D-glucose 6-phosphate.

The terms “UDP-N-acetylglucosamine 4-epimerase,” “UDP acetylglucosamine epimerase,” “uridine diphosphoacetylglucosamine epimerase,” “uridine diphosphate N-acetylglucosamine-4-epimerase,” “uridine 5′-diphospho-N-acetylglucosamine-4-epimerase” and “UDP-N-acetyl-D-glucosamine 4-epimerase” are used interchangeably and refer to an enzyme that catalyzes the epimerization of UDP-N-acetylglucosamine (UDP-GlcNAc) to UDP-N-acetylgalactosamine (UDP-GalNAc).

The terms “N-acetylgalactosamine kinase,” “GALK2,” “GK2,” “GalNAc kinase,” “N-acetylgalactosamine (GalNAc)-1-phosphate kinase” and “ATP:N-acetyl-D-galactosamine 1-phosphotransferase” are used interchangeably and refer to an enzyme that catalyzes the synthesis of N-acetylgalactosamine 1-phosphate (GalNAc-1-P) from N-acetylgalactosamine (GalNAc) using ATP.

The terms “UDP-N-acetylgalactosamine pyrophosphorylase” and “UDP-GalNAc pyrophosphorylase” are used interchangeably and refer to an enzyme that catalyzes the conversion of N-acetylgalactosamine 1-phosphate (GalNAc-1-P) into UDP-N-acetylgalactosamine (UDP-GalNAc) using UTP.

The terms “N-acetylneuraminate kinase,” “ManNAc kinase,” “N-acetyl-D-mannosamine kinase” and “nanK” are used interchangeably and refer to an enzyme that phosphorylates ManNAc to synthesize N-acetylmannosamine-phosphate (ManNAc-6-P).

The term “glycosyltransferase” as used herein refers to an enzyme capable to catalyze the transfer of sugar moieties from activated donor molecules to specific acceptor molecules, forming glycosidic bonds. A classification of glycosyltransferases using nucleotide diphospho-sugar, nucleotide monophospho-sugar and sugar phosphates and related proteins into distinct sequence-based families has been described (Campbell et al., Biochem. J. 326, 929-939 (1997)) and is available on the CAZy (CArbohydrate-Active EnZymes) website (www.cazy.org).

As used herein the glycosyltransferase can be selected from the list comprising but not limited to: fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases.

Fucosyltransferases are glycosyltransferases that transfer a fucose residue (Fuc) from a GDP-fucose (GDP-Fuc) donor onto a glycan acceptor. Fucosyltransferases comprise alpha-1,2-fucosyltransferases, alpha-1,3-fucosyltransferases, alpha-1,4-fucosyltransferases and alpha-1,6-fucosyltransferases that catalyze the transfer of a Fuc residue from GDP-Fuc onto a glycan acceptor via alpha-glycosidic bonds. Fucosyltransferases can be found but are not limited to the GT10, GT11, GT23, GT65 and GT68 CAZy families. Sialyltransferases are glycosyltransferases that transfer a sialic acid (like Neu5Ac or Neu5Gc) from a donor (like CMP-Neu5Ac or CMP-Neu5Gc) onto a glycan acceptor. Sialyltransferases comprise alpha-2,3-sialyltransferases, alpha-2,6-sialyltransferases and alpha-2,8-sialyltransferases that catalyze the transfer of a sialic acid onto a glycan acceptor via alpha-glycosidic bonds. Sialyltransferases can be found but are not limited to the GT29, GT42, GT80 and GT97 CAZy families. Galactosyltransferases are glycosyltransferases that transfer a galactosyl group (Gal) from an UDP-galactose (UDP-Gal) donor onto a glycan acceptor. Galactosyltransferases comprise beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases and alpha-1,4-galactosyltransferases that transfer a Gal residue from UDP-Gal onto a glycan acceptor via alpha- or beta-glycosidic bonds. Galactosyltransferases can be found but are not limited to the GT2, GT6, GT8, GT25 and GT92 CAZy families. Glucosyltransferases are glycosyltransferases that transfer a glucosyl group (Glc) from an UDP-glucose (UDP-Glc) donor onto a glycan acceptor. Glucosyltransferases comprise alpha-glucosyltransferases, beta-1,2-glucosyltransferases, beta-1,3-glucosyltransferases and beta-1,4-glucosyltransferases that transfer a Glc residue from UDP-Glc onto a glycan acceptor via alpha- or beta-glycosidic bonds. Glucosyltransferases can be found but are not limited to the GT1, GT4 and GT25 CAZy families. Mannosyltransferases are glycosyltransferases that transfer a mannose group (Man) from a GDP-mannose (GDP-Man) donor onto a glycan acceptor. Mannosyltransferases comprise alpha-1,2-mannosyltransferases, alpha-1,3-mannosyltransferases and alpha-1,6-mannosyltransferases that transfer a Man residue from GDP-Man onto a glycan acceptor via alpha-glycosidic bonds. Mannosyltransferases can be found but are not limited to the GT22, GT39, GT62 and GT69 CAZy families. N-acetylglucosaminyltransferases are glycosyltransferases that transfer an N-acetylglucosamine group (GlcNAc) from an UDP-N-acetylglucosamine (UDP-GlcNAc) donor onto a glycan acceptor. N-acetylglucosaminyltransferases can be found but are not limited to GT2 and GT4 CAZy families. Galactoside beta-1,3-N-acetylglucosaminyltransferases are part of N-acetylglucosaminyltransferases and transfer GlcNAc from an UDP-GlcNAc donor onto a terminal galactose unit present in a glycan acceptor via a beta-1,3-linkage. Beta-1,6-N-acetylglucosaminyltransferases are N-acetylglucosaminyltransferases that transfer GlcNAc from an UDP-GlcNAc donor onto a glycan acceptor via a beta-1,6-linkage. N-acetylgalactosaminyltransferases are glycosyltransferases that transfer an N-acetylgalactosamine group (GalNAc) from an UDP-N-acetylgalactosamine (UDP-GalNAc) donor onto a glycan acceptor. N-acetylgalactosaminyltransferases can be found but are not limited to GT7, GT12 and GT27 CAZy families. Alpha-1,3-N-acetylgalactosaminyltransferases are part of the N-acetylgalactosaminyltransferases and transfer GalNAc from an UDP-GalNAc donor to a glycan acceptor via an alpha-1,3-linkage. N-acetylmannosaminyltransferases are glycosyltransferases that transfer an N-acetylmannosamine group (ManNAc) from an UDP-N-acetylmannosamine (UDP-ManNAc) donor onto a glycan acceptor. Xylosyltransferases are glycosyltransferases that transfer a xylose residue (Xyl) from an UDP-xylose (UDP-Xyl) donor onto a glycan acceptor. Xylosyltransferases can be found but are not limited to GT14, GT61 and GT77 CAZy families. Glucuronyltransferases are glycosyltransferases that transfer a glucuronate from an UDP-glucuronate donor onto a glycan acceptor via alpha- or beta-glycosidic bonds. Glucuronyltransferases can be found but are not limited to GT4, GT43 and GT93 CAZy families. Galacturonyltransferases are glycosyltransferases that transfer a galacturonate from an UDP-galacturonate donor onto a glycan acceptor. N-glycolylneuraminyltransferases are glycosyltransferases that transfer an N-glycolylneuraminic acid group (Neu5Gc) from a CMP-Neu5Gc donor onto a glycan acceptor. Rhamnosyltransferases are glycosyltransferases that transfer a rhamnose residue from a GDP-rhamnose donor onto a glycan acceptor. Rhamnosyltransferases can be found but are not limited to the GT1, GT2 and GT102 CAZy families. N-acetylrhamnosyltransferases are glycosyltransferases that transfer an N-acetylrhamnosamine residue from an UDP-N-acetyl-L-rhamnosamine donor onto a glycan acceptor. UDP-4-amino-4,6-dideoxy-N-acetyl-beta-L-altrosamine transaminases are glycosyltransferases that use an UDP-2-acetamido-2,6-dideoxy-L-arabino-4-hexulose in the biosynthesis of pseudaminic acid, which is a sialic acid-like sugar that is used to modify flagellin. UDP-N-acetylglucosamine enolpyruvyl transferases (murA) are glycosyltransferases that transfer an enolpyruvyl group from phosphoenolpyruvate (PEP) to UDP-N-acetylglucosamine (UDPAG) to form UDP-N-acetylglucosamine enolpyruvate. Fucosaminyltransferases are glycosyltransferases that transfer an N-acetylfucosamine residue from a dTDP-N-acetylfucosamine or an UDP-N-acetylfucosamine donor onto a glycan acceptor.

The terms “activated monosaccharide,” “nucleotide-activated sugar,” “nucleotide-sugar,” “activated sugar,” “nucleoside” or “nucleotide donor” are used herein interchangeably and refer to activated forms of monosaccharides. Examples of activated monosaccharides include but are not limited to UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), GDP-L-quinovose, CMP-sialic acid (CMP-Neu5Ac or CMP-N-acetylneuraminic acid), CMP-N-glycolylneuraminic acid (CMP-Neu5Gc), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, GDP-fucose (GDP-Fuc), GDP-rhamnose and UDP-xylose. Nucleotide-sugars act as glycosyl donors in glycosylation reactions. Glycosylation reactions are reactions that are catalyzed by glycosyltransferases.

The term “monosaccharide” as used herein refers to a sugar that is not decomposable into simpler sugars by hydrolysis, is classed either an aldose or ketose, and contains one or more hydroxyl groups per molecule. Monosaccharides are saccharides containing only one simple sugar. Examples of monosaccharides comprise Hexose, D-Glucopyranose, D-Galactofuranose, D-Galactopyranose, L-Galactopyranose, D-Mannopyranose, D-Allopyranose, L-Altropyranose, D-Gulopyranose, L-Idopyranose, D-Talopyranose, D-Ribofuranose, D-Ribopyranose, D-Arabinofuranose, D-Arabinopyranose, L-Arabinofuranose, L-Arabinopyranose, D-Xylopyranose, D-Lyxopyranose, D-Erythrofuranose, D-Threofuranose, Heptose, L-glycero-D-manno-Heptopyranose (LDmanHep), D-glycero-D-manno-Heptopyranose (DDmanHep), 6-Deoxy-L-altropyranose, 6-Deoxy-D-gulopyranose, 6-Deoxy-D-talopyranose, 6-Deoxy-D-galactopyranose, 6-Deoxy-L-galactopyranose, 6-Deoxy-D-mannopyranose, 6-Deoxy-L-mannopyranose, 6-Deoxy-D-glucopyranose, 2-Deoxy-D-arabino-hexose, 2-Deoxy-D-erythro-pentose, 2,6-Dideoxy-D-arabino-hexopyranose, 3,6-Dideoxy-D-arabino-hexopyranose, 3,6-Dideoxy-L-arabino-hexopyranose, 3,6-Dideoxy-D-xylo-hexopyranose, 3,6-Dideoxy-D-ribo-hexopyranose, 2,6-Dideoxy-D-ribo-hexopyranose, 3,6-Dideoxy-L-xylo-hexopyranose, 2-Amino-2-deoxy-D-glucopyranose, 2-Amino-2-deoxy-D-galactopyranose, 2-Amino-2-deoxy-D-mannopyranose, 2-Amino-2-deoxy-D-allopyranose, 2-Amino-2-deoxy-L-altropyranose, 2-Amino-2-deoxy-D-gulopyranose, 2-Amino-2-deoxy-L-idopyranose, 2-Amino-2-deoxy-D-talopyranose, 2-Acetamido-2-deoxy-D-glucopyranose, 2-Acetamido-2-deoxy-D-galactopyranose, 2-Acetamido-2-deoxy-D-mannopyranose, 2-Acetamido-2-deoxy-D-allopyranose, 2-Acetamido-2-deoxy-L-altropyranose, 2-Acetamido-2-deoxy-D-gulopyranose, 2-Acetamido-2-deoxy-L-idopyranose, 2-Acetamido-2-deoxy-D-talopyranose, 2-Acetamido-2,6-dideoxy-D-galactopyranose, 2-Acetamido-2,6-dideoxy-L-galactopyranose, 2-Acetamido-2,6-dideoxy-L-mannopyranose, 2-Acetamido-2,6-dideoxy-D-glucopyranose, 2-Acetamido-2,6-dideoxy-L-altropyranose, 2-Acetamido-2,6-dideoxy-D-talopyranose, D-Glucopyranuronic acid, D-Galactopyranuronic acid, D-Mannopyranuronic acid, D-Allopyranuronic acid, L-Altropyranuronic acid, D-Gulopyranuronic acid, L-Gulopyranuronic acid, L-Idopyranuronic acid, D-Talopyranuronic acid, sialic acid, 5-Amino-3,5-dideoxy-D-glycero-D-galacto-non-2-ulosonic acid, 5-Acetamido-3,5-dideoxy-D-glycero-D-galacto-non-2-ulosonic acid, 5-Glycolylamido-3,5-dideoxy-D-glycero-D-galacto-non-2-ulosonic acid, Erythritol, Arabinitol, Xylitol, Ribitol, Glucitol, Galactitol, Mannitol, D-ribo-Hex-2-ulopyranose, D-arabino-Hex-2-ulofuranose (D-fructofuranose), D-arabino-Hex-2-ulopyranose, L-xylo-Hex-2-ulopyranose, D-lyxo-Hex-2-ulopyranose, D-threo-Pent-2-ulopyranose, D-altro-Hept-2-ulopyranose, 3-C-(Hydroxymethyl)-D-erythofuranose, 2,4,6-Trideoxy-2,4-diamino-D-glucopyranose, 6-Deoxy-3-O-methyl-D-glucose, 3-O-Methyl-D-rhamnose, 2,6-Dideoxy-3-methyl-D-ribo-hexose, 2-Amino-3-O-[(R)-1-carboxyethyl]-2-deoxy-D-glucopyranose, 2-Acetamido-3-O-[(R)-carboxyethyl]-2-deoxy-D-glucopyranose, 2-Glycolylamido-3-O-[(R)-1-carboxyethyl]-2-deoxy-D-glucopyranose, 3-Deoxy-D-lyxo-hept-2-ulopyranosaric acid, 3-Deoxy-D-manno-oct-2-ulopyranosonic acid, 3-Deoxy-D-glycero-D-galacto-non-2-ulopyranosonic acid, 5,7-Diamino-3,5,7,9-tetradeoxy-L-glycero-L-manno-non-2-ulopyranosonic acid, 5,7-Diamino-3,5,7,9-tetradeoxy-L-glycero-L-altro-non-2-ulopyranosonic acid, 5,7-Diamino-3,5,7,9-tetradeoxy-D-glycero-D-galacto-non-2-ulopyranosonic acid, 5,7-Diamino-3,5,7,9-tetradeoxy-D-glycero-D-talo-non-2-ulopyranosonic acid, 2-acetamido-2,6-dideoxy-L-arabino-4-hexulose, 2-acetamido-2,6-dideoxy-L-lyxo-4-hexulose, N-acetyl-L-rhamnosamine, N-acetyl-D-fucosamine, N-acetyl-L-pneumosamine, N-acetylmuramic acid, N-acetyl-L-quinovosamine, glucose (Glc), galactose (Gal), N-acetylglucosamine (GlcNAc), glucosamine (Glcn), mannose (Man), xylose (Xyl), N-acetylmannosamine (ManNAc), N-glycolylneuraminic acid, N-acetylgalactosamine (GalNAc), galactosamine (Galn), fucose (Fuc), rhamnose (Rha), glucuronic acid, gluconic acid, fructose (Fru) and polyols.

With the term polyol is meant an alcohol containing multiple hydroxyl groups. For example, glycerol, sorbitol, or mannitol.

The terms “sialic acid,” “N-acetylneuraminate,” “N-acylneuraminate,” “N-acetylneuraminic acid” are used interchangeably and refer to an acidic sugar with a nine-carbon backbone comprising but not limited to Neu4Ac; Neu5Ac; Neu4,5Ac2; Neu5,7Ac2; Neu5,8Ac2; Neu5,9Ac2; Neu4,5,9Ac3; Neu5,7,9Ac3; Neu5,8,9Ac3; Neu4,5,7,9Ac4; Neu5,7,8,9Ac4, Neu4,5,7,8,9Ac5 and Neu5Gc.

Neu4Ac is also known as 4-O-acetyl-5-amino-3,5-dideoxy-D-glycero-D-galacto-non-2-ulopyranosonic acid or 4-O-acetyl neuraminic acid and has C₁₁H₁₉NO₉ as molecular formula. Neu5Ac is also known as 5-acetamido-3,5-dideoxy-D-glycero-D-galacto-non-2-ulopyranosonic acid, D-glycero-5-acetamido-3,5-dideoxy-D-galacto-non-2-ulo-pyranosonic acid, 5-(acetylamino)-3,5-dideoxy-D-glycero-D-galacto-2-nonulopyranosonic acid, 5-(acetylamino)-3,5-dideoxy-D-glycero-D-galacto-2-nonulosonic acid, 5-(acetylamino)-3,5-dideoxy-D-glycero-D-galacto-non-2-nonulosonic acid or 5-(acetylamino)-3,5-dideoxy-D-glycero-D-galacto-non-2-ulopyranosonic acid and has C₁₁H₁₉NO₉ as molecular formula. Neu4,5Ac2 is also known as N-acetyl-4-O-acetylneuraminic acid, 4-O-acetyl-N-acetylneuraminic acid, 4-O-acetyl-N-acetylneuraminate, 4-acetate 5-acetamido-3,5-dideoxy-D-glycero-D-galacto-nonulosonate, 4-acetate 5-(acetylamino)-3,5-dideoxy-D-glycero-D-galacto-2-nonulosonate, 4-acetate 5-acetamido-3,5-dideoxy-D-glycero-D-galacto-nonulosonic acid or 4-acetate 5-(acetylamino)-3,5-dideoxy-D-glycero-D-galacto-2-nonulosonic acid and has C₁₃H₂₁NO₁₀ as molecular formula. Neu5,7Ac2 is also known as 7-O-acetyl-N-acetylneuraminic acid, N-acetyl-7-O-acetylneuraminic acid, 7-O-acetyl-N-acetylneuraminate, 7-acetate 5-acetamido-3,5-dideoxy-D-glycero-D-galacto-nonulosonate, 7-acetate 5-(acetylamino)-3,5-dideoxy-D-glycero-D-galacto-2-nonulosonate, 7-acetate 5-acetamido-3,5-dideoxy-D-glycero-D-galacto-nonulosonic acid or 7-acetate 5-(acetylamino)-3,5-dideoxy-D-glycero-D-galacto-2-nonulosonic acid and has C₁₃H₂₁NO₁₀ as molecular formula. Neu5,8Ac2 is also known as 5-n-acetyl-8-o-acetyl neuraminic acid and has C₁₃H₂₁NO₁₀ as molecular formula. Neu5,9Ac2 is also known as N-acetyl-9-O-acetylneuraminic acid, 9-anana, 9-O-acetylsialic acid, 9-O-acetyl-N-acetylneuraminic acid, 5-n-acetyl-9-O-acetyl neuraminic acid, N,9-O-diacetylneuraminate or N,9-O-diacetylneuraminate and has C₁₃H₂₁NO₁₀ as molecular formula. Neu4,5,9Ac3 is also known as 5-N-acetyl-4,9-di-O-acetylneuraminic acid. Neu5,7,9Ac3 is also known as 5-N-acetyl-7,9-di-O-acetylneuraminic acid. Neu5,8,9Ac3 is also known as 5-N-acetyl-8,9-di-O-acetylneuraminic acid. Neu4,5,7,9Ac4 is also known as 5-N-acetyl-4,7,9-tri-O-acetylneuraminic acid. Neu5,7,8,9Ac4 is also known as 5-N-acetyl-7,8,9-tri-O-acetylneuraminic acid. Neu4,5,7,8,9Ac5 is also known as 5-N-acetyl-4,7,8,9-tetra-O-acetylneuraminic acid. Neu5Gc is also known as N-glycolyl-neuraminic acid, N-glycolylneuraminic acid, N-glycolylneuraminate, N-glycoloyl-neuraminate, N-glycoloylneuraminic acid, N-glycoloylneuraminic acid, 3,5-dideoxy-5-((hydroxyacetyl)amino)-D-glycero-D-galacto-2-nonulosonic acid, 3,5-dideoxy-5-(glycoloylamino)-D-glycero-D-galacto-2-nonulopyranosonic acid, 3,5-dideoxy-5-(glycoloylamino)-D-glycero-D-galacto-non-2-ulopyranosonic acid, 3,5-dideoxy-5-[(hydroxyacetyl)amino]-D-glycero-D-galacto-non-2-ulopyranosonic acid, D-glycero-5-glycolylamido-3,5-dideoxy-D-galacto-non-2-ulo-pyranosonic acid and has C₁₁H₁₉NO₁₀ as molecular formula.

The term “disaccharide” as used herein refers to a saccharide polymer containing two simple sugars, i.e., monosaccharides. Such disaccharides contain monosaccharides preferably selected from the list of monosaccharides as used herein above. Examples of disaccharides comprise lactose (Gal-b1,4-Glc), lacto-N-biose (Gal-b1,3-GlcNAc), N-acetyllactosamine (Gal-b1,4-GlcNAc), LacDiNAc (GalNAc-b1,4-GlcNAc), N-acetylgalactosaminylglucose (GalNAc-b1,4-Glc), Neu5Ac-a2,3-Gal, Neu5Ac-a2,6-Gal and fucopyranosyl-(1-4)-N-glycolylneuraminic acid (Fuc-(1-4)-Neu5Gc).

“Oligosaccharide” as the term is used herein and as generally understood in the state of the art, refers to a saccharide polymer containing a small number, typically three to twenty, of simple sugars, i.e., monosaccharides. Preferably the oligosaccharide as described herein contains monosaccharides selected from the list as used herein above. The oligosaccharide as used in the disclosure can be a linear structure or can include branches. The linkage (e.g., glycosidic linkage, galactosidic linkage, glucosidic linkage, etc.) between two sugar units can be expressed, for example, as 1,4, 1->4, or (1-4), used interchangeably herein. For example, the terms “Gal-b1,4-Glc,” “b-Gal-(1->4)-Glc,” “Galbeta1-4-Glc” and “Gal-b(1-4)-Glc” have the same meaning, i.e., a beta-glycosidic bond links carbon-1 of galactose (Gal) with the carbon-4 of glucose (Glc). Each monosaccharide can be in the cyclic form (e.g., pyranose or furanose form). Linkages between the individual monosaccharide units may include alpha 1->2, alpha 1->3, alpha 1->4, alpha 1->6, alpha 2->1, alpha 2->3, alpha 2->4, alpha 2->6, beta 1->2, beta 1->3, beta 1->4, beta 1->6, beta 2->1, beta 2->3, beta 2->4, and beta 2->6. An oligosaccharide can contain both alpha- and beta-glycosidic bonds or can contain only alpha-glycosidic or only beta-glycosidic bonds. The term “polysaccharide” refers to a compound consisting of a large number, typically more than twenty, of monosaccharides linked glycosidically.

Examples of oligosaccharides include but are not limited to Lewis-type antigen oligosaccharides, mammalian (including human) milk oligosaccharides, 0-antigen, enterobacterial common antigen (ECA), the glycan chain present in lipopolysaccharides (LPS), the oligosaccharide repeats present in capsular polysaccharides, peptidoglycan (PG), amino-sugars and antigens of the human ABO blood group system.

The term “glycan acceptor” as used herein refers to mono-, di- and oligosaccharides as defined herein.

As used herein, “mammalian milk oligosaccharide” refers to oligosaccharides such as but not limited to 3-fucosyllactose, 2′-fucosyllactose, 6-fucosyllactose, 2′,3-difucosyllactose, 2′,2-difucosyllactose, 3,4-difucosyllactose, 6′-sialyllactose, 3′-sialyllactose, 3,6-disialyllactose, 6,6′-disialyllactose, 8,3-disialyllactose, 3,6-disialyllacto-N-tetraose, lactodifucotetraose, lacto-N-tetraose, lacto-N-neotetraose, lacto-N-fucopentaose II, lacto-N-fucopentaose I, lacto-N-fucopentaose III, lacto-N-fucopentaose V, lacto-N-fucopentaose VI, sialyllacto-N-tetraose c, sialyllacto-N-tetraose b, sialyllacto-N-tetraose a, lacto-N-difucohexaose I, lacto-N-difucohexaose II, lacto-N-hexaose, lacto-N-neohexaose, para-lacto-N-hexaose, monofucosylmonosialyllacto-N-tetraose c, monofucosyl para-lacto-N-hexaose, monofucosyllacto-N-hexaose III, isomeric fucosylated lacto-N-hexaose III, isomeric fucosylated lacto-N-hexaose I, sialyllacto-N-hexaose, sialyllacto-N-neohexaose II, difucosyl-para-lacto-N-hexaose, difucosyllacto-N-hexaose, difucosyllacto-N-hexaose a, difucosyllacto-N-hexaose c, galactosylated chitosan, fucosylated oligosaccharides, neutral oligosaccharide and/or sialylated oligosaccharides.

A ‘fucosylated oligosaccharide’ as used herein and as generally understood in the state of the art is an oligosaccharide that is carrying a fucose-residue. Examples comprise 2′-fucosyllactose (2′FL), 3-fucosyllactose (3FL), 4-fucosyllactose (4FL), 6-fucosyllactose (6FL), difucosyllactose (diFL), lactodifucotetraose (LDFT), Lacto-N-fucopentaose I (LNF I), Lacto-N-fucopentaose II (LNF II), Lacto-N-fucopentaose III (LNF III), lacto-N-fucopentaose V (LNF V), lacto-N-fucopentaose VI (LNF VI), lacto-N-neofucopentaose I, lacto-N-difucohexaose I (LDFH I), lacto-N-difucohexaose II (LDFH II), Monofucosyllacto-N-hexaose III (MFLNH III), Difucosyllacto-N-hexaose (DFLNHa), difucosyl-lacto-N-neohexaose.

As used herein, a ‘sialylated oligosaccharide’ is to be understood as a charged sialic acid containing oligosaccharide, i.e., an oligosaccharide having a sialic acid residue. It has an acidic nature. Some examples are 3-SL (3′-sialyllactose or 3′-SL or Neu5Ac-a2,3-Gal-b1,4-Glc), 3′-sialyllactosamine, 6-SL (6′-sialyllactose or 6′-SL or Neu5Ac-a2,6-Gal-b1,4-Glc), 3,6-disialyllactose (Neu5Ac-a2,3-(Neu5Ac-a2,6)-Gal-b1,4-Glc), 6,6′-disialyllactose (Neu5Ac-a2,6-Gal-b1,4-(Neu5Ac-a2,6)-Glc), 8,3-disialyllactose (Neu5Ac-a2,8-Neu5Ac-a2,3-Gal-b1,4-Glc), 6′-sialyllactosamine, oligosaccharides comprising 6′-sialyllactose, SGG hexasaccharide (Neu5Acα-2,3Galβ-1,3GalNacβ-1,3Galα-1,4Galβ-1,4Gal), sialylated tetrasaccharide (Neu5Acα-2,3Galβ-1,4GlcNacβ-14GlcNAc), pentasaccharide LSTD (Neu5Acα-2,3Galβ-1,4GlcNacβ-1,3Galβ-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residue(s), including but not limited to: oligosaccharide moieties of the gangliosides selected from GM3 (3′sialyllactose, Neu5Acα-2,3Galβ-4Glc) and oligosaccharides comprising the GM3 motif, GD3 Neu5Acα-2,8Neu5Acα-2,3Galβ-1,4Glc GT3 (Neu5Acα-2,8Neu5Acα-2,8Neu5Acα-2,3Galβ-1,4Glc); GM2 GalNAcβ-1,4(Neu5Acα-2,3)Galβ-1,4Glc, GM1 Galβ-1,3GalNAcβ-1,4(Neu5Acα-2,3)Galβ-1,4Glc, GD1a Neu5Acα-2,3Galβ-1,3GalNAcβ-1,4(Neu5Acα-2,3)Galβ-1,4Glc, GT1a Neu5Acα-2,8Neu5Acα-2,3Galβ-1,3GalNAcβ-1,4(Neu5Acα-2,3)Galβ-1,4Glc, GD2 GalNAcβ-1,4(Neu5Acα-2,8Neu5Acα2,3)Galβ-1,4Glc, GT2 GalNAcβ-1,4(Neu5Acα-2,8Neu5Acα-2,8Neu5Acα2,3)Galβ-1,4Glc, GD1b, Galβ-1,3GalNAcβ-1,4(Neu5Acα-2,8Neu5Acα2,3)Galβ-1,4Glc, GT1b Neu5Acα-2,3Galβ-1,3GalNAcβ-1,4(Neu5Acα-2,8Neu5Acα2,3)Galβ-1,4Glc, GQ1b Neu5Acα-2,8Neu5Acα-2,3Galβ-1,3GalNAcβ-1,4(Neu5Acα-2,8Neu5Acα2,3)Galβ-1,4Glc, GT1c Galβ-1,3GalNAcβ-1,4(Neu5Acα-2,8Neu5Acα-2,8Neu5Acα2,3)Galβ-1,4Glc, GQ1c Neu5Acα-2,3Galβ-1,3GalNAc β-1,4(Neu5Acα-2,8Neu5Acα-2,8Neu5Acα2,3)Galβ-1,4Glc, GP1c Neu5Acα-2,8Neu5Acα-2,3Galβ-1,3GalNAc β-1,4(Neu5Acα-2,8Neu5Acα-2,8Neu5Acα2,3)Galβ-1,4Glc, GD1a Neu5Acα-2,3Galβ-1,3(Neu5Acα-2,6)GalNAcβ-1,4Galβ-1,4Glc, Fucosyl-GM1 Fucα-1,2Galβ-1,3GalNAcβ-1,4(Neu5Acα-2,3)Galβ-1,4Glc; all of which may be extended to the production of the corresponding gangliosides by reacting the above oligosaccharide moieties with ceramide or synthetizing the above oligosaccharides on a ceramide.

A ‘neutral oligosaccharide’ as used herein and as generally understood in the state of the art is an oligosaccharide that has no negative charge originating from a carboxylic acid group. Examples of such neutral oligosaccharide are 2′-fucosyllactose (2′FL), 3-fucosyllactose (3FL), 2′,3-difucosyllactose (diFL), lacto-N-triose II, lacto-N-tetraose, lacto-N-neotetraose, lacto-N-fucopentaose I, lacto-N-neofucopentaose I, lacto-N-fucopentaose II, lacto-N-fucopentaose III, lacto-N-fucopentaose V, lacto-N-fucopentaose VI, lacto-N-neofucopentaose V, lacto-N-difucohexaose I, lacto-N-difucohexaose II, 6′-galactosyllactose, 3′-galactosyllactose, lacto-N-hexaose, lacto-N-neohexaose, para-lacto-N-hexaose, para-lacto-N-neohexaose, difucosyl-lacto-N-hexaose and difucosyl-lacto-N-neohexaose.

Mammalian milk oligosaccharides or MMOs comprise oligosaccharides present in milk found in any phase during lactation including colostrum milk from humans (i.e., human milk oligosaccharides or HMOs) and mammals including but not limited to cows (Bos Taurus), sheep (Ovis aries), goats (Capra aegagrus hircus), bactrian camels (Camelus bactrianus), horses (Equus ferus caballus), pigs (Sus scropha), dogs (Canis lupus familiaris), ezo brown bears (Ursus arctos yesoensis), polar bear (Ursus maritimus), Japanese black bears (Ursus thibetanus japonicus), striped skunks (Mephitis mephitis), hooded seals (Cystophora cristata), Asian elephants (Elephas maximus), African elephant (Loxodonta africana), giant anteater (Myrmecophaga tridactyla), common bottlenose dolphins (Tursiops truncates), northern minke whales (Balaenoptera acutorostrata), tammar wallabies (Macropus eugenii), red kangaroos (Macropus rufus), common brushtail possum (Trichosurus vulpecula), koalas (Phascolarctos cinereus), eastern quolls (Dasyurus viverrinus), platypus (Ornithorhynchus anatinus). Human milk oligosaccharides are also known as human identical milk oligosaccharides, which are chemically identical to the human milk oligosaccharides found in human breast milk, but which are biotechnologically-produced (e.g., using cell free systems or cells and organisms comprising a bacterium, a fungus, a yeast, a plant, animal, or protozoan cell, preferably genetically engineered cells and organisms). Human identical milk oligosaccharides are marketed under the name HiMO.

As used herein the term “Lewis-type antigens” comprise the following oligosaccharides: H1 antigen, which is Fucα1-2Galβ1-3GlcNAc, or in short 2′FLNB; Lewisa (Lea), which is the trisaccharide Galβ1-3[Fucα1-4]GlcNAc, or in short 4-FLNB; Lewisb (Leb), which is the tetrasaccharide Fucα1-2Galβ1-3[Fucα1-4]GlcNAc, or in short DiF-LNB; sialyl Lewisa (sialyl Lea), which is 5-acetylneuraminyl-(2-3)-galactosyl-(1-3)-(fucopyranosyl-(1-4))-N-acetylglucosamine, or written in short Neu5Acα2-3Galβ1-3[Fucα1-4]GlcNAc; H2 antigen, which is Fucα1-2Galβ1-4GlcNAc, or otherwise stated 2′fucosyl-N-acetyl-lactosamine, in short 2′FLacNAc; Lewisx (Lex), which is the trisaccharide Galβ1-4[Fucα1-3]GlcNAc, or otherwise known as 3-Fucosyl-N-acetyl-lactosamine, in short 3-FLacNAc; Lewisy (Ley), which is the tetrasaccharide Fucα1-2Galβ1-4[Fucα1-3]GlcNAc; and sialyl Lewisx (sialyl Lex) which is 5-acetylneuraminyl-(2-3)-galactosyl-(1-4)-(fucopyranosyl-(1-3))-N-acetylglucosamine, or written in short Neu5Acα2-3Galβ1-4[Fucα1-3]GlcNAc.

The terms “LNB” and “Lacto-N-biose” are used interchangeably and refer to the disaccharide Gal-b1,3-GlcNAc.

The terms “LacNAc” and “N-acetyllactosamine” are used interchangeably and refer to the disaccharide Gal-b1,4-GlcNAc.

As used herein, the term “O-antigen” refers to the repetitive glycan component of the surface lipopolysaccharide (LPS) of Gram-negative bacteria. The term “lipopolysaccharide” or “LPS” refers to glycolipids found in the outer membrane of Gram-negative bacteria, which are composed of a lipid A, a core oligosaccharide and the O-antigen. The term “enterobacterial common antigen” or “ECA” refers to a specific carbohydrate antigen built of repeating units of three amino sugars, i.e., N-acetylglucosamine, N-acetyl-d-mannosaminuronic acid and 4-acetamido-4,6-dideoxy-d-galactose, which is shared by all members of the Enterobacteriaceae and which is located in the outer leaflet of the outer membrane and in the periplasm. The term “capsular polysaccharides” refers to long-chain polysaccharides with oligosaccharide repeat structures that are present in bacterial capsules, the latter being a polysaccharide layer that lies outside the cell envelope. The terms “peptidoglycan” or “murein” refers to an essential structural element in the cell wall of most bacteria, being composed of sugars and amino acids, wherein the sugar components consist of alternating residues of beta-1,4 linked GlcNAc and N-acetylmuramic acid. The term “amino-sugar” as used herein refers to a sugar molecule in which a hydroxyl group has been replaced with an amine group. As used herein, an antigen of the human ABO blood group system is an oligosaccharide. Such antigens of the human ABO blood group system are not restricted to human structures. The structures involve the A determinant GalNAc-alpha1,3(Fuc-alpha1,2)-Gal-, the B determinant Gal-alpha1,3(Fuc-alpha1,2)-Gal- and the H determinant Fuc-alpha1,2-Gal- that are present on disaccharide core structures comprising Gal-beta1,3-GlcNAc, Gal-beta1,4-GlcNAc, Gal-beta1,3-GalNAc and Gal-beta1,4-Glc.

The terms “LNT II,” “LNT-II,” “LN3,” “lacto-N-triose II,” “lacto-N-triose II,” “lacto-N-triose,” “lacto-N-triose” or “GlcNAcβ1-3Galβ1-4GlC” as used in the disclosure, are used interchangeably.

The terms “LNT,” “lacto-N-tetraose,” “lacto-N-tetraose” or “Galβ1-3GlcNAcβ1-3Galβ1-4Glc” as used in the disclosure, are used interchangeably.

The terms “LNnT,” “lacto-N-neotetraose,” “lacto-N-neotetraose,” “neo-LNT” or “Galβ1-4GlcNAcβ1-3Galβ1-4Glc” as used in the disclosure, are used interchangeably.

The terms “LSTa,” “LS-Tetrasaccharide a,” “Sialyl-lacto-N-tetraose a,” “sialyllacto-N-tetraose a” or “Neu5Ac-a2,3-Gal-b1,3-GlcNAc-b1,3-Gal-b1,4-Glc” as used in the disclosure, are used interchangeably.

The terms “LSTb,” “LS-Tetrasaccharide b,” “Sialyl-lacto-N-tetraose b,” “sialyllacto-N-tetraose b” or “Gal-b1,3-(Neu5Ac-a2,6)-GlcNAc-b1,3-Gal-b1,4-Glc” as used in the disclosure, are used interchangeably.

The terms “LSTc,” “LS-Tetrasaccharide c,” “Sialyl-lacto-N-tetraose c,” “sialyllacto-N-tetraose c,” “sialyllacto-N-neotetraose c” or “Neu5Ac-a2,6-Gal-b1,4-GlcNAc-b1,3-Gal-b1,4-Glc” as used in the disclosure, are used interchangeably.

The terms “LSTd,” “LS-Tetrasaccharide d,” “Sialyl-lacto-N-tetraose d,” “sialyllacto-N-tetraose d,” “sialyllacto-N-neotetraose d” or “Neu5Ac-a2,3-Gal-b1,4-GlcNAc-b1,3-Gal-b1,4-Glc” as used in the disclosure, are used interchangeably.

The terms “DSLNnT” and “Disialyllacto-N-neotetraose” are used interchangeably and refer to Neu5Ac-a2,6-[Neu5Ac-a2,6-Gal-b1,4-GlcNAc-b1,3]-Gal-b1,4-Glc.

The terms “DSLNT” and “Disialyllacto-N-tetraose” are used interchangeably and refer to Neu5Ac-a2,6-[Neu5Ac-a2,3-Gal-b1,3-GlcNAc-b1,3]-Gal-b1,4-Glc. The terms “LNFP-I,” “lacto-N-fucopentaose I,” “LNFP I,” “LNF I OH type I determinant,” “LNF I,” “LNF1,” “LNF 1” and “Blood group H antigen pentaose type 1” are used interchangeably and refer to Fuc-a1,2-Gal-b1,3-GlcNAc-b1,3-Gal-b1,4-Glc.

The terms “GalNAc-LNFP-I” and “blood group A antigen hexaose type I” are used interchangeably and refer to GalNAc-a1,3-(Fuc-a1,2)-Gal-b1,3-GlcNAc-b1,3-Gal-b1,4-Glc.

The terms “LNFP-II” and “lacto-N-fucopentaose II” are used interchangeably and refer to Gal-b1,3-(Fuc-a1,4)-GlcNAc-b1,3-Gal-b1,4-Glc.

The terms “LNFP-III” and “lacto-N-fucopentaose III” are used interchangeably and refer to Gal-b1,4-(Fuc-a1,3)-GlcNAc-b1,3-Gal-b1,4-Glc.

The terms “LNFP-V” and “lacto-N-fucopentaose V” are used interchangeably and refer to Gal-b1,3-GlcNAc-b1,3-Gal-b1,4-(Fuc-a1,3)-Glc.

The terms “LNFP-VI,” “LNnFP V” and “lacto-N-neofucopentaose V” are used interchangeably and refer to Gal-b1,4-GlcNAc-b1,3-Gal-b1,4-(Fuc-a1,3)-Glc.

The terms “LNnFP I” and “Lacto-N-neofucopentaose I” are used interchangeably and refer to Fuc-a1,2-Gal-b1,4-GlcNAc-b1,3-Gal-b1,4-Glc.

The terms “LNDFH I,” “Lacto-N-difucohexaose I,” “LNDFH-I,” “LDFH I,” “Le^{b}-lactose” and “Lewis-b hexasaccharide” are used interchangeably and refer to Fuc-a1,2-Gal-b1,3-[Fuc-a1,4]-GlcNAc-b1,3-Gal-b1,4-Glc.

The terms “LNDFH II,” “Lacto-N-difucohexaose II,” “Lewis a-Lewis x” and “LDFH II” are used interchangeably and refer to Fuc-a1,4-(Gal-b1,3)-GlcNAc-b1,3-Gal-b1,4-(Fuc-a1,3)-Glc.

The terms “LNnDFH,” “Lacto-N-neoDiFucohexaose” and “Lewis x hexaose” are used interchangeably and refer to Gal-b1,4-(Fuc-a1,3)-GlcNAc-b1,3-Gal-b1,4-(Fuc-a1,3)-Glc.

The terms “alpha-tetrasaccharide” and “A-tetrasaccharide” are used interchangeably and refer to GalNAc-a1,3-(Fuc-a1,2)-Gal-b1,4-Glc.

The term “membrane transporter proteins” as used herein refers to proteins that are part of or interact with the cell membrane and control the flow of molecules and information across the cell. The membrane proteins are thus involved in transport, be it import into or export out of the cell.

Such membrane transporter proteins can be porters, P-P-bond-hydrolysis-driven transporters, β-Barrel Porins, auxiliary transport proteins, putative transport proteins and phosphotransfer-driven group translocators as defined by the Transporter Classification Database that is operated and curated by the Saier Lab Bioinformatics Group available via www.tcdb.org and providing a functional and phylogenetic classification of membrane transport proteins This Transporter Classification Database details a comprehensive IUBMB approved classification system for membrane transporter proteins known as the Transporter Classification (TC) system. The TCDB classification searches as described here are defined based on TCDB. org as released on 17 Jun. 2019.

Porters is the collective name of uniporters, symporters, and antiporters that utilize a carrier-mediated process (Saier et al., Nucleic Acids Res. 44 (2016) D372-D379). They belong to the electrochemical potential-driven transporters and are also known as secondary carrier-type facilitators. Membrane transporter proteins are included in this class when they utilize a carrier-mediated process to catalyze uniport when a single species is transported either by facilitated diffusion or in a membrane potential-dependent process if the solute is charged; antiport when two or more species are transported in opposite directions in a tightly coupled process, not coupled to a direct form of energy other than chemiosmotic energy; and/or symport when two or more species are transported together in the same direction in a tightly coupled process, not coupled to a direct form of energy other than chemiosmotic energy, of secondary carriers (Forrest et al., Biochim. Biophys. Acta 1807 (2011) 167-188). These systems are usually stereospecific. Solute:solute countertransport is a characteristic feature of secondary carriers. The dynamic association of porters and enzymes creates functional membrane transport metabolons that channel substrates typically obtained from the extracellular compartment directly into their cellular metabolism (Moraes and Reithmeier, Biochim. Biophys. Acta 1818 (2012), 2687-2706). Solutes that are transported via this porter system include but are not limited to cations, organic anions, inorganic anions, nucleosides, amino acids, polyols, phosphorylated glycolytic intermediates, osmolytes, siderophores.

Membrane transporter proteins are included in the class of P-P-bond hydrolysis-driven transporters if they hydrolyze the diphosphate bond of inorganic pyrophosphate, ATP, or another nucleoside triphosphate, to drive the active uptake and/or extrusion of a solute or solutes (Saier et al., Nucleic Acids Res. 44 (2016) D372-D379). The membrane transporter protein may or may not be transiently phosphorylated, but the substrate is not phosphorylated. Substrates that are transported via the class of P-P-bond hydrolysis-driven transporters include but are not limited to cations, heavy metals, beta-glucan, UDP-glucose, lipopolysaccharides, teichoic acid.

The β-Barrel porins membrane transporter proteins form transmembrane pores that usually allow the energy independent passage of solutes across a membrane. The transmembrane portions of these proteins consist exclusively of β-strands, which form a β-barrel (Saier et al., Nucleic Acids Res. 44 (2016) D372-D379). These porin-type proteins are found in the outer membranes of Gram-negative bacteria, mitochondria, plastids, and possibly acid-fast Gram-positive bacteria. Solutes that are transported via these β-Barrel porins include but are not limited to nucleosides, raffinose, glucose, beta-glucosides, oligosaccharides.

The auxiliary transport proteins are defined to be proteins that facilitate transport across one or more biological membranes but do not themselves participate directly in transport. These membrane transporter proteins always function in conjunction with one or more established transport systems such as but not limited to outer membrane factors (OMFs), polysaccharide (PST) porters, the ATP-binding cassette (ABC)-type transporters. They may provide a function connected with energy coupling to transport, play a structural role in complex formation, serve a biogenic or stability function or function in regulation (Saier et al., Nucleic Acids Res. 44 (2016) D372-D379). Examples of auxiliary transport proteins include but are not limited to the polysaccharide copolymerase family involved in polysaccharide transport, the membrane fusion protein family involved in bacteriocin and chemical toxin transport.

Putative transport protein comprises families that will either be classified elsewhere when the transport function of a member becomes established or will be eliminated from the Transporter Classification system if the proposed transport function is disproven. These families include a member or members for which a transport function has been suggested, but evidence for such a function is not yet compelling (Saier et al., Nucleic Acids Res. 44 (2016) D372-D379). Examples of putative transporters classified in this group under the TCDB system as released on 17 Jun. 2019 include but are not limited to copper transporters.

The phosphotransfer-driven group translocators are also known as the PEP-dependent phosphoryl transfer-driven group translocators of the bacterial phosphoenolpyruvate:sugar phosphotransferase system (PTS). The product of the reaction, derived from extracellular sugar, is a cytoplasmic sugar-phosphate. The enzymatic constituents, catalyzing sugar phosphorylation, are superimposed on the transport process in a tightly coupled process. The PTS system is involved in many different aspects comprising in regulation and chemotaxis, biofilm formation, and pathogenesis (Lengeler, J. Mol. Microbiol. Biotechnol. 25 (2015) 79-93; Saier, J. Mol. Microbiol. Biotechnol. 25 (2015) 73-78). Membrane transporter protein families classified within the phosphotransfer-driven group translocators under the TCDB system as released on 17 Jun. 2019 include PTS systems linked to transport of glucose-glucosides, fructose-mannitol, lactose-N,N′-diacetylchitobiose-beta-glucoside, glucitol, galactitol, mannose-fructose-sorbose and ascorbate.

The major facilitator superfamily (MFS) is a superfamily of membrane transporter proteins catalyzing uniport, solute:cation (H+, but seldom Na+) symport and/or solute:H+ or solute:solute antiport. Most are of 400-600 amino acyl residues in length and possess either 12, 14, or occasionally, 24 transmembrane α-helical spanners (TMSs) as defined by the Transporter Classification Database operated by the Saier Lab Bioinformatics Group (www.tcdb.org).

“SET” or “Sugar Efflux Transporter” as used herein refers to membrane proteins of the SET family, which are proteins with InterPRO domain IPR004750 and/or are proteins that belong to the eggNOGv4.5 family ENOG410XTE9. Identification of the InterPro domain can be done by using the online tool on www.ebi.ac.uk/interpro/or a standalone version of InterProScan (www.ebi.ac.uk/interpro/download.html) using the default values. Identification of the orthology family in eggNOGv4.5 can be done using the online version or a standalone version of eggNOG-mapperv1 (eggnogdb.embl.de/#/app/home).

The term “Siderophore” as used herein is referring to the secondary metabolite of various microorganisms, which are mainly ferric ion specific chelators. These molecules have been classified as catecholate, hydroxamate, carboxylate and mixed types. Siderophores are in general synthesized by a nonribosomal peptide synthetase (NRPS) dependent pathway or an NRPS independent pathway (NIS). The most important precursor in NRPS-dependent siderophore biosynthetic pathway is chorismate. 2,3-DHBA could be formed from chorismate by a three-step reaction catalyzed by isochorismate synthase, isochorismatase, and 2,3-dihydroxybenzoate-2,3-dehydrogenase. Siderophores can also be formed from salicylate, which is formed from isochorismate by isochorismate pyruvate lyase. When ornithine is used as precursor for siderophores, biosynthesis depends on the hydroxylation of ornithine catalyzed by L-ornithine N5-monooxygenase. In the NIS pathway, an important step in siderophore biosynthesis is N(6)-hydroxylysine synthase.

A transporter is needed to export the siderophore outside the cell. Four superfamilies of membrane proteins are identified so far in this process: the major facilitator superfamily (MFS); the Multidrug/Oligosaccharidyl-lipid/Polysaccharide Flippase Superfamily (MOP); the resistance, nodulation and cell division superfamily (RND); and the ABC superfamily. In general, the genes involved in siderophore export are clustered together with the siderophore biosynthesis genes. The term “siderophore exporter” as used herein refers to such transporters needed to export the siderophore outside of the cell.

The ATP-binding cassette (ABC) superfamily contains both uptake and efflux transport systems, and the members of these two groups generally cluster loosely together. ATP hydrolysis without protein phosphorylation energizes transport. There are dozens of families within the ABC superfamily, and family generally correlates with substrate specificity. Members are classified according to class 3.A.1 as defined by the Transporter Classification Database operated by the Saier Lab Bioinformatics Group available via www.tcdb.org and providing a functional and phylogenetic classification of membrane transporter proteins.

It should be understood for those skilled in the art that for the databases used herein, comprising eggnogdb 4.5.1 (released September 2016) and InterPro 75.0 (released 4 Jul. 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The term “cell for the production of a di- and/or oligosaccharide” within the context of the disclosure refers to a cell that comprises any one or more of i) one or more glycosyltransferase(s) necessary for the synthesis of the di- and/or oligosaccharide ii) one or more biosynthetic pathway(s) to produce one or more nucleotide donor(s) suitable to be transferred by the glycosyltransferase(s) to a carbohydrate acceptor, iii) one or more biosynthetic pathway(s) to produce one or more precursor(s) as defined herein, iv) a mechanism of internalization of one or more precursor(s) from the culture medium into the cell, v) a mechanism for enabled and/or enhanced efflux of the di- and/or oligosaccharide from the cell to the outside of the cell and vi) a mechanism for disabled and/or diminished efflux from the cell to the outside of the cell of any one or more metabolite(s) and/or by-product(s) that is/are synthesized during the production of the di- and/or oligosaccharide.

The term “pathway for production of a di- and/or oligosaccharide” as used herein is a biochemical pathway consisting of the enzymes and their respective genes involved in the synthesis of a di- and/or oligosaccharide as defined herein. The pathway for production of a di- and/or oligosaccharide may comprise any one or more of i) pathways involved in the synthesis of a nucleotide-activated sugar ii) the transfer of the nucleotide-activated sugar to an acceptor by one or more glycosyltransferase(s) to produce a di- and/or oligosaccharide of the disclosure, iii) a mechanism for enabled efflux of the produced di- and/or oligosaccharide, preferably a mechanism for enhanced efflux of the produced di- and/or oligosaccharide, and iv) a mechanism for disabled and/or diminished efflux of any one or more metabolite(s) and/or by-product(s) that is/are synthesized during the production of the di- and/or oligosaccharide. Examples of such pathway comprise but are not limited to a fucosylation, sialylation, galactosylation, N-acetylglucosaminylation, N-acetylgalactosaminylation, mannosylation, N-acetylmannosaminylation pathway.

A ‘fucosylation pathway’ as used herein is a biochemical pathway comprising at least one of the enzymes and their respective genes chosen from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase combined with a fucosyltransferase leading to α 1,2; α 1,3; α 1,4 and/or α 1,6 fucosylated oligosaccharides.

A ‘sialylation pathway’ is a biochemical pathway comprising at least one of the enzymes and their respective genes chosen from the list comprising N-acylglucosamine 2-epimerase, UDP-N-acetylglucosamine 2-epimerase, N-acetylmannosamine-6-phosphate 2-epimerase, UDP-GlcNAc 2-epimerase/kinase hydrolyzing, N-acylneuraminate-9-phosphate synthase, phosphatase, N-acetylneuraminate synthase, N-acylneuraminate cytidylyltransferase and sialic acid transporter combined with a sialyltransferase leading to a 2,3; a 2,6 and/or a 2,8 sialylated oligosaccharides.

A ‘galactosylation pathway’ as used herein is a biochemical pathway comprising at least one of the enzymes and their respective genes chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase combined with a galactosyltransferase leading to a galactosylated compound comprising a mono-, di-, or oligosaccharide having an alpha or beta bound galactose on any one or more of the 2, 3, 4 and 6 hydroxyl group of the mono-, di-, or oligosaccharide.

An ‘N-acetylglucosaminylation pathway’ as used herein is a biochemical pathway comprising at least one of the enzymes and their respective genes chosen from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase combined with a glycosyltransferase leading to a GlcNAc-modified compound comprising a mono-, di-, or oligosaccharide having an alpha or beta bound N-acetylglucosamine (GlcNAc) on any one or more of the 3, 4 and 6 hydroxyl group of the mono-, di- or oligosaccharide.

An ‘N-acetylgalactosaminylation pathway’ as used herein is a biochemical pathway comprising at least one of the enzymes and their respective genes chosen from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase and/or UDP-N-acetylgalactosamine pyrophosphorylase combined with a glycosyltransferase leading to a GalNAc-modified compound comprising a mono-, di- or oligosaccharide having an alpha or beta bound N-acetylgalactosamine on the mono-, di- or oligosaccharide.

A ‘mannosylation pathway’ as used herein is a biochemical pathway comprising at least one of the enzymes and their respective genes chosen from the list comprising mannose-6-phosphate isomerase, phosphomannomutase and/or mannose-1-phosphate guanylyltransferase combined with a glycosyltransferase leading to a mannosylated compound comprising a mono-, di- or oligosaccharide having an alpha or beta bound mannose on the mono-, di- or oligosaccharide.

An ‘N-acetylmannosaminylation pathway’ as used herein is a biochemical pathway comprising at least one of the enzymes and their respective genes chosen from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase and/or ManNAc kinase combined with a glycosyltransferase leading to a ManNAc-modified compound comprising a mono-, di- or oligosaccharide having an alpha or beta bound N-acetylmannosamine on the mono-, di- or oligosaccharide.

The term “enabled efflux” means to introduce the activity of transport of a solute over the cytoplasm membrane and/or the cell wall. The transport may be enabled by introducing and/or increasing the expression of a membrane transporter protein as described in the disclosure. The term “enhanced efflux” means to improve the activity of transport of a solute over the cytoplasm membrane and/or the cell wall. Transport of a solute over the cytoplasm membrane and/or cell wall may be enhanced by introducing and/or increasing the expression of a membrane transporter protein as described in the disclosure. “Expression” of a membrane transporter protein is defined as “overexpression” of the gene encoding the membrane transporter protein in the case the gene is an endogenous gene or “expression” in the case the gene encoding the membrane transporter protein is a heterologous gene that is not present in the wild type strain or cell.

The terms “acetyl-coenzyme A synthetase,” “acs,” “acetyl-CoA synthetase,” “AcCoA synthetase,” “acetate-CoA ligase,” “acyl-activating enzyme” and “yfaC” are used interchangeably and refer to an enzyme that catalyses the conversion of acetate into acetyl-coezyme A (AcCoA) in an ATP-dependent reaction.

The terms “pyruvate dehydrogenase,” “pyruvate oxidase,” “POX,” “poxB” and “pyruvate:ubiquinone-8 oxidoreductase” are used interchangeably and refer to an enzyme that catalyses the oxidative decarboxylation of pyruvate to produce acetate and CO₂.

The terms “lactate dehydrogenase,” “D-lactate dehydrogenase,” “ldhA,” “hslI,” “htpH,” “D-LDH,” “fermentative lactate dehydrogenase” and “D-specific 2-hydroxyacid dehydrogenase” are used interchangeably and refer to an enzyme that catalyzes the conversion of lactate into pyruvate hereby generating NADH.

As used herein, the term “cell productivity index (CPI)” refers to the mass of the product produced by the cells divided by the mass of the cells produced in the culture.

The term “purified” refers to material that is substantially or essentially free from components that interfere with the activity of the biological molecule. For cells, saccharides, nucleic acids, and polypeptides, the term “purified” refers to material that is substantially or essentially free from components that normally accompany the material as found in its native state. Typically, purified saccharides, oligosaccharides, proteins or nucleic acids of the disclosure are at least about 50%, 55%, 60%, 65%, 70%, 75%, 80% or 85% pure, usually at least about 90%, 91%, 92%, 93%, 94%, 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For di- and oligosaccharides, purity can be determined using methods such as but not limited to thin layer chromatography, gas chromatography, NMR, HPLC, capillary electrophoresis or mass spectroscopy.

The term “cultivation” refers to the culture medium wherein the cell is cultivated or fermented, the cell itself, and the di- and/or oligosaccharide that is produced by the cell in whole broth, i.e., inside (intracellularly) as well as outside (extracellularly) of the cell.

The term “precursor” as used herein refers to substances that are taken up and/or synthetized by the cell for the specific production of a di- and/or oligosaccharide according to the disclosure. In this sense a precursor can be an acceptor as defined herein, but can also be another substance, metabolite, which is first modified within the cell as part of the biochemical synthesis route of the di- and/or oligosaccharide. Examples of such precursors comprise the acceptors as defined herein, and/or glucose, galactose, fructose, glycerol, sialic acid, fucose, mannose, maltose, sucrose, lactose, dihydroxyacetone, glucosamine, N-acetyl-glucosamine, mannosamine, N-acetylmannosamine, galactosamine, N-acetylgalactosamine, phosphorylated sugars like e.g., but not limited to glucose-1-phosphate, galactose-1-phosphate, glucose-6-phosphate, fructose-6-phosphate, fructose-1,6-bisphosphate, mannose-6-phosphate, mannose-1-phosphate, glycerol-3-phosphate, glyceraldehyde-3-phosphate, dihydroxyacetone-phosphate, glucosamine-6-phosphate, N-acetyl-glucosamine-6-phosphate, N-acetylmannosamine-6-phosphate, N-acetylglucosamine-1-phosphate, N-acetyl-neuraminic acid-9-phosphate and/or nucleotide-activated sugars as defined herein like e.g., UDP-glucose, UDP-galactose, UDP-N-acetylglucosamine, CMP-sialic acid, GDP-mannose, GDP-4-dehydro-6-deoxy-α-D-mannose, GDP-fucose.

Optionally, the cell is transformed to comprise and to express at least one nucleic acid sequence encoding a protein selected from the group consisting of lactose transporter, fucose transporter, transporter for a nucleotide-activated sugar wherein the transporter internalizes a to the medium added precursor for the synthesis of the di- and/or oligosaccharide of disclosure.

The term “acceptor” as used herein refers to a mono-, di- or oligosaccharide that can be modified by a glycosyltransferase. Examples of such acceptors comprise glucose, galactose, fructose, glycerol, sialic acid, fucose, mannose, maltose, sucrose, lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, and oligosaccharide containing 1 or more N-acetyllactosamine units and/or 1 or more lacto-N-biose units or an intermediate into oligosaccharide, fucosylated and sialylated versions thereof.

### DETAILED DESCRIPTION

According to a first aspect, the disclosure provides a cell for the production of a di- and/or oligosaccharide. Herein, a cell comprising a pathway for the production of a di- and/or oligosaccharide is provided, which is genetically modified for expression and/or overexpression of at least one set of multiple coding DNA sequences wherein the multiple coding DNA sequences within one set differ in nucleotide sequence and each encode a polypeptide, wherein the polypeptides have the same function and/or activity of interest. Preferably, the polypeptides are essentially the same polypeptides, more preferably, the polypeptides are identical to each other.

According to a second aspect, the disclosure provides a method for the production of a di- and/or oligosaccharide by a cell. The method comprises the steps of:
1) providing a cell as described herein, and
2) cultivating the cell under conditions permissive to produce the di- and/or oligosaccharide.

Preferably, the di- and/or oligosaccharide is separated from the cultivation as explained herein.

In the scope of the disclosure, permissive conditions are understood to be conditions relating to physical or chemical parameters including but not limited to temperature, pH, pressure, osmotic pressure and product/precursor/acceptor concentration.

In a particular embodiment, the permissive conditions may include a temperature-range of 30+/−20 degrees centigrade, a pH-range of 7+/−3.

In a preferred embodiment of the method, the permissive conditions comprise use of a culture medium comprising at least one precursor and/or acceptor as defined herein for the production of the di- and/or oligosaccharide. In an alternative and/or additional preferred embodiment of the method, the permissive conditions comprise adding to the culture medium at least one precursor and/or acceptor feed for the production of the di- and/or oligosaccharide.

According to an embodiment of the method and/or cell of disclosure, the polypeptides that are encoded in the cell by expression and/or overexpression of one set of multiple coding DNA sequences are variants, fragments or derivatives of each other, as defined herein, that have the same function and/or activity of interest. According to a preferred embodiment of the method and/or cell of the disclosure, the polypeptides are functional variants of each other as defined herein, comprising functional homologs, orthologs and paralogs. The functional variants have the same function and/or activity of interest but can differ in any one or more of amino acid composition, sequence, three-dimensional structure, protein stability, regulatory properties and kinetic parameters comprising K_{M}, k_{cat}, catalytic efficiency, enzymatic rate and velocity. The functional variants may have different catalytic efficiencies to catalyze the same chemical reaction.

It should be understood that the polypeptides encoded in a cell by a set of multiple coding DNA sequences of disclosure do not comprise polypeptides lacking catalytic residues like e.g., non-enzymes, dead enzymes, prozymes or ‘zombie’ proteins.

The disclosure provides different types of cells for the production of a di- and/or oligosaccharide.

In a preferred embodiment of the method and/or cell of disclosure, the cell comprises a set of two coding DNA sequences that differ in nucleotide sequence and that each encode a polypeptide, wherein both polypeptides have the same function and/or activity of interest. In a more preferred embodiment of the method and/or cell of disclosure, the cell comprises a set of at least two coding DNA sequences that differ in nucleotide sequence and that each encode a polypeptide, wherein both polypeptides have the same function and/or activity of interest. In an even more preferred embodiment of the method and/or cell of disclosure, the cell comprises a set of more than two, in other words, at least three coding DNA sequences that differ in nucleotide sequence and that each encode a polypeptide, wherein the polypeptides have the same function and/or activity of interest. In an even more preferred embodiment, the cell comprises a set of at least four coding DNA sequences according to the disclosure. In a most preferred embodiment, the cell comprises a set of at least five coding DNA sequences according to the disclosure.

In a preferred embodiment of the method and/or cell of the disclosure, the cell comprises two sets of multiple coding DNA sequences 1) wherein each set of the two sets consists of multiple coding DNA sequences that differ in nucleotide sequence and each set of the two sets encode for a polypeptide, wherein the polypeptides have the same function and/or activity of interest and 2) wherein the polypeptides encoded by the first set of the two sets of multiple coding DNA sequences have a different function and/or activity of interest compared to the other polypeptides that are encoded by the second set of the two sets of multiple coding DNA sequences as defined herein. In a more preferred embodiment, the cell comprises at least two sets of multiple coding DNA sequences as defined herein wherein the polypeptides encoded by each set of multiple coding DNA sequences have a different function and/or activity of interest compared to the other polypeptides that are encoded by the other sets of multiple coding DNA sequences. In an even more preferred embodiment, the cell comprises more than two, in other words, at least three sets of multiple coding DNA sequences as defined herein wherein the polypeptides encoded by each set of multiple coding DNA sequences have a different function and/or activity of interest compared to the other polypeptides that are encoded by the other sets of multiple coding DNA sequences. In an even more preferred embodiment, the cell comprises more than three, in other words, at least four sets of multiple coding DNA sequences as defined herein wherein the polypeptides encoded by each set of multiple coding DNA sequences have a different function and/or activity of interest compared to the other polypeptides that are encoded by the other sets of multiple coding DNA sequences. In a most preferred embodiment, the cell comprises more than four, in other words, at least five sets of multiple coding DNA sequences as defined herein wherein the polypeptides encoded by each set of multiple coding DNA sequences have a different function and/or activity of interest compared to the other polypeptides that are encoded by the other sets of multiple coding DNA sequences.

The number of coding DNA sequences present in each of the sets can be identical but does not need to be identical. A cell of disclosure may consist of two sets of multiple coding DNA sequences, wherein the first set consists of two coding DNA sequences that differ in nucleotide sequence and each encode for a polypeptide having the same function and/or activity of interest and wherein the second set also consists of two coding DNA sequences that differ in nucleotide sequence and each encode for a polypeptide having the same function and/or activity of interest and wherein the polypeptides encoded by the first set of two coding DNA sequences have a different function and/or activity of interest compared to the polypeptides encoded by the second set of two coding DNA sequences. Alternatively, a cell of disclosure may consist of two sets of multiple coding DNA sequences, wherein the first set consists of two coding DNA sequences that differ in nucleotide sequence and each encode for a polypeptide having the same function and/or activity of interest and wherein the second set consists of three or more coding DNA sequences that differ in nucleotide sequence and each encode for a polypeptide having the same function and/or activity of interest wherein the polypeptides encoded by the first set of two coding DNA sequences have a different function and/or activity of interest compared to the polypeptides encoded by the second set of three coding DNA sequences. Alternatively, a cell of disclosure may consist of more than two sets of multiple coding DNA sequences as defined herein, wherein the number of coding DNA sequences within each set can be two, three, four, five or more than five.

In a preferred embodiment of the method and/or cell of disclosure, the polypeptides that are encoded in the cell by expression and/or overexpression of a set of multiple coding DNA sequences are essentially the same polypeptides. In an exemplary embodiment, essentially the same polypeptides are polypeptides having conservative amino acid residues at certain positions in the polypeptide sequence wherein the substitutive conservative amino acid residues have a neglective effect on the polypeptide's function and/or activity of interest. By conservative substitutions is intended substitutions of one hydrophobic amino acid for another or substitution of one polar amino acid for another or substitution of one acidic amino acid for another or substitution of one basic amino acid for another etc. In another and/or additional exemplary embodiment, essentially the same polypeptides are polypeptides comprising an additional N- and/or C-terminal tag like a solubility enhancer tag or an affinity tag like e.g., a SUMO-tag, an MBP-tag, a His tag, a FLAG® tag, a Strep-II tag, a Halo-tag, a NusA tag, thioredoxin, a GST tag and a Fh8-tag, which have a neglective effect on the polypeptide's function and/or activity of interest. In another and/or additional exemplary embodiment, essentially the same polypeptides are truncated polypeptides lacking amino acid residues at certain positions in the polypeptide sequence without affecting the polypeptide's function and/or activity of interest.

In a more preferred embodiment, the polypeptides are identical to each other. In an exemplary embodiment, the cell comprises one set of multiple coding DNA sequences that encode two polypeptides that differ in amino acid sequence and that catalyze the same enzymatic reaction but with a different enzymatic rate. In another exemplary embodiment, the cell comprises one set of multiple coding DNA sequences that encode three or more polypeptides wherein all polypeptides differ in amino acid sequence and catalyze the same enzymatic reaction but with a different enzymatic rate. In another exemplary embodiment, the cell comprises one set of multiple coding DNA sequences that encode two or more polypeptides wherein two or more of the polypeptides are identical to each other in amino acid sequence and catalyze the same enzymatic reaction with an comparable/identical enzymatic rate. In another exemplary embodiment, the cells comprises two or more sets of multiple coding DNA sequences wherein each set comprises at least two coding DNA sequences that encode two or more polypeptides wherein two or more of the polypeptides are identical to each other in amino acid sequence and catalyze the same enzymatic reaction with an comparable/identical enzymatic rate.

In the context of the disclosure, polypeptides that constitute different subunits of one multi-subunit polypeptide complex and that function together to obtain a functional active form of the multi-subunit polypeptide complex are no functional variants of each other according to the disclosure. Each subunit polypeptide of such a complex is considered to fulfil a different function and/or activity. For example, the different subunit polypeptides of an ATP-binding cassette (ABC)-type transporter comprising transmembrane polypeptide subunits and membrane-associated AAA ATPase polypeptide subunits are no functional variants of each other. As such, one set of multiple coding DNA sequences in a cell of disclosure may encode for one single polypeptide subunit of a multi-subunit complex polypeptide and/or for functional variants of the single polypeptide subunit but may not encode different subunits that constitute one multi-subunit complex. In an exemplary embodiment, the cell of disclosure comprises one set of multiple coding DNA sequences that encodes one AAA ATPase polypeptide subunit of an ABC transporter. However, in the context of the disclosure, the cell of disclosure may comprise more than one set of multiple coding DNA sequences wherein each set of multiple coding DNA sequences encode for a different single polypeptide subunit of a multi-subunit complex polypeptide and/or functional variants of the single polypeptide subunit. In an exemplary embodiment, the cell of disclosure comprises multiple sets of multiple coding DNA sequences wherein each set of multiple coding DNA sequences encode for a different single polypeptide subunit of one ABC transporter comprising one set of multiple coding DNA sequences that encode one AAA ATPase polypeptide subunit of the ABC transporter and one set of multiple coding DNA sequences that encode one transmembrane polypeptide subunit of the same ABC transporter.

According to a preferred embodiment of the disclosure, the multiple coding DNA sequences within a set of multiple coding DNA sequences are integrated in the genome of the cell and/or presented to the cell on one or more vectors. A cell of disclosure may comprise all the different coding DNA sequences of one set integrated in its genome. Alternatively, a cell of disclosure may comprise all the different coding DNA sequences of one set integrated in one or more vectors that is/are stably transformed into the cell. Alternatively, a cell of disclosure may comprise one part of the different coding DNA sequences of one set integrated in its genome and another part of the different coding DNA sequences of the same set integrated in one or more vectors that is/are stably transformed into the cell. Alternatively, a cell of disclosure may comprise more than one set of multiple coding DNA sequences as defined herein, wherein the multiple coding DNA sequences of each set are integrated in the genome of the cell and/or presented to the cell on one or more vectors.

The vector can be present in the form of a plasmid, cosmid, artificial chromosome, phage, liposome or virus, which is/are to be stably transformed/transfected into the cell. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses, and vectors derived from combinations thereof, such as those derived from plasmid and bacteriophage genetic elements, such as cosmids and phagemids. These vectors may contain selection markers such as but not limited to antibiotic markers, auxotrophic markers, toxin-antitoxin markers, RNA sense/antisense markers. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted by any of a variety of well-known and routine techniques, such as, for example, those set forth in Davis et al., Basic Methods in Molecular Biology, (1986), and Sambrook et al., 2001, Molecular Cloning: a laboratory manual, 3rd Edition, Cold Spring Harbor Laboratory Press, CSH, New York or to Current Protocols in Molecular Biology, John Wiley and Sons, N.Y. (1989 and yearly updates).

According to a preferred embodiment of the disclosure, the multiple coding DNA sequences within a set are presented to the cell in one or more location(s) on one or more chromosome(s).

According to another preferred embodiment of the method and/or cell of the disclosure, the multiple coding DNA sequences within a set are presented to the cell within a biosynthetic gene cluster encoding polypeptides participating in a pathway for production of the di- and/or oligosaccharide.

According to another preferred embodiment of the method and/or cell of the disclosure, the multiple coding DNA sequences within a set are presented to the cell in one or more gene expression modules comprising one or more regulatory gene sequences regulating expression of the multiple coding DNA sequences. The expression modules are also known as transcriptional units and comprise polynucleotides for expression of recombinant genes including the coding DNA sequences and appropriate transcriptional and/or translational control signals that are operably linked to the coding DNA sequences. The control signals comprise promoter sequences, untranslated regions, ribosome binding sites, terminator sequences. The expression modules can contain elements for expression of one single recombinant gene of interest but can also contain elements for expression of more recombinant genes of interest or can be organized in an operon structure for integrated expression of two or more recombinant genes of interest.

The cell of disclosure may be additionally genetically modified with one or more expression module(s) that do(es) not comprise a set of multiple coding DNA sequences as defined herein but that comprise only one coding DNA sequence or two or more identical coding DNA sequences for expression of at least one recombinant gene of interest. Alternatively and/or additionally, the cell may be genetically modified with one or more expression module(s) that comprise different coding DNA sequences encoding for different polypeptides wherein the different polypeptides have a different function and/or activity of interest compared to each other.

According to a preferred embodiment of the method and/or cell of the disclosure, the multiple coding DNA sequences within a set are organized within any one or more of the list comprising co-expression module, operon, regulon, stimulon and modulon, as defined herein.

According to another preferred embodiment of the disclosure, the expression of the multiple coding DNA sequences within a set is regulated by one or more promoter sequence(s) that is/are constitutive and/or inducible upon a natural inducer, as defined herein.

The coding DNA sequences and expression modules, comprising co-expression module, operon, regulon, stimulon and modulon, may be produced by recombinant DNA technology using techniques well-known in the art. Methods that are well known to those skilled in the art to construct expression modules include, for example, in vitro recombinant DNA techniques, synthetic techniques, and in vivo genetic recombination. See, for example, the techniques described in Davis et al. (supra) and Sambrook et al. (supra). As used herein, the multiple coding DNA sequences within a set encode endogenous proteins with a modified expression or activity, preferably the endogenous proteins are overexpressed; or the multiple coding DNA sequences within a set encode heterologous proteins that are heterogeneously introduced and expressed in the modified cell, preferably overexpressed. Alternatively, the multiple coding DNA sequences within a set encode endogenous polypeptides with a modified expression or activity, as well as heterologous polypeptides that are heterogeneously introduced and expressed in the modified cell. Within the scope of disclosure, the multiple coding DNA sequences within a set do not encode endogenous polypeptides with a native expression or native activity.

According to an embodiment of the method and/or cell of the disclosure, the cell comprises a pathway for production of a di- and/or oligosaccharide. The pathway for production of a di- and/or oligosaccharide as used herein is a biochemical pathway consisting of the enzymes and their respective genes directly involved in the synthesis of a di- and/or oligosaccharide as defined herein. The pathway may comprise any one or more of one or more pathway(s) to produce one or more nucleotide donor(s) and one or more glycosyltransferase(s) for the transfer of the one or more nucleotide donor(s) to an acceptor as defined herein, one or more biosynthetic pathway(s) to produce in the cell one or more precursor(s) as defined herein and involved in the production of a di- and/or oligosaccharide, a mechanism of internalization of one or more precursor(s) from the culture medium into the cell, a mechanism for enabled and/or enhanced efflux of the di- and/or oligosaccharide from the cell to the outside of the cell, and a mechanism for disabled and/or diminished efflux from the cell to the outside of the cell of any one or more metabolite(s) and/or by-product(s) that is/are synthesized during the production of the di- and/or oligosaccharide of disclosure.

According to a preferred embodiment of the method and/or cell of the disclosure, the cell comprises a pathway for production of a di- and/or oligosaccharide wherein the pathway comprises any one or more of fucosylation, sialylation, galactosylation, N-acetylglucosaminylation, N-acetylgalactosaminylation, mannosylation and N-acetylmannosaminylation pathway as defined herein. According to another preferred embodiment of the method and/or cell of the disclosure, the cell comprises two or more pathways as defined herein for production of a di- and/or oligosaccharide. In an exemplary embodiment, the cell comprises a fucosylation and a sialylation pathway as defined herein for production of a di- and/or oligosaccharide. In another exemplary embodiment, the cell comprises a fucosylation and a galactosylation pathway as defined herein for production of a di- and/or oligosaccharide. In another exemplary embodiment, the cell comprises a fucosylation and an N-acetylglucosaminylation pathway as defined herein for production of a di- and/or oligosaccharide. In another exemplary embodiment, the cell comprises a sialylation, a fucosylation, a galactosylation and an N-acetylglucosaminylation pathway as defined herein for production of a di- and/or oligosaccharide.

According to another embodiment of the method and/or cell of the disclosure, the cell is genetically modified for the production of the di- and/or oligosaccharide.

In a preferred embodiment of the method and/or cell of the disclosure, the cell is genetically modified by introducing a pathway for the production of the di- and/or oligosaccharide. In a more preferred embodiment, the cell is genetically modified for expression of one or more polypeptides that are directly involved in a pathway for the production of the di- and/or oligosaccharide. In another more preferred embodiment, the cell is genetically modified by introducing more than one pathway for the production of the di- and/or oligosaccharide. The pathway that is introduced in the cell may comprise any one or more of one or more pathway(s) to produce one or more nucleotide donor(s) and one or more glycosyltransferase(s) for the transfer of the one or more nucleotide donor(s) to an acceptor as defined herein, one or more biosynthetic pathway(s) to produce in the cell one or more precursor(s) as defined herein and involved in the production of a di- and/or oligosaccharide, a mechanism of internalization of one or more precursor(s) from the culture medium into the cell, a mechanism for enabled and/or enhanced efflux of the di- and/or oligosaccharide from the cell to the outside of the cell, and a mechanism for disabled and/or diminished efflux from the cell to the outside of the cell of any one or more metabolite(s) and/or by-product(s) that is/are synthesized during the production of the di- and/or oligosaccharide of disclosure. According to a preferred embodiment of the method and/or cell of the disclosure, the cell is genetically modified by introducing a pathway for production of a di- and/or oligosaccharide wherein the pathway comprises any one or more of fucosylation, sialylation, galactosylation, N-acetylglucosaminylation, N-acetylgalactosaminylation, mannosylation and N-acetylmannosaminylation pathway as defined herein. In an exemplary embodiment, the cell is genetically modified by introducing a fucosylation and a sialylation pathway as defined herein for production of a di- and/or oligosaccharide. In another exemplary embodiment, the cell is genetically modified by introducing a fucosylation and a galactosylation pathway as defined herein for production of a di- and/or oligosaccharide. In another exemplary embodiment, the cell is genetically modified by introducing a fucosylation and an N-acetylglucosaminylation pathway as defined herein for production of a di- and/or oligosaccharide. In another exemplary embodiment, the cell is genetically modified by introducing a sialylation, a fucosylation, a galactosylation and an N-acetylglucosaminylation pathway as defined herein for production of a di- and/or oligosaccharide.

According to another preferred embodiment of the method and/or cell of the disclosure, the cell is genetically modified for expression and/or over-expression of one set of multiple coding DNA sequences that differ in nucleotide sequence and encode polypeptides that have the same function and/or activity of interest and that are directly involved in a pathway for production of the di- and/or oligosaccharide as defined herein.

According to another preferred embodiment of the method and/or cell of the disclosure, the cell is genetically modified for expression and/or over-expression of more than one set of multiple coding DNA sequences (1) wherein each set of multiple coding DNA sequences differ in nucleotide sequence and encode polypeptides that have the same function and/or activity of interest, and (2) wherein each set of multiple coding DNA sequences encodes polypeptides having a different function and/or activity of interest compared to the other sets of multiple coding DNA sequences and (3) wherein the polypeptides encoded by one set of multiple coding DNA sequences are directly involved in a pathway for production of the di- and/or oligosaccharide as defined herein.

According to another preferred embodiment of the method and/or cell of the disclosure, the cell is genetically modified for expression and/or over-expression of more than one set of multiple coding DNA sequences (1) wherein each set of multiple coding DNA sequences differ in nucleotide sequence and encode polypeptides that have the same function and/or activity of interest, and (2) wherein each set of multiple coding DNA sequences encodes polypeptides having a different function and/or activity of interest compared to the other sets of multiple coding DNA sequences and (3) wherein the polypeptides encoded by more than one set of multiple coding DNA sequences are directly involved in a pathway for production of the di- and/or oligosaccharide as defined herein. The sets of multiple coding DNA sequences may encode polypeptides that are directly involved in the same pathway for production of the di- and/or oligosaccharide as defined herein. Alternatively, the sets of multiple coding DNA sequences may encode polypeptides that are directly involved in different pathways for production of the di- and/or oligosaccharide as defined herein.

According to another preferred embodiment of the method and/or cell of the disclosure, the cell is genetically modified for expression and/or over-expression of more than one set of multiple coding DNA sequences (1) wherein each set of multiple coding DNA sequences differ in nucleotide sequence and encode polypeptides that have the same function and/or activity of interest, and (2) wherein each set of multiple coding DNA sequences encodes polypeptides having a different function and/or activity of interest compared to the other sets of multiple coding DNA sequences and (3) wherein the polypeptides encoded by all of the sets of multiple coding DNA sequences are directly involved in one or more pathway(s) for production of the di- and/or oligosaccharide as defined herein. The sets of multiple coding DNA sequences may encode polypeptides that are directly involved in the same pathway for production of the di- and/or oligosaccharide as defined herein. Alternatively, the sets of multiple coding DNA sequences may encode polypeptides that are directly involved in different pathways for production of the di- and/or oligosaccharide as defined herein.

According to a more preferred embodiment of the method and/or cell of the disclosure, the cell is genetically modified for expression and/or over-expression of at least two of the sets of multiple coding DNA sequences as defined herein. In an even more preferred embodiment of the method and/or cell, the cell is genetically modified for expression and/or over-expression of two or more of the sets of multiple coding DNA sequences as defined herein.

According to another embodiment of the method and/or cell of the disclosure, the polypeptides that are encoded by a set of multiple coding DNA sequences are endogenous polypeptides of the cell with a modified expression or activity, preferably over-expressed or higher activity.

According to an alternative embodiment of the method and/or cell of the disclosure, the polypeptides that are encoded by a set of multiple coding DNA sequences are heterologous polypeptides that are heterogeneously introduced and expressed in the cell, preferably overexpressed. According to an alternative embodiment of the method and/or cell of the disclosure, the polypeptides that are encoded by a set of multiple coding DNA sequences are a combination of endogenous polypeptides of the cell with a modified expression or activity, preferably over-expressed or higher activity and heterologous polypeptides that are heterogeneously introduced and expressed in the cell, preferably overexpressed.

According to a preferred aspect of the disclosure, the expression of each of the polypeptides is constitutive or inducible upon a natural inducer as defined herein.

According to a preferred embodiment of the method and/or cell of the disclosure, the pathway for production of a di- and/or oligosaccharide comprises or consists of a fucosylation pathway as defined herein. In a more preferred embodiment, the polypeptides that are encoded by the multiple coding DNA sequences within a set are directly involved in the fucosylation pathway. In an even more preferred embodiment, the polypeptides that are encoded by the multiple coding DNA sequences within a set are selected from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, and fucosyltransferase.

According to another preferred embodiment of the method and/or cell of the disclosure, the pathway for production of a di- and/or oligosaccharide comprises or consists of a sialylation pathway as defined herein. In a more preferred embodiment, the polypeptides that are encoded by the multiple coding DNA sequences within a set are directly involved in the sialylation pathway. In an even more preferred embodiment, the polypeptides that are encoded by the multiple coding DNA sequences within a set are selected from the list comprising N-acylglucosamine 2-epimerase, UDP-N-acetylglucosamine 2-epimerase, N-acetylmannosamine-6-phosphate 2-epimerase, UDP-N-acetylglucosamine 2-epimerase/kinase hydrolyzing, N-acylneuraminate-9-phosphate synthase, phosphatase, N-acetylneuraminate synthase, N-acylneuraminate cytidylyltransferase, sialyltransferase and sialic acid transporter.

According to another preferred embodiment of the method and/or cell of the disclosure, the pathway for production of a di- and/or oligosaccharide comprises or consists of a galactosylation pathway as defined herein. In a more preferred embodiment, the polypeptides that are encoded by the multiple coding DNA sequences within a set are directly involved in the galactosylation pathway. In an even more preferred embodiment, the polypeptides that are encoded by the multiple coding DNA sequences within a set are selected from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase. According to a more preferred embodiment of the method and/or cell of the disclosure, the cell is genetically modified to express, preferably over-express, any one or more polypeptides chosen from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase.

According to another preferred embodiment of the method and/or cell of the disclosure, the pathway for production of a di- and/or oligosaccharide comprises or consists of an N-acetylglucosaminylation pathway as defined herein. In a more preferred embodiment, the polypeptides that are encoded by the multiple coding DNA sequences within a set are directly involved in the N-acetylglucosaminylation pathway. In an even more preferred embodiment, the polypeptides that are encoded by the multiple coding DNA sequences within a set are selected from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase and N-acetylglucosaminyltransferase.

According to another preferred embodiment of the method and/or cell of the disclosure, the pathway for production of a di- and/or oligosaccharide comprises or consists of an N-acetylgalactosaminylation pathway as defined herein. In a more preferred embodiment, the polypeptides that are encoded by the multiple coding DNA sequences within a set are directly involved in the N-acetylgalactosaminylation pathway. In an even more preferred embodiment, the polypeptides that are encoded by the multiple coding DNA sequences within a set are selected from the list comprising L-glutamine-D-fructose-6-phosphate aminotransferase, phosphoglucosamine mutase, N-acetylglucosamine 1-phosphate uridylyltransferase, glucosamine-1-phosphate acetyltransferase, UDP-N-acetylglucosamine 4-epimerase, UDP-glucose 4-epimerase, N-acetylgalactosamine kinase, UDP-N-acetylgalactosamine pyrophosphorylase and N-acetylgalactosaminyltransferase.

According to another preferred embodiment of the method and/or cell of the disclosure, the pathway for production of a di- and/or oligosaccharide comprises or consists of a mannosylation pathway as defined herein. In a more preferred embodiment, the polypeptides that are encoded by the multiple coding DNA sequences within a set are directly involved in the mannosylation pathway. In an even more preferred embodiment, the polypeptides that are encoded by the multiple coding DNA sequences within a set are selected from the list comprising mannose-6-phosphate isomerase, phosphomannomutase, mannose-1-phosphate guanylyltransferase and mannosyltransferase.

According to another preferred embodiment of the method and/or cell of the disclosure, the pathway for production of a di- and/or oligosaccharide comprises or consists of an N-acetylmannosaminylation pathway as defined herein. In a more preferred embodiment, the polypeptides that are encoded by the multiple coding DNA sequences within a set are directly involved in the N-acetylmannosaminylation pathway In an even more preferred embodiment, the polypeptides that are encoded by the multiple coding DNA sequences within a set are selected from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase, ManNAc kinase and N-acetylmannosaminyltransferase.

According to a further embodiment of the method and/or cell of disclosure, the cell may be genetically modified for expression of one or more recombinant genes that encode for one or more polypeptides that is/are not needed for the production of the di- and/or oligosaccharide.

According to another and/or additional further embodiment of the method and/or cell of disclosure, the cell may be genetically modified with one or more additional pathways that are not needed for the production of the di- and/or oligosaccharide.

According to a preferred embodiment of the method and/or cell of disclosure, the cell is genetically modified for expression and/or over-expression of at least one set of multiple coding DNA sequences that differ in nucleotide sequence and encode polypeptides having the same function and/or activity of interest in the synthesis of a nucleotide-activated sugar, wherein the nucleotide-activated sugar is to be used in the production of the di- and/or oligosaccharide. Preferably, the nucleotide-activated sugar is chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-fucose (GDP-Fuc), GDP-rhamnose and UDP-xylose.

In a more preferred embodiment of the method and/or cell of the disclosure, the cell is genetically modified for expression and/or over-expression of one set of multiple coding DNA sequences that differ in nucleotide sequence and encode polypeptides having the same function and/or activity of interest in the synthesis of a nucleotide-activated sugar that are chosen from the list comprising mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase or UDP-N-acetylgalactosamine pyrophosphorylase.

In another more preferred embodiment of the method and/or cell, the cell is genetically modified with two or more sets of multiple coding DNA sequences wherein (1) the multiple coding DNA sequences within each set differ in nucleotide sequence and encode polypeptides having the same function and/or activity of interest in the synthesis of a nucleotide-activated sugar wherein the nucleotide-activated sugar is to be used in the production of a di- and/or oligosaccharide, (2) each of the sets of multiple coding DNA sequences encodes polypeptides having a different function and/or activity of interest in the synthesis of a nucleotide-activated sugar compared to the other sets of multiple coding DNA sequences and (3) the polypeptides encoded by each of the sets of multiple coding DNA sequences either have mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase or UDP-N-acetylgalactosamine pyrophosphorylase activity.

In a more preferred embodiment of the method and/or cell of disclosure, the cell is modified to produce UDP-GlcNAc from e.g., GlcNAc by expression of enzymes like e.g., an N-acetylglucosamine kinase, an N-acetylglucosamine-6-phosphate deacetylase, a phosphoglucosamine mutase, and an N-acetylglucosamine-1-phosphate uridylyltransferase/glucosamine-1-phosphate acetyltransferase from several species including Homo sapiens, Escherichia coli. More preferably, the cell is modified for enhanced UDP-GlcNAc production. The modification can be any one or more chosen from the group comprising knock-out of an N-acetylglucosamine-6-phosphate deacetylase, over-expression of an L-glutamine-D-fructose-6-phosphate aminotransferase, over-expression of a phosphoglucosamine mutase, and over-expression of an N-acetylglucosamine-1-phosphate uridyltransferase/glucosamine-1-phosphate acetyltransferase. In a more preferred embodiment of the method and/or cell of disclosure, the cell is modified to produce UDP-GlcNAc from e.g., GlcNAc by expression of one or more polypeptides comprising but not limited to an N-acetylglucosamine kinase, an N-acetylglucosamine-6-phosphate deacetylase, a phosphoglucosamine mutase, an N-acetylglucosamine-1-phosphate uridylyltransferase/glucosamine-1-phosphate acetyltransferase and L-glutamine-D-fructose-6-phosphate aminotransferase wherein at least one of the polypeptides is encoded by one set of multiple coding DNA sequences, preferably at least two of the polypeptides are encoded by a different set of multiple coding DNA sequences according to the disclosure, more preferably wherein each of the polypeptides are encoded by a different set of multiple coding DNA sequences according to the disclosure.

In another more preferred embodiment of the method and/or cell of disclosure, the cell is modified to express de novo synthesis of CMP-sialic acid like e.g., CMP-Neu5Ac or CMP-Neu5Gc. Such cell producing CMP-Neu5Ac can express an enzyme converting, e.g., sialic acid to CMP-Neu5Ac. This enzyme may be a CMP-sialic acid synthetase, like the N-acylneuraminate cytidylyltransferase from several species including Homo sapiens, Neisseria meningitidis, and Pasteurella multocida. More preferably, the cell is modified for enhanced CMP-Neu5Ac production. The modification can be any one or more chosen from the group comprising knock-out of an N-acetylglucosamine-6-phosphate deacetylase, knock-out of an glucosamine-6-phosphate deaminase, over-expression of a CMP-sialic acid synthetase, and over-expression of an N-acetyl-D-glucosamine-2-epimerase encoding gene. CMP-Neu5Gc can be synthesized directly from CMP-Neu5Ac via a hydroxylation reaction performed by a vertebrate CMP-Neu5Ac hydroxylase (CMAH) enzyme. More preferably, the cell is modified for enhanced CMP-Neu5Gc production. In a more preferred embodiment of the method and/or cell of disclosure, the cell is modified to produce CMP-sialic acid by expression of one or more polypeptides comprising but not limited to N-acylneuraminate cytidylyltransferase, N-acetyl-D-glucosamine-2-epimerase and CMP-Neu5Ac hydroxylase wherein at least one of the polypeptides is encoded by one set of multiple coding DNA sequences, preferably at least two of the polypeptides are encoded by a different set of multiple coding DNA sequences according to the disclosure, more preferably wherein each of the polypeptides are encoded by a different set of multiple coding DNA sequences according to the disclosure.

In another more preferred embodiment of the method and/or cell of disclosure, the host cell used herein is genetically modified to express the de novo synthesis of GDP-fucose. GDP-fucose can be provided by an enzyme expressed in the cell or by the metabolism of the cell. Such cell producing GDP-fucose can express an enzyme converting, e.g., fucose, which is to be added to the cell, to GDP-fucose. This enzyme may be, e.g., a bifunctional fucose kinase/fucose-1-phosphate guanylyltransferase, like Fkp from Bacteroides fragilis, or the combination of one separate fucose kinase together with one separate fucose-1-phosphate guanylyltransferase like they are known from several species including Homo sapiens, Sus scrofa and Rattus norvegicus. Preferably, the cell is modified to produce GDP-fucose. More preferably, the cell is modified for enhanced GDP-fucose production. The modification can be any one or more chosen from the group comprising knock-out of an UDP-glucose:undecaprenyl-phosphate glucose-1-phosphate transferase encoding gene, over-expression of a GDP-L-fucose synthase encoding gene, over-expression of a GDP-mannose 4,6-dehydratase encoding gene, over-expression of a mannose-1-phosphate guanylyltransferase encoding gene, over-expression of a phosphomannomutase encoding gene and over-expression of a mannose-6-phosphate isomerase encoding gene. In a more preferred embodiment of the method and/or cell of disclosure, the cell is modified to produce GDP-fucose by expression of one or more polypeptides comprising but not limited to bifunctional fucose kinase/fucose-1-phosphate guanylyltransferase, fucose kinase, fucose-1-phosphate guanylyltransferase, GDP-L-fucose synthase, a GDP-mannose 4,6-dehydratase a mannose-1-phosphate guanylyltransferase, a phosphomannomutase and a mannose-6-phosphate isomerase wherein at least one of the polypeptides is encoded by one set of multiple coding DNA sequences, preferably at least two of the polypeptides are encoded by a different set of multiple coding DNA sequences according to the disclosure, more preferably wherein each of the polypeptides are encoded by a different set of multiple coding DNA sequences according to the disclosure.

In another more preferred embodiment of the method and/or cell of disclosure, the host cell used herein is genetically modified to express the de novo synthesis of UDP-Gal. UDP-Gal can be provided by an enzyme expressed in the cell or by the metabolism of the cell. Such cell producing UDP-Gal can express an enzyme converting, e.g., UDP-glucose, to UDP-Gal. This enzyme may be, e.g., the UDP-glucose-4-epimerase GalE like as known from several species including Homo sapiens, Escherichia coli, and Rattus norvegicus. Preferably, the cell is modified to produce UDP-Gal. More preferably, the cell is modified for enhanced UDP-Gal production. The modification can be any one or more chosen from the group comprising knock-out of an bifunctional 5′-nucleotidase/UDP-sugar hydrolase encoding gene, knock-out of a galactose-1-phosphate uridylyltransferase encoding gene and over-expression of an UDP-glucose-4-epimerase encoding gene. In a more preferred embodiment of the method and/or cell of disclosure, the cell is modified to produce UDP-Gal by expression of one or more polypeptides being UDP-glucose-4-epimerase or having UDP-glucose-4-epimerase activity wherein at least one of the polypeptides is encoded by one set of multiple coding DNA sequences, preferably at least two of the polypeptides are encoded by a different set of multiple coding DNA sequences according to the disclosure, more preferably wherein each of the polypeptides are encoded by a different set of multiple coding DNA sequences according to the disclosure.

In another more preferred embodiment of the method and/or cell of disclosure, the host cell used herein is genetically modified to express the de novo synthesis of UDP-GalNAc. UDP-GalNAc can be synthesized from UDP-GlcNAc by the action of a single-step reaction using an UDP-N-acetylglucosamine 4-epimerase like e.g., wbgU from Plesiomonas shigelloides, gne from Yersinia enterocolitica or wbpP from Pseudomonas aeruginosa serotype 06. Preferably, the cell is modified to produce UDP-GalNAc. More preferably, the cell is modified for enhanced UDP-GalNAc production. In a more preferred embodiment of the method and/or cell of disclosure, the cell is modified to produce UDP-GalNAc by expression of one or more polypeptides being UDP-N-acetylglucosamine 4-epimerase or having UDP-N-acetylglucosamine 4-epimerase activity wherein at least one of the polypeptides is encoded by one set of multiple coding DNA sequences, preferably at least two of the polypeptides are encoded by a different set of multiple coding DNA sequences according to the disclosure, more preferably wherein each of the polypeptides are encoded by a different set of multiple coding DNA sequences according to the disclosure.

In another more preferred embodiment of the method and/or cell of disclosure, the host cell used herein is genetically modified to express the de novo synthesis of UDP-ManNAc. UDP-ManNAc can be synthesized directly from UDP-GlcNAc via an epimerization reaction performed by an UDP-GlcNAc 2-epimerase (like e.g., cap5P from Staphylococcus aureus, RffE from E. coli, Cps19fK from S. pneumoniae, and RfbC from S. enterica). Preferably, the cell is modified to produce UDP-ManNAc. More preferably, the cell is modified for enhanced UDP-ManNAc production. In a more preferred embodiment of the method and/or cell of disclosure, the cell is modified to produce UDP-ManNAc by expression of one or more polypeptides being UDP-GlcNAc 2-epimerase or having UDP-GlcNAc 2-epimerase activity wherein at least one of the polypeptides is encoded by one set of multiple coding DNA sequences, preferably at least two of the polypeptides are encoded by a different set of multiple coding DNA sequences according to the disclosure, more preferably wherein each of the polypeptides are encoded by a different set of multiple coding DNA sequences according to the disclosure.

According to an alternative and/or additional preferred embodiment of the method and/or cell of disclosure, the cell is genetically modified for expression and/or over-expression of at least one set of multiple coding DNA sequences that differ in nucleotide sequence and each encoding a polypeptide, wherein the polypeptides have the same function and/or activity of interest and are glycosyltransferases wherein the glycosyltransferases transfer a monosaccharide from a nucleotide-activated sugar donor to a glycan acceptor.

Preferably, the multiple coding DNA sequences within a set encode glycosyltransferases or polypeptides having glycosyltransferase activity that are either fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases or fucosaminyltransferases.

In a more preferred embodiment of the method and/or cell of the disclosure, the fucosyltransferases expressed by the multiple coding DNA sequences within one set are alpha-1,2-fucosyltransferases, alpha-1,3-fucosyltransferases, alpha-1,4-fucosyltransferases or alpha-1,6-fucosyltransferases. In another more preferred embodiment of the method and/or cell of the disclosure, the sialyltransferases expressed by the multiple coding DNA sequences within one set are alpha-2,3-sialyltransferases, alpha-2,6-sialyltransferases or alpha-2,8-sialyltransferases. In another more preferred embodiment of the method and/or cell of the disclosure, the galactosyltransferases expressed by the multiple coding DNA sequences within one set are beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases or alpha-1,4-galactosyltransferases. In another more preferred embodiment of the method and/or cell of the disclosure, the glucosyltransferases expressed by the multiple coding DNA sequences within one set are alpha-glucosyltransferases, beta-1,2-glucosyltransferases, beta-1,3-glucosyltransferases or beta-1,4-glucosyltransferases. In another more preferred embodiment of the method and/or cell of the disclosure, the mannosyltransferases expressed by the multiple coding DNA sequences within one set are alpha-1,2-mannosyltransferases, alpha-1,3-mannosyltransferases or alpha-1,6-mannosyltransferases. In another more preferred embodiment of the method and/or cell of the disclosure, the N-acetylglucosaminyltransferases expressed by the multiple coding DNA sequences within one set are galactoside beta-1,3-N-acetylglucosaminyltransferases or beta-1,6-N-acetylglucosarninyltr-ansferases. In another more preferred embodiment of the method and/or cell of the disclosure, the N-acetylgalactosaminyltransferases expressed by the multiple coding DNA sequences within one set are alpha-1,3-N-acetylgalactosaminyltransferases.

In another preferred embodiment of the method and/or cell, the cell is genetically modified with different sets of multiple coding DNA sequences wherein at least one of the sets encode alpha-1,2-fucosyltransferases, alpha-1,3-fucosyltransferases, alpha-1,4-fucosyltransferases, alpha-1,6-fucosyltransferases, alpha-2,3-sialyltransferases, alpha-2,6-sialyltransferases, alpha-2,8-sialyltransferases, beta-1,3-galactosyltransferases, N-acetylglucosamine beta-1,3-galactosyltransferases, beta-1,4-galactosyltransferases, N-acetylglucosamine beta-1,4-galactosyltransferases, alpha-1,3-galactosyltransferases, alpha-1,4-galactosyltransferases, alpha-glucosyltransferases, beta-1,2-glucosyltransferases, beta-1,3-glucosyltransferases, beta-1,4-glucosyltransferases, alpha-1,2-mannosyltransferases, alpha-1,3-mannosyltransferases, alpha-1,6-mannosyltransferases, galactoside beta-1,3-N-acetylglucosaminyltransferases, beta-1,6-N-acetylglucosaminyltransferases or alpha-1,3-N-acetylgalactosaminyltransferases. In a more preferred embodiment of the method and/or cell, the cell is modified with different sets of multiple coding DNA sequences wherein at least two of the sets encode glycosyltransferases as described herein that have a different function and/or activity of interest compared to each other. In an even more preferred embodiment of the method and/or cell, the cell is modified with different sets of multiple coding DNA sequences wherein each set encodes one or more glycosyltransferases as described herein that have a different function and/or activity of interest compared to the glycosyltransferases encoded by the other sets of multiple coding DNA sequences.

According to an alternative and/or additional preferred embodiment of the method and/or cell of disclosure, the cell is genetically modified for expression and/or over-expression of at least one set of multiple coding DNA sequences that differ in nucleotide sequence and each encoding a polypeptide, wherein the polypeptides have the same function and/or activity and are membrane transporter proteins or polypeptides having transport activity hereby transporting compounds across the outer membrane of the cell wall. Preferably, the membrane transporter proteins or polypeptides having transport activity control the flow over the outer membrane of the cell wall of the di- and/or oligosaccharide produced by the cell. In another and/or additional preferred embodiment of the method and/or cell of the disclosure, the membrane transporter proteins and polypeptides having transport activity control the flow over the outer membrane of the cell wall of any one or more precursor(s) to be used in the production of the di- and/or oligosaccharide. In another and/or additional preferred embodiment of the method and/or cell of the disclosure, the membrane transporter proteins and polypeptides having transport activity control the flow over the outer membrane of the cell wall of any one or more acceptor(s) to be used in the production of the di- and/or oligosaccharide. According to a further preferred embodiment of the method and/or cell of the disclosure, the membrane transporter proteins and polypeptides having transport activity hereby transporting compounds across the outer membrane of the cell wall and encoded by at least one set of multiple coding DNA sequences provide improved production and/or enabled and/or enhanced efflux of the di- and/or oligosaccharide.

In a more preferred embodiment of the method and/or cell of the disclosure, the multiple coding DNA sequences within a set encode polypeptides that are membrane transporter proteins or polypeptides having transport activity hereby transporting compounds across the outer membrane of the cell wall that are chosen from the list of transporters comprising porters, P-P-bond-hydrolysis-driven transporters, b-barrel porins, auxiliary transport proteins, putative transport proteins and phosphotransfer-driven group translocators. In a further preferred embodiment of the method and/or cell of the disclosure, the porters comprise MFS transporters, sugar efflux transporters and siderophore exporters. In another further preferred embodiment of the method and/or cell of the disclosure, the P-P-bond-hydrolysis-driven transporters comprise ABC transporters and siderophore exporters.

In a more preferred embodiment of the method and/or cell of the disclosure, the cell comprises at least two sets of multiple coding DNA sequences wherein each set encodes membrane transporter proteins or polypeptides having transport activity hereby transporting compounds across the outer membrane of the cell wall that are different between the sets and wherein the membrane transporter proteins or polypeptides having transport activity hereby transporting compounds across the outer membrane of the cell wall are chosen from the list comprising porters, P-P-bond-hydrolysis-driven transporters, b-barrel porins, auxiliary transport proteins, putative transport proteins and phosphotransfer-driven group translocators as defined herein.

In an exemplary embodiment of the method and/or cell of the disclosure, the cell comprises at least one set of multiple coding DNA sequences encoding MFS transporters having the same function and/or activity of interest like e.g., homologs of the multidrug transporter MdfA family from species comprising E. coli (UniProt ID POAEY8), Cronobacter muytjensii (UniProt ID A0A2T7ANQ9), Citrobacter youngae (UniProt ID D4BC23) and Yokenella regensburgei (UniProt ID G9Z5F4).

In another exemplary embodiment of the method and/or cell of the disclosure, the cell comprises at least one set of multiple coding DNA sequences encoding sugar efflux transporters having the same function and/or activity of interest like e.g., homologs of the SetA family from species comprising E. coli (UniProt ID P31675), Citrobacter koseri (UniProt ID AOA078LM16) and Klebsiella pneumoniae (UniProt ID A0A0C4MGS7).

In another exemplary embodiment of the method and/or cell of the disclosure, the cell comprises at least one set of multiple coding DNA sequences encoding siderophore exporters having the same function and/or activity of interest like e.g., the E. coli entS (UniProt ID P24077), the E. coli MdfA (UniProt ID POAEY8) and the E. coli iceT (UniProt ID A0A024L207).

In another exemplary embodiment of the method and/or cell of the disclosure, the cell comprises at least one set of multiple coding DNA sequences encoding a subunit of an ABC transporter having the same function and/or activity of interest like e.g., oppF from E. coli (UniProt ID P77737), lmrA from Lactococcus lactis subsp. lactis bv. diacetylactis (UniProt ID A0A1V0NEL4) and Blon_2475 from Bifidobacterium longum subsp. infantis (UniProt ID B7GPD4).

In another exemplary embodiment of the method and/or cell of the disclosure, the cell comprises at least one set of multiple coding DNA sequences encoding MFS transporters having the same function and/or activity of interest like e.g., homologs of the multidrug transporter MdfA family from species comprising E. coli (UniProt ID POAEY8), Cronobacter muytjensii (UniProt ID A0A2T7ANQ9), Citrobacter youngae (UniProt ID D4BC23) and Yokenella regensburgei (UniProt ID G9Z5F4) and at least one set of multiple coding DNA sequences comprising coding DNA sequences encoding sugar efflux transporters having the same function and/or activity of interest like e.g., homologs of the SetA family from species comprising E. coli (UniProt ID P31675), Citrobacter koseri (UniProt ID A0A078LM16) and Klebsiella pneumoniae (UniProt ID A0A0C4MGS7).

In another exemplary embodiment of the method and/or cell of the disclosure, the cell comprises at least one set of multiple coding DNA sequences encoding MFS transporters having the same function and/or activity of interest like e.g., homologs of the multidrug transporter MdfA family from species comprising E. coli (UniProt ID P0AEY8), Cronobacter muytjensii (UniProt ID A0A2T7ANQ9), Citrobacter youngae (UniProt ID D4BC23) and Yokenella regensburgei (UniProt ID G9Z5F4) and at least one set of multiple coding DNA sequences encoding a subunit of an ABC transporter having the same function and/or activity of interest like e.g., oppF from E. coli (UniProt ID P77737), lmrA from Lactococcus lactis subsp. lactis bv. diacetylactis (UniProt ID A0A1V0NEL4) and Blon_2475 from Bifidobacterium longum subsp. infantis (UniProt ID B7GPD4).

In another exemplary embodiment of the method and/or cell of the disclosure, the cell comprises at least one set of multiple coding DNA sequences encoding sugar efflux transporters having the same function and/or activity of interest like e.g., homologs of the SetA family from species comprising E. coli (UniProt ID P31675), Citrobacter koseri (UniProt ID A0A078LM16) and Klebsiella pneumoniae (UniProt ID A0A0C4MGS7) and at least one set of multiple coding DNA sequences encoding a subunit of an ABC transporter having the same function and/or activity of interest like e.g., oppF from E. coli (UniProt ID P77737), lmrA from Lactococcus lactis subsp. lactis bv. diacetylactis (UniProt ID A0A1V0NEL4) and Blon_2475 from Bifidobacterium longum subsp. infantis (UniProt ID B7GPD4).

In another exemplary embodiment of the method and/or cell of the disclosure, the cell comprises at least 1) one set of multiple coding DNA sequences encoding MFS transporters having the same function and/or activity of interest like e.g., homologs of the multidrug transporter MdfA family from species comprising E. coli (UniProt ID P0AEY8), Cronobacter muytjensii (UniProt ID A0A2T7ANQ9), Citrobacter youngae (UniProt ID D4BC23) and Yokenella regensburgei (UniProt ID G9Z5F4), 2) at least one set of multiple coding DNA sequences encoding sugar efflux transporters having the same function and/or activity of interest like e.g., homologs of the SetA family from species comprising E. coli (UniProt ID P31675), Citrobacter koseri (UniProt ID A0A078LM16), and Klebsiella pneumoniae (UniProt ID A0A0C4MGS7) and 3) at least one other set of multiple coding DNA sequences encoding a subunit of an ABC transporter having the same function and/or activity of interest like e.g., oppF from E. coli (UniProt ID P77737), lmrA from Lactococcus lactis subsp. lactis bv. diacetylactis (UniProt ID A0A1V0NEL4) and Blon_2475 from Bifidobacterium longum subsp. infantis (UniProt ID B7GPD4).

In a preferred embodiment of the method and/or cell of disclosure, the cell comprises at least two sets of multiple coding DNA sequences wherein at least one set of multiple coding DNA sequences encodes polypeptides having the same function and/or activity in the synthesis of a nucleotide-activated sugar and at least one other set of multiple coding DNA sequences encodes glycosyltransferases or polypeptides having glycosyltransferase activity as described herein.

In another preferred embodiment of the method and/or cell of disclosure, the cell comprises at least two sets of multiple coding DNA sequences wherein at least one set of multiple coding DNA sequences encodes polypeptides having the same function and/or activity in the synthesis of a nucleotide-activated sugar and at least one other set of multiple coding DNA sequences encodes membrane transporter proteins or polypeptides having transport activity hereby transporting compounds across the outer membrane of the cell wall as described herein.

In another preferred embodiment of the method and/or cell of disclosure, the cell comprises at least two sets of multiple coding DNA sequences wherein at least one set of multiple coding DNA sequences encodes glycosyltransferases or polypeptides having glycosyltransferase activity and at least one other set of multiple coding DNA sequences encodes membrane transporter proteins or polypeptides having transport activity hereby transporting compounds across the outer membrane of the cell wall.

In another preferred embodiment of the method and/or cell of disclosure, the cell comprises at least three sets of multiple coding DNA sequences wherein a first set of multiple coding DNA sequences encodes polypeptides having the same function and/or activity in the synthesis of a nucleotide-activated sugar, a second set of multiple coding DNA sequences encodes glycosyltransferases or polypeptides having glycosyltransferase activity and a third set of multiple coding DNA sequences encodes membrane transporter proteins or polypeptides having transport activity hereby transporting compounds across the outer membrane of the cell wall.

In another preferred embodiment of the method and/or cell of disclosure, the cell comprises at least three sets of multiple coding DNA sequences wherein at least one set of multiple coding DNA sequences encodes polypeptides having the same function and/or activity in the synthesis of a nucleotide-activated sugar, at least one other set of multiple coding DNA sequences encodes glycosyltransferases or polypeptides having glycosyltransferase activity and at least another set of multiple coding DNA sequences encodes membrane transporter proteins or polypeptides having transport activity hereby transporting compounds across the outer membrane of the cell wall.

According to another embodiment of the method and/or cell of the disclosure, the di- and/or oligosaccharide is chosen from the list comprising a milk oligosaccharide, O-antigen, enterobacterial common antigen (ECA), the oligosaccharide repeats present in capsular polysaccharides, peptidoglycan, amino-sugars, Lewis-type antigen oligosaccharide and antigens of the human ABO blood group system. In a preferred embodiment, the milk oligosaccharide is a mammalian milk oligosaccharide. In a more preferred embodiment, the milk oligosaccharide is a human milk oligosaccharide.

Preferably, the di- and/or oligosaccharide is an oligosaccharide, more preferably a milk oligosaccharide, even more preferably a mammalian milk oligosaccharide, most preferably a human milk oligosaccharide.

According to another embodiment of the method and/or cell of the disclosure, the cell is capable to produce phosphoenolpyruvate (PEP). According to another embodiment of the method and/or cell of the disclosure, the cell comprises a pathway for production of a di- and/or oligosaccharide comprising a pathway for production of PEP. In a preferred embodiment of the method and/or cell of the disclosure, the cell is modified for enhanced production and/or supply of PEP.

In another preferred embodiment, the cell comprises a pathway for production of a di- and/or oligosaccharide comprising any one or more modifications for enhanced production and/or supply of PEP.

In a preferred embodiment and as a means for enhanced production and/or supply of PEP, one or more PEP-dependent, sugar-transporting phosphotransferase system(s) is/are disrupted such as but not limited to: 1) the N-acetyl-D-glucosamine Npi-phosphotransferase (EC 2.7.1.193), which is, for instance, encoded by the nagE gene (or the cluster nagABCD) in E. coli or Bacillus species, 2) ManXYZ, which encodes the Enzyme 11 Man complex (mannose PTS permease, protein-Npi-phosphohistidine-D-mannose phosphotransferase) that imports exogenous hexoses (mannose, glucose, glucosamine, fructose, 2-deoxyglucose, mannosamine, N-acetylglucosamine, etc.) and releases the phosphate esters into the cell cytoplasm, 3) the glucose-specific PTS transporter (for instance, encoded by PtsG/Crr) which takes up glucose and forms glucose-6-phosphate in the cytoplasm, 4) the sucrose-specific PTS transporter, which takes up sucrose and forms sucrose-6-phosphate in the cytoplasm, 5) the fructose-specific PTS transporter (for instance, encoded by the genes fruA and fruB and the kinase fruK, which takes up fructose and forms in a first step fructose-1-phosphate and in a second step fructose1,6 bisphosphate, 6) the lactose PTS transporter (for instance, encoded by lacE in Lactococcus casei) which takes up lactose and forms lactose-6-phosphate, 7) the galactitol-specific PTS enzyme, which takes up galactitol and/or sorbitol and forms galactitol-1-phosphate or sorbitol-6-phosphate respectively, 8) the mannitol-specific PTS enzyme, which takes up mannitol and/or sorbitol and forms mannitol-1-phosphate or sorbitol-6-phosphate respectively, and 9) the trehalose-specific PTS enzyme, which takes up trehalose and forms trehalose-6-phosphate.

In another and/or additional preferred embodiment and as a means for enhanced production and/or supply of PEP, the full PTS system is disrupted by disrupting the PtsIH/Crr gene cluster. PtsI (Enzyme I) is a cytoplasmic protein that serves as the gateway for the phosphoenolpyruvate:sugar phosphotransferase system (PTS^{sugar}) of E. coli K-12. PtsI is one of two (PtsI and PtsH) sugar non-specific protein constituents of the PTS^{sugar}, which along with a sugar-specific inner membrane permease effects a phosphotransfer cascade that results in the coupled phosphorylation and transport of a variety of carbohydrate substrates. HPr (histidine containing protein) is one of two sugar-non-specific protein constituents of the PTS^{sugar} It accepts a phosphoryl group from phosphorylated Enzyme I (PtsI-P) and then transfers it to the EIIA domain of any one of the many sugar-specific enzymes (collectively known as Enzymes II) of the PTS^{sugar}. Crr or EIIA^{Glc} is phosphorylated by PEP in a reaction requiring PtsH and PtsI.

In another and/or additional preferred embodiment, the cell is further modified to compensate for the deletion of a PTS system of a carbon source by the introduction and/or overexpression of the corresponding permease. These are e.g., permeases or ABC transporters that comprise but are not limited to transporters that specifically import lactose such as e.g., the transporter encoded by the LacY gene from E. coli, sucrose such as e.g., the transporter encoded by the cscB gene from E. coli, glucose such as e.g., the transporter encoded by the galP gene from E. coli, fructose such as e.g., the transporter encoded by the fruI gene from Streptococcus mutans, or the Sorbitol/mannitol ABC transporter such as the transporter encoded by the cluster SmoEFGK of Rhodobacter sphaeroides, the trehalose/sucrose/maltose transporter such as the transporter encoded by the gene cluster ThuEFGK of Sinorhizobium me/iloti and the N-acetylglucosamine/galactose/glucose transporter such as the transporter encoded by NagP of Shewanella oneidensis. Examples of combinations of PTS deletions with overexpression of alternative transporters are: 1) the deletion of the glucose PTS system, e.g., ptsG gene, combined with the introduction and/or overexpression of a glucose permease (e.g., galP of glcP), 2) the deletion of the fructose PTS system, e.g., one or more of the fruB, fruA, fruK genes, combined with the introduction and/or overexpression of fructose permease, e.g., fruI, 3) the deletion of the lactose PTS system, combined with the introduction and/or overexpression of lactose permease, e.g., LacY, and/or 4) the deletion of the sucrose PTS system, combined with the introduction and/or overexpression of a sucrose permease, e.g., cscB.

In a further preferred embodiment, the cell is modified to compensate for the deletion of a PTS system of a carbon source by the introduction of carbohydrate kinases, such as glucokinase (EC 2.7.1.1, EC 2.7.1.2, EC 2.7.1.63), galactokinase (EC 2.7.1.6), and/or fructokinase (EC 2.7.1.3, EC 2.7.1.4). Examples of combinations of PTS deletions with overexpression of alternative transporters and a kinase are: 1) the deletion of the glucose PTS system, e.g., ptsG gene, combined with the introduction and/or overexpression of a glucose permease (e.g., galP of glcP), combined with the introduction and/or overexpression of a glucokinase (e.g., glk), and/or 2) the deletion of the fructose PTS system, e.g., one or more of the fruB, fruA, fruK genes, combined with the introduction and/or overexpression of fructose permease, e.g., fruI, combined with the introduction and/or overexpression of a fructokinase (e.g., frk or mak).

In another and/or additional preferred embodiment and as a means for enhanced production and/or supply of PEP, the cell is modified by the introduction of or modification in any one or more of the list comprising phosphoenolpyruvate synthase activity (EC: 2.7.9.2 encoded, for instance, in E. coli by ppsA), phosphoenolpyruvate carboxykinase activity (EC 4.1.1.32 or EC 4.1.1.49 encoded, for instance, in Corynebacterium glutamicum by PCK or in E. coli by pckA, resp.), phosphoenolpyruvate carboxylase activity (EC 4.1.1.31 encoded, for instance, in E. coli by ppc), oxaloacetate decarboxylase activity (EC 4.1.1.112 encoded, for instance, in E. coli by eda), pyruvate kinase activity (EC 2.7.1.40 encoded, for instance, in E. coli by pykA and pykF), pyruvate carboxylase activity (EC 6.4.1.1 encoded, for instance, in B. subtilis by pyc) and malate dehydrogenase activity (EC 1.1.1.38 or EC 1.1.1.40 encoded, for instance, in E. coli by maeA or maeB, resp.).

In a more preferred embodiment, the cell is modified to overexpress any one or more of the polypeptides comprising ppsA from E. coli (UniProt ID P23538), PCK from C. glutamicum (UniProt ID Q6F5A5), pcka from E. coli (UniProt ID P22259), eda from E. coli (UniProt ID P0A955), maeA from E. coli (UniProt ID P26616) and maeB from E. coli (UniProt ID P76558).

In another and/or additional preferred embodiment, the cell is modified to express any one or more polypeptide having phosphoenolpyruvate synthase activity, phosphoenolpyruvate carboxykinase activity, oxaloacetate decarboxylase activity, or malate dehydrogenase activity.

In another and/or additional preferred embodiment and as a means for enhanced production and/or supply of PEP, the cell is modified by a reduced activity of phosphoenolpyruvate carboxylase activity, and/or pyruvate kinase activity, preferably a deletion of the genes encoding for phosphoenolpyruvate carboxylase, the pyruvate carboxylase activity and/or pyruvate kinase.

In an exemplary embodiment, the cell is genetically modified by different adaptations such as the overexpression of phosphoenolpyruvate synthase combined with the deletion of a pyruvate kinase gene, the overexpression of phosphoenolpyruvate synthase combined with the deletion of a phosphoenolpyruvate carboxylase gene, the overexpression of phosphoenolpyruvate synthase combined with the deletion of a pyruvate carboxylase gene, the overexpression of phosphoenolpyruvate carboxykinase combined with the deletion of a pyruvate kinase gene, the overexpression of phosphoenolpyruvate carboxykinase combined with the deletion of a phosphoenolpyruvate carboxylase gene, the overexpression of phosphoenolpyruvate carboxykinase combined with the deletion of a pyruvate carboxylase gene, the overexpression of oxaloacetate decarboxylase combined with the deletion of a pyruvate kinase gene, the overexpression of oxaloacetate decarboxylase combined with the deletion of a phosphoenolpyruvate carboxylase gene, the overexpression of oxaloacetate decarboxylase combined with the deletion of a pyruvate carboxylase gene, the overexpression of malate dehydrogenase combined with the deletion of a pyruvate kinase gene, the overexpression of malate dehydrogenase combined with the deletion of a phosphoenolpyruvate carboxylase gene and/or the overexpression of malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase, the overexpression of phosphoenolpyruvate synthase combined with the overexpression of an oxaloacetate decarboxylase, the overexpression of phosphoenolpyruvate synthase combined with the overexpression of a malate dehydrogenase, the overexpression of phosphoenolpyruvate carboxykinase combined with the overexpression of an oxaloacetate decarboxylase, the overexpression of phosphoenolpyruvate carboxykinase combined with the overexpression of a malate dehydrogenase, the overexpression of oxaloacetate decarboxylase combined with the overexpression of a malate dehydrogenase, the overexpression of phosphoenolpyruvate synthase combined with the overexpression of a phosphoenolpyruvate carboxykinase and the overexpression of an oxaloacetate decarboxylase, the overexpression of phosphoenolpyruvate synthase combined with the overexpression of a phosphoenolpyruvate carboxykinase and the overexpression of a malate dehydrogenase, the overexpression of phosphoenolpyruvate synthase combined with the overexpression of a phosphoenolpyruvate carboxykinase and the overexpression of an oxaloacetate decarboxylase and the overexpression of a malate dehydrogenase, the overexpression of phosphoenolpyruvate carboxykinase combined with the overexpression of an oxaloacetate decarboxylase and the overexpression of a malate dehydrogenase and/or the overexpression of phosphoenolpyruvate synthase combined with the overexpression of an oxaloacetate decarboxylase and the overexpression of a malate dehydrogenase.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene, the overexpression of phosphoenolpyruvate synthase combined with the overexpression of an oxaloacetate decarboxylase combined with the deletion of a pyruvate kinase gene, the overexpression of phosphoenolpyruvate synthase combined with the overexpression of a malate dehydrogenase combined with the deletion of a pyruvate kinase gene, the overexpression of phosphoenolpyruvate carboxykinase combined with the overexpression of an oxaloacetate decarboxylase combined with the deletion of a pyruvate kinase gene, the overexpression of phosphoenolpyruvate carboxykinase combined with the overexpression of a malate dehydrogenase combined with the deletion of a pyruvate kinase gene, the overexpression of an oxaloacetate decarboxylase combined with the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene, the overexpression of phosphoenolpyruvate synthase combined with the overexpression of a phosphoenolpyruvate carboxykinase and the overexpression of a malate dehydrogenase combined with the deletion of a pyruvate kinase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene, the overexpression of a phosphoenolpyruvate carboxykinase combined with the overexpression of an oxaloacetate decarboxylase and the overexpression of a malate dehydrogenase combined with the deletion of a pyruvate kinase gene, the overexpression of phosphoenolpyruvate synthase combined the overexpression of oxaloacetate decarboxylase and the overexpression of a malate dehydrogenase combined with the deletion of a pyruvate kinase gene.

In another exemplary embodiment, the cell is genetically modified by different adaptations such as the overexpression of phosphoenolpyruvate synthase combined with the overexpression of a phosphoenolpyruvate carboxykinase combined with the deletion of a phosphoenolpyruvate carboxylase gene, the overexpression of phosphoenolpyruvate synthase combined with the overexpression of an oxaloacetate decarboxylase combined with the deletion of a phosphoenolpyruvate carboxylase gene, the overexpression of phosphoenolpyruvate synthase combined with the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase combined with the deletion of a phosphoenolpyruvate carboxylase gene, the overexpression of a phosphoenolpyruvate carboxykinase combined with the overexpression of a malate dehydrogenase combined with the deletion of a phosphoenolpyruvate carboxylase gene, the overexpression of an oxaloacetate decarboxylase combined with the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a phosphoenolpyruvate carboxylase gene, the overexpression of phosphoenolpyruvate synthase combined the overexpression of an oxaloacetate decarboxylase and the overexpression of a malate dehydrogenase combined with the deletion of a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate carboxylase gene, the overexpression of phosphoenolpyruvate synthase combined with the overexpression of an oxaloacetate decarboxylase combined with the deletion of a pyruvate carboxylase gene, the overexpression of phosphoenolpyruvate synthase combined with the overexpression of a malate dehydrogenase combined with the deletion of a pyruvate carboxylase gene, the overexpression of a phosphoenolpyruvate carboxykinase combined with the overexpression of an oxaloacetate decarboxylase combined with the deletion of a pyruvate carboxylase gene, the overexpression of a phosphoenolpyruvate carboxykinase combined with the overexpression of a malate dehydrogenase combined with the deletion of a pyruvate carboxylase gene, the overexpression of an oxaloacetate decarboxylase combined with the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate carboxylase gene, the overexpression of phosphoenolpyruvate synthase combined with the overexpression of an oxaloacetate decarboxylase and the overexpression of a malate dehydrogenase combined with the deletion of a pyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a phosphoenolpyruvate carboxylase gene, the overexpression of phosphoenolpyruvate synthase combined with the overexpression of an oxaloacetate decarboxylase combined with the deletion of a pyruvate kinase gene and a phosphoenolpyruvate carboxylase gene, the overexpression of phosphoenolpyruvate synthase combined with the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a phosphoenolpyruvate carboxylase gene, the overexpression of a phosphoenolpyruvate carboxykinase combined with the overexpression of a malate dehydrogenase combined with the deletion of a pyruvate kinase gene and a phosphoenolpyruvate carboxylase gene, the overexpression of an oxaloacetate decarboxylase combined with the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of a malate dehydrogenase combined with the deletion of a pyruvate kinase gene and a phosphoenolpyruvate carboxylase gene, the overexpression of a phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a phosphoenolpyruvate carboxylase gene, the overexpression of a phosphoenolpyruvate synthase combined with the overexpression of an oxaloacetate decarboxylase and the overexpression of a malate dehydrogenase combined with the deletion of a pyruvate kinase gene and a phosphoenolpyruvate carboxylase gene.

In another exemplary embodiment, the cell is genetically modified by different adaptations such as the overexpression of phosphoenolpyruvate synthase combined with the overexpression of a phosphoenolpyruvate carboxykinase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a malate dehydrogenase combined with the deletion of a pyruvate kinase gene and a pyruvate carboxylase gene and a phosphoenolpyruvate carboxylase gene, the overexpression of a phosphoenolpyruvate carboxykinase combined with the overexpression of an oxaloacetate decarboxylase combined with the deletion of a pyruvate kinase gene and a pyruvate carboxylase gene and a phosphoenolpyruvate carboxylase gene, the overexpression of a phosphoenolpyruvate carboxykinase combined with the overexpression of a malate dehydrogenase combined with the deletion of a pyruvate kinase gene and a pyruvate carboxylase gene and a phosphoenolpyruvate carboxylase gene, the overexpression of an oxaloacetate decarboxylase combined with the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of a phosphoenolpyruvate carboxykinase and the overexpression of an oxaloacetate decarboxylase and the overexpression of a malate dehydrogenase combined with the deletion of a pyruvate kinase gene and pyruvate carboxylase gene and a phosphoenolpyruvate carboxylase gene, the overexpression of a phosphoenolpyruvate carboxykinase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene, the overexpression of phosphoenolpyruvate synthase combined with the overexpression of an oxaloacetate decarboxylase and the overexpression of a malate dehydrogenase combined with the deletion of a pyruvate kinase gene and a pyruvate carboxylase gene and a phosphoenolpyruvate carboxylase gene. In an even more preferred embodiment of the method and/or cell of the disclosure, the polypeptides that are encoded by the multiple coding DNA sequences within a set are directly involved in the synthesis and/or supply of PEP.

According to another embodiment of the method and/or cell of the disclosure, the cell comprises one or more sets of multiple coding DNA sequences wherein the multiple coding DNA sequences within a set differ in nucleotide sequence and wherein each set of the multiple coding DNA sequences encode polypeptides that have a different function and/or activity of interest compared to the other sets of multiple coding DNA sequences. In a preferred embodiment of the method and/or cell, the cell comprises a set of multiple coding DNA sequences wherein the multiple coding DNA sequences differ in nucleotide sequence and each encode a polypeptide having galactoside beta-1,3-N-acetylglucosaminyltransferase activity, and wherein each of the coding DNA sequences is chosen from the list comprising SEQ ID NOs:01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57. In an alternative and/or additional preferred embodiment of the method and/or cell, the cell comprises a set of multiple coding DNA sequences wherein the multiple coding DNA sequences differ in nucleotide sequence and wherein each of the coding DNA sequences is a fragment of any one of SEQ ID NOs:01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57 encoding a polypeptide having galactoside beta-1,3-N-acetylglucosaminyltransferase activity. In an alternative and/or additional preferred embodiment of the method and/or cell, the cell comprises a set of multiple coding DNA sequences wherein the multiple coding DNA sequences differ in nucleotide sequence and wherein each of the coding DNA sequences comprises or consists of a nucleotide sequence having 80% or more sequence identity to the full-length nucleotide sequence of any one of SEQ ID NO:01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57 and encoding a polypeptide having galactoside beta-1,3-N-acetylglucosaminyltransferase activity.

In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a polypeptide chosen from the list comprising SEQ ID NOs:79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 and 131. In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a functional fragment of a polypeptide according to any one of SEQ ID NOs:79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 or 131 and having galactoside beta-1,3-N-acetylglucosaminyltransferase activity. In an alternative and/or additional preferred embodiment of the method and/or cell, the cell comprises a set of multiple coding DNA sequences wherein each of the coding DNA sequences encodes a polypeptide comprising or consisting of an amino acid sequence having 80% or more sequence identity to the full-length amino acid sequence of any one of SEQ ID NO:79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 or 131 and having galactoside beta-1,3-N-acetylglucosaminyltransferase activity.

In an alternative and/or additional preferred embodiment of the method and/or cell, the cell comprises a set of multiple coding DNA sequences wherein the multiple coding DNA sequences differ in nucleotide sequence and each encode a polypeptide having N-acetylglucosamine beta-1,3-galactosyltransferase activity, and wherein each of the coding DNA sequences is chosen from the list comprising SEQ ID NOs:58, 59, 60, 61, 62, 63, 64, 65 and 66. In an alternative and/or additional preferred embodiment of the method and/or cell, the cell comprises a set of multiple coding DNA sequences wherein the multiple coding DNA sequences differ in nucleotide sequence and wherein each of the coding DNA sequences is a fragment of any one of SEQ ID NOs:58, 59, 60, 61, 62, 63, 64, 65 and 66 encoding a polypeptide having N-acetylglucosamine beta-1,3-galactosyltransferase activity. In an alternative and/or additional preferred embodiment of the method and/or cell, the cell comprises a set of multiple coding DNA sequences wherein the multiple coding DNA sequences differ in nucleotide sequence and wherein each of the coding DNA sequences comprises or consists of a nucleotide sequence having 80% or more sequence identity to the full-length nucleotide sequence of any one of SEQ ID NO:58, 59, 60, 61, 62, 63, 64, 65 or 66 and encoding a polypeptide having N-acetylglucosamine beta-1,3-galactosyltransferase activity.

In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a polypeptide chosen from the list comprising SEQ ID NOs:132, 133, 134 and 135. In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a functional fragment of a polypeptide according to any one of SEQ ID NOs:132, 133, 134 or 135 and having N-acetylglucosamine beta-1,3-galactosyltransferase activity. In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a polypeptide comprising or consisting of an amino acid sequence having 80% or more sequence identity to the full-length amino acid sequence of any one of SEQ ID NO:132, 133, 134 or 135 and having N-acetylglucosamine beta-1,3-galactosyltransferase activity.

In an alternative and/or additional preferred embodiment of the method and/or cell, the cell comprises a set of multiple coding DNA sequences wherein the multiple coding DNA sequences differ in nucleotide sequence and each encode a polypeptide having N-acetylglucosamine beta-1,4-galactosyltransferase activity, and wherein each of the coding DNA sequences is chosen from the list comprising SEQ ID NOs:67, 68, 69, 70, 71, 72, 73, 74, 75, 76, 77 and 78. In an alternative and/or additional preferred embodiment of the method and/or cell, the cell comprises a set of multiple coding DNA sequences wherein the multiple coding DNA sequences differ in nucleotide sequence and wherein each of the coding DNA sequences is a fragment of any one of SEQ ID NOs:67, 68, 69, 70, 71, 72, 73, 74, 75, 76, 77 and 78 encoding a polypeptide having N-acetylglucosamine beta-1,4-galactosyltransferase activity. In an alternative and/or additional preferred embodiment of the method and/or cell, the cell comprises a set of multiple coding DNA sequences wherein the multiple coding DNA sequences differ in nucleotide sequence and wherein each of the coding DNA sequences comprises or consists of a nucleotide sequence having 80% or more sequence identity to the full-length nucleotide sequence of any one of SEQ ID NO:67, 68, 69, 70, 71, 72, 73, 74, 75, 76, 77 or 78 and encoding a polypeptide having N-acetylglucosamine beta-1,4-galactosyltransferase activity.

In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a polypeptide chosen from the list comprising SEQ ID NOs:136, 137, 138, 139, 140, 141, 142, 143, 144 and 145. In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a functional fragment of a polypeptide according to any one of SEQ ID NO:136, 137, 138, 139, 140, 141, 142, 143, 144 or 145 and having N-acetylglucosamine beta-1,4-galactosyltransferase activity. In an alternative and/or additional preferred embodiment of the method and/or cell, the cell comprises a set of multiple coding DNA sequences wherein the multiple coding DNA sequences differ in nucleotide sequence and wherein each of the coding DNA sequences encodes a polypeptide comprising or consisting of an amino acid sequence having 80% or more sequence identity to the full-length amino acid sequence of any one of SEQ ID NO:136, 137, 138, 139, 140, 141, 142, 143, 144 or 145 and having N-acetylglucosamine beta-1,4-galactosyltransferase activity.

According to another aspect of the method and/or cell of the disclosure, the cell comprises a set of multiple coding DNA sequences wherein the multiple coding DNA sequences differ in nucleotide sequence and wherein each of the multiple coding DNA sequences encodes a polypeptide having N-acylneuraminate cytidylyltransferase activity. In a preferred embodiment of the method and/or cell, each of the coding DNA sequences in the set encodes a polypeptide chosen from the list comprising the polypeptide from Campylobacter jejuni with UniProt ID Q93MP7, the polypeptide from Haemophilus influenzae with GenBank No. AGV11798.1 and the polypeptide from Pasteurella multocida with GenBank No. AMK07891.1 and having N-acylneuraminate cytidylyltransferase activity. In an alternative and/or additional preferred embodiment of the method and/or cell, each of the coding DNA sequences in the set encodes a functional fragment of any one of the polypeptide from C. jejuni with UniProt ID Q93MP7, H. influenzae with GenBank No. AGV11798.1 or P. multocida with GenBank No. AMK07891.1 and having N-acylneuraminate cytidylyltransferase activity. In an alternative and/or additional preferred embodiment of the method and/or cell, each of the coding DNA sequences in the set encodes a polypeptide comprising or consisting of an amino acid sequence having 80% or more sequence identity to the full-length amino acid sequence of any one of the polypeptides from C. jejuni with UniProt ID Q93MP7, H. influenzae with GenBank No. AGV11798.1 or P. multocida with GenBank No. AMK07891.1 and having N-acylneuraminate cytidylyltransferase activity.

According to a further aspect of the method and/or cell of the disclosure, the cell further comprises at least one coding DNA sequence encoding a polypeptide having N-acetylneuraminate synthase activity and/or two or more copies of one or more coding DNA sequences of an alpha-2,3-sialyltransferase, an alpha-2,6-sialyltransferase, and/or an alpha-2,8-sialyltransferase. In a preferred embodiment of the method and/or cell, the polypeptide having N-acetylneuraminate synthase activity is any one of the polypeptides chosen from the list comprising the polypeptide from Neisseria meningitidis with UniProt ID E0NCD4, the polypeptide from Campylobacter jejuni with UniProt ID Q93MP9, the polypeptide from Aeromonas caviae with UniProt ID Q9R9S2, the polypeptide from Candidatus koribacter versatilis with UniProt ID Q1IMQ8, the polypeptide from Legionella pneumophila with UniProt ID Q9RDX5, the polypeptide from Methanocaldococcus jannaschii with UniProt ID Q58465 and the polypeptide from Moritella viscosa with UniProt ID A0A090IMH4 and having N-acetylneuraminate synthase activity. In an alternative and/or additional preferred embodiment of the method and/or cell, the polypeptide having N-acetylneuraminate synthase activity is a functional fragment of any one of the polypeptide from N. meningitidis with UniProt ID E0NCD4, C. jejuni with UniProt ID Q93MP9, A. caviae with UniProt ID Q9R9S2, C. koribacter versatilis with UniProt ID Q1IMQ8, L. pneumophila with UniProt ID Q9RDX5, M. jannaschii with UniProt ID Q58465 or M. viscosa with UniProt ID A0A090IMH4 and having N-acetylneuraminate synthase activity. In an alternative and/or additional preferred embodiment of the method and/or cell, the polypeptide having N-acetylneuraminate synthase activity is any one of the polypeptides comprising or consisting of an amino acid sequence having 80% or more sequence identity to the full-length amino acid sequence of any one of the polypeptides from N. meningitidis with UniProt ID E0NCD4, C. jejuni with UniProt ID Q93MP9, A. caviae with UniProt ID Q9R9S2, C. koribacter versatilis with UniProt ID Q1IMQ8, L. pneumophila with UniProt ID Q9RDX5, M. jannaschii with UniProt ID Q58465 or M. viscosa with UniProt ID A0A090IMH4 and having N-acetylneuraminate synthase activity.

According to another preferred embodiment of the method and/or cell of the disclosure, the cell comprises a modification for reduced production of acetate. The modification can be any one or more chosen from the group comprising overexpression of an acetyl-coenzyme A synthetase, a full or partial knock-out or rendered less functional pyruvate dehydrogenase and a full or partial knock-out or rendered less functional lactate dehydrogenase.

In a further embodiment of the method and/or cell of the disclosure, the cell is modified in the expression or activity of at least one acetyl-coenzyme A synthetase like e.g., acs from E. coli, S. cerevisiae, H. sapiens, M. musculus. In a preferred embodiment, the acetyl-coenzyme A synthetase is an endogenous protein of the cell with a modified expression or activity, preferably the endogenous acetyl-coenzyme A synthetase is overexpressed; alternatively, the acetyl-coenzyme A synthetase is a heterologous protein that is heterogeneously introduced and expressed in the cell, preferably overexpressed. The endogenous acetyl-coenzyme A synthetase can have a modified expression in the cell, which also expresses a heterologous acetyl-coenzyme A synthetase. In a more preferred embodiment, the cell is modified in the expression or activity of the acetyl-coenzyme A synthetase acs from E. coli (UniProt ID P27550). In another and/or additional preferred embodiment, the cell is modified in the expression or activity of a functional homolog, variant or derivative of acs from E. coli (UniProt ID P27550) having at least 80% overall sequence identity to the full-length of the polypeptide from E. coli (UniProt ID P27550) and having acetyl-coenzyme A synthetase activity.

In an alternative and/or additional further embodiment of the method and/or cell of the disclosure, the cell is modified in the expression or activity of at least one pyruvate dehydrogenase like e.g., from E. coli, S. cerevisiae, H. sapiens and R. norvegicus. In a preferred embodiment, the cell has been modified to have at least one partially or fully knocked out or mutated pyruvate dehydrogenase encoding gene by means generally known by the person skilled in the art resulting in at least one protein with less functional or being disabled for pyruvate dehydrogenase activity. In a more preferred embodiment, the cell has a full knock-out in the poxB encoding gene resulting in a cell lacking pyruvate dehydrogenase activity.

In an alternative and/or additional further embodiment of the method and/or cell of the disclosure, the cell is modified in the expression or activity of at least one lactate dehydrogenase like e.g., from E. coli, S. cerevisiae, H. sapiens and R. norvegicus. In a preferred embodiment, the cell has been modified to have at least one partially or fully knocked out or mutated lactate dehydrogenase encoding gene by means generally known by the person skilled in the art resulting in at least one protein with less functional or being disabled for lactate dehydrogenase activity. In a more preferred embodiment, the cell has a full knock-out in the ldhA encoding gene resulting in a cell lacking lactate dehydrogenase activity.

According to another preferred embodiment of the method and/or cell of the disclosure, the cell comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose undecaprenyl-phosphate glucose-1-phosphatetransferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

According to another preferred embodiment of the method and/or cell of the disclosure, the cell comprises a catabolic pathway for selected mono-, di- or oligosaccharides, which is at least partially inactivated, the mono-, di-, or oligosaccharides being involved in and/or required for the production of a di- and/or oligosaccharide.

According to another preferred embodiment of the method and/or cell of the disclosure, the cell is using a precursor for the production of a di- and/or oligosaccharide, preferably the precursor being fed to the cell from the cultivation medium. According to a more preferred aspect of the method and/or cell, the cell is using at least two precursors for the production of the di- and/or oligosaccharide, preferably the precursors being fed to the cell from the cultivation medium. According to another preferred aspect of the method and/or cell of the disclosure, the cell is producing at least one precursor, preferably at least two precursors, for the production of the di- and/or oligosaccharide. In a preferred embodiment of the method and/or cell, the precursor that is used by the cell for the production of a di- and/or oligosaccharide is completely converted into the di- and/or oligosaccharide.

According to another preferred embodiment of the method and/or cell of the disclosure, the cell produces a di- and/or oligosaccharide intracellularly. According to a more preferred embodiment of the method and/or cell, a fraction of the produced di- and/or oligosaccharide remains intracellularly in the cell. According to an alternative more preferred embodiment of the method and/or cell, substantially all of the produced di- and/or oligosaccharide remains intracellularly. According to an alternative and/or additional more preferred embodiment of the method and/or cell, a fraction of the produced di- and/or oligosaccharide remains intracellularly in the cell and another fraction of the produced di- and/or oligosaccharide is excreted outside the cell via passive or active transport. According to an alternative and/or additional more preferred embodiment of the method and/or cell, substantially all of the produced di- and/or oligosaccharide is excreted outside the cell via passive or active transport.

According to another preferred embodiment of the method and/or cell of the disclosure, the cell produces 90 g/L or more of a di- and/or oligosaccharide in the whole broth and/or supernatant. In a more preferred embodiment, the di- and/or oligosaccharide produced in the whole broth and/or supernatant has a purity of at least 80% measured on the total amount of di- and/or oligosaccharide and its precursor produced by the cell in the whole broth and/or supernatant, respectively.

Another aspect of the disclosure provides for a method and a cell wherein a di- and/or oligosaccharide is produced in and/or by a bacterial, fungal, yeast, insect, plant, animal or protozoan expression system or cell as described herein. The expression system or cell is chosen from the list comprising a bacterium, a fungus, or a yeast, or, refers to a plant, animal, or protozoan cell. The latter bacterium preferably belongs to the phylum of the Proteobacteria or the phylum of the Firmicutes or the phylum of the Cyanobacteria or the phylum Deinococcus—Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the disclosure specifically relates to a mutated and/or transformed Escherichia coli strain as indicated above wherein the E. coli strain is a K12 strain. More specifically, the disclosure relates to a mutated and/or transformed Escherichia co/i strain as indicated above wherein the K12 strain is E. coli MG1655. The latter bacterium belonging to the phylum Firmicutes belongs preferably to the Bacilli, preferably from the species Bacillus. The latter fungus belongs preferably to the genus Rhizopus, Dictyostelium, Penicillium, Mucor or Aspergillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces (with members like e.g., Saccharomyces cerevisiae, S. bayanus, S. boulardii), Zygosaccharomyces, Pichia (with members like e.g., Pichia pastoris, P. anomala, P. kluyveri), Komagataella, Hansenula, Yarrowia (like e.g., Yarrowia lipolytica), Starmerella (like e.g., Starmerella bombicola), Kluyveromyces (with members like e.g., Pichia pastoris, P. anomala, P. kluyveri) or Debaromyces. Plant cells include cells of flowering and non-flowering plants, as well as algal cells, for example, Chlamydomonas, Chlorella, etc. Preferably, the plant is a tobacco, alfalfa, rice, tomato, cotton, rapeseed, soy, maize or corn plant. The latter animal cell is preferably derived from non-human mammals (e.g., cattle, buffalo, pig, sheep, mouse, rat), birds (e.g., chicken, duck, ostrich, turkey, pheasant), fish (e.g., swordfish, salmon, tuna, sea bass, trout, catfish), invertebrates (e.g., lobster, crab, shrimp, clams, oyster, mussel, sea urchin), reptiles (e.g., snake, alligator, turtle), amphibians (e.g., frogs) or insects (e.g., fly, nematode) or is a genetically modified cell line derived from human cells excluding embryonic stem cells. Both human and non-human mammalian cells are preferably chosen from the list comprising an epithelial cell like e.g., a mammary epithelial cell, an embryonic kidney cell (e.g., HEK 293 or HEK 293T cell), a fibroblast cell, a COS cell, a Chinese hamster ovary (CHO) cell, a murine myeloma cell like e.g., an N20, SP2/0 or YB2/0 cell, an NIH-3T3 cell, a non-mammary adult stem cell or derivatives thereof such as described in WO 2021067641. The latter insect cell is preferably derived from Spodoptera frugiperda like e.g., Sf9 or Sf21 cells, Bombyx mori, Mamestra brassicae, Trichoplusia ni like e.g., BTI-TN-5B1-4 cells or Drosophila melanogaster like e.g., Drosophila S2 cells. The latter protozoan cell preferably is a Leishmania tarentolae cell.

In a preferred embodiment of the method and/or cell of the disclosure, the cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose.

In a more preferred embodiment of the method and/or cell, the reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose is provided by a mutation in any one or more glycosyltransferases involved in the synthesis of any one of the poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose, wherein the mutation provides for a deletion or lower expression of any one of the glycosyltransferases. The glycosyltransferases comprise glycosyltransferase genes encoding poly-N-acetyl-D-glucosamine synthase subunits, UDP-N-acetylglucosamine-undecaprenyl-phosphate N-acetylglucosaminephosphotransferase, Fuc4NAc (4-acetamido-4,6-dideoxy-D-galactose) transferase, UDP-N-acetyl-D-mannosaminuronic acid transferase, the glycosyltransferase genes encoding the cellulose synthase catalytic subunits, the cellulose biosynthesis protein, colanic acid biosynthesis glucuronosyltransferase, colanic acid biosynthesis galactosyltransferase, colanic acid biosynthesis fucosyltransferase, UDP-glucose:undecaprenyl-phosphate glucose-1-phosphate transferase, putative colanic biosynthesis glycosyl transferase, UDP-glucuronate:LPS(HepIII) glycosyltransferase, ADP-heptose-LPS heptosyltransferase 2, ADP-heptose:LPS heptosyltransferase 1, putative ADP-heptose:LPS heptosyltransferase 4, lipopolysaccharide core biosynthesis protein, UDP-glucose:(glucosyl)LPS α-1,2-glucosyltransferase, UDP-D-glucose:(glucosyl)LPS α-1,3-glucosyltransferase, UDP-D-galactose:(glucosyl)lipopolysaccharide-1,6-D-galactosyltransferase, lipopolysaccharide glucosyltransferase I, lipopolysaccharide core heptosyltransferase 3, β-1,6-galactofuranosyltransferase, undecaprenyl-phosphate 4-deoxy-4-formamido-L-arabinose transferase, lipid IVA 4-amino-4-deoxy-L-arabinosyltransferase, bactoprenol glucosyl transferase, putative family 2 glycosyltransferase, the osmoregulated periplasmic glucans (OPG) biosynthesis protein G, OPG biosynthesis protein H, glucosylglycerate phosphorylase, glycogen synthase, 1,4-α-glucan branching enzyme, 4-α-glucanotransferase and trehalose-6-phosphate synthase. In an exemplary embodiment, the cell is mutated in any one or more of the glycosyltransferases comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, glgA, glgB, malQ, otsA and yaiP, wherein the mutation provides for a deletion or lower expression of any one of the glycosyltransferases.

In an alternative and/or additional preferred embodiment of the method and/or cell, the reduced or abolished synthesis of poly-N-acetyl-glucosamine (PNAG) is provided by over-expression of a carbon storage regulator encoding gene, deletion of a Na+/H+ antiporter regulator encoding gene and/or deletion of the sensor histidine kinase encoding gene.

Another embodiment provides for a cell to be stably cultured in a medium, wherein the medium can be any type of growth medium as well-known to the skilled person comprising minimal medium, complex medium or growth medium enriched in certain compounds, for example, but not limited to vitamins, trace elements, amino acids and/or, precursors and/or acceptors as defined herein.

The cell as used herein is capable to grow on a monosaccharide, disaccharide, oligosaccharide, polysaccharide, polyol, glycerol, a complex medium including molasses, corn steep liquor, peptone, tryptone, yeast extract or a mixture thereof like e.g., a mixed feedstock, preferably a mixed monosaccharide feedstock like e.g., hydrolysed sucrose as the main carbon source. With the term “complex medium” is meant a medium for which the exact constitution is not determined. With the term “main” is meant the most important carbon source for the cell for the production of the di- and/or oligosaccharide of interest, biomass formation, carbon dioxide and/or by-products formation (such as acids and/or alcohols, such as acetate, lactate, and/or ethanol), i.e., 20, 30, 40, 50, 60, 70, 75, 80, 85, 90, 95, 98, 99% of all the required carbon is derived from the above-indicated carbon source. In one embodiment of the disclosure, the carbon source is the sole carbon source for the organism, i.e., 100% of all the required carbon is derived from the above-indicated carbon source. Common main carbon sources comprise but are not limited to glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate. As used herein, a precursor as defined herein cannot be used as a carbon source for the production of the di- and/or oligosaccharide.

In another embodiment of the method and/or cell of the disclosure, the cell resists the phenomenon of lactose killing when grown in an environment in which lactose is combined with one or more other carbon source(s). With the term “lactose killing” is meant the hampered growth of the cell in medium in which lactose is present together with another carbon source. In a preferred embodiment, the cell is genetically modified such that it retains at least 50% of the lactose influx without undergoing lactose killing, even at high lactose concentrations, as is described in WO 2016/075243. The genetic modification comprises expression and/or over-expression of an exogenous and/or an endogenous lactose transporter gene by a heterologous promoter that does not lead to a lactose killing phenotype and/or modification of the codon usage of the lactose transporter to create an altered expression of the lactose transporter that does not lead to a lactose killing phenotype. The content of WO 2016/075243 in this regard is incorporated by reference.

According to another embodiment of the method and/or cell of the disclosure, the cell is capable to produce a mixture of di- and/or oligosaccharides. Preferably, the cell is capable to produce a mixture of di- and oligosaccharides. In another embodiment of the method and/or cell of the disclosure, the cell is capable to produce a mixture of charged and/or neutral di- and/or oligosaccharides. Preferably, the cell is capable to produce a mixture of charged and/or neutral di- and oligosaccharides. In a preferred embodiment of the method and/or cell, the charged di- and/or oligosaccharides comprise at least one sialylated di- and/or oligosaccharide. In a preferred embodiment of the method and/or cell, the neutral di- and/or oligosaccharides are fucosylated. In another preferred embodiment of the method and/or cell, the neutral di- and/or oligosaccharides are not fucosylated. In another preferred embodiment of the method and/or cell, the neutral di- and/or oligosaccharides are a mixture of fucosylated and non-fucosylated neutral di- and/or oligosaccharides.

In an alternative and/or additional embodiment, the cell is capable to produce a mixture of charged di- and/or oligosaccharides. In a preferred embodiment of the method and/or cell, the charged di- and/or oligosaccharides comprise at least one sialylated di- and/or oligosaccharide.

According to the disclosure, a mixture comprises or consists of at least two different ‘di- and/or oligosaccharide’, preferably at least three different ‘di- and/or oligosaccharide’, more preferably at least four different ‘di- and/or oligosaccharide’.

Throughout the disclosure, unless explicitly specified otherwise, the term “di- and/or oligosaccharide” can be preferably replaced with the term “oligosaccharide,” more preferably “milk oligosaccharide,” even more preferably “mammalian milk oligosaccharide,” most preferably “human milk oligosaccharide.”

According to another embodiment of the method of the disclosure, the conditions permissive to produce the di- and/or oligosaccharide comprise the use of a culture medium comprising at least one precursor and/or acceptor for the production of the di- and/or oligosaccharide. Preferably, the culture medium contains at least one precursor selected from the group comprising lactose, galactose, fucose, sialic acid, GlcNAc, GalNAc, lacto-N-biose (LNB), N-acetyllactosamine (LacNAc).

According to an alternative and/or additional embodiment of the method of the disclosure, the conditions permissive to produce the di- and/or oligosaccharide comprise adding to the culture medium at least one precursor and/or acceptor feed for the production of the di- and/or oligosaccharide.

According to an alternative embodiment of the method of the disclosure, the conditions permissive to produce the di- and/or oligosaccharide comprise the use of a culture medium to cultivate a cell of disclosure for the production of a di- and/or oligosaccharide wherein the culture medium lacks any precursor and/or acceptor for the production of the di- and/or oligosaccharide and is combined with a further addition to the culture medium of at least one precursor and/or acceptor feed for the production of the di- and/or oligosaccharide.

In a preferred embodiment, the method for the production of a di- and/or oligosaccharide as described herein comprises at least one of the following steps:
i) Use of a culture medium comprising at least one precursor and/or acceptor;
ii) Adding to the culture medium in a reactor at least one precursor and/or acceptor feed wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than two-fold of the volume of the culture medium before the addition of the precursor and/or acceptor feed;
iii) Adding to the culture medium in a reactor at least one precursor and/or acceptor feed wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than two-fold of the volume of the culture medium before the addition of the precursor and/or acceptor feed and wherein preferably, the pH of the precursor and/or acceptor feed is set between 3 and 7 and wherein preferably, the temperature of the precursor and/or acceptor feed is kept between 20° C. and 80° C.;
iv) Adding at least one precursor and/or acceptor feed in a continuous manner to the culture medium over the course of 1 day, 2 days, 3 days, 4 days, 5 days by means of a feeding solution;
v) Adding at least one precursor and/or acceptor feed in a continuous manner to the culture medium over the course of 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
the method resulting in a di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

In another and/or additional preferred embodiment, the method for the production of a di- and/or oligosaccharide as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10,000 m³ (cubic meter);
ii) Adding to the culture medium at least one precursor and/or acceptor in one pulse or in a discontinuous (pulsed) manner wherein the total reactor volume ranges from 250 mL (millilitre) to 10,000 m³ (cubic meter), preferably so that the final volume of the culture medium is not more than three-fold, preferably not more than two-fold, more preferably less than two-fold of the volume of the culture medium before the addition of the precursor and/or acceptor feed pulse(s);
iii) Adding to the culture medium in a reactor at least one precursor and/or acceptor feed in one pulse or in a discontinuous (pulsed) manner wherein the total reactor volume ranges from 250 mL (millilitre) to 10,000 m³ (cubic meter), preferably so that the final volume of the culture medium is not more than three-fold, preferably not more than two-fold, more preferably less than two-fold of the volume of the culture medium before the addition of the precursor and/or acceptor feed pulse(s) and wherein preferably, the pH of the precursor and/or acceptor feed pulse(s) is set between 3 and 7 and wherein preferably, the temperature of the precursor and/or acceptor feed pulse(s) is kept between 20° C. and 80° C.;
iv) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution;
v) Adding at least one precursor and/or acceptor feed in a discontinuous (pulsed) manner to the culture medium over the course of 5 minutes, 10 minutes, 30 minutes, 1 hour, 2 hours, 4 hours, 10 hours, 12 hours, 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
the method resulting in a di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

In a further, more preferred embodiment, the method for the production of a di- and/or oligosaccharide as described herein comprises at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10,000 m³ (cubic meter);
ii) Adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than 2-fold of the volume of the culture medium before the addition of the lactose feed;
iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than 2-fold of the volume of the culture medium before the addition of the lactose feed and wherein preferably the pH of the lactose feed is set between 3 and 7 and wherein preferably the temperature of the lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of the feeding solution is set between 3 and 7 and wherein preferably the temperature of the feeding solution is kept between 20° C. and 80° C.;
the method resulting in an oligosaccharide produced from the lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

Preferably the lactose feed is accomplished by adding lactose from the beginning of the cultivation at a concentration of at least 5 mM, preferably in a concentration of 30, 40, 50, 60, 70, 80, 90, 100, 150 mM, more preferably at a concentration >300 mM.

In another embodiment the lactose feed is accomplished by adding lactose to the cultivation in a concentration, such that throughout the production phase of the cultivation a lactose concentration of at least 5 mM, preferably 10 mM or 30 mM is obtained.

In a further embodiment of the methods described herein the host cells are cultivated for at least about 60, 80, 100, or about 120 hours or in a continuous manner.

In a preferred embodiment, a carbon source is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per litre of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon source, preferably glucose or sucrose, to the culture medium before the lactose is added to the cultivation in a second phase.

In another preferred embodiment of the method of disclosure, a first phase of exponential cell growth is provided by adding a carbon-based substrate, preferably glucose or sucrose, to the culture medium comprising a precursor, preferably lactose, followed by a second phase wherein only a carbon-based substrate, preferably glucose or sucrose, is added to the culture medium.

In another preferred embodiment of the method of disclosure, a first phase of exponential cell growth is provided by adding a carbon-based substrate, preferably glucose or sucrose, to the culture medium comprising a precursor, preferably lactose, followed by a second phase wherein a carbon-based substrate, preferably glucose or sucrose, and a precursor, preferably lactose, are added to the culture medium.

In an alternative preferable embodiment, in the method as described herein, the lactose is added already in the first phase of exponential growth together with the carbon-based substrate.

According to the disclosure, the methods as described herein preferably comprises a step of separating the di- and/or oligosaccharide from the cultivation.

The terms “separating from the cultivation” means harvesting, collecting, or retrieving the di- and/or oligosaccharide from the cell and/or the medium of its growth.

The di- and/or oligosaccharide can be separated in a conventional manner from the aqueous culture medium, in which the cell was grown. In case the di- and/or oligosaccharide is still present in the cells producing the di- and/or oligosaccharide, conventional manners to free or to extract the di- and/or oligosaccharide out of the cells can be used, such as cell destruction using high pH, heat shock, sonication, French press, homogenization, enzymatic hydrolysis, chemical hydrolysis, solvent hydrolysis, detergent, hydrolysis, . . . . The culture medium and/or cell extract together and separately can then be further used for separating the di- and/or oligosaccharide.

This preferably involves clarifying the di- and/or oligosaccharide to remove suspended particulates and contaminants, particularly cells, cell components, insoluble metabolites and debris produced by culturing the genetically modified cell. In this step, the di- and/or oligosaccharide can be clarified in a conventional manner. Preferably, the di- and/or oligosaccharide is clarified by centrifugation, flocculation, decantation and/or filtration. Another step of separating the di- and/or oligosaccharide preferably involves removing substantially all the proteins, peptides, amino acids, RNA and DNA, and any endotoxins and glycolipids that could interfere with the subsequent separation step, from the di- and/or oligosaccharide, preferably after it has been clarified. In this step, proteins and related impurities can be removed from the di- and/or oligosaccharide in a conventional manner. Preferably, proteins, salts, by-products, color, endotoxins and other related impurities are removed from the di- and/or oligosaccharide by ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis (e.g., using slab-polyacrylamide or sodium dodecyl sulphate-polyacrylamide gel electrophoresis (PAGE)), affinity chromatography (using affinity ligands including e.g., DEAE-SEPHAROSE®, poly-L-lysine and polymyxin-B, endotoxin-selective adsorber matrices), ion exchange chromatography (such as but not limited to cation exchange, anion exchange, mixed bed ion exchange, inside-out ligand attachment), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, remaining proteins and related impurities are retained by a chromatography medium or a selected membrane.

In a further preferred embodiment, the methods as described herein also provide for a further purification of the di- and/or oligosaccharide as produced according to a method of disclosure. A further purification of the di- and/or oligosaccharide may be accomplished, for example, by use of (activated) charcoal or carbon, nanofiltration, ultrafiltration, electrophoresis, enzymatic treatment or ion exchange to remove any remaining DNA, protein, LPS, endotoxins, or other impurity. Alcohols, such as ethanol, and aqueous alcohol mixtures can also be used. Another purification step is accomplished by crystallization, evaporation or precipitation of the di- and/or oligosaccharide. Another purification step is to dry, e.g., spray dry, lyophilize, spray freeze dry, freeze spray dry, band dry, belt dry, vacuum band dry, vacuum belt dry, drum dry, roller dry, vacuum drum dry or vacuum roller dry the produced di- and/or oligosaccharide.

In an exemplary embodiment, the separation and purification of the di- and/or oligosaccharide is made in a process, comprising the following steps in any order:
a) contacting the cultivation or a clarified version thereof with a nanofiltration membrane with a molecular weight cut-off (MWCO) of 600-3500 Da ensuring the retention of the produced di- and/or oligosaccharide and allowing at least a part of the proteins, salts, by-products, colour and other related impurities to pass,
b) conducting a diafiltration process on the retentate from step a), using the membrane, with an aqueous solution of an inorganic electrolyte, followed by optional diafiltration with pure water to remove excess of the electrolyte,
c) and collecting the retentate enriched in the di- and/or oligosaccharide in the form of a salt from the cation of the electrolyte.

In an alternative exemplary embodiment, the separation and purification of the di- and/or oligosaccharide is made in a process, comprising the following steps in any order: subjecting the cultivation or a clarified version thereof to two membrane filtration steps using different membranes, wherein
one membrane has a molecular weight cut-off of between about 300 Dalton to about 500 Dalton, and
the other membrane as a molecular weight cut-off of between about 600 Dalton to about 800 Dalton.

In an alternative exemplary embodiment, the separation and purification of the di- and/or oligosaccharide is made in a process, comprising the following steps in any order comprising the step of treating the cultivation or a clarified version thereof with a strong cation exchange resin in H+-form and a weak anion exchange resin in free base form.

In an alternative exemplary embodiment, the separation and purification of the di- and/or oligosaccharide is made in the following way. The cultivation comprising the produced di- and/or oligosaccharide, biomass, medium components and contaminants is applied to the following purification steps:
i) separation of biomass from the cultivation,
ii) cationic ion exchanger treatment for the removal of positively charged material,
iii) anionic ion exchanger treatment for the removal of negatively charged material,
iv) nanofiltration step and/or electrodialysis step,
wherein a purified solution comprising the produced di- and/or oligosaccharide at a purity of greater than or equal to 80 percent is provided. Optionally the purified solution is dried by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying.

In an alternative exemplary embodiment, the separation and purification of the di- and/or oligosaccharide is made in a process, comprising the following steps in any order: enzymatic treatment of the cultivation; removal of the biomass from the cultivation; ultrafiltration; nanofiltration; and a column chromatography step. Preferably such column chromatography is a single column or a multiple column. Further preferably the column chromatography step is simulated moving bed chromatography. Such simulated moving bed chromatography preferably comprises i) at least 4 columns, wherein at least one column comprises a weak or strong cation exchange resin; and/or ii) four zones I, II, III and IV with different flow rates; and/or iii) an eluent comprising water; and/or iv) an operating temperature of 15 degrees to 60 degrees centigrade.

In a specific embodiment, the disclosure provides the produced di- and/or oligosaccharide, which is dried to powder by any one or more drying steps chosen from the list comprising spray drying, lyophilization, spray freeze drying, freeze spray drying, band drying, belt drying, vacuum band drying, vacuum belt drying, drum drying, roller drying, vacuum drum drying and vacuum roller drying, wherein the dried powder contains <15 percent-wt. of water, preferably <10 percent-wt. of water, more preferably <7 percent-wt. of water, most preferably <5 percent-wt. of water.

Another aspect of the disclosure provides the use of a cell as defined herein, in a method for the production of a di- and/or oligosaccharide, preferably in a method for the production of a di- and/or oligosaccharide according to the disclosure. An alternative and/or additional embodiment of the disclosure provides the use of a cell as defined herein, in a method for the production of a mixture of di- and/or oligosaccharide. A preferred aspect provides the use of a cell of disclosure in a method for the production of a mixture of mammalian milk oligosaccharides (MMOs). An alternative and/or additional aspect of the disclosure provides the use of a cell as defined herein, in a method for the production of a mixture of di- and/or oligosaccharides. An alternative and/or additional aspect of the disclosure provides the use of a cell as defined herein, in a method for the production of a mixture of charged and/or neutral di- and/or oligosaccharides. A preferred aspect provides the use of a cell of disclosure in a method for the production of a mixture of sialylated and/or neutral di- and/or oligosaccharides. An alternative and/or additional aspect of the disclosure provides the use of a cell as defined herein, in a method for the production of a mixture of charged di- and/or oligosaccharides. A preferred aspect provides the use of a cell of disclosure in a method for the production of a mixture of sialylated di- and/or oligosaccharides. An alternative and/or additional aspect of the disclosure provides the use of a cell as defined herein, in a method for the production of a mixture of oligosaccharides comprising at least two different oligosaccharides. A preferred aspect provides the use of a cell of disclosure in a method for the production of a mixture of oligosaccharides comprising at least three different oligosaccharides.

A further aspect of the disclosure provides the use of a method as defined herein for the production of a di- and/or oligosaccharide.

Furthermore, the disclosure also relates to the di- and/or oligosaccharide obtained by the methods according to the disclosure, as well as to the use of a polynucleotide, the vector, host cells or the polypeptide as described above for the production of the di- and/or oligosaccharide. The di- and/or oligosaccharide may be used as food additive, prebiotic, symbiotic, for the supplementation of baby food, adult food or feed, or as either therapeutically or pharmaceutically active compound or in cosmetic applications. With the novel methods, the di- and/or oligosaccharide can easily and effectively be provided, without the need for complicated, time and cost consuming synthetic processes.

For identification of the di- and/or oligosaccharide produced in the cell as described herein, the monomeric building blocks (e.g., the monosaccharide or glycan unit composition), the anomeric configuration of side chains, the presence and location of substituent groups, degree of polymerization/molecular weight and the linkage pattern can be identified by standard methods known in the art, such as, e.g., methylation analysis, reductive cleavage, hydrolysis, GC-MS (gas chromatography-mass spectrometry), MALDI-MS (Matrix-assisted laser desorption/ionization-mass spectrometry), ESI-MS (Electrospray ionization-mass spectrometry), HPLC (High-Performance Liquid chromatography with ultraviolet or refractive index detection), HPAEC-PAD (High-Performance Anion-Exchange chromatography with Pulsed Amperometric Detection), CE (capillary electrophoresis), IR (infrared)/Raman spectroscopy, and NMR (Nuclear magnetic resonance) spectroscopy techniques. The crystal structure can be solved using, e.g., solid-state NMR, FT-IR (Fourier transform infrared spectroscopy), and WAXS (wide-angle X-ray scattering). The degree of polymerization (DP), the DP distribution, and polydispersity can be determined by, e.g., viscosimetry and SEC (SEC-HPLC, high performance size-exclusion chromatography). To identify the monomeric components of the di- and/or oligosaccharide methods such as e.g., acid-catalyzed hydrolysis, HPLC (high performance liquid chromatography) or GLC (gas-liquid chromatography) (after conversion to alditol acetates) may be used. To determine the glycosidic linkages, the di- and/or oligosaccharide is methylated with methyl iodide and strong base in DMSO, hydrolysis is performed, a reduction to partially methylated alditols is achieved, an acetylation to methylated alditol acetates is performed, and the analysis is carried out by GLC/MS (gas-liquid chromatography coupled with mass spectrometry). To determine the glycan sequence, a partial depolymerization is carried out using an acid or enzymes to determine the structures. To identify the anomeric configuration, the di- and/or oligosaccharide is subjected to enzymatic analysis, e.g., it is contacted with an enzyme that is specific for a particular type of linkage, e.g., beta-galactosidase, or alpha-glucosidase, etc., and NMR may be used to analyze the products.

The separated and preferably also purified di- and/or oligosaccharide as described herein is incorporated into a food (e.g., human food or feed), dietary supplement, pharmaceutical ingredient, cosmetic ingredient or medicine. In some embodiments, the di- and/or oligosaccharide is mixed with one or more ingredients suitable for food, feed, dietary supplement, pharmaceutical ingredient, cosmetic ingredient or medicine.

In some embodiments, the dietary supplement comprises at least one prebiotic ingredient and/or at least one probiotic ingredient.

A “prebiotic” is a substance that promotes growth of microorganisms beneficial to the host, particularly microorganisms in the gastrointestinal tract. In some embodiments, a dietary supplement provides multiple prebiotics, including the di- and/or oligosaccharide being a prebiotic produced and/or purified by a process disclosed in this specification, to promote growth of one or more beneficial microorganisms. Examples of prebiotic ingredients for dietary supplements include other prebiotic molecules (such as HMOs) and plant polysaccharides (such as inulin, pectin, b-glucan and xylooligosaccharide). A “probiotic” product typically contains live microorganisms that replace or add to gastrointestinal microflora, to the benefit of the recipient. Examples of such microorganisms include Lactobacillus species (for example, L. acidophilus and L. bulgaricus), Bifidobacterium species (for example, B. animalis, B. longum and B. infantis (e.g., Bi-26)), and Saccharomyces boulardii. In some embodiments, a di- and/or oligosaccharide produced and/or purified by a process of this specification is orally administered in combination with such microorganism.

Examples of further ingredients for dietary supplements include oligosaccharides (such as 2′-fucosyllactose, 3-fucosyllactose, 3′-sialyllactose, 6′-sialyllactose), disaccharides (such as lactose), monosaccharides (such as glucose, galactose, L-fucose, sialic acid, glucosamine and N-acetylglucosamine), thickeners (such as gum arabic), acidity regulators (such as trisodium citrate), water, skimmed milk, and flavourings.

In some embodiments, the oligosaccharide is incorporated into a human baby food (e.g., infant formula). Infant formula is generally a manufactured food for feeding to infants as a complete or partial substitute for human breast milk. In some embodiments, infant formula is sold as a powder and prepared for bottle- or cup-feeding to an infant by mixing with water. The composition of infant formula is typically designed to be roughly mimic human breast milk. In some embodiments, a oligosaccharide produced and/or purified by a process in this specification is included in infant formula to provide nutritional benefits similar to those provided by the oligosaccharides in human breast milk. In some embodiments, the oligosaccharide is mixed with one or more ingredients of the infant formula. Examples of infant formula ingredients include non-fat milk, carbohydrate sources (e.g., lactose), protein sources (e.g., whey protein concentrate and casein), fat sources (e.g., vegetable oils—such as palm, high oleic safflower oil, rapeseed, coconut and/or sunflower oil; and fish oils), vitamins (such as vitamins A, B6, B12, C and D), minerals (such as potassium citrate, calcium citrate, magnesium chloride, sodium chloride, sodium citrate and calcium phosphate) and possibly human milk oligosaccharides (HMOs). Such HMOs may include, for example, DiFL, lacto-N-triose II, LNT, LNnT, lacto-N-fucopentaose I, lacto-N-neofucopentaose, lacto-N-fucopentaose II, lacto-N-fucopentaose III, lacto-N-fucopentaose V, lacto-N-neofucopentaose V, lacto-N-difucohexaose I, lacto-N-difucohexaose II, 6′-galactosyllactose, 3′-galactosyllactose, lacto-N-hexaose and lacto-N-neohexaose.

In some embodiments, the one or more infant formula ingredients comprise non-fat milk, a carbohydrate source, a protein source, a fat source, and/or a vitamin and mineral.

In some embodiments, the one or more infant formula ingredients comprise lactose, whey protein concentrate and/or high oleic safflower oil.

In some embodiments, the concentration of the oligosaccharide in the infant formula is approximately the same concentration as the concentration of the oligosaccharide generally present in human breast milk.

In some embodiments, the oligosaccharide is incorporated into a feed preparation, wherein the feed is chosen from the list comprising pet food, animal milk replacer, veterinary product, post weaning feed, or creep feed.

As will be shown in the examples herein, the method and the cell of the disclosure preferably provide at least one of the following surprising advantages:
Higher titers of the di- and/or oligosaccharide (g/L),
Higher production rate r (g di- and/or oligosaccharide/L/h),
Higher cell performance index CPI (g di- and/or oligosaccharide/g X),
Higher specific productivity Qp (g di- and/or oligosaccharide/g X/h),
Higher yield on sucrose Ys (g di- and/or oligosaccharide/g sucrose),
Higher sucrose uptake/conversion rate Qs (g sucrose/g X/h),
Higher lactose conversion/consumption rate rs (g lactose/h),
Higher secretion of the di- and/or oligosaccharide, and/or
Higher growth speed of the production host,
when compared to a host for production of a di- and/or oligosaccharide lacking expression and/or overexpression of at least one set of multiple coding DNA sequences encoding one or more polypeptides that have the same function and/or activity of interest.

Unless defined otherwise, all technical and scientific terms used herein generally have the same meaning as commonly understood by one of ordinary skill in the art to which this disclosure belongs. Generally, the nomenclature used herein and the laboratory procedures in cell culture, molecular genetics, organic chemistry and nucleic acid chemistry and hybridization described above and below are those well-known and commonly employed in the art. Standard techniques are used for nucleic acid and peptide synthesis. Generally, purification steps are performed according to the manufacturer's specifications.

Further advantages follow from the specific embodiments and the examples. It goes without saying that the abovementioned features and the features that are still to be explained below can be used not only in the respectively specified combinations, but also in other combinations or on their own, without departing from the scope of the disclosure.

Moreover, the disclosure relates to the following specific embodiments:

1. A cell for production of a di- and/or oligosaccharide, the cell comprising a pathway for production of the di- and/or oligosaccharide, characterized in that the cell is genetically modified for expression and/or overexpression of at least one set of multiple coding DNA sequences, wherein the multiple coding DNA sequences within one set:
i) differ in nucleotide sequence, and
ii) each encode a polypeptide, wherein the polypeptides have the same function and/or activity of interest,
preferably, wherein the polypeptides are essentially the same polypeptides,
more preferably, wherein the polypeptides are identical to each other.

2. Cell according to embodiment 1, wherein the polypeptides within a set are functional variants, the variants comprising a functional homolog, ortholog and paralog.

3. Cell according to any one of embodiment 1 or 2, wherein multiple is at least 2, preferably at least 3, more preferably at least 4, even more preferably at least 5.

4. Cell according to any one of the previous embodiments, wherein the cell comprises at least 2, preferably at least 3, more preferably at least 4, even more preferably at least 5 sets of multiple coding DNA sequences as defined in embodiment 1, wherein each set of multiple coding DNA sequences encodes polypeptides having a different function and/or activity of interest compared to the other sets of multiple coding DNA sequences.

5. Cell according to any one of the previous embodiments, wherein the multiple coding DNA sequences within a set are integrated in the genome of the cell and/or presented to the cell on one or more vectors comprising plasmid, cosmid, artificial chromosome, phage, liposome or virus, which is/are to be stably transformed into the cell.

6. Cell according to any one of the previous embodiments, wherein the multiple coding DNA sequences within a set are presented to the cell in one or more location(s) on one or more chromosome(s).

7. Cell according to any one of the previous embodiments, wherein the multiple coding DNA sequences within a set are presented to the cell within a biosynthetic gene cluster encoding polypeptides participating in the pathway for production of the di- and/or oligosaccharide.

8. Cell according to any one of the previous embodiments, wherein the multiple coding DNA sequences within a set are presented to the cell in one or more gene expression modules comprising one or more regulatory gene sequences regulating expression of the multiple coding DNA sequences.

9. Cell according to any one of the previous embodiments, wherein the multiple coding DNA sequences within a set are organized within any one or more of the list comprising co-expression module, operon, regulon, stimulon and modulon.

10. Cell according to any one of the previous embodiments, wherein expression of the multiple coding DNA sequences within a set is regulated by one or more promoter sequence(s) that is/are constitutive and/or inducible upon a natural inducer.

11. Cell according to any one of the previous embodiments, wherein the cell is genetically modified for the production of the di- and/or oligosaccharide.

12. Cell according to any one of the previous embodiments, wherein the cell is genetically modified by introducing a pathway for the production of the di- and/or oligosaccharide.

13. Cell according to any one of the previous embodiments, wherein the polypeptides encoded by at least one set of multiple coding DNA sequences are directly involved in the pathway for production of the di- and/or oligosaccharide,
preferably, wherein the polypeptides encoded by all sets of multiple coding DNA sequences are directly involved in the pathway for production of the di- and/or oligosaccharide.

14. Cell according to any one of the previous embodiments, wherein the polypeptides that are encoded by the multiple coding DNA sequences within a set have the same function and/or activity and wherein the function and/or activity is:
i) directly involved in the synthesis of a nucleotide-activated sugar, wherein the nucleotide-activated sugar is to be used in the production of the di- and/or oligosaccharide,
ii) a glycosyltransferase activity hereby transferring a monosaccharide from a nucleotide-activated sugar donor to a disaccharide/oligosaccharide acceptor, or
iii) a transport activity hereby transporting compounds across the outer membrane of the cell wall.

15. Cell according to embodiment 14, wherein the nucleotide-activated sugar is chosen from the list comprising UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-fucose (GDP-Fuc), GDP-rhamnose and UDP-xylose.

16. Cell according to any one of embodiment 14 or 15, wherein the multiple coding DNA sequences within a set encode polypeptides having the same function and/or activity in the synthesis of a nucleotide-activated sugar and that are chosen from the list comprising mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, fucose-1-phosphate guanylyltransferase, L-glutamine D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine kinase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase and UDP-N-acetylgalactosamine pyrophosphorylase.

17. Cell according to any one of the embodiments 14 to 16, wherein the multiple coding DNA sequences within a set encode glycosyltransferases or polypeptides having glycosyltransferase activity that are chosen from the list comprising fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases and fucosaminyltransferases,
preferably, the fucosyltransferase is chosen from the list comprising alpha-1,2-fucosyltransferase, alpha-1,3-fucosyltransferase, alpha-1,4-fucosyltransferase and alpha-1,6-fucosyltransferase,
preferably, the sialyltransferase is chosen from the list comprising alpha-2,3-sialyltransferase, alpha-2,6-sialyltransferase and alpha-2,8-sialyltransferase,
preferably, the galactosyltransferase is chosen from the list comprising beta-1,3-galactosyltransferase, N-acetylglucosamine beta-1,3-galactosyltransferase, beta-1,4-galactosyltransferase, N-acetylglucosamine beta-1,4-galactosyltransferase, alpha-1,3-galactosyltransferase and alpha-1,4-galactosyltransferase,
preferably, the glucosyltransferase is chosen from the list comprising alpha-glucosyltransferase, beta-1,2-glucosyltransferase, beta-1,3-glucosyltransferase and beta-1,4-glucosyltransferase,
preferably, the mannosyltransferase is chosen from the list comprising alpha-1,2-mannosyltransferase, alpha-1,3-mannosyltransferase and alpha-1,6-mannosyltransferase,
preferably, the N-acetylglucosaminyltransferase is chosen from the list comprising galactoside beta-1,3-N-acetylglucosaminyltransferase and beta-1,6-N-acetylglucosaminyltransferase,
preferably, the N-acetylgalactosaminyltransferase is an alpha-1,3-N-acetylgalactosaminyltransferase.

18. Cell according to any one of the embodiments 14 to 17, wherein the multiple coding DNA sequences within a set encode polypeptides that are membrane transporter proteins or polypeptides having transport activity hereby transporting compounds across the outer membrane of the cell wall.

19. Cell according to any one of the embodiments 14 to 18, wherein the membrane transporter proteins or polypeptides having transport activity are chosen from the list of transporters comprising porters, P-P-bond-hydrolysis-driven transporters, b-barrel porins, auxiliary transport proteins, putative transport proteins and phosphotransfer-driven group translocators.

20. Cell according to embodiment 19, wherein the porters comprise MFS transporters, sugar efflux transporters and siderophore exporters.

21. Cell according to embodiment 19, wherein the P-P-bond-hydrolysis-driven transporters comprise ABC transporters and siderophore exporters.

22. Cell according to any one of the previous embodiments, wherein the cell is using one or more precursor(s) for the production of the di- and/or oligosaccharide the precursor(s) being fed to the cell from the cultivation medium.

23. Cell according to any one of the previous embodiments, wherein the cell is producing one or more precursor(s) for the production of the di- and/or oligosaccharide.

24. Cell according to any one of the embodiments 14 to 23, wherein the membrane transporter proteins or polypeptides having transport activity control the flow over the outer membrane of the cell wall of i) the di- and/or oligosaccharide and/or ii) any one or more precursor(s) and/or acceptor(s) to be used in the production of the di- and/or oligosaccharide.

25. Cell according to any one of embodiments 14 to 24, wherein the membrane transporter proteins provide improved production and/or enabled and/or enhanced efflux of the di- and/or oligosaccharide.

26. Cell according to any one of the previous embodiments, wherein the di- and/or oligosaccharide is chosen from the list comprising a milk oligosaccharide, O-antigen, enterobacterial common antigen (ECA), the oligosaccharide repeats present in capsular polysaccharides, peptidoglycan, amino-sugars, Lewis-type antigen oligosaccharide and antigens of the human ABO blood group system,
preferably, the oligosaccharide is a milk oligosaccharide, more preferably a mammalian milk oligosaccharide, even more preferably, a human milk oligosaccharide.

27. Cell according to any one of the previous embodiments, wherein the pathway comprises a fucosylation pathway,
preferably, wherein the polypeptides that are encoded by the multiple coding DNA sequences within a set are directly involved in the fucosylation pathway and are preferably selected from the list comprising mannose-6-phosphate isomerase, phosphomannomutase, mannose-1-phosphate guanylyltransferase, GDP-mannose 4,6-dehydratase, GDP-L-fucose synthase, fucose permease, fucose kinase, fucose-1-phosphate guanylyltransferase, and fucosyltransferase.

28. Cell according to any one of the previous embodiments, wherein the pathway comprises a sialylation pathway,
preferably, wherein the polypeptides that are encoded by the multiple coding DNA sequences within a set are directly involved in the sialylation pathway and are preferably selected from the list comprising N-acylglucosamine 2-epimerase, UDP-N-acetylglucosamine 2-epimerase, N-acetylmannosamine-6-phosphate 2-epimerase, UDP-N-acetylglucosamine 2-epimerase/kinase hydrolyzing, N-acylneuraminate-9-phosphate synthase, phosphatase, N-acetylneuraminate synthase, N-acylneuraminate cytidylyltransferase, sialyltransferase and sialic acid transporter.

29. Cell according to any one of the previous embodiments, wherein the pathway comprises a galactosylation pathway,
preferably, wherein the polypeptides that are encoded by the multiple coding DNA sequences within a set are directly involved in the galactosylation pathway and are preferably selected from the list comprising galactose-1-epimerase, galactokinase, glucokinase, galactose-1-phosphate uridylyltransferase, UDP-glucose 4-epimerase, glucose-1-phosphate uridylyltransferase, phosphoglucomutase and galactosyltransferase.

30. Cell according to any one of the previous embodiments, wherein the pathway comprises an N-acetylglucosaminylation pathway,
preferably, wherein the polypeptides that are encoded by the multiple coding DNA sequences within a set are directly involved in the N-acetylglucosaminylation pathway and are preferably selected from the list comprising L-glutamine-D-fructose-6-phosphate aminotransferase, N-acetylglucosamine-6-phosphate deacetylase, phosphoglucosamine mutase, N-acetylglucosamine-1-phosphate uridylyltransferase, glucosamine-1-phosphate acetyltransferase and N-acetylglucosaminyltransferase.

31. Cell according to any one of the previous embodiments, wherein the pathway comprises an N-acetylgalactosaminylation pathway,
preferably, wherein the polypeptides that are encoded by the multiple coding DNA sequences within a set are directly involved in the N-acetylgalactosaminylation pathway and are preferably selected from the list comprising L-glutamine-D-fructose-6-phosphate aminotransferase, phosphoglucosamine mutase, N-acetylglucosamine-1-phosphate uridylyltransferase, glucosamine-1-phosphate acetyltransferase, UDP-N-acetylglucosamine 4-epimerase, UDP-glucose 4-epimerase, N-acetylgalactosamine kinase, UDP-N-acetylgalactosamine pyrophosphorylase and N-acetylgalactosaminyltransferase.

32. Cell according to any one of the previous embodiments, wherein the pathway comprises a mannosylation pathway,
preferably, wherein the polypeptides that are encoded by the multiple coding DNA sequences within a set are directly involved in the mannosylation pathway and are preferably selected from the list comprising mannose-6-phosphate isomerase, phosphomannomutase, mannose-1-phosphate guanylyltransferase and mannosyltransferase.

33. Cell according to any one of the previous embodiments, wherein the pathway comprises an N-acetylmannosaminylation pathway,
preferably, wherein the polypeptides that are encoded by the multiple coding DNA sequences within a set are directly involved in the N-acetylmannosaminylation pathway and are preferably selected from the list comprising L-glutamine-D-fructose-6-phosphate aminotransferase, glucosamine-6-phosphate deaminase, phosphoglucosamine mutase, N-acetylglucosamine-6-phosphate deacetylase, glucosamine 6-phosphate N-acetyltransferase, N-acetylglucosamine-1-phosphate uridyltransferase, glucosamine-1-phosphate acetyltransferase, glucosamine-1-phosphate acetyltransferase, UDP-GlcNAc 2-epimerase, ManNAc kinase and N-acetylmannosaminyltransferase.

34. Cell according to any one of the previous embodiments, wherein the cell is capable to produce phosphoenolpyruvate (PEP).

35. Cell according to any one of the previous embodiments, wherein the cell is modified for enhanced production and/or supply of PEP.

36. Cell according to any one of the previous embodiments, wherein the polypeptides that are encoded by the multiple coding DNA sequences within a set are directly involved in the production and/or supply of PEP.

37. Cell according to any one of the previous embodiments, wherein the cell comprises:
i) a set of multiple coding DNA sequences wherein the multiple coding DNA sequences differ in nucleotide sequence and each encode a polypeptide having galactoside beta-1,3-N-acetylglucosaminyltransferase activity, and wherein each of the coding DNA sequences:
   is chosen from the list comprising SEQ ID NOs:01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57, and/or
   is a fragment of any one of SEQ ID NOs:01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57 encoding a polypeptide having galactoside beta-1,3-N-acetylglucosaminyltransferase activity, and/or
   comprises and/or consists of a nucleotide sequence having 80% or more sequence identity to the full-length nucleotide sequence of any one of SEQ ID NO:01, 02, 03, 04, 05, 06, 07, 08, 09, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57 and encoding a polypeptide having galactoside beta-1,3-N-acetylglucosaminyltransferase activity, and/or
   encodes a polypeptide chosen from the list comprising SEQ ID NOs:79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 and 131, and/or
   encodes a functional fragment of a polypeptide according to any one of SEQ ID NOs:79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 or 131 and having galactoside beta-1,3-N-acetylglucosaminyltransferase activity, and/or
   encodes a polypeptide comprising or consisting of an amino acid sequence having 80% or more sequence identity to the full-length amino acid sequence of any one of SEQ ID NO:79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 or 131 and having galactoside beta-1,3-N-acetylglucosaminyltransferase activity, and/or
ii) a set of multiple coding DNA sequences wherein the multiple coding DNA sequences differ in nucleotide sequence and each encode a polypeptide having N-acetylglucosamine beta-1,3-galactosyltransferase activity, and wherein each of the coding DNA sequences:
   is chosen from the list comprising SEQ ID NOs:58, 59, 60, 61, 62, 63, 64, 65 and 66, and/or
   is a fragment of any one of SEQ ID NOs:58, 59, 60, 61, 62, 63, 64, 65 and 66 encoding a polypeptide having N-acetylglucosamine beta-1,3-galactosyltransferase activity, and/or
   comprises and/or consists of a nucleotide sequence having 80% or more sequence identity to the full-length nucleotide sequence of any one of SEQ ID NO:58, 59, 60, 61, 62, 63, 64, 65 or 66 and encoding a polypeptide having N-acetylglucosamine beta-1,3-galactosyltransferase activity, and/or
   encodes a polypeptide chosen from the list comprising SEQ ID NOs:132, 133, 134 and 135, and/or
   encodes a functional fragment of a polypeptide according to any one of SEQ ID NOs:132, 133, 134 or 135 and having N-acetylglucosamine beta-1,3-galactosyltransferase activity, and/or
   encodes a polypeptide comprising or consisting of an amino acid sequence having 80% or more sequence identity to the full-length amino acid sequence of any one of SEQ ID NO:132, 133, 134 or 135 and having N-acetylglucosamine beta-1,3-galactosyltransferase activity, and/or
iii) a set of multiple coding DNA sequences wherein the multiple coding DNA sequences differ in nucleotide sequence and each encode a polypeptide having N-acetylglucosamine beta-1,4-galactosyltransferase activity, and wherein each of the coding DNA sequences:
   is chosen from the list comprising SEQ ID NOs:67, 68, 69, 70, 71, 72, 73, 74, 75, 76, 77 and 78, and/or
   is a fragment of any one of SEQ ID NOs:67, 68, 69, 70, 71, 72, 73, 74, 75, 76, 77 and 78 encoding a polypeptide having N-acetylglucosamine beta-1,4-galactosyltransferase activity, and/or
   comprises and/or consists of a nucleotide sequence having 80% or more sequence identity to the full-length nucleotide sequence of any one of SEQ ID NO:67, 68, 69, 70, 71, 72, 73, 74, 75, 76, 77, or 78 and encoding a polypeptide having N-acetylglucosamine beta-1,4-galactosyltransferase activity, and/or
   encodes a polypeptide chosen from the list comprising SEQ ID NOs:136, 137, 138, 139, 140, 141, 142, 143, 144, and 145, and/or
   encodes a functional fragment of a polypeptide according to any one of SEQ ID NO:136, 137, 138, 139, 140, 141, 142, 143, 144, or 145 and having N-acetylglucosamine beta-1,4-galactosyltransferase activity, and/or
   encodes a polypeptide comprising or consisting of an amino acid sequence having 80% or more sequence identity to the full-length amino acid sequence of any one of SEQ ID NO:136, 137, 138, 139, 140, 141, 142, 143, 144, or 145 and having N-acetylglucosamine beta-1,4-galactosyltransferase activity.

38. Cell according to any one of the previous embodiments, wherein the cell comprises a set of multiple coding DNA sequences wherein the multiple coding DNA sequences differ in nucleotide sequence and each encode a polypeptide having N-acylneuraminate cytidylyltransferase activity, and wherein each of the coding DNA sequences encodes:
a polypeptide chosen from the list comprising the polypeptide from Campylobacter jejuni with UniProt ID Q93MP7, the polypeptide from Haemophilus influenzae with GenBank No. AGV11798.1 and the polypeptide from Pasteurella multocida with GenBank No. AMK07891.1 and having N-acylneuraminate cytidylyltransferase activity, and/or
a functional fragment of any one of the polypeptide from C. jejuni with UniProt ID Q93MP7, H. influenzae with GenBank No. AGV11798.1 or P. multocida with GenBank No. AMK07891.1 and having N-acylneuraminate cytidylyltransferase activity, and/or
a polypeptide comprising or consisting of an amino acid sequence having 80% or more sequence identity to the full-length amino acid sequence of any one of the polypeptides from C. jejuni with UniProt ID Q93MP7, H. influenzae with GenBank No. AGV11798.1 or P. multocida with GenBank No. AMK07891.1 and having N-acylneuraminate cytidylyltransferase activity.

39. Cell according to embodiment 38, wherein the cell further comprises:
i) at least one coding DNA sequence encoding a:
   polypeptide chosen from the list comprising the polypeptide from Neisseria meningitidis with UniProt ID E0NCD4, the polypeptide from Campylobacter jejuni with UniProt ID Q93MP9, the polypeptide from Aeromonas caviae with UniProt ID Q9R9S2, the polypeptide from Candidatus koribacter versatilis with UniProt ID Q1IMQ8, the polypeptide from Legionella pneumophila with UniProt ID Q9RDX5, the polypeptide from Methanocaldococcus jannaschii with UniProt ID Q58465 and the polypeptide from Moritella viscosa with UniProt ID A0A090INM4 and having N-acetylneuraminate synthase activity, and/or
   a functional fragment of any one of the polypeptide from N. meningitidis with UniProt ID E0NCD4, C. jejuni with UniProt ID Q93MP9, A. caviae with UniProt ID Q9R9S2, C. koribacter versatilis with UniProt ID Q1IMQ8, L. pneumophila with UniProt ID Q9RDX5, M. jannaschii with UniProt ID Q58465 or M. viscosa with UniProt ID A0A090IMH4 and having N-acetylneuraminate synthase activity, and/or
   a polypeptide comprising or consisting of an amino acid sequence having 80% or more sequence identity to the full-length amino acid sequence of any one of the polypeptides from N. meningitidis with UniProt ID E0NCD4, C. jejuni with UniProt ID Q93MP9, A. caviae with UniProt ID Q9R9S2, C. koribacter versatilis with UniProt ID Q1IMQ8, L. pneumophila with UniProt ID Q9RDX5, M. jannaschii with UniProt ID Q58465 or M. viscosa with UniProt ID A0A090IMH4 and having N-acetylneuraminate synthase activity, and/or
ii) two or more copies of one or more coding DNA sequences of an alpha-2,3-sialyltransferase, an alpha-2,6-sialyltransferase, and/or an alpha-2,8-sialyltransferase.

40. Cell according to any one of the previous embodiments, wherein the cell comprises a modification for reduced production of acetate.

41. Cell according to any one of the previous embodiments, wherein the cell further comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose undecaprenyl-phosphate glucose-1-phosphatetransferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

42. Cell according to any one of the previous embodiments, wherein the cell comprises a catabolic pathway for selected mono-, di- or oligosaccharides, which is at least partially inactivated, the mono-, di-, or oligosaccharides being involved in and/or required for the production of the di- and/or oligosaccharide.

43. Cell according to any one of the previous embodiments, wherein the cell produces the di- and/or oligosaccharide intracellularly and wherein a fraction or substantially all of the produced di- and/or oligosaccharide remains intracellularly and/or is excreted outside the cell via passive or active transport.

44. Cell according to any one of the previous embodiments, wherein the cell produces 90 g/L or more of the di- and/or oligosaccharide in the whole broth and/or supernatant and/or wherein the di- and/or oligosaccharide in the whole broth and/or supernatant has a purity of at least 80% measured on the total amount of di- and/or oligosaccharide and its precursor(s) in the whole broth and/or supernatant, respectively.

45. Cell according to any one of the previous embodiments, wherein the cell is a bacterium, fungus, yeast, a plant cell, an animal cell, or a protozoan cell,
preferably the bacterium is an Escherichia coli strain, more preferably an Escherichia coli strain, which is a K-12 strain, even more preferably the Escherichia coli K-12 strain is E. coli MG1655,
preferably the fungus belongs to a genus chosen from the group comprising Rhizopus, Dictyostelium, Penicillium, Mucor or Aspergillus,
preferably the yeast belongs to a genus chosen from the group comprising Saccharomyces, Zygosaccharomyces, Pichia, Komagataella, Hansenula, Yarrowia, Starmerella, Kluyveromyces or Debaromyces,
preferably the plant cell is an algal cell or is derived from tobacco, alfalfa, rice, tomato, cotton, rapeseed, soy, maize, or corn plant,
preferably the animal cell is derived from non-human mammals, birds, fish, invertebrates, reptiles, amphibians or insects or is a genetically modified cell line derived from human cells excluding embryonic stem cells, more preferably the human and non-human mammalian cell is an epithelial cell, an embryonic kidney cell, a fibroblast cell, a COS cell, a Chinese hamster ovary (CHO) cell, a murine myeloma cell, an NIH-3T3 cell, a non-mammary adult stem cell or derivatives thereof, more preferably the insect cell is derived from Spodoptera frugiperda, Bombyx mori, Mamestra brassicae, Trichoplusia ni or Drosophila melanogaster,
preferably the protozoan cell is a Leishmania tarentolae cell.

46. Cell according to embodiment 45, wherein the cell is a viable Gram-negative bacterium that comprises a reduced or abolished synthesis of poly-N-acetyl-glucosamine (PNAG), Enterobacterial Common Antigen (ECA), cellulose, colanic acid, core oligosaccharides, Osmoregulated Periplasmic Glucans (OPG), Glucosylglycerol, glycan, and/or trehalose.

47. Cell according to any one of the previous embodiments, wherein the cell is stably cultured in a medium.

48. Cell according to any one of the previous embodiments, wherein the cell resists the phenomenon of lactose killing when grown in an environment in which lactose is combined with one or more other carbon source(s).

49. Cell according to any one of the previous embodiments, wherein the cell is capable to produce a mixture of di- and/or oligosaccharides, preferably a mixture of di- and oligosaccharides.

50. Cell according to any one of the previous embodiments, wherein the cell is capable to produce a mixture of charged and/or neutral di- and/or oligosaccharides, wherein preferably the charged di- and/or oligosaccharides comprise at least one sialylated di- and/or oligosaccharide.

51. Cell according to any one of the previous embodiments, wherein the cell is capable to produce a mixture of di- and oligosaccharides comprising at least two different oligosaccharides, preferably comprising at least three different oligosaccharides.

52. Cell according to any one of the previous embodiments, wherein the cell is capable to produce a mixture of oligosaccharides, preferably a mixture comprising at least three different oligosaccharides.

53. Cell according to any one of the previous embodiments, wherein the cell is capable to produce a mixture of charged and/or neutral mammalian milk oligosaccharides (MMOs), wherein preferably the charged MMOs comprise at least one sialylated MMO.

54. Method to produce a di- and/or oligosaccharide by a cell, the method comprising the steps of:
i) providing a cell according to any one of embodiments 1 to 53, and
ii) cultivating the cell under conditions permissive to produce the di- and/or oligosaccharide,
iii) preferably, separating the di- and/or oligosaccharide from the cultivation.

55. Method according to embodiment 54, wherein the conditions comprise:
use of a culture medium comprising at least one precursor and/or acceptor for the production of the di- and/or oligosaccharide, and/or
adding to the culture medium at least one precursor and/or acceptor feed for the production of the di- and/or oligosaccharide.

56. Method according to any one of embodiment 54 or 55, the method comprising at least one of the following steps:
i) Use of a culture medium comprising at least one precursor and/or acceptor;
ii) Adding to the culture medium in a reactor at least one precursor and/or acceptor feed wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than two-fold of the volume of the culture medium before the addition of the precursor and/or acceptor feed;
iii) Adding to the culture medium in a reactor at least one precursor and/or acceptor feed wherein the total reactor volume ranges from 250 mL (millilitre) to 10,000 m³ (cubic meter), preferably in a continuous manner, and preferably so that the final volume of the culture medium is not more than three-fold, preferably not more than two-fold, more preferably less than two-fold of the volume of the culture medium before the addition of the precursor and/or acceptor feed and wherein preferably, the pH of the precursor and/or acceptor feed is set between 3 and 7 and wherein preferably, the temperature of the precursor and/or acceptor feed is kept between 20° C. and 80° C.;
iv) Adding at least one precursor and/or acceptor feed in a continuous manner to the culture medium over the course of 1 day, 2 days, 3 days, 4 days, 5 days by means of a feeding solution;
v) Adding at least one precursor and/or acceptor feed in a continuous manner to the culture medium over the course of 1 day, 2 days, 3 days, 4 days, 5 days by means of a feeding solution and wherein preferably, the pH of the feeding solution is set between 3 and 7 and wherein preferably, the temperature of the feeding solution is kept between 20° C. and 80° C.;
the method resulting in a di- and/or oligosaccharide with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

57. Method according to any one of embodiment 54 or 55, the method comprising at least one of the following steps:
i) Use of a culture medium comprising at least 50, more preferably at least 75, more preferably at least 100, more preferably at least 120, more preferably at least 150 gram of lactose per litre of initial reactor volume wherein the reactor volume ranges from 250 mL to 10,000 m³ (cubic meter);
ii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than two-fold of the volume of the culture medium before the addition of the lactose feed;
iii) Adding to the culture medium a lactose feed comprising at least 50, more preferably at least 75, more preferably at least 100, more preferably at least 120, more preferably at least 150 gram of lactose per litre of initial reactor volume wherein the reactor volume ranges from 250 mL to 10,000 m³ (cubic meter), preferably in a continuous manner, and preferably so that the final volume of the culture medium is not more than three-fold, preferably not more than two-fold, more preferably less than two-fold of the volume of the culture medium before the addition of the lactose feed and wherein preferably the pH of the lactose feed is set between 3 and 7 and wherein preferably the temperature of the lactose feed is kept between 20° C. and 80° C.;
iv) Adding a lactose feed in a continuous manner to the culture medium over the course of 1 day, 2 days, 3 days, 4 days, 5 days by means of a feeding solution;
v) Adding a lactose feed in a continuous manner to the culture medium over the course of 1 day, 2 days, 3 days, 4 days, 5 days by means of a feeding solution and wherein the concentration of the lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L and wherein preferably the pH of the feeding solution is set between 3 and 7 and wherein preferably the temperature of the feeding solution is kept between 20° C. and 80° C.;
the method resulting in an oligosaccharide produced from the lactose with a concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final cultivation.

58. Method according to embodiment 57, wherein the lactose feed is accomplished by adding lactose from the beginning of the cultivation in a concentration of at least 5 mM, preferably in a concentration of 30, 40, 50, 60, 70, 80, 90, 100, 150 mM, more preferably in a concentration >300 mM.

59. Method according to any one of embodiment 57 or 58, wherein the lactose feed is accomplished by adding lactose to the cultivation in a concentration, such, that throughout the production phase of the cultivation a lactose concentration of at least 5 mM, preferably 10 mM or 30 mM is obtained.

60. Method according to any one of embodiment 54 to 59, wherein the host cells are cultivated for at least about 60, 80, 100, or about 120 hours or in a continuous manner.

61. Method according to any one of embodiment 54 to 60, wherein the cell is cultivated in a culture medium comprising a carbon source comprising a monosaccharide, disaccharide, oligosaccharide, polysaccharide, polyol, glycerol, a complex medium including molasses, corn steep liquor, peptone, tryptone or yeast extract; preferably, wherein the carbon source is chosen from the list comprising glucose, glycerol, fructose, sucrose, maltose, lactose, arabinose, malto-oligosaccharides, maltotriose, sorbitol, xylose, rhamnose, galactose, mannose, methanol, ethanol, trehalose, starch, cellulose, hemi-cellulose, molasses, corn-steep liquor, high-fructose syrup, acetate, citrate, lactate and pyruvate.

62. Method according to any one of embodiment 54 to 61, wherein the cell uses at least one precursor for the production of the di- and/or oligosaccharide, preferably the cell uses two or more precursors for the production of the di- and/or oligosaccharide.

63. Method according to any one of embodiment 54 to 62, wherein the culture medium contains at least one precursor selected from the group comprising lactose, galactose, fucose, sialic acid, GlcNAc, GalNAc, lacto-N-biose (LNB), N-acetyllactosamine (LacNAc).

64. Method according to any one of embodiment 54 to 63, wherein a first phase of exponential cell growth is provided by adding a carbon-based substrate, preferably glucose or sucrose, to the culture medium before the precursor, preferably lactose, is added to the culture medium in a second phase.

65. Method according to any one of embodiment 54 to 64, wherein a first phase of exponential cell growth is provided by adding a carbon-based substrate, preferably glucose or sucrose, to the culture medium comprising a precursor, preferably lactose, followed by a second phase wherein only a carbon-based substrate, preferably glucose or sucrose, is added to the culture medium.

66. Method according to any one of embodiment 54 to 64, wherein a first phase of exponential cell growth is provided by adding a carbon-based substrate, preferably glucose or sucrose, to the culture medium comprising a precursor, preferably lactose, followed by a second phase wherein a carbon-based substrate, preferably glucose or sucrose, and a precursor, preferably lactose, are added to the culture medium.

67. Method according to any one of embodiment 54 to 66, wherein the cell is producing at least one precursor for the production of the di- and/or oligosaccharide.

68. Method according to any one of embodiment 54 to 67, wherein the precursor for the production of the di- and/or oligosaccharide is completely converted into the di- and/or oligosaccharide.

69. Method according to any one of embodiment 54 to 68, wherein the di- and/or oligosaccharide is separated from the cultivation.

70. Method according to any one of embodiment 54 to 69, wherein the separation comprises at least one of the following steps: clarification, ultrafiltration, nanofiltration, two-phase partitioning, reverse osmosis, microfiltration, activated charcoal or carbon treatment, treatment with non-ionic surfactants, enzymatic digestion, tangential flow high-performance filtration, tangential flow ultrafiltration, electrophoresis, affinity chromatography, ion exchange chromatography, hydrophobic interaction chromatography and/or gel filtration, ligand exchange chromatography.

71. Method according to any one of embodiment 54 to 70, wherein the method further comprises purification of the di- and/or oligosaccharide.

72. Method according to embodiment 71, wherein the purification comprises at least one of the following steps: use of activated charcoal or carbon, use of charcoal, nanofiltration, ultrafiltration, electrophoresis, enzymatic treatment or ion exchange, use of alcohols, use of aqueous alcohol mixtures, crystallization, evaporation, precipitation, drying, spray drying, lyophilization, spray freeze drying, freeze spray drying, band drying, belt drying, vacuum band drying, vacuum belt drying, drum drying, roller drying, vacuum drum drying or vacuum roller drying.

73. Use of a cell according to any one of embodiment 1 to 48 for production of a di- and/or oligosaccharide.

74. Use of a cell according to embodiment 49 for production of a mixture of di- and/or oligosaccharides, preferably a mixture of di- and oligosaccharides.

75. Use of a cell according to embodiment 50 for production of a mixture of charged and/or neutral di- and/or oligosaccharides, wherein preferably the charged di- and/or oligosaccharides comprise at least one sialylated di- and/or oligosaccharide.

76. Use of a cell according to embodiment 51 for production of a mixture of di- and oligosaccharides comprising at least two different oligosaccharides, preferably comprising at least three different oligosaccharides.

77. Use of a cell according to embodiment 52 for production of a mixture of oligosaccharides, preferably a mixture comprising at least three different oligosaccharides.

78. Use of a cell according to embodiment 53 for production of a mixture of charged and/or neutral mammalian milk oligosaccharides (MMOs), wherein preferably the charged MMOs comprise at least one sialylated MMO.

79. Use of a method according to any one of embodiment 54 to 72 for production of a di- and/or oligosaccharide.

The disclosure will be described in more detail in the examples. The following examples will serve as further illustration and clarification of the disclosure and are not intended to be limiting.

### EXAMPLES

### Example 1. Calculation of Percentage Identity Between Nucleotide or Polypeptide Sequences

Methods for the alignment of sequences for comparison are well known in the art, such methods include GAP, BESTFIT, BLAST, FASTA and TFASTA. GAP uses the algorithm of Needleman and Wunsch (J. Mol. Biol. (1970) 48: 443-453) to find the global (i.e., spanning the full-length sequences) alignment of two sequences that maximizes the number of matches and minimizes the number of gaps. The BLAST algorithm (Altschul et al., J. Mol. Biol. (1990) 215: 403-10) calculates the global percentage sequence identity (i.e., over the full-length sequence) and performs a statistical analysis of the similarity between the two sequences. The software for performing BLAST analysis is publicly available through the National Centre for Biotechnology Information (NCBI). Homologs may readily be identified using, for example, the ClustalW multiple sequence alignment algorithm (version 1.83), with the default pairwise alignment parameters, and a scoring method in percentage. Global percentages of similarity and identity ((i.e., spanning the full-length sequences) may also be determined using one of the methods available in the MatGAT software package (Campanella et al., BMC Bioinformatics (2003) 4:29). Minor manual editing may be performed to optimize alignment between conserved motifs, as would be apparent to a person skilled in the art. Furthermore, instead of using full-length sequences for the identification of homologs, specific domains may also be used, to determine the so-called local sequence identity. The sequence identity values may be determined over the entire nucleic acid or amino acid sequence (=local sequence identity search over the full-length sequence resulting in a global sequence identity score) or over selected domains or conserved motif(s) (=local sequence identity search over a partial sequence resulting in a local sequence identity score), using the programs mentioned above using the default parameters. For local alignments, the Smith-Waterman algorithm is particularly useful (Smith T F, Waterman M S (1981) J. Mol. Biol 147(1); 195-7).

### Example 2. Materials and Methods Escherichia coli

### Media

The Luria Broth (LB) medium consisted of 1% tryptone peptone (Difco, Erembodegem, Belgium), 0.5% yeast extract (Difco) and 0.5% sodium chloride (VWR, Leuven, Belgium). The minimal medium used in the cultivation experiments in 96-well plates or in shake flasks contained 2.00 g/L NH₄Cl, 5.00 g/L (NH₄)₂SO₄, 2.993 g/L KH₂PO₄, 7.315 g/L K₂HPO₄, 8.372 g/L MOPS, 0.5 g/L NaCl, 0.5 g/L MgSO₄·7H₂O, 30 g/L sucrose or 30 g/L glycerol, 1 ml/L vitamin solution, 100 μL/L molybdate solution, and 1 mL/L selenium solution. As specified in the respective examples, 0.30 g/L sialic acid, 20 g/L lactose, 20 g/L LacNAc and/or 20 g/L LNB were additionally added to the medium as precursor(s). The minimal medium was set to a pH of 7 with 1M KOH. Vitamin solution consisted of 3.6 g/L FeCl₂·4H₂O, 5 g/L CaCl₂·2H₂O, 1.3 g/L MnCl₂·2H₂O, 0.38 g/L CuCl₂·2H₂O, 0.5 g/L COC₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L SeO₂.

The minimal medium for fermentations contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H₂O, 30 g/L sucrose or 30 g/L glycerol, 1 mL/L vitamin solution, 100 μL/L molybdate solution, and 1 mL/L selenium solution with the same composition as described above. As specified in the respective examples, 0.30 g/L sialic acid, 20 g/L lactose, 20 g/L LacNAc, and/or 20 g/L LNB were additionally added to the medium as precursor(s).

Complex medium was sterilized by autoclaving (121° C., 21 min) and minimal medium by filtration (0.22 μm Sartorius). When necessary, the medium was made selective by adding an antibiotic: e.g., chloramphenicol (20 mg/L), carbenicillin (100 mg/L), spectinomycin (40 mg/L) and/or kanamycin (50 mg/L).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007). Plasmids were maintained in the host E. coli DH5alpha (F⁻, phi80dlacZΔM15, Δ(lacZYA-argF) U169, deoR, recA1, endA1, hsdR17(rk⁻, mk⁺), phoA, supE44, lambda⁻, thi-1, gyrA96, relA1) bought from Invitrogen.

### Strains and Mutations

Escherichia coli K12 MG1655 [λ⁻, F⁻, rph-1] was obtained from the Coli Genetic Stock Center (US), CGSC Strain #: 7740, in March 2007. Gene disruptions, gene introductions and gene replacements were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain. Transformants carrying a Red helper plasmid pKD46 were grown in 10 mL LB media with ampicillin, (100 mg/L) and L-arabinose (10 mM) at 30° C. to an OD₆₀₀ nm of 0.6. The cells were made electrocompetent by washing them with 50 mL of ice-cold water, a first time, and with 1 mL ice cold water, a second time. Then, the cells were resuspended in 50 μL of ice-cold water. Electroporation was done with 50 μL of cells and 10-100 ng of linear double-stranded-DNA product by using a Gene Pulser™ (BioRad) (600Ω, 25 μFD, and 250 volts). After electroporation, cells were added to 1 mL LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity. The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, re-purified from an agarose gel, and suspended in elution buffer (5 mM Tris, pH 8.0). Selected mutants were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock outs and knock-ins are checked with control primers.

In one example for sialic acid production, the mutant strain was derived from E. coli K12 MG1655 comprising genomic knock-ins of constitutive transcriptional units containing one or more copies of a glucosamine 6-phosphate N-acetyltransferase like e.g., GNA1 from Saccharomyces cerevisiae (UniProt ID P43577), an N-acetylglucosamine 2-epimerase like e.g., AGE from Bacteroides ovatus (UniProt ID A7LVG6) and one or more copies of an N-acetylneuraminate synthase like e.g., from Neisseria meningitidis (UniProt ID E0NCD4), Campylobacter jejuni (UniProt ID Q93MP9), Aeromonas caviae (UniProt ID Q9R9S2), Candidatus koribacter versatilis (UniProt ID Q1IMQ8), Legionella pneumophila (UniProt ID Q9RDX5), Methanocaldococcus jannaschii (UniProt ID Q58465) and Moritella viscosa (UniProt ID A0A090IMH4).

Alternatively, and/or additionally, sialic acid production can be obtained by genomic knock-ins of constitutive transcriptional units containing an UDP-N-acetylglucosamine 2-epimerase like e.g., NeuC from C. jejuni (UniProt ID Q93MP8) and one or more copies of an N-acetylneuraminate synthase like e.g., from Neisseria meningitidis (UniProt ID E0NCD4), Campylobacter jejuni (UniProt ID Q93MP9), Aeromonas caviae (UniProt ID Q9R9S2), Candidatus koribacter versatilis (UniProt ID Q1IMQ8), Legionella pneumophila (UniProt ID Q9RDX5), Methanocaldococcus jannaschii (UniProt ID Q58465) and Moritella viscosa (UniProt ID A0A090IMH4).

Alternatively and/or additionally, sialic acid production can be obtained by genomic knock-ins of constitutive transcriptional units containing a phosphoglucosamine mutase like e.g., glmM from E. coli (UniProt ID P31120), an N-acetylglucosamine-1-phosphate uridyltransferase/glucosamine-1-phosphate acetyltransferase like e.g., glmU from E. coli (UniProt ID P0ACC7), an UDP-N-acetylglucosamine 2-epimerase like e.g., NeuC from C. jejuni (UniProt ID Q93MP8) and one or more copies of an N-acetylneuraminate synthase like e.g., from Neisseria meningitidis (UniProt ID E0NCD4), Campylobacter jejuni (UniProt ID Q93MP9), Aeromonas caviae (UniProt ID Q9R9S2), Candidatus koribacter versatilis (UniProt ID Q1IMQ8), Legionella pneumophila (UniProt ID Q9RDX5), Methanocaldococcus jannaschii (UniProt ID Q58465) and Moritella viscosa (UniProt ID A0A090IMH4).

Alternatively, and/or additionally, sialic acid production can be obtained by genomic knock-ins of constitutive transcriptional units containing a bifunctional UDP-GlcNAc 2-epimerase/N-acetylmannosamine kinase like e.g., from Mus musculus (strain C57BL/6J) (UniProt ID Q91WG8), an N-acylneuraminate-9-phosphate synthetase like e.g., from Pseudomonas sp. UW4 (UniProt ID K9NPH9) and an N-acylneuraminate-9-phosphatase like e.g., from Candidatus magnetomorum sp. HK-1 (UniProt ID KPA15328.1) and/or from Bacteroides thetaiotaomicron (UniProt ID Q8A712).

Alternatively, and/or additionally, sialic acid production can be obtained by genomic knock-ins of constitutive transcriptional units containing a phosphoglucosamine mutase like e.g., glmM from E. coli (UniProt ID P31120), an N-acetylglucosamine-1-phosphate uridyltransferase/glucosamine-1-phosphate acetyltransferase like e.g., glmU from E. coli (UniProt ID P0ACC7), a bifunctional UDP-GlcNAc 2-epimerase/N-acetylmannosamine kinase like e.g., from M. musculus (strain C57BL/6J) (UniProt ID Q91WG8), an N-acylneuraminate-9-phosphate synthetase like e.g., from Pseudomonas sp. UW4 (UniProt ID K9NPH9) and an N-acylneuraminate-9-phosphatase like e.g., from Candidatus magnetomorum sp. HK-1 (UniProt ID KPA15328.1) and/or from Bacteroides thetaiotaomicron (UniProt ID Q8A712).

Sialic acid production can further be optimized in the mutant E. coli strain with genomic knock-outs of the E. coli genes comprising any one or more of nagA, nagB, nagC, nagD, nagE, nanA, nanE, nanK, manX, manY and manZ as described in WO 2018122225, and/or genomic knock-outs of the E. coli genes comprising any one or more of nanT, poxB, ldhA, adhE, aldB, pflA, pflC, ybiY, ackA and/or pta and with genomic knock-ins of constitutive transcriptional units comprising one or more copies of an L-glutamine-D-fructose-6-phosphate aminotransferase like e.g., the mutant glmS*54 from E. coli (differing from the wild-type E. coli glmS, having UniProt ID P17169, by an A39T, an R250C and an G472S mutation as described by Deng et al. (Biochimie 88, 419-29 (2006)), preferably a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonasputida, ScDOG1 from S. cerevisiae and BsAraL from Bacillus subtilis as described in WO 2018122225 and an acetyl-CoA synthetase like e.g., acs from E. coli (UniProt ID P27550).

For sialylated oligosaccharide production, the sialic acid production strains were further modified to express two or more orthologs with N-acylneuraminate cytidylyltransferase activity like e.g., the NeuA enzyme from C. jejuni (UniProt ID Q93MP7), the NeuA enzyme from Haemophilus influenzae (GenBank No. AGV11798.1) and the NeuA enzyme from Pasteurella multocida (GenBank No. AMK07891.1) and to express one or more copies of a beta-galactoside alpha-2,3-sialyltransferase like e.g., PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity, NmeniST3 from N. meningitidis (GenBank No. ARC07984.1) or PmultST2 from P. multocida subsp. multocida str. Pm70 (GenBank No. AAK02592.1), a beta-galactoside alpha-2,6-sialyltransferase like e.g., PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g., from M. musculus (UniProt ID Q64689). Constitutive transcriptional units of the N-acylneuraminate cytidylyltransferases and the sialyltransferases can be delivered to the mutant strain either via genomic knock-in or via expression plasmids. If the mutant strains producing sialic acid and CMP-sialic acid were intended to make sialylated lactose structures, the strains were additionally modified with genomic knock-outs of the E. coli LacZ, LacY and LacA genes and with a genomic knock-in of a constitutive transcriptional unit for a lactose permease like e.g., E. coli LacY (UniProt ID P02920). All mutant strains producing sialic acid, CMP-sialic acid and/or sialylated oligosaccharides could optionally be adapted for growth on sucrose via genomic knock-ins of constitutive transcriptional units containing a sucrose transporter like e.g., CscB from E. coli W (UniProt ID E0IXR1), a fructose kinase like e.g., Frk originating from Z. mobilis (UniProt ID Q03417) and a sucrose phosphorylase like e.g., BaSP from B. adolescentis (UniProt ID A0ZZH6).

Alternatively, and/or additionally, sialic acid and/or sialylated oligosaccharide production can further be optimized in the mutant E. coli strains with genomic knock-ins of constitutive transcriptional units comprising two or more different coding DNA sequences, each one encoding the same membrane transporter protein and/or encoding two or more functional membrane transporter proteins or functional fragments thereof with the same function in membrane transport like e.g., a sialic acid transporter like e.g., nanT from E. coli K-12 MG1655 (UniProt ID P41036), nanT from E. coli O6:H1 (UniProt ID Q8FD59), nanT from E. coli O157:H7 (UniProt ID Q8X9G8), nanT from E. albertii (UniProt ID B1EFH1) or a porter like e.g., EntS from E. coli (UniProt ID P24077), EntS from Kluyvera ascorbata (UniProt ID A0A378GQ13) and EntS from Salmonella enterica subsp. arizonae (UniProt ID A0A6Y2K4E8), MdfA from Cronobacter muytjensii (UniProt ID A0A2T7ANQ9), MdfA from Citrobacter youngae (UniProt ID D4BC23), MdfA from E. coli (UniProt ID P0AEY8), MdfA from Yokenella regensburgei (UniProt ID G9Z5F4), iceT from E. coli (UniProt ID A0A024L207), iceT from Citrobacter youngae (UniProt ID D4B8A6), SetA from E. coli (UniProt ID P31675), SetB from E. coli (UniProt ID P33026) and SetC from E. coli (UniProt ID P31436) or an ABC transporter like e.g., oppF from E. coli (UniProt ID P77737), lmrA from Lactococcus lactis subsp. lactis bv. diacetylactis (UniProt ID A0A1V0NEL4), or Blon_2475 from Bifidobacterium longum subsp. infantis (UniProt ID B7GPD4).

In one example for GDP-fucose production, the mutant strain was derived from E. coli K12 MG1655 comprising knock-outs of the E. coli wcaJ and thyA genes and genomic knock-ins of constitutive transcriptional units containing a sucrose transporter like e.g., CscB from E. coli W (UniProt ID E0IXR1), a fructose kinase like e.g., Frk originating from Zymomonas mobilis (UniProt ID Q03417) and a sucrose phosphorylase like e.g., BaSP originating from Bifidobacterium adolescentis (UniProt ID A0ZZH6). GDP-fucose production can further be optimized in the mutant E. coli strain by genomic knock-outs of any one or more of the E. coli genes comprising glgC, agp, pfkA, pfkB, pgi, arcA, icR, pgi and ion as described in WO 2016075243 and WO 2012007481. GDP-fucose production can additionally be optimized comprising genomic knock-ins of constitutive transcriptional units for one or more mannose-6-phosphate isomerases like e.g., manA from E. coli (UniProt ID P00946), phosphomannomutases like e.g., manB from E. coli (UniProt ID P24175), mannose-1-phosphate guanylyltransferases like e.g., manC from E. coli (UniProt ID P24174), GDP-mannose 4,6-dehydratases like e.g., gmd from E. coli (UniProt ID P0AC88) and GDP-L-fucose synthases like e.g., fcl from E. coli (UniProt ID P32055). GDP-fucose production can also be obtained by genomic knock-outs of the E. coli fucK and fucI genes and genomic knock-ins of constitutive transcriptional units containing one or more fucose permeases like e.g., fucP from E. coli (UniProt ID P11551) and one or more bifunctional enzymes with fucose kinase/fucose-1-phosphate guanylyltransferase activity like e.g., fkp from Bacteroides fragilis (UniProt ID SUV40286.1). All mutant strains can be additionally modified with genomic knock-outs of the E. coli LacZ, LacY and LacA genes and with a genomic knock-in of a constitutive transcriptional unit for a lactose permease like e.g., the E. coli LacY (UniProt ID P02920).

For production of fucosylated oligosaccharides, the mutant GDP-fucose production strain was additionally modified with expression plasmids comprising constitutive transcriptional units for an alpha-1,2-fucosyltransferase like e.g., HpFutC from H. pylori (GenBank No. AAD29863.1) and/or an alpha-1,3-fucosyltransferase like e.g., HpFucT from H. pylori (UniProt ID 030511) and with a constitutive transcriptional unit for the E. coli thyA (UniProt ID P0A884) as selective marker. Additionally, and/or alternatively, the constitutive transcriptional units of the fucosyltransferase genes could be present in the mutant E. coli strain via genomic knock-ins.

Alternatively, and/or additionally, GDP-fucose and/or fucosylated oligosaccharide production can further be optimized in the mutant E. coli strains with genomic knock-ins of constitutive transcriptional units comprising two or more different coding DNA sequences, each one encoding the same membrane transporter protein and/or encoding two or more functional membrane transporter proteins or functional fragments thereof with the same function in membrane transport like e.g., MdfA from Cronobacter muytjensii (UniProt ID A0A2T7ANQ9), MdfA from Citrobacter youngae (UniProt ID D4BC23), MdfA from E. coli (UniProt ID P0AEY8), MdfA from Yokenella regensburgei (UniProt ID G9Z5F4), iceT from E. coli (UniProt ID A0A024L207) or iceT from Citrobacter youngae (UniProt ID D4B8A6).

In an example to produce lacto-N-triose (LN3, GlcNAc-b1,3-Gal-b1,4-Glc), the mutant strain was derived from E. coli K12 MG1655 and modified with a knock-out of the E. coli lacZ, lacY, lacA and nagB genes and with genomic knock-ins of constitutive transcriptional units for a lactose permease like e.g., the E. coli LacY (UniProt ID P02920) and at least two different coding DNA sequences chosen from the list comprising SEQ ID NOs:1 to 57 and encoding one or more proteins with a galactoside beta-1,3-N-acetylglucosaminyltransferase activity.

In an example for production of LN3 derived oligosaccharides like lacto-N-tetraose (LNT, Gal-b1,3-GlcNAc-b1,3-Gal-b1,4-Glc), the mutant LN3 producing strains were further modified with constitutive transcriptional units delivered to the strain either via genomic knock-in or from an expression plasmid comprising at least two different coding DNA sequences chosen from the list comprising SEQ ID NOs:58 to 66 and encoding one or more proteins with an N-acetylglucosamine beta-1,3-galactosyltransferase activity.

In an example for production of LN3 derived oligosaccharides like lacto-N-neotetraose (LNnT, Gal-b1,4-GlcNAc-b1,3-Gal-b1,4-Glc) the mutant LN3 producing strains were further modified with constitutive transcriptional units delivered to the strain either via genomic knock-in or from an expression plasmid comprising at least two different coding DNA sequences chosen from the list comprising SEQ ID NOs:67 to 78 and encoding one or more proteins with an N-acetylglucosamine beta-1,4-galactosyltransferase activity.

The expression plasmids further comprised a constitutive transcriptional unit for the E. coli thyA (UniProt ID P0A884) as selective marker. Prior to transformation with any one of the expression plasmids, the E. coli strains were modified with an additional genomic knock-out of the E. coli thyA gene.

LN3, LNT and/or LNnT production can further be optimized in the mutant E. coli strains with genomic knock-outs of the E. coli genes comprising any one or more of galT, ushA, ldhA and agp.

The mutant LN3, LNT and LNnT producing strains can also be optionally modified for enhanced UDP-GlcNAc production with a genomic knock-in of a constitutive transcriptional unit for an L-glutamine-D-fructose-6-phosphate aminotransferase like e.g., the mutant glmS*54 from E. coli (differing from the wild-type E. coli glmS protein, having UniProt ID P17169, by an A39T, an R250C and an G472S mutation as described by Deng et al. (Biochimie 2006, 88: 419-429).

The mutant E. coli strains can also optionally be adapted with a genomic knock-in of a constitutive transcriptional unit for an UDP-glucose-4-epimerase like e.g., galE from E. coli (UniProt ID P09147), a phosphoglucosamine mutase like e.g., glmM from E. coli (UniProt ID P31120) and an N-acetylglucosamine-1-phosphate uridylyltransferase/glucosamine-1-phosphate acetyltransferase like e.g., glmU from E. coli (UniProt ID P0ACC7).

The mutant mutant LN3, LNT and LNnT producing E. coli strains can also optionally be adapted for growth on sucrose via genomic knock-ins of constitutive transcriptional units containing a sucrose transporter like e.g., CscB from E. coli W (UniProt ID E0IXR1), a fructose kinase like e.g., Frk originating from Zymomonas mobilis (UniProt ID Q03417) and a sucrose phosphorylase like e.g., BaSP originating from Bifidobacterium adolescentis (UniProt ID A0ZZH6).

Alternatively, and/or additionally, production of LN3, LNT, LNnT and oligosaccharides derived thereof can further be optimized in the mutant E. coli strains with genomic knock-ins of constitutive transcriptional units comprising two or more different coding DNA sequences, each one encoding the same membrane transporter protein and/or encoding two or more functional membrane transporter proteins or functional fragments thereof with the same function in membrane transport like e.g., MdfA from Cronobacter muytjensii (UniProt ID A0A2T7ANQ9), MdfA from Citrobacter youngae (UniProt ID D4BC23), MdfA from E. coli (UniProt ID P0AEY8), MdfA from Yokenella regensburgei (UniProt ID G9Z5F4), iceT from E. coli (UniProt ID A0A024L207) or iceT from Citrobacter youngae (UniProt ID D4B8A6).

Preferably but not necessarily, any one or more of the glycosyltransferases, the proteins involved in nucleotide-activated sugar synthesis and/or membrane transporter proteins were N- and/or C-terminally fused to a solubility enhancer tag like e.g., a SUMO-tag, an MBP-tag, His, FLAG®, Strep-II, Halo-tag, NusA, thioredoxin, GST and/or the Fh8-tag to enhance their solubility (Costa et al., Front. Microbiol. 2014, doi.org/10.3389/fmicb.2014.00063; Fox et al., Protein Sci. 2001, 10(3), 622-630; Jia and Jeaon, Open Biol. 2016, 6: 160196).

Optionally, the mutant E. coli strains are modified with one or more genomic knock-ins of one or more constitutive transcriptional units encoding one or more chaperone proteins like e.g., DnaK, DnaJ, GrpE and the GroEL/ES chaperonin system (Baneyx F., Palumbo J. L. (2003) Improving Heterologous Protein Folding via Molecular Chaperone and Foldase Co-Expression. In: Vaillancourt P. E. (eds) E. coli Gene Expression Protocols. Methods in Molecular Biology™, vol 205. Humana Press).

Optionally, the mutant E. coli strains are modified to create a glycominimized E. coli strain comprising genomic knock-out of any one or more of non-essential glycosyltransferase genes comprising pgaC, pgaD, rfe, rffT, rffM, bcsA, bcsB, bcsC, wcaA, wcaC, wcaE, wcaI, wcaJ, wcaL, waaH, waaF, waaC, waaU, waaZ, waaJ, waaO, waaB, waaS, waaG, waaQ, wbbl, arnC, arnT, yfdH, wbbK, opgG, opgH, ycjM, glgA, glgB, malQ, otsA and yaiP.

All constitutive promoters, UTRs and terminator sequences originated from the libraries described by Cambray et al. (Nucleic Acids Res. 2013, 41(9), 5139-5148), Dunn et al. (Nucleic Acids Res. 1980, 8, 2119-2132), Edens et al. (Nucleic Acids Res. 1975, 2, 1811-1820), Kim and Lee (FEBS Letters 1997, 407, 353-356) and Mutalik et al. (Nat. Methods 2013, No. 10, 354-360). The SEQ ID NOs described in disclosure are summarized in Table 1.

All genes were ordered synthetically at Twist Bioscience (twistbioscience.com) or IDT (eu.idtdna.com) and the codon usage was adapted using the tools of the supplier.

All strains were stored in cryovials at −80° C. (overnight LB culture mixed in a 1:1 ratio with 70% glycerol).

TABLE 1
Overview of SEQ ID NOs described in the disclosure

| | | | | |
|---|---|---|---|---|
| SEQ ID | SEQ ID | | | Country of |
| NO: | NO: | | | origin of digital |
| (nucleotide | (protein | | | sequence |
| sequence) | sequence) | Organism | Origin | information |

Enzymes with galactoside beta-1,3-N-acetylglucosaminyltransferase activity

| | | | | |
|---|---|---|---|---|
| 01 | 79 | Pasteurella multocida | Synthetic | USA |
| 02 | 80 | Neisseria meningitidis MC58 | Synthetic | United Kingdom |
| 03 | 80 | Neisseria meningitidis MC58 | Synthetic | United Kingdom |
| 04 | 80 | Neisseria meningitidis MC58 | Synthetic | United Kingdom |
| 05 | 80 | Neisseria meningitidis MC58 | Synthetic | United Kingdom |
| 06 | 80 | Neisseria meningitidis MC58 | Synthetic | United Kingdom |
| 07 | 81 | Neisseria meningitidis M22425 | Synthetic | Burkina Faso |
| 08 | 82 | Neisseria gonorrhoeae | Synthetic | Germany |
| 09 | 83 | Neisseria gonorrhoeae | Synthetic | Germany |
| 10 | 84 | Neisseria lactamica | Synthetic | Unknown |
| 11 | 85 | Neisseria lactamica | Synthetic | Unknown |
| 12 | 86 | Pasteurella dagmatis | Synthetic | Unknown |
| 13 | 87 | Neisseria gonorrhoeae | Synthetic | Germany |
| 14 | 88 | Neisseria gonorrhoeae | Synthetic | Germany |
| 15 | 89 | Neisseria meningitidis | Synthetic | United Kingdom |
| 16 | 90 | Neisseria meningitidis | Synthetic | United Kingdom |
| 17 | 91 | Neisseria meningitidis | Synthetic | United Kingdom |
| 18 | 92 | Neisseria meningitidis | Synthetic | United Kingdom |
| 19 | 93 | Artificial sequence | Synthetic | Not applicable |
| 20 | 94 | Artificial sequence | Synthetic | Not applicable |
| 21 | 95 | Artificial sequence | Synthetic | Not applicable |
| 22 | 96 | Artificial sequence | Synthetic | Not applicable |
| 23 | 97 | Artificial sequence | Synthetic | Not applicable |
| 24 | 98 | Artificial sequence | Synthetic | Not applicable |
| 25 | 99 | Artificial sequence | Synthetic | Not applicable |
| 26 | 100 | Artificial sequence | Synthetic | Not applicable |
| 27 | 101 | Pasteurella multocida | Synthetic | USA |
| 28 | 102 | Neisseria lactamica | Synthetic | Unknown |
| 29 | 103 | Neisseria meningitidis | Synthetic | United Kingdom |
| 30 | 104 | Neisseria polysaccharea | Synthetic | Unknown |
| 31 | 105 | Neisseria subflava | Synthetic | Unknown |
| 32 | 106 | Neisseria meningitidis | Synthetic | United Kingdom |
| 33 | 107 | Neisseria meningitidis | Synthetic | United Kingdom |
| 34 | 108 | Neisseria meningitidis | Synthetic | United Kingdom |
| 35 | 109 | Neisseria meningitidis | Synthetic | United Kingdom |
| 36 | 110 | Helicobacter pylori | Synthetic | United Kingdom |
| 37 | 111 | Helicobacter pylori | Synthetic | United Kingdom |
| 38 | 112 | Helicobacter pylori | Synthetic | United Kingdom |
| 39 | 113 | Helicobacter pylori | Synthetic | United Kingdom |
| 40 | 114 | Helicobacter pylori | Synthetic | United Kingdom |
| 41 | 115 | Helicobacter pylori | Synthetic | United Kingdom |
| 42 | 116 | Helicobacter pylori | Synthetic | United Kingdom |
| 43 | 117 | Helicobacter pylori | Synthetic | United Kingdom |
| 44 | 118 | Helicobacter pylori | Synthetic | United Kingdom |
| 45 | 119 | Helicobacter pylori | Synthetic | United Kingdom |
| 46 | 120 | Helicobacter pylori | Synthetic | United Kingdom |
| 47 | 121 | Helicobacter cetorum | Synthetic | USA |
| 48 | 122 | Neisseria meningitidis | Synthetic | United Kingdom |
| 49 | 123 | Neisseria meningitidis | Synthetic | United Kingdom |
| 50 | 124 | Neisseria meningitidis | Synthetic | United Kingdom |
| 51 | 125 | Neisseria meningitidis | Synthetic | United Kingdom |
| 52 | 126 | Helicobacter pylori | Synthetic | United Kingdom |
| 53 | 127 | Helicobacter pylori | Synthetic | United Kingdom |
| 54 | 128 | Helicobacter pylori | Synthetic | United Kingdom |
| 55 | 129 | Helicobacter pylori | Synthetic | United Kingdom |
| 56 | 130 | Helicobacter pylori | Synthetic | United Kingdom |
| 57 | 131 | Escherichia coli upec-202 | Synthetic | USA |

Enzymes with N-acetylglucosamine beta-1,3-galactosyltransferase activity

| | | | | |
|---|---|---|---|---|
| 58 | 132 | Escherichia coli | Synthetic | USA |
| 59 | 132 | Escherichia coli | Synthetic | USA |
| 60 | 133 | Pseudogulbenkiania ferrooxidans | Synthetic | USA |
| 61 | 133 | Pseudogulbenkiania ferrooxidans | Synthetic | USA |
| 62 | 133 | Pseudogulbenkiania ferrooxidans | Synthetic | USA |
| 63 | 134 | Salmonella enterica | Synthetic | United Kingdom |
| 64 | 134 | Salmonella enterica | Synthetic | United Kingdom |
| 65 | 134 | Salmonella enterica | Synthetic | United Kingdom |
| 66 | 135 | Corynebacterium glutamicum | Synthetic | Unknown |

Enzymes with N-acetylglucosamine beta-1,4-galactosyltransferase activity

| | | | | |
|---|---|---|---|---|
| 67 | 136 | Neisseria meningitidis | Synthetic | United Kingdom |
| 68 | 137 | Neisseria meningitidis | Synthetic | United Kingdom |
| 69 | 137 | Neisseria meningitidis | Synthetic | United Kingdom |
| 70 | 137 | Neisseria meningitidis | Synthetic | United Kingdom |
| 71 | 138 | Streptococcus agalactiae | Synthetic | Czech Republic |
| 72 | 139 | Helicobacter pylori | Synthetic | United Kingdom |
| 73 | 140 | Helicobacter pylori | Synthetic | United Kingdom |
| 74 | 141 | Aggregatibacter aphrophilus | Synthetic | United Kingdom |
| 75 | 142 | Pasteurella multocida | Synthetic | USA |
| 76 | 143 | Helicobacter pylori | Synthetic | United Kingdom |
| 77 | 144 | Pasteurella multocida | Synthetic | USA |
| 78 | 145 | Kingella denitrificans | Synthetic | Unknown |

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL minimal medium by diluting 400×. These final 96-well culture plates were then incubated at 37° C. on an orbital shaker at 800 rpm for 72 h, or shorter, or longer. To measure sugar concentrations at the end of the cultivation experiment whole broth samples were taken from each well by boiling the culture broth for 15 min at 60° C. before spinning down the cells (=average of intra- and extracellular sugar concentrations).

A preculture for the bioreactor was started from an entire 1 mL cryovial of a certain strain, inoculated in 250 mL or 500 mL minimal medium in a 1 L or 2.5 L shake flask and incubated for 24 h at 37° C. on an orbital shaker at 200 rpm. A 5 L bioreactor (having a 5 L working volume) was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H₂SO₄ and 20% NH₄OH. The exhaust gas was cooled. 10% solution of silicone antifoaming agent was added when foaming raised during the fermentation.

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Analytical Analysis

Standards such as but not limited to sucrose, lactose, N-acetyllactosamine (LacNAc, Gal-b1,4-GlcNAc), lacto-N-biose (LNB, Gal-b1,3-GlcNAc), fucosylated LacNAc (2′FLacNAc, 3-FLacNAc), sialylated LacNAc, (3′SLacNAc, 6′SLacNAc), fucosylated LNB (2′FLNB, 4′FLNB), lacto-N-triose II (LN3), lacto-N-tetraose (LNT), lacto-N-neo-tetraose (LNnT), LNFP-I, LNFP-II, LNFP-III, LNFP-V, LNFP-VI, LSTa, LSTc and LSTd were purchased from Carbosynth (UK), Elicityl (France) and IsoSep (Sweden). Other compounds were analyzed with in-house made standards.

Neutral oligosaccharides were analyzed on a Waters Acquity H-class UPLC with Evaporative Light Scattering Detector (ELSD) or a Refractive Index (RI) detection. A volume of 0.7 μL sample was injected on a Waters Acquity UPLC BEH Amide column (2.1×100 mm; 130 Å; 1.7 μm) column with an Acquity UPLC BEH Amide VanGuard column, 130 Å, 2.1×5 mm. The column temperature was 50° C. The mobile phase consisted of a ¼ water and ¾ acetonitrile solution to which 0.2% triethylamine was added. The method was isocratic with a flow of 0.130 mL/min. The ELS detector had a drift tube temperature of 50° C. and the N₂ gas pressure was 50 psi, the gain 200 and the data rate 10 pps. The temperature of the RI detector was set at 35° C.

Sialylated oligosaccharides were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 70% acetonitrile, 26% ammonium acetate buffer (150 mM) and 4% methanol to which 0.05% pyrrolidine was added. The method was isocratic with a flow of 0.150 mL/min. The temperature of the RI detector was set at 35° C.

Both neutral and sialylated sugars were analyzed on a Waters Acquity H-class UPLC with Refractive Index (RI) detection. A volume of 0.5 μL sample was injected on a Waters Acquity UPLC BEH Amide column (2.1×100 mm; 130 Å; 1.7 μm). The column temperature was 50° C. The mobile phase consisted of a mixture of 72% acetonitrile and 28% ammonium acetate buffer (100 mM) to which 0.1% triethylamine was added. The method was isocratic with a flow of 0.260 mL/min. The temperature of the RI detector was set at 35° C.

For analysis on a mass spectrometer, a Waters Xevo TQ-MS with Electron Spray Ionisation (ESI) was used with a desolvation temperature of 450° C., a nitrogen desolvation gas flow of 650 L/h and a cone voltage of 20 V. The MS was operated in selected ion monitoring (SIM) in negative mode for all oligosaccharides. Separation was performed on a Waters Acquity UPLC with a Thermo Hypercarb column (2.1×100 mm; 3 μm) on 35° C. A gradient was used wherein eluent A was ultrapure water with 0.1% formic acid and wherein eluent B was acetonitrile with 0.1% formic acid. The oligosaccharides were separated in 55 min using the following gradient: an initial increase from 2 to 12% of eluent B over 21 min, a second increase from 12 to 40% of eluent B over 11 min and a third increase from 40 to 100% of eluent B over 5 min. As a washing step 100% of eluent B was used for 5 min. For column equilibration, the initial condition of 2% of eluent B was restored in 1 min and maintained for 12 min.

Both neutral and sialylated sugars at low concentrations (below 50 mg/L) were analysed on a Dionex HPAEC system with pulsed amperometric detection (PAD). A volume of 5 μL of sample was injected on a Dionex CarboPac PA200 column 4×250 mm with a Dionex CarboPac PA200 guard column 4×50 mm. The column temperature was set to 30° C. A gradient was used wherein eluent A was deionized water, wherein eluent B was 200 mM Sodium hydroxide and wherein eluent C was 500 mM Sodium acetate. The oligosaccharides were separated in 60 min while maintaining a constant ratio of 25% of eluent B using the following gradient: an initial isocratic step maintained for 10 min of 75% of eluent A, an initial increase from 0 to 4% of eluent C over 8 min, a second isocratic step maintained for 6 min of 71% of eluent A and 4% of eluent C, a second increase from 4 to 12% of eluent C over 2.6 min, a third isocratic step maintained for 3.4 min of 63% of eluent A and 12% of eluent C and a third increase from 12 to 48% of eluent C over 5 min. As a washing step 48% of eluent C was used for 3 min. For column equilibration, the initial condition of 75% of eluent A and 0% of eluent C was restored in 1 min and maintained for 11 min. The applied flow was 0.5 mL/min.

### Example 3. Materials and Methods Saccharomyces cerevisiae

### Media

Strains were grown on Synthetic Defined yeast medium with Complete Supplement Mixture (SD CSM) or CSM drop-out (SD CSM-Ura, SD CSM-Trp, SD CSM-His) containing 6.7 g/L Yeast Nitrogen Base without amino acids (YNB w/o AA, Difco), 20 g/L agar (Difco) (solid cultures), 22 g/L glucose monohydrate or 20 g/L lactose and 0.79 g/L CSM or 0.77 g/L CSM-Ura, 0.77 g/L CSM-Trp, or 0.77 g/L CSM-His (MP Biomedicals).

### Strains

S. cerevisiae BY4742 created by Brachmann et al. (Yeast (1998) 14:115-32) was used, available in the Euroscarf culture collection. All mutant strains were created by homologous recombination or plasmid transformation using the method of Gietz (Yeast 11:355-360, 1995).

### Plasmids

In one example to produce sialic acid and CMP-sialic acid, a yeast expression plasmid can be derived from the pRS420-plasmid series (Christianson et al., 1992, Gene 110: 119-122) containing the TRP1 selection marker and constitutive transcriptional units for one or more copies of an L-glutamine-D-fructose-6-phosphate aminotransferase like e.g., the mutant glmS*54 from E. coli (differing from the wild-type E. coli glmS, having UniProt ID P17169, by an A39T, an R250C and an G472S mutation as described by Deng et al. (Biochimie 88, 419-29 (2006)), a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from Pseudomonas putida, ScDOG1 from S. cerevisiae and BsAraL from Bacillus subtilis as described in WO 2018122225, an N-acetylglucosamine 2-epimerase like e.g., AGE from B. ovatus (UniProt ID A7LVG6), one or more copies of an N-acetylneuraminate synthase like e.g., from Neisseria meningitidis (UniProt ID E0NCD4), Campylobacter jejuni (UniProt ID Q93MP9), Aeromonas caviae (UniProt ID Q9R9S2), Candidatus koribacter versatilis (UniProt ID Q1IMQ8), Legionella pneumophila (UniProt ID Q9RDX5), Methanocaldococcus jannaschii (UniProt ID Q58465) and Moritella viscosa (UniProt ID A0A090INM4), and two or more orthologs with N-acylneuraminate cytidylyltransferase activity like e.g., the NeuA enzyme from C. jejuni (UniProt ID Q93MP7), the NeuA enzyme from Haemophilus influenzae (GenBank No. AGV11798.1) and the NeuA enzyme from Pasteurella multocida (GenBank No. AMK07891.1). Optionally, a constitutive transcriptional unit comprising one or more copies for a glucosamine 6-phosphate N-acetyltransferase like e.g., GNA1 from S. cerevisiae (UniProt ID P43577) was/were added as well. To produce sialylated oligosaccharides, the plasmid further comprised constitutive transcriptional units for a lactose permease like e.g., LAC12 from Kluyveromyces lactis (UniProt ID P07921), and one or more copies of a beta-galactoside alpha-2,3-sialyltransferase like e.g., PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity, NmeniST3 from N. meningitidis (GenBank No. ARC07984.1) or PmultST2 from P. multocida subsp. multocida str. Pm70 (GenBank No. AAK02592.1), a beta-galactoside alpha-2,6-sialyltransferase like e.g., PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g., from M. musculus (UniProt ID Q64689).

In one example to produce GDP-fucose, a yeast expression plasmid like p2a_2μ_Fuc (Chan 2013, Plasmid 70, 2-17) can be used for expression of foreign genes in S. cerevisiae. This plasmid contains an ampicillin resistance gene and a bacterial origin of replication to allow for selection and maintenance in E. coli and the 2μ yeast ori and the Ura3 selection marker for selection and maintenance in yeast. This plasmid is further modified with constitutive transcriptional units for a lactose permease like e.g., LAC12 from K. lactis (UniProt ID P07921), one or more GDP-mannose 4,6-dehydratases like e.g., gmd from E. coli (UniProt ID P0AC88) and one or more GDP-L-fucose synthases like e.g., fcl from E. coli (UniProt ID P32055). The yeast expression plasmid p2a_2μ_Fuc2 can be used as an alternative expression plasmid of the p2a_2μ_Fuc plasmid comprising next to the ampicillin resistance gene, the bacterial ori, the 2p yeast ori and the Ura3 selection marker constitutive transcriptional units for a lactose permease like e.g., LAC12 from K. lactis (UniProt ID P07921), one or more fucose permeases like e.g., fucP from E. coli (UniProt ID P11551) and one or more bifunctional enzymes with fucose kinase/fucose-1-phosphate guanylyltransferase activity like e.g., fkp from Bacteroides fragilis (UniProt ID SUV40286.1). To further produce fucosylated oligosaccharides, the p2a_2μ_Fuc and its variant the p2a_2μ_Fuc2, additionally contained (a) constitutive transcriptional unit(s) for one or more fucosyltransferases.

In one example to produce UDP-galactose, a yeast expression plasmid can be derived from the pRS420-plasmid series (Christianson et al., 1992, Gene 110: 119-122) containing the HIS3 selection marker and a constitutive transcriptional unit for an UDP-glucose-4-epimerase like e.g., galE from E. coli (UniProt ID P09147). This plasmid can be further modified with constitutive transcriptional units for a lactose permease like e.g., LAC12 from K. lactis (UniProt ID P07921) and at least two different coding DNA sequences chosen from the list comprising SEQ ID NOs:1 to 57 and encoding one or more proteins with a galactoside beta-1,3-N-acetylglucosaminyltransferase activity to produce LN3. To further produce LN3-derived oligosaccharides like LNT, the mutant LN3 producing strains were further modified with constitutive transcriptional units comprising at least two different coding DNA sequences chosen from the list comprising SEQ ID NOs:58 to 66 and encoding one or more proteins with an N-acetylglucosamine beta-1,3-galactosyltransferase activity. To further produce LN3-derived oligosaccharides like LNnT, the mutant LN3 producing strains were further modified with constitutive transcriptional units comprising at least two different coding DNA sequences chosen from the list comprising SEQ ID NOs:67 to 78 and encoding one or more proteins with an N-acetylglucosamine beta-1,4-galactosyltransferase activity.

Preferably but not necessarily, any one or more of the glycosyltransferase and/or the proteins involved in nucleotide-activated sugar synthesis were N- and/or C-terminally fused to a SUMOstar tag (e.g., obtained from pYSUMOstar, Life Sensors, Malvern, PA) to enhance their solubility.

Optionally, the mutant yeast strains were modified with one or more genomic knock-ins of one or more constitutive transcriptional units encoding one or more chaperone proteins like e.g., Hsp31, Hsp32, Hsp33, Sno4, Kar2, Ssb1, Sse1, Sse2, Ssa1, Ssa2, Ssa3, Ssa4, Ssb2, Ecm10, Ssc1, Ssq1, Ssz1, Lhs1, Hsp82, Hsc82, Hsp78, Hsp104, Tcp1, Cct4, Cct8, Cct2, Cct3, Cct5, Cct6, and Cct7 (Gong et al., 2009, Mol. Syst. Biol. 5: 275). Plasmids were maintained in the host E. coli DH5alpha (F⁻, phi80dlacZdeltaM15, delta(lacZYA-argF)U169, deoR, recA1, endA1, hsdR17(rk⁻, mk⁺), phoA, supE44, lambda⁻, thi-1, gyrA96, relA1) bought from Invitrogen.

### Heterologous and Homologous Expression

Genes that needed to be expressed, be it from a plasmid or from the genome were synthetically synthetized with one of the following companies: DNA2.0, Gen9, IDT or Twist Bioscience. Expression could be further facilitated by optimizing the codon usage to the codon usage of the expression host. Genes were optimized using the tools of the supplier.

### Cultivations Conditions

In general, yeast strains were initially grown on SD CSM plates to obtain single colonies. These plates were grown for 2-3 days at 30° C. Starting from a single colony, a preculture was grown over night in 5 mL at 30° C., shaking at 200 rpm. Subsequent 125 mL shake flask experiments were inoculated with 2% of this preculture, in 25 mL media. These shake flasks were incubated at 30° C. with an orbital shaking of 200 rpm.

### Gene Expression Promoters

Genes were expressed using synthetic constitutive promoters, as described by Blazeck (Biotechnology and Bioengineering, Vol. 109, No. 11, 2012).

### Example 4. Production of 6′-sialyllactose (6′-SL) or 3′-sialyllactose (3′-SL) with a Modified E. coli Strain

An E. coli K-12 strain MG1655 is modified for sialic acid production as described in Example 2 comprising knock-outs of the E. coli nagA, nagB, nanA, nanT, nanE, nanK, LacZ, LacY and LacA genes and genomic knock-ins of constitutive transcriptional units comprising genes encoding the lactose permease (LacY) from E. coli (UniProt ID P02920), the sialic acid transporter (nanT) from E. coli (UniProt ID P41036), the mutant L-glutamine D-fructose-6-phosphate aminotransferase glmS*54 from E. coli (differing from the wild-type E. coli glmS, having UniProt ID P17169, by an A39T, an R250C and an G472S mutation), the glucosamine 6-phosphate N-acetyltransferase (GNA1) from S. cerevisiae (UniProt ID P43577), the N-acetylglucosamine 2-epimerase (AGE) from B. ovatus (UniProt ID A7LVG6), the N-acetylneuraminate synthase (NeuB) from N. meningitidis (UniProt ID E0NCD4), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). The thus obtained mutant E. coli strain sB is further modified with a genomic knock-in of a constitutive transcriptional unit comprising either the gene encoding the alpha-2,6-sialyltransferase PdbST from P. damselae (UniProt ID 066375) resulting in strain sB6 or the gene encoding the alpha-2,3-sialyltransferase PmultST3 from P. multocida (UniProt ID Q9CLP3) resulting in strain sB3. Both strain sB6 and sB3 were in a next step further modified with either 1) a genomic knock-in of a constitutive transcriptional unit comprising the gene encoding the N-acylneuraminate cytidylyltransferase NeuA from C. jejuni (UniProt ID Q93MP7) to obtain strains SB6A and SB3A, 2) a genomic knock-in of constitutive transcriptional units comprising the genes encoding two N-acylneuraminate cytidylyltransferase enzymes, i.e., NeuA from C. jejuni (UniProt ID Q93MP7) and NeuA from H. influenzae (GenBank No. AGV11798.1), to obtain strains SB6B and SB3B, 3) a genomic knock-in of constitutive transcriptional units comprising the genes encoding three N-acylneuraminate cytidylyltransferase enzymes, i.e., NeuA from C. jejuni (UniProt ID Q93MP7), NeuA from H. influenzae (GenBank No. AGV11798.1) and NeuA from P. multocida (GenBank No. AMK07891.1) to obtain strains SB6C and SB3C, 4) an expression plasmid comprising a constitutive transcriptional unit comprising the gene encoding the N-acylneuraminate cytidylyltransferase NeuA from C. jejuni (UniProt ID Q93MP7) to obtain strains SB6D and SB3D, 5) an expression plasmid comprising constitutive transcriptional units comprising the genes encoding two N-acylneuraminate cytidylyltransferase enzymes, i.e., NeuA from C. jejuni (UniProt ID Q93MP7) and NeuA from H. influenzae (GenBank No. AGV11798.1), to obtain strains SB6E and SB3E, 6) an expression plasmid comprising constitutive transcriptional units comprising the genes encoding three N-acylneuraminate cytidylyltransferase enzymes, i.e., NeuA from C. jejuni (UniProt ID Q93MP7), NeuA from H. influenzae (GenBank No. AGV11798.1) and NeuA from P. multocida (GenBank No. AMK07891.1), to obtain strains SB6F and SB3F, 7) a genomic knock-in of a constitutive transcriptional unit comprising the gene encoding the N-acylneuraminate cytidylyltransferase NeuA from C. jejuni (UniProt ID Q93MP7) and an expression plasmid comprising a constitutive transcriptional unit comprising the gene encoding the N-acylneuraminate cytidylyltransferase NeuA from H. influenzae (GenBank No. AGV11798.1) to obtain strains SB6G and SB3G, or 8) a genomic knock-in of constitutive transcriptional units comprising the genes encoding two N-acylneuraminate cytidylyltransferase enzymes, i.e., NeuA from C. jejuni (UniProt ID Q93MP7) and NeuA from H. influenzae (GenBank No. AGV11798.1), and an expression plasmid comprising a constitutive transcriptional unit comprising the gene encoding the N-acylneuraminate cytidylyltransferase NeuA from P. multocida (GenBank No. AMK07891.1) to obtain strains SB6H and SB3H, for production of 6′-SL in case of the strains from the sB6 lineage comprising strains SB6A, SB6B, SB6C, SB6D, SB6E, SB6F, SB6G and SB6H, or for production of 3′-SL in case of the strains from the sB3 lineage comprising strains SB3A, SB3B, SB3C, SB3D, SB3E, SB3F, SB3G and SB3H. All novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 5. Production of 6′-sialyllactose (6′-SL) with a Modified E. coli Strain

An E. coli K-12 strain MG1655 was modified for sialic acid and 6′-siayllactose production as described in Example 2 comprising knock-outs of the E. coli nagA, nagB, nanA, nanT, nanE, nanK, LacZ, LacY and LacA genes and genomic knock-ins of constitutive transcriptional units comprising genes encoding the lactose permease (LacY) from E. coli (UniProt ID P02920), the sialic acid transporter (nanT) from E. coli (UniProt ID P41036), the mutant L-glutamine-D-fructose-6-phosphate aminotransferase glmS*54 from E. coli (differing from the wild-type E. coli glmS, having UniProt ID P17169, by an A39T, an R250C and an G472S mutation), the glucosamine 6-phosphate N-acetyltransferase (GNA1) from S. cerevisiae (UniProt ID P43577), the N-acetylglucosamine 2-epimerase (AGE) from B. ovatus (UniProt ID A7LVG6), the N-acetylneuraminate synthase (NeuB) from C. jejuni (UniProt ID Q93MP9), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). The thus obtained mutant E. coli strain S0 was further modified with genomic knock-ins and/or expression plasmids with constitutive transcriptional units to express
a) one N-acylneuraminate cytidylyltransferase enzyme NeuA from C. jejuni (UniProt ID Q93MP7) and one polypeptide consisting of amino acid residues 108 to 497 of PdbST from P. damselae (UniProt ID 066375) having beta-galactoside alpha-2,6-sialyltransferase activity,
b) two N-acylneuraminate cytidylyltransferases consisting of the NeuA enzyme from C. jejuni (UniProt ID Q93MP7) and the NeuA enzyme from H. influenzae (GenBank No. AGV11798.1), and two copies of the polypeptide consisting of amino acid residues 108 to 497 of PdbST from P. damselae (UniProt ID 066375) having beta-galactoside alpha-2,6-sialyltransferase activity, or
c) three N-acylneuraminate cytidylyltransferases consisting of the NeuA enzyme from C. jejuni (UniProt ID Q93MP7), the NeuA enzyme from H. influenzae (GenBank No. AGV11798.1) and the NeuA enzyme from P. multocida (GenBank No. AMK07891.1), and three copies of the polypeptide consisting of amino acid residues 108 to 497 of PdbST from P. damselae (UniProt ID 066375) having beta-galactoside alpha-2,6-sialyltransferase activity,
creating the mutant E. coli strains S1, S2 and S3, respectively, as summarized in Table 2. Details on the promoter, UTR and terminator sequences used to express the NeuA enzymes or the polypeptide with beta-galactoside alpha-2,6-sialyltransferase activity is summarized in Table 3. The novel strains were evaluated in a growth experiment according to the culture conditions provided in Example 2, in which the culture medium contained 30 g/L sucrose and 20 g/L lactose. Each strain was grown in four biological replicates in a 96-well plate. After 72 h of incubation, the culture broth was harvested, and the sugars were analysed on UPLC.

The experiment demonstrated all strains produced sialic acid and 6′-SL. Herein, strain S2 expressing two enzymes with N-acylneuraminate cytidylyltransferase activity and two copies of a polypeptide having beta-galactoside alpha-2,6-sialyltransferase activity produced 2.60 times more 6′-SL compared to strain S1 expressing one N-acylneuraminate cytidylyltransferase and one polypeptide having beta-galactoside alpha-2,6-sialyltransferase activity. In the same experiment, strain S3 expressing three enzymes with N-acylneuraminate cytidylyltransferase activity and three copies of a polypeptide having beta-galactoside alpha-2,6-sialyltransferase activity produced 11.50 times more 6′-SL compared to strain S1 expressing one N-acylneuraminate cytidylyltransferase and one polypeptide having beta-galactoside alpha-2,6-sialyltransferase activity. The experiment further demonstrated the mutant strains S1, S2 and S3 had a similar growth rate and did not suffer from any genomic or plasmid DNA instability or reorganisation during cultivation (Results not shown).

TABLE 2
Additional transcriptional units present in E. coli strains
S1, S2 and S3 compared to the parental E. coli strain S0

| | | Transcriptional unit | | | |
|---|---|---|---|---|---|
| | | Promoter | UTR | Coding DNA | Terminator |
| Strain | Location | sequence* | sequence* | sequence | sequence* |
| S1 | Genomic knock- | P35 | U18 | NeuA from C. jejuni | T2 |
| | in | | | (UniProt ID Q93MP7) | |
| | Genomic knock- | P50 | U10 | Fragment from PdbST | T1 |
| | in | | | (UniProt ID O66375)** | |
| S2 | Genomic knock- | P35 | U18 | NeuA from C. jejuni | T2 |
| | in | | | (UnipProt ID Q93MP7) | |
| | Genomic knock- | P26 | U18 | NeuA from H. influenzae | T9 |
| | in | | | (GenBank No. | |
| | | | | AGV11798.1) | |
| | Genomic knock- | P50 | U10 | Fragment from PdbST | T1 |
| | in | | | (UniProt ID O66375)** | |
| | Genomic knock- | P5 | U10 | Fragment from PdbST | T7 |
| | in | | | (UniProt ID O66375)** | |
| S3 | Genomic knock- | P35 | U18 | NeuA from C. jejuni | T2 |
| | in | | | (UnipProt ID Q93MP7) | |
| | Genomic knock- | P26 | U18 | NeuA from H. influenzae | T9 |
| | in | | | (GenBank No. | |
| | | | | AGV11798.1) | |
| | Plasmid | P26 | U18 | NeuA from P. multocida | T9 |
| | | | | (GenBank No. | |
| | | | | AMK07891.1) | |
| | Genomic knock- | P50 | U10 | Fragment from PdbST | T1 |
| | in | | | (UniProt ID O66375)** | |
| | Genomic knock- | P5 | U10 | Fragment from PdbST | T7 |
| | in | | | (UniProt ID O66375)** | |
| | Plasmid | P5 | U10 | Fragment from PdbST | T7 |
| | | | | (UniProt ID O66375)** | |

*See Table 3
**The fragment consisted of amino acid residues 108 to 497 from PdbST (UniProt ID O66375) and showed beta-galactoside alpha-2,6-sialyltransferase activity on lactose.

TABLE 3
Promoter, UTR and terminator sequences used to express the NeuA enzymes or a polypeptide
consisting of amino acid residues 108 to 497 of PdbST from P. damselae (UniProt
ID O66375) having beta-galactoside alpha-2,6-sialyltransferase activity on lactose
in the mutant E. coli strains S1, S2 and S3 as given in Table 2.

| Promoter sequence | Reference |
|---|---|
| P5 = PROM0005 = Mutalik_P5 | Mutalik et al. (Nat. Methods 2013, 10, 354-360) |
| P26 PROM0026 = | Mutalik et al. (Nat. Methods 2013, 10, 354-360) |
| Mutalik_apFAB110 | |
| P35 = PROM0035 = Mutalik_apFAB37 | Mutalik et al. (Nat. Methods 2013, 10, 354-360) |
| P50 = PROM0050 = Mutalik_apFAB82 | Mutalik et al. (Nat. Methods 2013, 10, 354-360) |

| UTR sequence | Reference |
|---|---|
| U10 = UTR0010_GalE_BCD12 | Mutalik et al. (Nat. Methods 2013, 10, 354-360) |
| U18 = UTR0018_GalE_BCD18 | Mutalik et al. (Nat. Methods 2013, 10, 354-360) |

| Terminator sequence | Reference |
|---|---|
| T1 = TER0001_TT5-T7 | Dunn et al. (Nucleic Acids Res. 1980, 8, 2119-2132) |
| T2 = TER0002_rrnBT1_rrnBT2 | Kim and Lee (FEBS Letters 1997, 407, 353-356) |
| T7 = TER0007_ilvGEDA | Cambray et al. (Nucleic Acids Res. 2013, 41, 5139-5148) |
| T9 = TER0009_M13_central | Edens et al. (Nucleic Acids Res. 1975, 2, 1811-1820) |

### Example 6. Production of 6′-sialyllactose (6′-SL) or 3′-sialyllactose (3′-SL) with a Modified E. coli Strain

In a next experiment, the mutant E. coli strains SB6A, SB6B, SB6C, SB6D, SB6E, SB6F, SB6G, SB6H, SB3A, SB3B, SB3C, SB3D, SB3E, SB3F, SB3G and SB3H as described in Example 4 are further modified with genomic knock-ins of constitutive transcriptional units to express two enterobactin exporter orthologs consisting of EntS from Kluyvera ascorbata (UniProt ID A0A378GQ13) and EntS from Salmonella enterica subsp. arizonae (UniProt ID A0A6Y2K4E8). The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 7. Production of 6′-sialyllactose (6′-SL) with a Modified E. coli Strain

In another experiment, the mutant E. coli strain SB6H as described in Example 4 was further modified with additional knock-outs of the genes comprising ackA-pta, ldhA, poxB and the O-antigen cluster comprising all genes between wbbK and wcaN with wbbK and wcaN and with additional genomic knock-ins of constitutive transcriptional units comprising genes encoding an extra copy of the L-glutamine D-fructose-6-phosphate aminotransferase (glmS*54) from E. coli (differing from the wild-type E. coli glmS, having UniProt ID P17169, by an A39T, an R250C and an G472S mutation), an extra copy of the glucosamine 6-phosphate N-acetyltransferase (GNA1) from S. cerevisiae (UniProt ID P43577), two extra copies of the alpha-2,6-sialyltransferase PdbST from P. damselae (UniProt ID 066375) and the acetyl-coenzyme A synthetase (acs) from E. coli (UniProt ID P27550). The novel strain was evaluated in a growth experiment according to the culture conditions provided in Example 2, in which the culture medium contained 30 g/L sucrose and 20 g/L lactose. The strain was grown in four biological replicates in a 96-well plate. After 72 h of incubation, the culture broth was harvested, and the sugars were analysed on UPLC. The experiment demonstrated the novel strain produced sialic acid (Neu5Ac) and 6′-SL and did not suffer from any genomic or plasmid DNA instability or reorganisation during cultivation.

### Example 8. Production of 6′-sialyllactose (6′-SL) or 3′-sialyllactose (3′-SL) with a Modified E. coli Strain

An E. coli K-12 strain MG1655 is modified for sialic acid production as described in Example 2 comprising knock-outs of the E. coli nagA, nagB, nanA, nanT, nanE, nanK, LacZ, LacY and LacA genes and genomic knock-ins of constitutive transcriptional units comprising the genes encoding the lactose permease (LacY) from E. coli (UniProt ID P02920), the sialic acid transporter (nanT) from E. coli ((UniProt ID P41036), two copies of the L-glutamine D-fructose-6-phosphate aminotransferase (glmS*54) from E. coli (differing from the wild-type E. coli glmS, having UniProt ID P17169, by an A39T, an R250C and an G472S mutation), the phosphoglucosamine mutase (glmM) from E. coli (UniProt ID P31120), the N-acetylglucosamine-1-phosphate uridyltransferase/glucosamine-1-phosphate acetyltransferase (glmU) from E. coli (UniProt ID P0ACC7), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). The thus created strain sINB8 is further modified with genomic knock-ins of constitutive transcriptional units comprising either the genes encoding the UDP-N-acetylglucosamine 2-epimerase (NeuC) from C. jejuni (UniProt ID Q93MP8) and the N-acetylneuraminate synthase (NeuB) from N. meningitidis (UniProt ID E0NCD4) resulting in strain sINB8CB, or the genes encoding the bifunctional UDP-GlcNAc 2-epimerase/N-acetylmannosamine kinase from M. musculus (strain C57BL/6J) (UniProt ID Q91WG8), the N-acylneuraminate-9-phosphate synthetase from Pseudomonas sp. UW4 (UniProt ID K9NPH9) and the N-acylneuraminate-9-phosphatase from Candidatus magnetomorum sp. HK-1 (UniProt ID KPA15328.1) resulting in strain sINB8PS. The thus obtained mutant E. coli strains sINB8CB and sINB8PS are further modified with genomic knock-ins and an expression plasmid with constitutive transcriptional units to express three N-acylneuraminate cytidylyltransferases consisting of the NeuA enzyme from C. jejuni (UniProt ID Q93MP7), the NeuA enzyme from H. influenzae (GenBank No. AGV11798.1) and the NeuA enzyme from P. multocida (GenBank No. AMK07891.1) and either three copies of the polypeptide consisting of amino acid residues 108 to 497 of PdbST from P. damselae (UniProt ID 066375) having beta-galactoside alpha-2,6-sialyltransferase activity to produce 6′-SL or three copies of the polypeptide consisting of amino acid residues 1 to 268 of PmultST3 from P. multocida (UniProt ID Q9CLP3) having beta-galactoside alpha-2,3-sialyltransferase activity to produce 3′-SL. The final strains are evaluated in a growth experiment according to the culture conditions provided in Example 2, in which the culture medium contains 30 g/L sucrose and 20 g/L lactose. The strains are grown in four biological replicates in a 96-well plate. After 72 h of incubation, the culture broth is harvested, and the sugars are analysed on UPLC.

### Example 9. Evaluation of Mutant E. coli 6′-SL Production Strains in Fed-Batch Fermentations

The mutant E. coli strains as described in Example 5 were evaluated in a fed-batch fermentation process. Fed-batch fermentations at bioreactor scale were performed as described in Example 2. Sucrose was used as a carbon source and lactose was added in the batch medium as a precursor. No sialic acid (Neu5Ac) was added to the fermentation process. In contrast to the cultivation experiments that are described herein and wherein only end samples were taken at the end of cultivation (i.e., 72 hours as described herein), regular broth samples were taken at several time points during the fermentation process and the production of sialic acid (Neu5Ac) and 6′-sialyllactose at each of the time points was measured using UPLC as described in Example 2. The experiment demonstrated that broth samples taken e.g., at the end of the batch phase and during fed-batch phase comprised sialic acid production together with 6′-sialyllactose and unmodified lactose. Broth samples taken at the end of the fed-batch phase comprised 6′-sialyllactose and almost no or a very low concentration of Neu5Ac and almost no or a very low concentration of unmodified lactose demonstrating almost all or all of the precursor lactose was modified with almost all or all Neu5Ac produced during the fermentation of the mutant cells producing 6′-SL. The experiment further showed the mutant strains did not suffer from any genomic or plasmid DNA instability or reorganisation during cultivation.

### Example 10. Evaluation of Mutant E. coli 6′-SL or 3′-SL Production Strains in Fed-Batch Fermentations

The mutant E. coli strains as described in Examples 4, 6, 7 and 8 are evaluated in a fed-batch fermentation process. Fed-batch fermentations at bioreactor scale are performed as described in Example 2. Sucrose is used as a carbon source and lactose is added in the batch medium as a precursor. No sialic acid (Neu5Ac) is added to the fermentation process. In contrast to the cultivation experiments that are described herein and wherein only end samples were taken at the end of cultivation (i.e., 72 hours as described herein), regular broth samples are taken at several time points during the fermentation process and the production of 6′-sialyllactose or 3′-sialyllactose at each of the time points is measured using UPLC as described in Example 2.

### Example 11. Production of an Oligosaccharide Mixture Comprising 6′-SL, LacNAc, Sialylated LacNAc, LN3, Sialylated LN3, LNnT and LSTc with a Modified E. coli Host

Mutant E. coli strains modified for sialic acid production (Neu5Ac) and 6′-siayllactose as described in Examples 4, 5, 6 and 7 are further modified with genomic knock-ins comprising constitutive transcriptional units with two different coding DNA sequences chosen from the list comprising SEQ ID NOs:01 to 57 encoding one or two proteins with galactoside beta-1,3-N-acetylglucosaminyltransferase activity, and either 1) one or 2) two different coding DNA sequences chosen from the list comprising SEQ ID NOs:67 to 78 and encoding, respectively, 1) one or 2) one or two proteins with N-acetylglucosamine beta-1,4-galactosyltransferase activity to produce a mixture of oligosaccharides comprising 6′-SL, LacNAc, sialylated LacNAc, LN3, sialylated LN3, LNnT and LSTc (Neu5Ac-a2,6-Gal-b1,4-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 12. Production of an Oligosaccharide Mixture Comprising 6′-SL, LN3, Sialylated LN3, LNnT and LSTc with a Modified E. coli Host

Mutant E. coli strains modified for sialic acid production (Neu5Ac) and 6′-siayllactose as described in Example 8 are further modified with genomic knock-ins comprising constitutive transcriptional units with two different coding DNA sequences chosen from the list comprising SEQ ID NOs:01 to 57 encoding one or two proteins with galactoside beta-1,3-N-acetylglucosaminyltransferase activity, and either 1) one or 2) two different coding DNA sequences chosen from the list comprising SEQ ID NOs:67 to 78 and encoding, respectively, 1) one or 2) one or two proteins with N-acetylglucosamine beta-1,4-galactosyltransferase activity to produce a mixture of oligosaccharides comprising 6′-SL, LN3, sialylated LN3, LNnT and LSTc (Neu5Ac-a2,6-Gal-b1,4-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 13. Production of an Oligosaccharide Mixture LN3, Sialylated LN3, LNT, LNB, Sialylated LNB, 3′-SL and LSTa with a Modified E. coli Host

Mutant E. coli strains modified for sialic acid production (Neu5Ac) and 3′-siayllactose as described in Examples 4 and 6 are further modified with genomic knock-ins comprising constitutive transcriptional units with two different coding DNA sequences chosen from the list comprising SEQ ID NOs:01 to 57 encoding one or two proteins with galactoside beta-1,3-N-acetylglucosaminyltransferase activity and either 1) one or 2) two different coding DNA sequences chosen from the list comprising SEQ ID NOs:58 to 66 and encoding, respectively, 1) one or 2) one or two proteins with N-acetylglucosamine beta-1,3-galactosyltransferase activity to produce a mixture of oligosaccharides comprising LN3, 3′-sialylated LN3 (Neu5Ac-a2,3-GlcNAc-b1,3-Gal-b1,4-Glc), LNT, LNB, sialylated LNB, 3′-SL and LSTa (Neu5Ac-a2,3-Gal-b1,3-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 14. Production of an Oligosaccharide Mixture LN3, Sialylated LN3, LNT, 3′-SL and LSTa with a Modified E. coli Host

Mutant E. coli strains modified for sialic acid production (Neu5Ac) and 3′-siayllactose as described in Example 8 are further modified with genomic knock-ins comprising constitutive transcriptional units with two different coding DNA sequences chosen from the list comprising SEQ ID NOs:01 to 57 encoding one or two proteins with galactoside beta-1,3-N-acetylglucosaminyltransferase activity and either 1) one or 2) two different coding DNA sequences chosen from the list comprising SEQ ID NOs:58 to 66 and encoding, respectively, 1) one or 2) one or two proteins with N-acetylglucosamine beta-1,3-galactosyltransferase activity to produce a mixture of oligosaccharides comprising LN3, 3′-sialylated LN3 (Neu5Ac-a2,3-GlcNAc-b1,3-Gal-b1,4-Glc), LNT, 3′-SL and LSTa (Neu5Ac-a2,3-Gal-b1,3-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 15. Production of an Oligosaccharide Mixture Comprising LN3, Sialylated LN3, LNnT, LacNAc, Sialylated LacNAc, 3′-SL and LSTd with a Modified E. coli Host

Mutant E. coli strains modified for sialic acid production (Neu5Ac) and 3′-siayllactose as described in Examples 4 and 6 are further modified with genomic knock-ins comprising constitutive transcriptional units with two different coding DNA sequences chosen from the list comprising SEQ ID NOs:01 to 57 encoding one or two proteins with galactoside beta-1,3-N-acetylglucosaminyltransferase activity, and either 1) one or 2) two different coding DNA sequences chosen from the list comprising SEQ ID NOs:67 to 78 and encoding, respectively, 1) one or 2) one or two proteins with N-acetylglucosamine beta-1,4-galactosyltransferase activity to produce a mixture of oligosaccharides comprising 3′-SL, LN3, 3′-sialylated LN3 (Neu5Ac-a2,3-GlcNAc-b1,3-Gal-b1,4-Glc), LNnT, LacNAc, sialylated LacNAc and LSTd (Neu5Ac-a2,3-Gal-b1,4-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 16. Production of an Oligosaccharide Mixture Comprising LN3, Sialylated LN3, LNnT, 3′-SL and LSTd with a Modified E. coli Host

Mutant E. coli strains modified for sialic acid production (Neu5Ac) and 3′-siayllactose as described in Example 8 are further modified with genomic knock-ins comprising constitutive transcriptional units with two different coding DNA sequences chosen from the list comprising SEQ ID NOs:01 to 57 encoding one or two proteins with galactoside beta-1,3-N-acetylglucosaminyltransferase activity, and either 1) one or 2) two different coding DNA sequences chosen from the list comprising SEQ ID NOs:67 to 78 and encoding, respectively, 1) one or 2) one or two proteins with N-acetylglucosamine beta-1,4-galactosyltransferase activity to produce a mixture of oligosaccharides comprising 3′-SL, LN3, 3′-sialylated LN3 (Neu5Ac-a2,3-GlcNAc-b1,3-Gal-b1,4-Glc), LNnT and LSTd (Neu5Ac-a2,3-Gal-b1,4-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 17. Production of LN3 with a Modified E. coli Strain

An E. coli K-12 strain MG1655 is modified as described in Example 2 comprising knock-outs of the E. coli nagB, galT, ushA, agp, ldhA, LacZ, LacY and LacA genes and genomic knock-ins of constitutive transcriptional units comprising the genes encoding the lactose permease (LacY) from E. coli (UniProt ID P02920), the sucrose transporter (CscB) from E. coli W (UniProt ID E0IXR1), the fructose kinase (Frk) from Z. mobilis (UniProt ID Q03417) and the sucrose phosphorylase BaSP from B. adolescentis (UniProt ID A0ZZH6). In a next step, the mutant E. coli strain is modified for LN3 production with genomic knock-ins of constitutive transcriptional units comprising at least two different coding DNA sequences chosen from the list comprising SEQ ID NO:01 to 57 encoding one or more proteins with galactoside beta-1,3-N-acetylglucosaminyltransferase activity. The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 18. Production of Lacto-N-Tetraose (LNT) with a Modified E. coli Strain

In a next experiment, the LN3 producing E. coli strains described in Example 17 are further modified with constitutive transcriptional units delivered to the strain via genomic knock-in and/or from an expression plasmid comprising at least two different coding DNA sequences chosen from the list comprising SEQ ID NOs:58 to 66 and encoding one or more proteins with an N-acetylglucosamine beta-1,3-galactosyltransferase activity. The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 19. Production of Lacto-N-Neotetraose (LNnT) with a Modified E. coli Strain

In a next experiment, the LN3 producing E. coli strains described in Example 17 are further modified with constitutive transcriptional units delivered to the strain via genomic knock-in and/or from an expression plasmid comprising at least two different coding DNA sequences chosen from the list comprising SEQ ID NOs:67 to 78 and encoding one or more proteins with an N-acetylglucosamine beta-1,4-galactosyltransferase activity. The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 20. Production of LNT with a Modified E. coli Strain

An E. coli K-12 strain MG1655 was modified as described in Example 2 comprising knock-outs of the E. coli nagB, galT, ushA, ldhA, LacZ, LacY and LacA genes and genomic knock-ins of constitutive transcriptional units comprising the genes encoding the lactose permease (LacY) from E. coli (UniProt ID P02920), the sucrose transporter (CscB) from E. coli W (UniProt ID E0IXR1), the fructose kinase (Frk) from Z. mobilis (UniProt ID Q03417), the sucrose phosphorylase BaSP from B. adolescentis (UniProt ID A0ZZH6), the coding DNA sequence with SEQ ID NO:03 encoding the galactoside beta-1,3-N-acetylglucosaminyltransferase lgtA from N. meningitidis with SEQ ID NO:80, the coding DNA sequence with SEQ ID NO:60 from Pseudogulbenkiania ferrooxidans encoding the N-acetylglucosamine beta-1,3-galactosyltransferase with SEQ ID NO:133 and the coding DNA sequence with SEQ ID NO:63 from Salmonella enterica encoding the N-acetylglucosamine beta-1,3-galactosyltransferase with SEQ ID NO:134, respectively, resulting in strain sINB010952 (Table 4). In a next step, the mutant strain sINB010952 was further modified with a genomic knock-in of a constitutive transcriptional unit with the coding DNA sequence with SEQ ID NO:6 encoding for an additional copy of the galactoside beta-1,3-N-acetylglucosaminyltransferase lgtA from N. meningitidis with SEQ ID NO:80, resulting in strain sINB011744 (Table 4). The novel strains were evaluated in a growth experiment according to the culture conditions provided in Example 2, in which the culture medium contained 30 g/L sucrose and 20 g/L lactose. Each strain was grown in four biological replicates in a 96-well plate. After 72 h of incubation, the culture broth was harvested, and the sugars were analysed on UPLC. Both strains demonstrated to produce LN3 and LNT and did not suffer from any genomic or plasmid DNA instability or reorganisation during cultivation. Hereby, strain sINB011744 having two different coding DNA sequences encoding the same lgtA polypeptide with SEQ ID NO:80 produced almost double titers of LNT compared to strain sINB010952 having only one coding DNA sequence for lgtA with SEQ ID NO:80. As shown in Table 5, also the relative production of LNT (in %, compared to the total sum of LNT and LN3 produced) was higher in strain sINB011744 than in strain sINB010952.

TABLE 4
Mutant E. coli strains with one or two galactoside
beta-1,3-N-acetylglucosaminyltransferase(s) (B3GlcNAcT) and two N-acetylglucosamine
beta-1,3-galactosyltransferases (B3GalT) for LN3 and LNT production.
SEQ ID NOs correspond to the corresponding coding DNA sequences.

| | First | Second | | |
|---|---|---|---|---|
| Strain | B3GlcNAcT | B3GlcNAcT | First B3GalT | Second B3GalT |
| sINB010952 | SEQ ID NO: 03 | / | SEQ ID NO: 60 | SEQ ID NO: 63 |
| sINB011744 | SEQ ID NO: 03 | SEQ ID NO: 06 | SEQ ID NO: 60 | SEQ ID NO: 63 |

TABLE 5
Relative production of LN3 (%) and LNT (%) compared
to the total sum of LN3 and LNT produced in mutant
E. coli strains expressing one or two galactoside
beta-1,3-N-acetylglucosaminyltransferase(s) (B3GlcNAcT)
and two N-acetylglucosamine beta-1,3-galactosyltransferases
(B3GalT) as shown in Table 4, when evaluated in
a growth experiment according to the culture conditions
provided in Example 2, in which the culture medium
contained 30 g/L sucrose as carbon source and 20
g/L lactose as precursor.

| Strain | LN3 (%) | LNT (%) |
|---|---|---|
| sINB010952 | 21.7 | 78.3 |
| sINB011744 | 17.0 | 83.0 |

### Example 21. Production of LNT with a Modified E. coli Strain

In another experiment, an E. coli K-12 strain MG1655 was modified as described in Example 2 comprising knock-outs of the E. coli nagB, galT, ushA, ldhA, LacZ, LacY and LacA genes and genomic knock-ins of constitutive transcriptional units comprising the genes encoding the lactose permease (LacY) from E. coli (UniProt ID P02920), the sucrose transporter (CscB) from E. coli W (UniProt ID E0IXR1), the fructose kinase (Frk) from Z. mobilis (UniProt ID Q03417), the sucrose phosphorylase BaSP from B. adolescentis (UniProt ID A0ZZH6, the coding DNA sequence with SEQ ID NO:63 from Salmonella enterica encoding the N-acetylglucosamine beta-1,3-galactosyltransferase with SEQ ID NO:134 and either the coding DNA sequences with SEQ ID NO:03 and SEQ ID NO:07 encoding the galactoside beta-1,3-N-acetylglucosaminyltransferases from N. meningitidis with SEQ ID NOs:80 and 81, respectively, or the coding DNA sequences with SEQ ID NO:03 and SEQ ID NO:06 encoding the galactoside beta-1,3-N-acetylglucosaminyltransferase from N. meningitidis with SEQ ID NO:80, resulting in strains sINB010938 and sINB011126, respectively (Table 6). In a next step, both mutant strains were further modified with a genomic knock-in of a constitutive transcriptional unit with the coding DNA sequence with SEQ ID NO:60 from Pseudogulbenkiania ferrooxidans encoding for a second N-acetylglucosamine beta-1,3-galactosyltransferase with SEQ ID NO:133, resulting in strains sINB011450 and sINB011744, respectively (Table 6). The novel strains were evaluated in a growth experiment according to the culture conditions provided in Example 2, in which the culture medium contained 30 g/L sucrose and 20 g/L lactose. Each strain was grown in four biological replicates in a 96-well plate. After 72 h of incubation, the culture broth was harvested, and the sugars were analysed on UPLC. All strains demonstrated to produce LN3 and LNT and did not suffer from any genomic or plasmid DNA instability or reorganisation during cultivation. Hereby, the strains sINB011450 and sINB011744, both having two different coding DNA sequences encoding N-acetylglucosamine beta-1,3-galactosyltransferases, produced 10% more LNT compared to their respective reference strains, sINB010938 and sINB011126 respectively, having only one coding DNA sequence encoding an N-acetylglucosamine beta-1,3-galactosyltransferase. As shown in Table 7, also the relative production of LNT (in %, compared to the total sum of LNT and LN3 produced) was higher in strains sINB011450 and sINB011744 than in their respective strains sINB010938 and sINB011126 respectively.

TABLE 6
Mutant E. coli strains with two galactoside beta-1,3-
N-acetylglucosaminyltransferases (B3GlcNAcT) and one
or two N-acetylglucosamine beta-1,3-galactosyltransferase(s)
(B3GalT) for LN3 and LNT production. SEQ ID NOs correspond
to the corresponding coding DNA sequences.

| Strain | B3GlcNAcTs present | B3GalTs present |
|---|---|---|
| sINB010938 | SEQ ID NOs: 03 + 07 | SEQ ID NO: 63 |
| sINB011450 | SEQ ID NOs: 03 + 07 | SEQ ID NOs: 63 + 60 |
| sINB011126 | SEQ ID NOs: 03 + 06 | SEQ ID NO: 63 |
| sINB011744 | SEQ ID NOs: 03 + 06 | SEQ ID NOs: 63 + 60 |

TABLE 7
Relative production of LN3 (%) and LNT (%) compared
to the total sum of LN3 and LNT produced in mutant
E. coli strains expressing one or two galactoside
beta-1,3-N-acetylglucosaminyltransferase(s) (B3GlcNAcT)
and two N-acetylglucosamine beta-1,3-galactosyltransferases
(B3GalT) as shown in Table 6, when evaluated in
a growth experiment according to the culture conditions
provided in Example 2, in which the culture medium
contained 30 g/L sucrose as carbon source and 20
g/L lactose as precursor.

| Strain | LN3 (%) | LNT (%) |
|---|---|---|
| sINB010938 | 25.6 | 74.4 |
| sINB011450 | 21.3 | 78.7 |
| sINB011126 | 23.1 | 76.9 |
| sINB011744 | 17.0 | 83.0 |

### Example 22. Production of LNnT with a Modified E. coli Strain

An E. coli K-12 strain MG1655 was modified for LN3 production as described in Example 2 comprising knock-outs of the E. coli nagB, galT, ushA, ldhA, LacZ, LacY and LacA genes and genomic knock-ins of constitutive transcriptional units comprising the lactose permease (LacY) from E. coli (UniProt ID P02920), the sucrose transporter (CscB) from E. coli W (UniProt ID E0IXR1), the fructose kinase (Frk) from Z. mobilis (UniProt ID Q03417), the sucrose phosphorylase BaSP from B. adolescentis (UniProt ID A0ZZH6) and the two coding DNA sequences with SEQ ID NO:03 and SEQ ID NO:06, both encoding the galactoside beta-1,3-N-acetylglucosaminyltransferase lgtA from N. meningitidis with SEQ ID NO:80. In a next step for LNnT production, the mutant LN3 strain was further modified with a genomic knock-in of a constitutive transcriptional unit with the coding DNA sequence with SEQ ID NO:68 and encoding the N-acetylglucosamine beta-1,4-galactosyltransferase lgtB from N. meningitidis with SEQ ID NO:137, resulting in strain sINB010632 (Table 8). In a further step, the strain sINB010632 was modified with a genomic knock-in of a constitutive transcriptional unit with the coding DNA sequence with either SEQ ID NO:71 or 72, each encoding a second N-acetylglucosamine beta-1,4-galactosyltransferase, being either CpsIaJ from Streptococcus agalactiae with SEQ ID NO:138 or GalT from Helicobacter pylori with SEQ ID NO:139, respectively, resulting in strains sINB010949 and sINB010950 (Table 8). The novel strains were evaluated in a growth experiment according to the culture conditions provided in Example 2, in which the culture medium contained 30 g/L sucrose and 20 g/L lactose. Each strain was grown in four biological replicates in a 96-well plate. After 72 h of incubation, the culture broth was harvested, and the sugars were analysed on UPLC. The novel strains demonstrated to produce LNnT and did not suffer from any genomic or plasmid DNA instability or reorganisation during cultivation. Hereby, the strains sINB010949 and sINB010950, both having two different coding DNA sequences encoding N-acetylglucosamine beta-1,4-galactosyltransferases, produced 10% more LNnT compared to the reference strain sINB010632, having only one coding DNA sequence encoding an N-acetylglucosamine beta-1,4-galactosyltransferase. As shown in Table 9, also the relative production of LNnT (in %, compared to the total sum of LNnT and LN3 produced) was higher in strains sINB010949 and sINB010950 and no LN3 leftover was detectable in the strains compared to the reference strain sINB010632.

TABLE 8
Mutant E. coli strains with two galactoside beta-1,3-
N-acetylglucosaminyltransferases (B3GlcNAcT) and one or
two N-acetylglucosamine beta-1,4-galactosyltransferase(s)
(B4GalT) for LN3 and LNnT production. SEQ ID NOs correspond
to the corresponding coding DNA sequences.

| Strain | B3GlcNAcTs present | B3GalTs present |
|---|---|---|
| sINB010632 | SEQ ID NOs: 03 + 06 | SEQ ID NO: 68 |
| sINB010949 | SEQ ID NOs: 03 + 06 | SEQ ID NOs: 68 + 71 |
| sINB010950 | SEQ ID NOs: 03 + 06 | SEQ ID NOs: 68 + 72 |

TABLE 9
Relative production of LN3 (%) and LNnT (%) compared to the total
sum of LN3 and LNnT produced in mutant E. coli strains expressing
two galactoside beta-1,3-N-acetylglucosaminyltransferases (B3GlcNAcT)
and one or two N-acetylglucosamine beta-1,4-galactosyltransferases
(B4GalT) as shown in Table 8, when evaluated in a growth experiment
according to the culture conditions provided in Example 2, in
which the culture medium contained 30 g/L sucrose as carbon source
and 20 g/L lactose as precursor.

| Strain | LN3 (%) | LNnT (%) |
|---|---|---|
| sINB010632 | 28.0 | 72.0 |
| sINB010949 | 0 | 100 |
| sINB010950 | 0 | 100 |

### Example 23. Production of LNnT with a Modified E. coli Strain

In a next experiment, the mutant strain sINB010950 as described in Example 22, was further modified with a knock-out of the E. coli agp gene. The novel strain sINB011969 was evaluated in a growth experiment according to the culture conditions provided in Example 2, in which the culture medium contained 30 g/L sucrose and 20 g/L lactose. The strain was grown in four biological replicates in a 96-well plate. After 72 h of incubation, the culture broth was harvested, and the sugars were analysed on UPLC. The novel strain demonstrated to produce 0.01±0.01 g/L LN3 and 0.52±0.20 g/L LNnT and did not suffer from any genomic DNA instability or reorganisation during cultivation.

### Example 24. Evaluation of a Mutant E. coli LNT Production Strain in Fed-Batch Fermentations

The mutant E. coli strain sINB011744 as described in Example 20 was evaluated in a fed-batch fermentation process. Fed-batch fermentations at bioreactor scale were performed as described in Example 2. Sucrose was used as a carbon source and lactose was added in the batch medium as a precursor. In contrast to the cultivation experiments that are described herein and wherein only end samples were taken at the end of cultivation (i.e., 72 hours as described herein), regular broth samples were taken at several time points during the fermentation process and the production of LN3 and LNT at each of the time points was measured using UPLC as described in Example 2. The experiment demonstrated the strain obtained a relative production of 21.0% of LN3 and 79.0% of LNT in broth samples taken after 72 h of fermentation (calculated by dividing the average production titre of LN3 or of LNT by the sum of the average production titers of LN3 and LNT produced).

### Example 25. Evaluation of Mutant E. coli LNnT Production Strains in Fed-Batch Fermentations

The mutant E. coli strains sINB010949 and sINB011969 as described in Examples 22 and 23, respectively, were evaluated in a fed-batch fermentation process. Fed-batch fermentations at bioreactor scale are performed as described in Example 2. Sucrose is used as a carbon source and lactose is added in the batch medium as a precursor. In contrast to the cultivation experiments that are described herein and wherein only end samples were taken at the end of cultivation (i.e., 72 hours as described herein), regular broth samples are taken at several time points during the fermentation process and the production of LN3 and LNnT at each of the time points is measured using UPLC as described in Example 2. The experiment demonstrated the strains obtained a relative production of 5-7% of LN3 and 95-97% of LNnT in broth samples taken after 72 hours of fermentation (calculated by dividing the average production titre of LN3 or of LNnT by the sum of the average production titers of LN3 and LNnT produced).

### Example 26. Production of LNFP-I with a Modified E. coli Strain

The mutant LNT producing E. coli strains as described in Examples 18, 20 and 21 are further modified for production of lacto-N-fucopentaose I (LNFP-I, Fuc-a1,2-Gal-b1,3-GlcNAc-b1,3-Gal-b1,4-Glc) by adding a constitutive transcriptional unit, either expressed from a plasmid or integrated into the genome, for an a1,2-fucosyltransferase enzyme able to transfer fucose from GFP-fucose to the terminal galactose of LNT in an alpha-1,2 linkage like e.g., from Brachyspira pilosicoli (UniProt ID A0A2N5RQ26), Dysgonomonas mossii (UniProt ID F8X274), Dechlorosoma suillum (UniProt ID G8QLF4), Polaribacter vadi (UniProt ID A0A1B8TNT0) or Desulfovibrio alaskensis (UniProt ID Q316B5). The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 27. Production of LNFP-II with a Modified E. coli Strain

The mutant LNT producing E. coli strains as described in Examples 18, 20 and 21 are further modified for production of lacto-N-fucopentaose II (LNFP-II, Gal-b1,3-(Fuc-a1,4)-GlcNAc-b1,3-Gal-b1,4-Glc) by adding a constitutive transcriptional unit, either expressed from a plasmid or integrated into the genome, for a mutant a1,3/4 fucosidase from Bifidobacterium longum subsp. infantis ATCC 15697 as described in WO 2016/063261. The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 28. Production of LNFP-V with a Modified E. coli Strain

The mutant LNT producing E. coli strains as described in Examples 18, 20 and 21 are further modified for production of lacto-N-fucopentaose V (LNFP-V, Gal-b1,3-GlcNAc-b1,3-Gal-b1,4-(Fuca1,3)-Glc) by adding a constitutive transcriptional unit, either expressed from a plasmid or integrated into the genome, for a truncated form missing 66 amino acid residues at the C-terminus of the alpha-1,3-fucosyltransferase HpFucT from Helicobacter pylori (UniProt ID 030511) as described by Bai et al. (Carb. Res. 2019, 480, 1-6). The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 29. Production of LNFP-III with a Modified E. coli Strain

The mutant LNnT producing E. coli strains as described in Examples 19, 22 and 23 are further modified for production of lacto-N-fucopentaose III (LNFP-III, Gal-b1,4-(Fuc-a1,3)-GlcNAc-b1,3-Gal-b1,4-Glc) by adding a constitutive transcriptional unit, either expressed from a plasmid or integrated into the genome, for a truncated form missing 66 amino acid residues at the C-terminus of the alpha-1,3-fucosyltransferase HpFucT from Helicobacter pylori (UniProt ID 030511) as described by Bai et al. (Carb. Res. 2019, 480, 1-6). The novel strains are evaluated in a growth experiment according to the culture conditions provided in Example 2, in which the culture medium contains 30 g/L sucrose and 20 g/L lactose. Each strain is grown in four biological replicates in a 96-well plate. After 72 h of incubation, the culture broth is harvested, and the sugars are analysed on UPLC.

### Example 30. Production of GalNAc-LNFPI with a Modified E. coli Strain

The mutant LNFP-I producing E. coli strains as described in Example 26 were further adapted for UDP-N-acetylgalactosamine (UDP-GalNAc) production with a genomic knock-in of a constitutive transcriptional unit for the 4-epimerase (WbpP) of Pseudomonas aeruginosa (UniProt ID Q8KN66). In a next step to allow the strains to produce GalNAc-LNFPI (GalNAc-a1,3-(Fuc-a1,2)-Gal-b1,3-GlcNAc-b1,3-Gal-b1,4-Glc), the strains were further modified with constitutive transcriptional units encoding the glycoprotein-fucosylgalactoside alpha-N-acetylgalactosaminyltransferases from Helicobacter mustelae (GenBank No. SQH71958), Bacteroides ovatus (UniProt ID A7LVT2 and/or A0A395VXC9), Lachnospiraceae bacterium (UniProt ID A0A1I3AV07) and/or Roseburia inulinivorans (UniProt ID A0A3R5VYF4). The novel strains were evaluated in a growth experiment according to the culture conditions provided in Example 2, in which the culture medium contained 30 g/L sucrose and 20 g/L lactose. Each strain was grown in four biological replicates in a 96-well plate. After 72 h of incubation, the culture broth was harvested, and the sugars were analysed on UPLC. The novel strains demonstrated to produce LN3, LNT, LNFPI and GalNAc-LNFPI and did not suffer from any genomic or plasmid DNA instability or reorganisation during cultivation.

### Example 31. Production of Gal-LNFP-I with a Modified E. coli Strain

The mutant LNFPI producing E. coli strains as described in Example 26 are further adapted to produce Gal-LNFP-I (Gal-a1,3-(Fuc-a1,2)-Gal-b1,3-GlcNAc-b1,3-Gal-b1,4-Glc) with a genomic knock-in of a constitutive expression unit for the alpha-1,3-galactosyltransferase WbnI from E. coli (UniProt ID Q5JBG6). The novel strains are evaluated in a growth experiment according to the culture conditions provided in Example 2, in which the culture medium contains 30 g/L sucrose and 20 g/L lactose. The strains are grown in four biological replicates in a 96-well plate. After 72 h of incubation, the culture broth is harvested, and the sugars are analysed on UPLC.

### Example 32. Production of an Oligosaccharide Mixture LN3, Sialylated LN3, LNT, 3′-SL and LSTa with a Modified E. coli Host

The mutant LNT producing E. coli strains as described in Examples 18, 20 and 21 are further modified with genomic knock-ins of constitutive expression units comprising the genes encoding the L-glutamine D-fructose-6-phosphate aminotransferase (glmS*54) from E. coli (differing from the wild-type E. coli glmS, having UniProt ID P17169, by an A39T, an R250C and an G472S mutation), the phosphoglucosamine mutase (glmM) from E. coli (UniProt ID P31120), the N-acetylglucosamine-1-phosphate uridyltransferase/glucosamine-1-phosphate acetyltransferase (glmU) from E. coli (UniProt ID P0ACC7), the UDP-N-acetylglucosamine 2-epimerase (NeuC) from C. jejuni (UniProt ID Q93MP8), the N-acetylneuraminate synthase (NeuB) from N. meningitidis (UniProt ID E0NCD4), the sialic acid transporter (nanT) from E. coli ((UniProt ID P41036), the N-acylneuraminate cytidylyltransferases from C. jejuni (UniProt ID Q93MP7), H. influenzae (GenBank No. AGV11798.1) and P. multocida (GenBank No. AMK07891.1) and the beta-galactoside alpha-2,3-sialyltransferase PmultST3 from P. multocida (UniProt ID Q9CLP3) to produce a mixture of oligosaccharides comprising LN3, 3′-sialylated LN3 (Neu5Ac-a2,3-GlcNAc-b1,3-Gal-b1,4-Glc), LNT, 3′-SL and LSTa (Neu5Ac-a2,3-Gal-b1,3-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 33. Production of an Oligosaccharide Mixture Comprising 6′-SL, LN3, Sialylated LN3, LNnT and LSTc with a Modified E. coli Host

The mutant LNnT producing E. coli strains as described in Examples 22 and 23 are further modified with genomic knock-ins of constitutive expression units comprising the genes encoding the L-glutamine D-fructose-6-phosphate aminotransferase (glmS*54) from E. coli (differing from the wild-type E. coli glmS, having UniProt ID P17169, by an A39T, an R250C and an G472S mutation), the phosphoglucosamine mutase (glmM) from E. coli (UniProt ID P31120), the N-acetylglucosamine-1-phosphate uridyltransferase/glucosamine-1-phosphate acetyltransferase (glmU) from E. coli (UniProt ID P0ACC7), the UDP-N-acetylglucosamine 2-epimerase (NeuC) from C. jejuni (UniProt ID Q93MP8), the N-acetylneuraminate synthase (NeuB) from N. meningitidis (UniProt ID E0NCD4), the sialic acid transporter (nanT) from E. coli ((UniProt ID P41036), the N-acylneuraminate cytidylyltransferases from C. jejuni (UniProt ID Q93MP7), H. influenzae (GenBank No. AGV11798.1) and P. multocida (GenBank No. AMK07891.1) and the beta-galactoside alpha-2,6-sialyltransferase PdbST from P. damselae (UniProt ID 066375) to produce a mixture of oligosaccharides comprising 6′-SL, LN3, sialylated LN3, LNnT and LSTc (Neu5Ac-a2,6-Gal-b1,4-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 34. Production of an Oligosaccharide Mixture Comprising LN3, Sialylated LN3, LNnT, 3′-SL and LSTd with a Modified E. coli Host

The mutant LNnT producing E. coli strains as described in Examples 22 and 23 are further modified with genomic knock-ins of constitutive expression units comprising the genes encoding the L-glutamine D-fructose-6-phosphate aminotransferase (glmS*54) from E. coli (differing from the wild-type E. coli glmS, having UniProt ID P17169, by an A39T, an R250C and an G472S mutation), the phosphoglucosamine mutase (glmM) from E. coli (UniProt ID P31120), the N-acetylglucosamine-1-phosphate uridyltransferase/glucosamine-1-phosphate acetyltransferase (glmU) from E. coli (UniProt ID P0ACC7), the UDP-N-acetylglucosamine 2-epimerase (NeuC) from C. jejuni (UniProt ID Q93MP8), the N-acetylneuraminate synthase (NeuB) from N. meningitidis (UniProt ID E0NCD4), the sialic acid transporter (nanT) from E. coli ((UniProt ID P41036), the N-acylneuraminate cytidylyltransferases from C. jejuni (UniProt ID Q93MP7), H. influenzae (GenBank No. AGV11798.1) and P. multocida (GenBank No. AMK07891.1) and the beta-galactoside alpha-2,3-sialyltransferase PmultST3 from P. multocida (UniProt ID Q9CLP3) to produce a mixture of oligosaccharides comprising 3′-SL, LN3, 3′-sialylated LN3 (Neu5Ac-a2,3-GlcNAc-b1,3-Gal-b1,4-Glc), LNnT and LSTd (Neu5Ac-a2,3-Gal-b1,4-GlcNAc-b1,3-Gal-b1,4-Glc). The novel strains are evaluated in a growth experiment according to the culture conditions provided in Example 2, in which the culture medium contains sucrose and lactose. The strains are grown in four biological replicates in a 96-well plate. After 72 h of incubation, the culture broth is harvested, and the sugars are analysed on UPLC.

### Example 35. Production of LNnT with a Modified E. coli Strain

The mutant LNnT producing E. coli strains as described in Examples 19, 22 and 23 are further modified with genomic knock-ins of constitutive transcriptional units comprising the genes encoding the membrane transporter proteins MdfA from Citrobacter youngae (UniProt ID D4BC23) and MdfA from Yokenella regensburgei (UniProt ID G9Z5F4). The novel strains are evaluated in a growth experiment according to the culture conditions provided in Example 2, in which the culture medium contains 30 g/L sucrose and 20 g/L lactose. The strains are grown in four biological replicates in a 96-well plate. After 72 h of incubation, the culture broth is harvested, and the sugars are analysed on UPLC.

### Example 36. Production of 6′-sialyllactose (6′-SL) with a Modified S. cerevisiae Strain

An S. cerevisiae strain is adapted for sialic acid (Neu5Ac) and sialylated lactose production as described in Example 3 with a pRS420-derived yeast expression plasmid comprising the TRP1 selection marker and constitutive transcriptional units for two copies of the mutant L-glutamine D-fructose-6-phosphate aminotransferase (glmS*54) from E. coli (differing from the wild-type E. coli glmS, having UniProt ID P17169, by an A39T, an R250C and an G472S mutation as described by Deng et al. (Biochimie 88, 419-29 (2006)), a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae and BsAraL from B. subtilis as described in WO 2018122225, the N-acetylglucosamine 2-epimerase (AGE) from B. ovatus (UniProt ID A7LVG6), the N-acetylneuraminate synthase (NeuB) from N. meningitidis (UniProt ID E0NCD4), three N-acylneuraminate cytidylyltransferases consisting of the NeuA enzyme from C. jejuni (UniProt ID Q93MP7), the NeuA enzyme from Haemophilus influenzae (GenBank No. AGV11798.1) and the NeuA enzyme from Pasteurella multocida (GenBank No. AMK07891.1), three copies of the PdST6-like polypeptide from Photobacterium damselae consisting of amino acid residues 108 to 497 of UniProt ID 066375 and the lactose permease (LAC12) from K. lactis (UniProt ID P07921). The novel strain is evaluated in a growth experiment on SD CSM-Trp drop-out medium comprising lactose as precursor according to the culture conditions provided in Example 3. After 72 h of incubation, the culture broth is harvested, and the sugars are analysed on UPLC.

### Example 37. Production of an Oligosaccharide Mixture Comprising 6′-SL, LN3, Sialylated LN3, LNnT and LSTc with a Modified S. cerevisiae Host

The mutant S. cerevisiae strain described in Example 36 is further modified with a second pRS420-derived yeast expression plasmid comprising the HIS3 selection marker and constitutive transcriptional units for galE from E. coli (UniProt ID P09147), two or more different coding DNA sequences chosen from the list comprising 01 to 57 and encoding one or more proteins with a galactoside beta-1,3-N-acetylglucosaminyltransferase activity and the N-acetylglucosamine beta-1,4-galactosyltransferase (lgtB) from N. meningitidis with SEQ ID NO:137 to produce a mixture of oligosaccharides comprising 6′-SL, LN3, sialylated LN3, LNnT and LSTc (Neu5Ac-a2,6-Gal-b1,4-GlcNAc-b1,3-Gal-b1,4-Glc). The novel strain is evaluated in a growth experiment on SD CSM-Trp-His drop-out medium comprising lactose as precursor according to the culture conditions provided in Example 3. After 72 h of incubation, the culture broth is harvested, and the sugars are analysed on UPLC.

### Example 38. Production of 3′-sialyllactose (3′-SL) with a Modified S. cerevisiae Strain

An S. cerevisiae strain is adapted for sialic acid (Neu5Ac) and sialylated lactose production as described in Example 3 with a pRS420-derived yeast expression plasmid comprising the TRP1 selection marker and constitutive transcriptional units for two copies of the mutant L-glutamine D-fructose-6-phosphate aminotransferase (glmS*54) from E. coli (differing from the wild-type E. coli glmS, having UniProt ID P17169, by an A39T, an R250C and an G472S mutation as described by Deng et al. (Biochimie 88, 419-29 (2006)), a phosphatase like any one or more of e.g., the E. coli genes comprising aphA, Cof, HisB, OtsB, SurE, Yaed, YcjU, YedP, YfbT, YidA, YigB, YihX, YniC, YqaB, YrbL, AppA, Gph, SerB, YbhA, YbiV, YbjL, Yfb, YieH, YjgL, YjjG, YrfG and YbiU or PsMupP from P. putida, ScDOG1 from S. cerevisiae and BsAraL from B. subtilis as described in WO 2018122225, the N-acetylglucosamine 2-epimerase (AGE) from B. ovatus (UniProt ID A7LVG6), the N-acetylneuraminate synthase (NeuB) from N. meningitidis (UniProt ID E0NCD4), three N-acylneuraminate cytidylyltransferases consisting of the NeuA enzyme from C. jejuni (UniProt ID Q93MP7), the NeuA enzyme from Haemophilus influenzae (GenBank No. AGV11798.1) and the NeuA enzyme from Pasteurella multocida (GenBank No. AMK07891.1), three copies of the PmultST3-like polypeptide from P. multocida consisting of amino acid residues 1 to 268 of UniProt ID Q9CLP3 and the lactose permease (LAC12) from K. lactis (UniProt ID P07921). The novel strain is evaluated in a growth experiment on SD CSM-Trp drop-out medium comprising lactose as precursor according to the culture conditions provided in Example 3. After 72 h of incubation, the culture broth is harvested, and the sugars are analysed on UPLC.

### Example 39. Production of an Oligosaccharide Mixture Comprising LN3, Sialylated LN3, LNT, 3′-SL and LSTa with a Modified S. cerevisiae Host

The mutant S. cerevisiae strain described in Example 38 is further modified with a second pRS420-derived yeast expression plasmid comprising the HIS3 selection marker and constitutive transcriptional units for galE from E. coli (UniProt ID P09147), two or more different coding DNA sequences chosen from the list comprising 01 to 57 and encoding one or more proteins with a galactoside beta-1,3-N-acetylglucosaminyltransferase activity and the N-acetylglucosamine beta-1,3-galactosyltransferase (wbgO) from E. coli O55:H7 with SEQ ID NO:132 to produce a mixture of oligosaccharides comprising LN3, 3′-sialylated LN3 (Neu5Ac-a2,3-GlcNAc-b1,3-Gal-b1,4-Glc), LNT, 3′-SL and LSTa (Neu5Ac-a2,3-Gal-b1,3-GlcNAc-b1,3-Gal-b1,4-Glc). The novel strain is evaluated in a growth experiment on SD CSM-Trp-His drop-out medium comprising lactose as precursor according to the culture conditions provided in Example 3. After 72 h of incubation, the culture broth is harvested, and the sugars are analysed on UPLC.

### Example 40. Production of an Oligosaccharide Mixture Comprising LN3, Sialylated LN3, LNnT, 3′-SL and LSTd with a Modified S. cerevisiae Host

The mutant S. cerevisiae strain described in Example 38 is further modified with a second pRS420-derived yeast expression plasmid comprising the HIS3 selection marker and constitutive transcriptional units for galE from E. coli (UniProt ID P09147), two or more different coding DNA sequences chosen from the list comprising 01 to 57 and encoding one or more proteins with a galactoside beta-1,3-N-acetylglucosaminyltransferase activity and the N-acetylglucosamine beta-1,4-galactosyltransferase (lgtB) from N. meningitidis with SEQ ID NO:137 to produce a mixture of oligosaccharides comprising 3′-SL, LN3, 3′-sialylated LN3 (Neu5Ac-a2,3-GlcNAc-b1,3-Gal-b1,4-Glc), LNnT and LSTd (Neu5Ac-a2,3-Gal-b1,4-GlcNAc-b1,3-Gal-b1,4-Glc). The novel strain is evaluated in a growth experiment on SD CSM-Trp-His drop-out medium comprising lactose as precursor according to the culture conditions provided in Example 3. After 72 h of incubation, the culture broth is harvested, and the sugars are analysed on UPLC.

### Example 41. Production of LN3 with a Modified S. cerevisiae Strain

An S. cerevisiae strain is adapted for LN3 production as described in Example 3 with a pRS420-derived yeast expression plasmid comprising the HIS3 selection marker and constitutive transcriptional units for the UDP-glucose-4-epimerase galE from E. coli (UniProt ID P09147), at least two different coding DNA sequences chosen from the list comprising SEQ ID NOs:1 to 57 and encoding one or more proteins with a galactoside beta-1,3-N-acetylglucosaminyltransferase activity and the lactose permease (LAC12) from K. lactis (UniProt ID P07921). The novel strains are evaluated in a growth experiment on SD CSM-His drop-out medium comprising lactose as precursor according to the culture conditions provided in Example 3. After 72 h of incubation, the culture broth is harvested, and the sugars are analysed on UPLC.

### Example 42. Production of LNT with a Modified S. cerevisiae Strain

The S. cerevisiae strains adapted for LN3 production as described in Example 41 are further modified with constitutive transcriptional units comprising at least one coding DNA sequence chosen from the list comprising SEQ ID NOs:58 to 66, encoding N-acetylglucosamine beta-1,3-galactosyltransferase proteins. The novel strains are evaluated in a growth experiment on SD CSM-His drop-out medium comprising lactose as precursor according to the culture conditions provided in Example 3. After 72 h of incubation, the culture broth is harvested, and the sugars are analysed on UPLC.

### Example 43. Production of LNnT with a Modified S. cerevisiae Strain

The S. cerevisiae strains adapted for LN3 production as described in Example 41 are further modified with constitutive transcriptional units comprising at least one coding DNA sequence chosen from the list comprising SEQ ID NOs:67 to 78, encoding N-acetylglucosamine beta-1,4-galactosyltransferase proteins. The novel strains are evaluated in a growth experiment on SD CSM-His drop-out medium comprising lactose as precursor according to the culture conditions provided in Example 3. After 72 h of incubation, the culture broth is harvested, and the sugars are analysed on UPLC.

### Example 44. Material and Methods Bacillus subtilis

### Media

Two different media are used, namely a rich Luria Broth (LB) and a minimal medium for shake flask (MMsf). The minimal medium uses a trace element mix.

Trace element mix consisted of 0.735 g/L CaCl₂·2H₂O, 0.1 g/L MnCl₂·2H₂O, 0.033 g/L CuCl₂·2H₂O, 0.06 g/L COCl₂·6H₂O, 0.17 g/L ZnCl₂, 0.0311 g/L H₃BO₄, 0.4 g/L Na₂EDTA·2H₂O and 0.06 g/L Na₂MoO₄. The Fe-citrate solution contained 0.135 g/L FeCl₃·6H₂O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR. Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L (NH₄)₂SO₄, 7.5 g/L KH₂PO₄, 17.5 g/L K₂HPO₄, 1.25 g/L Na-citrate, 0.25 g/L MgSO₄·7H₂O, 0.05 g/L tryptophan, from 10 up to 30 g/L glucose or another carbon source including but not limited to fructose, maltose, sucrose, glycerol and maltotriose when specified in the examples, 10 ml/L trace element mix and 10 ml/L Fe-citrate solution. The medium was set to a pH of 7 with 1M KOH. Depending on the experiment lactose, LNB or LacNAc could be added as a precursor.

Complex medium, e.g., LB, was sterilized by autoclaving (121° C., 21′) and minimal medium by filtration (0.22 μm Sartorius). When necessary, the medium was made selective by adding an antibiotic (e.g., zeocin (20 mg/L)).

### Strains, Plasmids and Mutations

Bacillus subtilis 168, available at Bacillus Genetic Stock Center (Ohio, USA).

Plasmids for gene deletion via Cre/lox are constructed as described by Yan et al. (Appl. & Environm. Microbial., September 2008, p 5556-5562). Gene disruption is done via homologous recombination with linear DNA and transformation via electroporation as described by Xue et al. (J. Microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: Bba_K143012, Bba_K823000, Bba_K823002 or Bba_K823003. Cloning can be performed using Gibson Assembly, Golden Gate assembly, Cliva assembly, LCR or restriction ligation.

In an example for the production of lactose-based oligosaccharides, Bacillus subtilis mutant strains are created to contain a gene coding for a lactose importer (such as the E. coli lacY with UniProt ID P02920). For 2′FL, 3FL and diFL production, an alpha-1,2- and/or alpha-1,3-fucosyltransferase expression construct is additionally added to the strains. For LN3 production, expression constructs are added that comprise at least two different coding DNA sequences chosen from the list comprising SEQ ID NOS:01 to 57 encoding one or more proteins with galactoside beta-1,3-N-acetylglucosaminyltransferase activity. For LNT production, the LN3 producing strains are further modified with expression constructs that comprise at least two different coding DNA sequences chosen from the list comprising SEQ ID NOs:58 to 66 encoding one or more proteins with N-acetylglucosamine beta-1,3-galactosyltransferase activity. For LNnT production, the LN3 producing strains are further modified with expression constructs that comprise at least two different coding DNA sequences chosen from the list comprising SEQ ID NOs:67 to 78 encoding one or more proteins with N-acetylglucosamine beta-1,4-galactosyltransferase activity.

For sialic acid production, a mutant B. subtilis strain is created by overexpressing the native fructose-6-P-aminotransferase (UniProt ID P0CI73) to enhance the intracellular glucosamine-6-phosphate pool. Further on, the enzymatic activities of the genes nagA, nagB and gamA are disrupted by genetic knockouts and a glucosamine-6-P-aminotransferase from S. cerevisiae (UniProt ID P43577), an N-acetylglucosamine-2-epimerase from B. ovatus (UniProt ID A7LVG6) and an N-acetylneuraminate synthase from C. jejuni (UniProt ID Q93MP9) are overexpressed on the genome. To allow sialylated oligosaccharide production, the sialic acid producing strain is further modified with expression constructs comprising two or more coding DNA sequences encoding orthologs with N-acylneuraminate cytidylyltransferase activity like e.g., the NeuA enzyme from C. jejuni (UniProt ID Q93MP7), the NeuA enzyme from H. influenzae (GenBank No. AGV11798.1) and the NeuA enzyme from P. multocida (GenBank No. AMK07891.1), and one or more copies of a beta-galactoside alpha-2,3-sialyltransferase like e.g., PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity, NmeniST3 from N. meningitidis (GenBank No. ARC07984.1) or PmultST2 from P. multocida subsp. multocida str. Pm70 (GenBank No. AAK02592.1), a beta-galactoside alpha-2,6-sialyltransferase like e.g., PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g., from M. musculus (UniProt ID Q64689).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 90° C. or for 60 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations by the biomass, in relative percentages compared to a reference strain. The biomass is empirically determined to be approximately ⅓rd of the optical density measured at 600 nm.

### Example 45. Production of LNT or LNnT with Modified B. subtilis Strains

A B. subtilis strain is first modified by genomic knock-out of the nagB, glmS, gamA and thyA genes and genomic knock-ins of constitutive transcriptional units comprising genes encoding the lactose permease (LacY) from E. coli (UniProt ID P02920), the native fructose-6-P-aminotransferase (UniProt ID P0CI73), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). The thus obtained mutant strain is further modified with genomic knock-ins of constitutive transcriptional units comprising at least two different coding DNA sequences chosen from the list comprising SEQ ID NO:01 to 57 encoding one or more proteins with galactoside beta-1,3-N-acetylglucosaminyltransferase activity to produce LN3. In a next step, the mutant LN3 producing strains are further transformed with an expression plasmid containing constitutive transcriptional units for E. coli thyA (UniProt ID P0A884) as selective marker and at least two different coding DNA sequences chosen from the list comprising either 1) SEQ ID NOs:58 to 66 encoding one or more proteins with N-acetylglucosamine beta-1,3-galactosyltransferase activity to produce LNT, or 2) SEQ ID NOs:67 to 78 encoding one or more proteins with N-acetylglucosamine beta-1,4-galactosyltransferase activity to produce LNnT. The novel strains are evaluated in a growth experiment on MMsf medium comprising lactose as precursor according to the culture conditions provided in Example 44. After 72 h of incubation, the culture broth is harvested, and the sugars are analyzed on UPLC.

### Example 46. Material and Methods Corynebacterium glutamicum

### Media

Two different media are used, namely a rich tryptone-yeast extract (TY) medium and a minimal medium for shake flask (MMsf). The minimal medium uses a 1000× stock trace element mix.

Trace element mix consisted of 10 g/L CaCl₂), 10 g/L FeSO₄·7H₂O, 10 g/L MnSO₄·H₂O, 1 g/L ZnSO₄·7H₂O, 0.2 g/L CuSO₄, 0.02 g/L NiCl₂·6H₂O, 0.2 g/L biotin (pH 7.0) and 0.03 g/L protocatechuic acid.

The minimal medium for the shake flasks (MMsf) experiments contained 20 g/L (NH₄)₂SO₄, 5 g/L urea, 1 g/L KH₂PO₄, 1 g/L K₂HPO₄, 0.25 g/L MgSO₄·7H₂O, 42 g/L MOPS, from 10 up to 30 g/L glucose or another carbon source including but not limited to fructose, maltose, sucrose, glycerol and maltotriose when specified in the examples and 1 ml/L trace element mix. Depending on the experiment lactose, LNB or LacNAc could be added as a precursor.

The TY medium consisted of 1.6% tryptone (Difco, Erembodegem, Belgium), 1% yeast extract (Difco) and 0.5% sodium chloride (VWR, Leuven, Belgium). TY agar (TYA) plates consisted of the TY media, with 12 g/L agar (Difco, Erembodegem, Belgium) added. Complex medium, e.g., TY, was sterilized by autoclaving (121° C., 21′) and minimal medium by filtration (0.22 μm Sartorius). When necessary, the medium was made selective by adding an antibiotic (e.g., kanamycin, ampicillin).

### Strains and Mutations

Corynebacterium glutamicum ATCC 13032, available at the American Type Culture Collection.

Integrative plasmid vectors based on the Cre/loxP technique as described by Suzuki et al. (Appl. Microbiol. Biotechnol., 2005 April, 67(2):225-33) and temperature-sensitive shuttle vectors as described by Okibe et al. (Journal of Microbiological Methods 85, 2011, 155-163) are constructed for gene deletions, mutations and insertions. Suitable promoters for (heterologous) gene expression can be derived from Yim et al. (Biotechnol. Bioeng., 2013 November, 110(11):2959-69). Cloning can be performed using Gibson Assembly, Golden Gate assembly, Cliva assembly, LCR or restriction ligation.

In an example for the production of lactose-based oligosaccharides, C. glutamicum mutant strains are created to contain a gene coding for a lactose importer (such as the E. coli lacY with UniProt ID P02920). For 2′FL, 3FL and diFL production, an alpha-1,2- and/or alpha-1,3-fucosyltransferase expression construct is additionally added to the strains. For LN3 production, expression constructs are added that comprise at least two different coding DNA sequences chosen from the list comprising SEQ ID NOs:01 to 57 encoding one or more proteins with galactoside beta-1,3-N-acetylglucosaminyltransferase activity. For LNT production, the LN3 producing strains are further modified with expression constructs that comprise at least two different coding DNA sequences chosen from the list comprising SEQ ID NOs:58 to 66 encoding one or more proteins with N-acetylglucosamine beta-1,3-galactosyltransferase activity. For LNnT production, the LN3 producing strains are further modified with expression constructs that comprise at least two different coding DNA sequences chosen from the list comprising SEQ ID NOs:67 to 78 encoding one or more proteins with N-acetylglucosamine beta-1,4-galactosyltransferase activity.

For sialic acid production, a mutant C. glutamicum strain is created by overexpressing the native fructose-6-P-aminotransferase (UniProt ID Q8NND3) to enhance the intracellular glucosamine-6-phosphate pool. Further on, the enzymatic activities of the genes nagA, nagB and gamA are disrupted by genetic knockouts and a glucosamine-6-P-aminotransferase from S. cerevisiae (UniProt ID P43577), an N-acetylglucosamine-2-epimerase from B. ovatus (UniProt ID A7LVG6) and an N-acetylneuraminate synthase from C. jejuni (UniProt ID Q93MP9) are overexpressed on the genome. To allow sialylated oligosaccharide production, the sialic acid producing strain is further modified with expression constructs comprising two or more coding DNA sequences encoding orthologs with N-acylneuraminate cytidylyltransferase activity like e.g., the NeuA enzyme from C. jejuni (UniProt ID Q93MP7), the NeuA enzyme from H. influenzae (GenBank No. AGV11798.1) and the NeuA enzyme from P. multocida (GenBank No. AMK07891.1), and one or more copies of a beta-galactoside alpha-2,3-sialyltransferase like e.g., PmultST3 from P. multocida (UniProt ID Q9CLP3) or a PmultST3-like polypeptide consisting of amino acid residues 1 to 268 of UniProt ID Q9CLP3 having beta-galactoside alpha-2,3-sialyltransferase activity, NmeniST3 from N. meningitidis (GenBank No. ARC07984.1) or PmultST2 from P. multocida subsp. multocida str. Pm70 (GenBank No. AAK02592.1), a beta-galactoside alpha-2,6-sialyltransferase like e.g., PdST6 from Photobacterium damselae (UniProt ID 066375) or a PdST6-like polypeptide consisting of amino acid residues 108 to 497 of UniProt ID 066375 having beta-galactoside alpha-2,6-sialyltransferase activity or P-JT-ISH-224-ST6 from Photobacterium sp. JT-ISH-224 (UniProt ID A8QYL1) or a P-JT-ISH-224-ST6-like polypeptide consisting of amino acid residues 18 to 514 of UniProt ID A8QYL1 having beta-galactoside alpha-2,6-sialyltransferase activity, and/or an alpha-2,8-sialyltransferase like e.g., from M. musculus (UniProt ID Q64689).

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a TY plate, in 150 μL TY and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the oligosaccharide concentrations, e.g., sialyllactose concentrations, measured in the whole broth by the biomass, in relative percentages compared to the reference strain. The biomass is empirically determined to be approximately ⅓rd of the optical density measured at 600 nm.

### Example 47. Production of 6′-SL or 3′-SL in Mutant C. glutamicum Strains

A wild-type C. glutamicum strain is first modified with genomic knockouts of the C. glutamicum genes ldh, cgl2645, nagB, gamA and nagA, together with genomic knock-ins of constitutive transcriptional units comprising genes encoding the lactose permease (LacY) from E. coli (UniProt ID P02920), native fructose-6-P-aminotransferase (UniProt ID Q8NND3), a glucosamine-6-P-aminotransferase from S. cerevisiae (UniProt ID P43577), an N-acetylglucosamine-2-epimerase from B. ovatus (UniProt ID A7LVG6), an N-acetylneuraminate synthase from C. jejuni (UniProt ID Q93MP9), the sucrose transporter (CscB) from E. coli W (UniProt ID E0IXR1), the fructose kinase (Frk) from Z. mobilis (UniProt ID Q03417) and the sucrose phosphorylase (BaSP) from B. adolescentis (UniProt ID A0ZZH6). In a next step, the novel strain is transformed with an expression plasmid comprising constitutive transcriptional units comprising the genes encoding the NeuA enzyme from C. jejuni (UniProt ID Q93MP7), the NeuA enzyme from H. influenzae (GenBank No. AGV11798.1) and the NeuA enzyme from P. multocida (GenBank No. AMK07891.1) combined with the gene encoding either 1) the beta-galactoside alpha-2,3-sialyltransferase PmultST3 from P. multocida (UniProt ID Q9CLP3) to produce 3′-SL or 2) the beta-galactoside alpha-2,6-sialyltransferase PdST6 from P. damselae (UniProt ID 066375) to produce 6′-SL. The novel strains are evaluated in a growth experiment on MMsf medium comprising lactose as precursor according to the culture conditions provided in Example 44. After 72 h of incubation, the culture broth is harvested, and the sugars are analyzed on UPLC.

### Example 48. Materials and Methods Chlamydomonas reinhardtii

### Media

C. reinhardtii cells were cultured in Tris-acetate-phosphate (TAP) medium (pH 7.0). The TAP medium uses a 1000× stock Hutner's trace element mix. Hutner's trace element mix consisted of 50 g/L Na₂EDTA·H₂O (Titriplex III), 22 g/L ZnSO₄·7H₂O, 11.4 g/L H₃BO₃, 5 g/L MnCl₂·4H₂O, 5 g/L FeSO₄·7H₂O, 1.6 g/L COCl₂·6H₂O, 1.6 g/L CuSO₄·5H₂O and 1.1 g/L (NH₄)₆MoO₃.

The TAP medium contained 2.42 g/L Tris (tris(hydroxymethyl)aminomethane), 25 mg/L salt stock solution, 0.108 g/L K₂HPO₄, 0.054 g/L KH₂PO₄ and 1.0 mL/L glacial acetic acid. The salt stock solution consisted of 15 g/L NH₄Cl, 4 g/L MgSO₄·7H₂O and 2 g/L CaCl₂)·2H₂O. As precursor for saccharide synthesis, precursors like e.g., galactose, glucose, fructose, fucose, GlcNAc could be added. Medium was sterilized by autoclaving (121° C., 21′). For stock cultures on agar slants TAP medium was used containing 1% agar (of purified high strength, 1000 g/cm²).

### Strains, Plasmids and Mutations

C. reinhardtii wild-type strains 21 gr (CC-1690, wild-type, mt+), 6145C (CC-1691, wild-type, mt−), CC-125 (137c, wild-type, mt+), CC-124 (137c, wild-type, mt−) as available from Chlamydomonas Resource Center (www.chlamycollection.org), University of Minnesota, U.S.A.

Expression plasmids originated from pSI103, as available from Chlamydomonas Resource Center. Cloning can be performed using Gibson Assembly, Golden Gate assembly, Cliva assembly, LCR or restriction ligation. Suitable promoters for (heterologous) gene expression can be derived from e.g., Scranton et al. (Algal Res. 2016, 15: 135-142). Targeted gene modification (like gene knock-out or gene replacement) can be carried using the Crispr-Cas technology as described e.g., by Jiang et al. (Eukaryotic Cell 2014, 13(11): 1465-1469).

Transformation via electroporation was performed as described by Wang et al. (Biosci. Rep. 2019, 39: BSR2018210). Cells were grown in liquid TAP medium under constant aeration and continuous light with a light intensity of 8000 Lx until the cell density reached 1.0-2.0×10⁷ cells/mL. Then, the cells were inoculated into fresh liquid TAP medium in a concentration of 1.0×10⁶ cells/mL and grown under continuous light for 18-20 h until the cell density reached 4.0×10⁶ cells/mL. Next, cells were collected by centrifugation at 1250 g for 5 min at room temperature, washed and resuspended with pre-chilled liquid TAP medium containing 60 mM sorbitol (Sigma, U.S.A.), and iced for 10 min. Then, 250 μL of cell suspension (corresponding to 5.0×10⁷ cells) were placed into a pre-chilled 0.4 cm electroporation cuvette with 100 ng plasmid DNA (400 ng/mL). Electroporation was performed with 6 pulses of 500 V each having a pulse length of 4 ms and pulse interval time of 100 ms using a BTX ECM830 electroporation apparatus (1575Ω, 50 μFD). After electroporation, the cuvette was immediately placed on ice for 10 min. Finally, the cell suspension was transferred into a 50 ml conical centrifuge tube containing 10 mL of fresh liquid TAP medium with 60 mM sorbitol for overnight recovery at dim light by slowly shaking. After overnight recovery, cells were recollected and plated with starch embedding method onto selective 1.5% (w/v) agar-TAP plates containing ampicillin (100 mg/L) or chloramphenicol (100 mg/L). Plates were then incubated at 23+/−0.5° C. under continuous illumination with a light intensity of 8000 Lx. Cells were analyzed 5-7 days later.

In an example for production of UDP-galactose, C. reinhardtii cells are modified with transcriptional units comprising the genes encoding the galactokinase from Arabidopsis thaliana (KIN, UniProt ID Q9SEE5) and the UDP-sugar pyrophosphorylase (USP) from A. thaliana (UniProt ID Q9C5I1). In a next step, the C. reinhardtii cells are transformed with an expression plasmid comprising transcriptional units comprising at least two different coding DNA sequences chosen from the list comprising either 1) SEQ ID NOs:58 to 66 encoding one or more proteins with N-acetylglucosamine beta-1,3-galactosyltransferase activity to produce LNB, or 2) SEQ ID NOs:67 to 78 encoding one or more proteins with N-acetylglucosamine beta-1,4-galactosyltransferase activity to produce LacNAc.

In an example for production of GDP-fucose, C. reinhardtii cells are modified with a transcriptional unit for a GDP-fucose synthase like e.g., from Arabidopsis thaliana (GER1, UniProt ID 049213).

In an example for fucosylation, C. reinhardtii cells can be modified with an expression plasmid comprising a constitutive transcriptional unit for an alpha-1,2-fucosyltransferase like e.g., HpFutC from H. pylori (GenBank No. AAD29863.1) and/or an alpha-1,3-fucosyltransferase like e.g., HpFucT from H. pylori (UniProt ID 030511).

In an example for CMP-sialic acid synthesis, C. reinhardtii cells are modified with constitutive transcriptional units for an UDP-N-acetylglucosamine-2-epimerase/N-acetylmannosamine kinase like e.g., GNE from Homo sapiens (UniProt ID Q9Y223) or a mutant form of the human GNE polypeptide comprising the R263L mutation, an N-acylneuraminate-9-phosphate synthetase like e.g., NANS from Homo sapiens (UniProt ID Q9NR45) and an N-acylneuraminate cytidylyltransferase like e.g., CMAS from Homo sapiens (UniProt ID Q8NFW8). In an example for production of sialylated oligosaccharides, C. reinhardtii cells are modified with a CMP-sialic acid transporter like e.g., CST from Mus musculus (UniProt ID Q61420), and a Golgi-localised sialyltransferase chosen from species like e.g., Homo sapiens, Mus musculus, Rattus norvegicus.

### Heterologous and Homologous Expression

Genes that needed to be expressed, be it from a plasmid or from the genome were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

Cells of C. reinhardtii were cultured in selective TAP-agar plates at 23+/−0.5° C. under 14/10 h light/dark cycles with a light intensity of 8000 Lx. Cells were analyzed after 5 to 7 days of cultivation.

For high-density cultures, cells could be cultivated in closed systems like e.g., vertical or horizontal tube photobioreactors, stirred tank photobioreactors or flat panel photobioreactors as described by Chen et al. (Bioresour. Technol. 2011, 102: 71-81) and Johnson et al. (Biotechnol. Prog. 2018, 34: 811-827).

### Example 49. Production of LNB or LacNAc in Mutant C. reinhardtii Cells

C. reinhardtii cells are engineered as described in Example 48, comprising genomic knock-ins of constitutive transcriptional units comprising the Arabidopsis thaliana genes encoding the galactokinase (KIN, UniProt ID Q9SEE5) and the UDP-sugar pyrophosphorylase (USP) (UniProt ID Q9C5I1). In a next step, the mutant cells are transformed with an expression plasmid comprising transcriptional units comprising at least two different coding DNA sequences chosen from the list comprising either 1) SEQ ID NOs:58 to 66 encoding one or more proteins with N-acetylglucosamine beta-1,3-galactosyltransferase activity to produce LNB, or 2) SEQ ID NOs:67 to 78 encoding one or more proteins with N-acetylglucosamine beta-1,4-galactosyltransferase activity to produce LacNAc. The novel strains are evaluated in a cultivation experiment on TAP-agar plates comprising galactose and GlcNAc as precursors according to the culture conditions provided in Example 48. After 5 days of incubation, the cells are harvested, and the production of LNB or LacNAc is analyzed on UPLC.

### Example 50. Materials and Methods Animal Cells

Isolation of Mesenchymal Stem Cells from Adipose Tissue of Different Mammals

Fresh adipose tissue is obtained from slaughterhouses (e.g., cattle, pigs, sheep, chicken, ducks, catfish, snake, frogs) or liposuction (e.g., in case of humans, after informed consent) and kept in phosphate buffer saline supplemented with antibiotics. Enzymatic digestion of the adipose tissue is performed followed by centrifugation to isolate mesenchymal stem cells. The isolated mesenchymal stem cells are transferred to cell culture flasks and grown under standard growth conditions, e.g., 370 C, 5% CO₂. The initial culture medium includes DMEM-F12, RPMI, and Alpha-MEM medium (supplemented with 15% foetal bovine serum), and 1% antibiotics. The culture medium is subsequently replaced with 10% FBS (foetal bovine serum)-supplemented media after the first passage. For example, Ahmad and Shakoori (2013, Stem Cell Regen. Med. 9(2): 29-36), which is incorporated herein by reference in its entirety for all purposes, describes certain variation(s) of the method(s) described herein in this example.

Isolation of Mesenchymal Stem Cells from Milk

This example illustrates isolation of mesenchymal stem cells from milk collected under aseptic conditions from human or any other mammal(s) such as described herein. An equal volume of phosphate buffer saline is added to diluted milk, followed by centrifugation for 20 min. The cell pellet is washed thrice with phosphate buffer saline and cells are seeded in cell culture flasks in DMEM-F12, RPMI, and Alpha-MEM medium supplemented with 10% foetal bovine serum and 1% antibiotics under standard culture conditions. For example, Hassiotou et al. (2012, Stem Cells. 30(10): 2164-2174), which is incorporated herein by reference in its entirety for all purposes, describes certain variation(s) of the method(s) described herein in this example.

### Differentiation of Stem Cells Using 2D and 3D Culture Systems

The isolated mesenchymal cells can be differentiated into mammary-like epithelial and luminal cells in 2D and 3D culture systems. See, for example, Huynh et al. 1991. Exp Cell Res. 197(2): 191-199; Gibson et al. 1991, In Vitro Cell Dev Biol Anim. 27(7): 585-594; Blatchford et al. 1999; Animal Cell Technology’: Basic & Applied Aspects, Springer, Dordrecht. 141-145; Williams et al. 2009, Breast Cancer Res 11(3): 26-43; and Arevalo et al. 2015, Am J Physiol Cell Physiol. 310(5): C348-C356; each of which is incorporated herein by reference in their entireties for all purposes.

For 2D culture, the isolated cells were initially seeded in culture plates in growth media supplemented with 10 ng/ml epithelial growth factor and 5 μg/ml insulin. At confluence, cells were fed with growth medium supplemented with 2% fetal bovine serum, 1% penicillin-streptomycin (100 U/ml penicillin, 100 μg/ml streptomycin), and 5 μg/ml insulin for 48 h. To induce differentiation, the cells were fed with complete growth medium containing 5 μg/ml insulin, 1 μg/ml hydrocortisone, 0.65 ng/ml triiodothyronine, 100 nM dexamethasone, and 1 μg/ml prolactin. After 24 h, serum is removed from the complete induction medium.

For 3D culture, the isolated cells were trypsinized and cultured in Matrigel, hyaluronic acid, or ultra-low attachment surface culture plates for six days and induced to differentiate and lactate by adding growth media supplemented with 10 ng/ml epithelial growth factor and 5 μg/ml insulin. At confluence, cells were fed with growth medium supplemented with 2% foetal bovine serum, 1% penicillin-streptomycin (100 U/ml penicillin, 100 μg/ml streptomycin), and 5 μg/ml insulin for 48 h. To induce differentiation, the cells were fed with complete growth medium containing 5 μg/ml insulin, 1 μg/ml hydrocortisone, 0.65 ng/ml triiodothyronine, 100 nM dexamethasone, and 1 μg/ml prolactin. After 24 h, serum is removed from the complete induction medium.

### Method of Making Mammary-Like Cells

Mammalian cells are brought to induced pluripotency by reprogramming with viral vectors encoding for Oct4, Sox2, Klf4, and c-Myc. The resultant reprogrammed cells are then cultured in Mammocult media (available from Stem Cell Technologies), or mammary cell enrichment media (DMEM, 3% FBS, estrogen, progesterone, heparin, hydrocortisone, insulin, EGF) to make them mammary-like, from which expression of select milk components can be induced. Alternatively, epigenetic remodelling are performed using remodelling systems such as CRISPR/Cas9, to activate select genes of interest, such as casein, a-lactalbumin to be constitutively on, to allow for the expression of their respective proteins, and/or to down-regulate and/or knock-out select endogenous genes as described e.g., in WO 2021067641, which is incorporated herein by reference in its entirety for all purposes.

### Cultivation

Completed growth media includes high glucose DMEM/F12, 10% FBS, 1% NEAA, 1% pen/strep, 1% ITS-X, 1% F-Glu, 10 ng/ml EGF, and 5 μg/ml hydrocortisone. Completed lactation media includes high glucose DMEM/F12, 1% NEAA, 1% pen/strep, 1% ITS-X, 1% F-Glu, 10 ng/ml EGF, 5 μg/ml hydrocortisone, and 1 μg/ml prolactin (5 ug/ml in Hyunh 1991). Cells are seeded at a density of 20,000 cells/cm² onto collagen coated flasks in completed growth media and left to adhere and expand for 48 hours in completed growth media, after which the media is switched out for completed lactation media. Upon exposure to the lactation media, the cells start to differentiate and stop growing. Within about a week, the cells start secreting lactation product(s) such as milk lipids, lactose, casein and whey into the media. A desired concentration of the lactation media can be achieved by concentration or dilution by ultrafiltration. A desired salt balance of the lactation media can be achieved by dialysis, for example, to remove unwanted metabolic products from the media. Hormones and other growth factors used can be selectively extracted by resin purification, for example, the use of nickel resins to remove His-tagged growth factors, to further reduce the levels of contaminants in the lactated product.

### Example 51. Evaluation of 2′-FL Production in a Non-Mammary Adult Stem Cell

Isolated mesenchymal cells and re-programmed into mammary-like cells as described in Example 50 are modified via CRISPR-CAS to over-express the beta-1,4-galactosyltransferase 1 B4GalT1 from Homo sapiens (UniProt ID P15291), the GDP-fucose synthase GFUS from Homo sapiens (UniProt ID Q13630) and the galactoside alpha-1,2-fucosyltransferases FUT2 from Homo sapiens (UniProt ID Q10981), FUT2 from Mus musculus (UniProt ID Q9JL27) and FUT2 from Caenorhabditis elegans (UniProt ID P91200). All genes introduced in the cells are codon-optimized to the host. Cells are seeded at a density of 20,000 cells/cm² onto collagen coated flasks in completed growth media and left to adhere and expand for 48 hours in completed growth media, after which the media is switched out for completed lactation media for about 7 days. After cultivation as described in Example 50, cells are subjected to UPLC to analyze for production of 2′FL.

### Example 52. Evaluation of LacNAc, Sialylated LacNAc and Sialyl-Lewis x Production in a Non-Mammary Adult Stem Cell

Isolated mesenchymal cells and re-programmed into mammary-like cells as described in Example 50 are modified via CRISPR-CAS to over-express the beta-1,4-galactosyltransferase 4 B4GalT4 from Homo sapiens (UniProt ID 060513), the GDP-fucose synthase GFUS from Homo sapiens (UniProt ID Q13630), the galactoside alpha-1,3-fucosyltransferase FUT3 from Homo sapiens (UniProt ID P21217), the N-acylneuraminate cytidylyltransferases from Mus musculus (UniProt ID Q99KK2), Danio rerio (UniProt ID Q0E671) and Homo sapiens (UniProt ID Q8NFW8) and the CMP-N-acetylneuraminate-beta-1,4-galactoside alpha-2,3-sialyltransferase ST3GAL3 from Homo sapiens (UniProt ID Q11203). All genes introduced in the cells are codon-optimized to the host. Cells are seeded at a density of 20,000 cells/cm² onto collagen coated flasks in completed growth media and left to adhere and expand for 48 hours in completed growth media, after which the media is switched out for completed lactation media for about 7 days. After cultivation as described in Example 50, cells are subjected to UPLC to analyze for production of LacNAc, sialylated LacNAc and sialyl-Lewis x.

1.-79. (canceled)

80. A cell for producing a di- and/or oligosaccharide, the cell comprising a pathway for production of the di- and/or oligosaccharide, wherein the cell is genetically modified for expression and/or overexpression of at least one set of multiple coding DNA sequences,
wherein the multiple coding DNA sequences within one set:
differ in nucleotide sequence, and
each encode a polypeptide,
wherein the polypeptides have the same function and/or activity of interest.

81. The cell of claim 80, wherein the polypeptides within a set are functional variants, the variants comprising a functional homolog, ortholog and paralog.

82. The cell of claim 80, wherein multiple is at least two (2).

83. The cell of claim 80, wherein the cell comprises at least two (2) sets of multiple coding DNA sequences, wherein each set of multiple coding DNA sequences encodes polypeptides having a different function and/or activity of interest compared to the other sets of multiple coding DNA sequences.

84. The cell of claim 80, wherein the multiple coding DNA sequences within a set are integrated in the genome of the cell and/or presented to the cell on one or more vectors comprising a plasmid, cosmid, artificial chromosome, phage, liposome or virus, which is/are to be stably transformed into the cell.

85. The cell of claim 80, wherein the multiple coding DNA sequences within a set are presented to the cell
in one or more location(s) on one or more chromosome(s),
within a biosynthetic gene cluster encoding polypeptides participating in the pathway for production of the di- and/or oligosaccharide, and/or
in one or more gene expression modules comprising one or more regulatory gene sequences regulating expression of the multiple coding DNA sequences.

86. The cell of claim 80, wherein the multiple coding DNA sequences within a set are organized within any one or more selected from the group consisting of co-expression module, operon, regulon, stimulon, and modulon.

87. The cell of claim 80, wherein expression of the multiple coding DNA sequences within a set is regulated by at least one promoter sequence that is constitutive or inducible upon a natural inducer.

88. The cell of claim 80, wherein the cell is genetically modified to produce the di- and/or oligosaccharide.

89. The cell of claim 80, wherein the cell is genetically modified by introducing a pathway for producing the di- and/or oligosaccharide.

90. The cell of claim 80, wherein the polypeptides encoded by at least one set of multiple coding DNA sequences are directly involved in the pathway for production of the di- and/or oligosaccharide.

91. The cell of claim 80, wherein the polypeptides that are encoded by multiple coding DNA sequences within a set have the same function and/or activity and wherein the function and/or activity is:
i) directly involved in the synthesis of a nucleotide-activated sugar, wherein the nucleotide-activated sugar is to be used in producing the di- and/or oligosaccharide,
ii) a glycosyltransferase activity for transferring a monosaccharide from a nucleotide-activated sugar donor to a disaccharide/oligosaccharide acceptor, or
iii) a transport activity hereby transporting compounds across the outer membrane of the cell wall.

92. The cell of claim 91, wherein the nucleotide-activated sugar is selected from the group consisting of UDP-N-acetylglucosamine (UDP-GlcNAc), UDP-N-acetylgalactosamine (UDP-GalNAc), UDP-N-acetylmannosamine (UDP-ManNAc), UDP-glucose (UDP-Glc), UDP-galactose (UDP-Gal), GDP-mannose (GDP-Man), UDP-glucuronate, UDP-galacturonate, UDP-2-acetamido-2,6-dideoxy-L-arabino-4-hexulose, UDP-2-acetamido-2,6-dideoxy-L-lyxo-4-hexulose, UDP-N-acetyl-L-rhamnosamine (UDP-L-RhaNAc or UDP-2-acetamido-2,6-dideoxy-L-mannose), dTDP-N-acetylfucosamine, UDP-N-acetylfucosamine (UDP-L-FucNAc or UDP-2-acetamido-2,6-dideoxy-L-galactose), UDP-N-acetyl-L-pneumosamine (UDP-L-PneNAC or UDP-2-acetamido-2,6-dideoxy-L-talose), UDP-N-acetylmuramic acid, UDP-N-acetyl-L-quinovosamine (UDP-L-QuiNAc or UDP-2-acetamido-2,6-dideoxy-L-glucose), CMP-sialic acid (CMP-Neu5Ac), CMP-Neu4Ac, CMP-Neu5Ac9N₃, CMP-Neu4,5Ac₂, CMP-Neu5,7Ac₂, CMP-Neu5,9Ac₂, CMP-Neu5,7(8,9)Ac₂, CMP-N-glycolylneuraminic acid (CMP-Neu5Gc), GDP-fucose (GDP-Fuc), GDP-rhamnose, and UDP-xylose.

93. The cell of claim 91, wherein the multiple coding DNA sequences within a set encode polypeptides having the same function and/or activity in the synthesis of a nucleotide-activated sugar and that are selected from the group consisting of mannose-6-phosphate isomerase, phosphomannomutase, mannose-1-phosphate guanylyltransferase, GDP-mannose 4,6-dehydratase, GDP-L-fucose synthase, L-fucokinase/GDP-fucose pyrophosphorylase, fucose-1-phosphate guanylyltransferase, L-glutamine-D-fructose-6-phosphate aminotransferase, glucosamine-6-phosphate deaminase, phosphoglucosamine mutase, N-acetylglucosamine-6-phosphate deacetylase, N-acylglucosamine 2-epimerase, UDP-N-acetylglucosamine 2-epimerase, N-acetylmannosamine-6-phosphate 2-epimerase, glucosamine 6-phosphate N-acetyltransferase, N-acetylglucosamine kinase, N-acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridylyltransferase, glucosamine-1-phosphate acetyltransferase, N-acetylneuraminate synthase, N-acetylneuraminate lyase, N-acylneuraminate-9-phosphate synthase, N-acylneuraminate-9-phosphate phosphatase, N-acylneuraminate cytidylyltransferase, galactose-1-epimerase, galactokinase, glucokinase, galactose-1-phosphate uridylyltransferase, UDP-glucose 4-epimerase, glucose-1-phosphate uridylyltransferase, phosphoglucomutase, UDP-N-acetylglucosamine 4-epimerase, N-acetylgalactosamine kinase, and UDP-N-acetylgalactosamine pyrophosphorylase.

94. The cell of claim 91, wherein the multiple coding DNA sequences within a set encode glycosyltransferases or polypeptides having glycosyltransferase activity that are selected from the group consisting of fucosyltransferases, sialyltransferases, galactosyltransferases, glucosyltransferases, mannosyltransferases, N-acetylglucosaminyltransferases, N-acetylgalactosaminyltransferases, N-acetylmannosaminyltransferases, xylosyltransferases, glucuronyltransferases, galacturonyltransferases, glucosaminyltransferases, N-glycolylneuraminyltransferases, rhamnosyltransferases, N-acetylrhamnosyltransferases, UDP-4-amino-4,6-dideoxy-N-acetyl-beta-L-altrosamine transaminases, UDP-N-acetylglucosamine enolpyruvyl transferases, and fucosaminyltransferases.

95. The cell of claim 91, wherein the multiple coding DNA sequences within a set encode polypeptides that are membrane transporter proteins or polypeptides having transport activity so as to transport compounds across the outer membrane of a cell wall.

96. The cell of claim 91, wherein the membrane transporter proteins or polypeptides having transport activity are selected from the group consisting of transporters comprising porters, P-P-bond-hydrolysis-driven transporters, b-barrel porins, auxiliary transport proteins, putative transport proteins, and phosphotransfer-driven group translocators.

97. The cell of claim 80, wherein the cell uses at least one precursor for producing the di- and/or oligosaccharide the precursor(s) being fed to the cell from the cultivation medium.

98. The cell of claim 80, wherein the cell produces at least one precursor for producing the di- and/or oligosaccharide.

99. The cell of claim 91, wherein the membrane transporter proteins or polypeptides having transport activity:
control the flow over the outer membrane of the cell wall of i) the di- and/or oligosaccharide and/or ii) any one or more precursor(s) and/or acceptor(s) to be used in producing the di- and/or oligosaccharide, and/or
provide improved production and/or enabled and/or enhanced efflux of the di- and/or oligosaccharide.

100. The cell of claim 80, wherein the di- and/or oligosaccharide is selected from the group consisting of a milk oligosaccharide, O-antigen, enterobacterial common antigen (ECA), oligosaccharide repeats present in capsular polysaccharides, peptidoglycan, amino-sugars, Lewis-type antigen oligosaccharide and antigens of the human ABO blood group system.

101. The cell of claim 80, wherein the pathway comprises:
a fucosylation pathway,
a sialylation pathway,
a galactosylation pathway,
an N-acetylglucosaminylation pathway,
an N-acetylgalactosaminylation pathway,
a mannosylation pathway, and/or
an N-acetylmannosaminylation pathway.

102. The cell of claim 80, wherein the cell is capable of producing phosphoenolpyruvate (PEP) and/or wherein the cell is modified for enhanced production and/or supply of PEP.

103. The cell of claim 80, wherein the polypeptides that are encoded by the multiple coding DNA sequences within a set are directly involved in the production and/or supply of PEP.

104. The cell of claim 80, wherein the cell comprises:
i) a set of multiple coding DNA sequences wherein the multiple coding DNA sequences differ in nucleotide sequence and each encode a polypeptide having galactoside beta-1,3-N-acetylglucosaminyltransferase activity, and wherein each of the coding DNA sequences:
is selected from the group consisting of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57;
is a fragment of any one of SEQ ID NOs: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 and 57 encoding a polypeptide having galactoside beta-1,3-N-acetylglucosaminyltransferase activity;
comprises and/or consists of a nucleotide sequence having 80% or more sequence identity to the full-length nucleotide sequence of any one of SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56 or 57 and encoding a polypeptide having galactoside beta-1,3-N-acetylglucosaminyltransferase activity;
encodes a polypeptide selected from the group consisting of SEQ ID NOs: 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, and 131;
encodes a functional fragment of a polypeptide according to any one of SEQ ID NOs: 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 or 131 and having galactoside beta-1,3-N-acetylglucosaminyltransferase activity; and/or
encodes a polypeptide comprising a peptide having 80% or more sequence identity to the full-length peptide of any one of SEQ ID NO: 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130 or 131 and having galactoside beta-1,3-N-acetylglucosaminyltransferase activity;
ii) a set of multiple coding DNA sequences wherein the multiple coding DNA sequences differ in nucleotide sequence and each encode a polypeptide having N-acetylglucosamine beta-1,3-galactosyltransferase activity, and wherein each of the coding DNA sequences:
is selected from the group consisting of SEQ ID NOs: 58, 59, 60, 61, 62, 63, 64, 65 and 66;
is a fragment of any one of SEQ ID NOs: 58, 59, 60, 61, 62, 63, 64, 65 and 66 encoding a polypeptide having N-acetylglucosamine beta-1,3-galactosyltransferase activity, and/or
comprises and/or consists of a nucleotide sequence having 80% or more sequence identity to the full-length nucleotide sequence of any one of SEQ ID NO: 58, 59, 60, 61, 62, 63, 64, 65 or 66 and encoding a polypeptide having N-acetylglucosamine beta-1,3-galactosyltransferase activity;
encodes a polypeptide selected from the group consisting of SEQ ID NOs: 132, 133, 134 and 135;
encodes a functional fragment of a polypeptide according to any one of SEQ ID NOs: 132, 133, 134 or 135 and having N-acetylglucosamine beta-1,3-galactosyltransferase activity; and/or
encodes a polypeptide comprising a peptide having 80% or more sequence identity to the full-length peptide of any one of SEQ ID NO: 132, 133, 134, or 135 and having N-acetylglucosamine beta-1,3-galactosyltransferase activity; and/or
iii) a set of multiple coding DNA sequences wherein the multiple coding DNA sequences differ in nucleotide sequence and each encode a polypeptide having N-acetylglucosamine beta-1,4-galactosyltransferase activity, and wherein each of the coding DNA sequences:
is selected from the group consisting of SEQ ID NOs: 67, 68, 69, 70, 71, 72, 73, 74, 75, 76, 77 and 78;
is a fragment of any one of SEQ ID NOs: 67, 68, 69, 70, 71, 72, 73, 74, 75, 76, 77 and 78 encoding a polypeptide having N-acetylglucosamine beta-1,4-galactosyltransferase activity;
comprises and/or consists of a nucleotide sequence having 80% or more sequence identity to the full-length nucleotide sequence of any one of SEQ ID NO: 67, 68, 69, 70, 71, 72, 73, 74, 75, 76, 77 or 78 and encoding a polypeptide having N-acetylglucosamine beta-1,4-galactosyltransferase activity;
encodes a polypeptide selected from the group consisting of SEQ ID NOs: 136, 137, 138, 139, 140, 141, 142, 143, 144 and 145;
encodes a functional fragment of a polypeptide according to any one of SEQ ID NO: 136, 137, 138, 139, 140, 141, 142, 143, 144 or 145 and having N-acetylglucosamine beta-1,4-galactosyltransferase activity, and/or
encodes a polypeptide comprising a peptide having 80% or more sequence identity to the full-length peptide of any one of SEQ ID NO: 136, 137, 138, 139, 140, 141, 142, 143, 144 or 145 and having N-acetylglucosamine beta-1,4-galactosyltransferase activity.

105. The cell of claim 80, wherein the cell comprises a set of multiple coding DNA sequences wherein the multiple coding DNA sequences differ in nucleotide sequence and each encode a polypeptide having N-acylneuraminate cytidylyltransferase activity, and wherein each of the coding DNA sequences encodes:
a polypeptide selected from the group consisting of the polypeptide from Campylobacter jejuni with UniProt ID Q93MP7, the polypeptide from Haemophilus influenzae with GenBank No. AGV11798.1 and the polypeptide from Pasteurella multocida with GenBank No. AMK07891.1 and having N-acylneuraminate cytidylyltransferase activity, and/or
a functional fragment of any one of the polypeptide from C. jejuni with UniProt ID Q93MP7, H. influenzae with GenBank No. AGV11798.1 or P. multocida with GenBank No. AMK07891.1 and having N-acylneuraminate cytidylyltransferase activity, and/or
a polypeptide comprising a peptide having 80% or more sequence identity to the full-length peptide of any one of the polypeptides from C. jejuni with UniProt ID Q93MP7, H. influenzae with GenBank No. AGV11798.1 or P. multocida with GenBank No. AMK07891.1 and having N-acylneuraminate cytidylyltransferase activity.

106. The cell of claim 95, wherein the cell further comprises:
i) at least one coding DNA sequence encoding a:
polypeptide selected from the group consisting of the polypeptide from Neisseria meningitidis with UniProt ID E0NCD4, the polypeptide from Campylobacter jejuni with UniProt ID Q93MP9, the polypeptide from Aeromonas caviae with UniProt ID Q9R9S2, the polypeptide from Candidatus koribacter versatilis with UniProt ID Q1IMQ8, the polypeptide from Legionella pneumophila with UniProt ID Q9RDX5, the polypeptide from Methanocaldococcus jannaschii with UniProt ID Q58465 and the polypeptide from Moritella viscosa with UniProt ID A0A090IMH4 and having N-acetylneuraminate synthase activity;
a functional fragment of any one of the polypeptide from N. meningitidis with UniProt ID E0NCD4, C. jejuni with UniProt ID Q93MP9, A. caviae with UniProt ID Q9R9S2, C. koribacter versatilis with UniProt ID Q1IMQ8, L. pneumophila with UniProt ID Q9RDX5, M. jannaschii with UniProt ID Q58465 or M. viscosa with UniProt ID A0A090INM4 and having N-acetylneuraminate synthase activity;
a polypeptide comprising a peptide having 80% or more sequence identity to the full-length peptide of any one of the polypeptides from N. meningitidis with UniProt ID E0NCD4, C. jejuni with UniProt ID Q93MP9, A. caviae with UniProt ID Q9R9S2, C. koribacter versatilis with UniProt ID Q1IMQ8, L. pneumophila with UniProt ID Q9RDX5, M. jannaschii with UniProt ID Q58465 or M. viscosa with UniProt ID A0A090INM4 and having N-acetylneuraminate synthase activity, and/or
ii) two or more copies of one or more coding DNA sequences of an alpha-2,3-sialyltransferase, an alpha-2,6-sialyltransferase, and/or an alpha-2,8-sialyltransferase.

107. The cell of claim 80, wherein the cell comprises a modification for reduced production of acetate.

108. The cell of claim 80, wherein the cell further comprises a lower or reduced expression and/or abolished, impaired, reduced or delayed activity of any one or more of the proteins comprising beta-galactosidase, galactoside O-acetyltransferase, N-acetylglucosamine-6-phosphate deacetylase, glucosamine-6-phosphate deaminase, N-acetylglucosamine repressor, ribonucleotide monophosphatase, EIICBA-Nag, UDP-glucose undecaprenyl-phosphate glucose-1-phosphate transferase, L-fuculokinase, L-fucose isomerase, N-acetylneuraminate lyase, N-acetylmannosamine kinase, N-acetylmannosamine-6-phosphate 2-epimerase, EIIAB-Man, EIIC-Man, EIID-Man, ushA, galactose-1-phosphate uridylyltransferase, glucose-1-phosphate adenylyltransferase, glucose-1-phosphatase, ATP-dependent 6-phosphofructokinase isozyme 1, ATP-dependent 6-phosphofructokinase isozyme 2, glucose-6-phosphate isomerase, aerobic respiration control protein, transcriptional repressor IclR, lon protease, glucose-specific translocating phosphotransferase enzyme IIBC component ptsG, glucose-specific translocating phosphotransferase (PTS) enzyme IIBC component malX, enzyme IIA^{Glc}, beta-glucoside specific PTS enzyme II, fructose-specific PTS multiphosphoryl transfer protein FruA and FruB, ethanol dehydrogenase aldehyde dehydrogenase, pyruvate-formate lyase, acetate kinase, phosphoacyltransferase, phosphate acetyltransferase, pyruvate decarboxylase.

109. The cell of claim 80, wherein the cell comprises a catabolic pathway for selected mono-, di- or oligosaccharides which is at least partially inactivated, the mono-, di-, or oligosaccharides being involved in and/or required for producing the di- and/or oligosaccharide.

110. The cell of claim 80, wherein the cell produces the di- and/or oligosaccharide intracellularly and wherein a fraction or substantially all of the produced di- and/or oligosaccharide remains intracellularly and/or is excreted outside the cell via passive or active transport.

111. The cell of claim 80, wherein the cell produces 90 g/L or more of the di- and/or oligosaccharide in the whole broth and/or supernatant and/or wherein the di- and/or oligosaccharide in the whole broth and/or supernatant has a purity of at least 80% measured on the total amount of di- and/or oligosaccharide and its precursor(s) in the whole broth and/or supernatant, respectively.

112. The cell of claim 80, wherein the cell is a bacterium, fungus, yeast, plant cell, animal cell, or protozoan cell.

113. The cell of claim 111, wherein the cell is a viable Gram-negative bacterium that comprises a reduced or abolished synthesis of poly-N-acetyl-glucosamine (PNAG), enterobacterial common antigen (ECA), cellulose, colanic acid, core oligosaccharides, osmoregulated periplasmic glucans (OPG), glucosylglycerol, glycan, and/or trehalose.

114. The cell of claim 80, wherein the cell resists a phenomenon of lactose killing when grown in an environment in which lactose is combined with one or more other carbon source(s).

115. The cell of claim 80, wherein the cell is capable of producing a mixture of di- and/or oligosaccharides.

116. The cell of claim 80, wherein the cell is capable of producing a mixture of charged and/or neutral di- and/or oligosaccharides.

117. The cell of claim 80, wherein the cell is capable of producing a mixture of di- and oligosaccharides comprising at least two different oligosaccharides.

118. The cell of claim 80, wherein the cell is capable of producing a mixture of oligosaccharides.

119. The cell of claim 80, wherein the cell is capable of producing a mixture of charged and/or neutral mammalian milk oligosaccharides (MMOs).

120. A method of producing a di- and/or oligosaccharide by a cell, the method comprising:
cultivating the cell of claim 80 under conditions permissive to produce the di- and/or oligosaccharide, and,
optionally, separating the di- and/or oligosaccharide from the cultivation.

121. The method according to claim 120, wherein the conditions comprise:
use of a culture medium comprising at least one precursor and/or acceptor for producing the di- and/or oligosaccharide, and/or
adding to the culture medium at least one precursor and/or acceptor feed for producing the di- and/or oligosaccharide.

122. The method according to claim 120, wherein the cell is cultivated in a culture medium comprising a carbon source comprising a monosaccharide, disaccharide, oligosaccharide, polysaccharide, polyol, glycerol, a complex medium including molasses, corn steep liquor, peptone, tryptone, or yeast extract.

123. The method according to claim 120, wherein the cell uses at least one precursor for producing the di- and/or oligosaccharide.

124. The method according to claim 120, wherein the culture medium contains at least one precursor selected from the group consisting of lactose, galactose, fucose, sialic acid, GlcNAc, GalNAc, lacto-N-biose (LNB), N-acetyllactosamine (LacNAc).

125. The method according to claim 120, wherein a first phase of exponential cell growth is provided by adding a carbon-based substrate to the culture medium before the precursor is added to the culture medium in a second phase.

126. The method according to claim 120, wherein a first phase of exponential cell growth is provided by adding a carbon-based substrate to the culture medium comprising a precursor followed by a second phase wherein:
only a carbon-based substrate is added to the culture medium, or
a carbon-based substrate and a precursor are added to the culture medium.

127. The method according to claim 120, wherein the cell produces at least one precursor for producing the di- and/or oligosaccharide.

128. The method according to claim 120, wherein the precursor for producing the di- and/or oligosaccharide is completely converted into the di- and/or oligosaccharide.

129. The method according to claim 120, wherein the di- and/or oligosaccharide is separated from the cultivation.

130. The method according to claim 120, wherein the method further comprises purification of the di- and/or oligosaccharide.

131. A method of using the cell of claim 80 for production of a di- and/or oligosaccharide, the method comprising:
cultivating the cell.

132. The method according to claim 131, wherein a mixture of di- and/or oligosaccharides is produced.

133. The method according to claim 132, wherein a mixture of charged and/or neutral di- and/or oligosaccharides is produced.

134. The method according to claim 133, wherein a mixture of di- and oligosaccharides comprising at least two different oligosaccharides is produced.

135. The method according to claim 134, wherein a mixture of oligosaccharides is produced.

136. The method according to claim 135, wherein a mixture of charged and/or neutral mammalian milk oligosaccharides (MMOs) is produced.
